SEQUENCE LISTING (5 sequences)

SEQ ID NO: 1 - DNA, 21 bp, synthetic construct
  misc_feature 1..19: note = RNA
  misc_feature 20..21: note = DNA
  source 1..21: mol_type = other DNA
gatgatgtttgaaactatttt

SEQ ID NO: 2 - DNA, 21 bp, synthetic construct
  misc_feature 1..19: note = RNA
  misc_feature 20..21: note = DNA
  source 1..21: mol_type = other DNA
aatagtttcaaacatcatctt

SEQ ID NO: 3 - RNA, 19 nt, synthetic construct
  misc_feature 1..19: note = RNA
  source 1..19: mol_type = other RNA
gatgatgtttgaaactatt

SEQ ID NO: 4 - RNA, 19 nt, synthetic construct
  source 1..19: mol_type = other RNA
aatagtttcaaacatcatc

SEQ ID NO: 5 - RNA, 21 nt, synthetic construct
  source 1..21: mol_type = other RNA
aatagtttcaaacatcatctt

(19) United States
(12) Patent Application Publication
(10) Pub. No.: US 2025/0215432 A1
(43) Pub. Date: Jul. 3, 2025

(54) BRANCHED TRIPLE LIPID-MODIFIED NUCLEIC ACID COMPOUNDS

(71) Applicant: Novartis AG, Basel (CH)

(72) Inventors: Charles ALLERSON, San Diego, CA (US); Arthur T. SUCKOW, San Diego, CA (US)

(21) Appl. No.: 18/708,780

(22) PCT Filed: Nov. 14, 2022
(86) PCT No.: PCT/US2022/079788

§ 371 Date: May 9, 2024

Related U.S. Application Data
(60) Provisional application No. 63/279,269, filed on Nov. 15, 2021.

(51) Int. Cl. C12N 15/113

(52) U.S. Cl. CPC C12N 15/1135; C12N 2310/14; C12N 2310/315; C12N 2310/321; C12N 2310/3515

(57) ABSTRACT

Disclosed herein are compounds including a nucleic acid (A), their preparation, and their use.

### CROSS-REFERENCE TO RELATED APPLICATIONS

This application claims the benefit of U.S. Provisional Application No. 63/279,269 filed Nov. 15, 2021, the contents of which is hereby incorporated herein in its entirety and for all purposes.

### REFERENCE TO AN ELECTRONIC SEQUENCE LISTING

The contents of the electronic sequence listing (052974-507001WO_Sequence-Listing_ST26.xml; Size 7,275 bytes; and Date of Creation: Nov. 10, 2022) are hereby incorporated by reference in their entirety.

### BACKGROUND

Delivering therapeutic nucleic acids into cells remains a challenging area of research. Thus, there is a need for improved nucleic acid compounds and strategies of introducing such compounds into cells.

### SUMMARY

Provided herein are, inter alia, compounds having the structure:

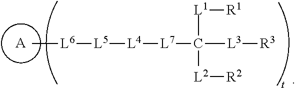

A is a nucleic acid.

L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁵ is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁷ is independently a bond, substituted or unsubstituted alkylene or substituted or unsubstituted heteroalkylene.

L¹ is independently a bond, substituted or unsubstituted 2 to 50 membered heteroalkylene or L^{1A}-L^{1B}-L^{1C}-L^{1D}-L^{1E}.

L² is independently a bond, substituted or unsubstituted 2 to 50 membered heteroalkylene or L^{2A}-L^{2B}-L^{2C}-L^{2D}-L^{2E}.

L³ is independently a bond, substituted or unsubstituted 2 to 50 membered heteroalkylene or L^{3A}-L^{3B}-L^{3C}-L^{3D}-L^{3E}.

L^{1A}, L^{1B}, L^{1C}, L^{1D}, L^{1E}, L^{2A}, L^{2B}, L^{2C}, L^{2D}, L^{2E}, L^{3A}, L^{3B}, L^{3C}, L^{3D} and L^{3E} are independently a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted or unsubstituted C₁-C₂₅ alkylene, substituted or unsubstituted 2 to 25 membered heteroalkylene, substituted or unsubstituted cycloalkylene, substituted or unsubstituted heterocycloalkylene, substituted or unsubstituted arylene or substituted or unsubstituted heteroarylene.

L^{1A}-L^{1B}-L^{1C}-L^{1D}-L^{1E} is not a bond, substituted or unsubstituted alkylene or a substituted or unsubstituted heteroalkylene with a terminal carbon atom and at least one of L^{1A}, L^{1B}, L^{1C}, L^{1D} or L^{1E} is not a bond or substituted or unsubstituted 2 to 25 membered heteroalkylene.

L^{2A}-L^{2B}-L^{2C}-L^{2D}-L^{2E} is not a bond, substituted or unsubstituted alkylene or a substituted or unsubstituted heteroalkylene with a terminal carbon atom and at least one of L^{2A}, L^{2B}, L^{2C}, L^{2D} or L^{2E} is not a bond or substituted or unsubstituted 2 to 25 membered heteroalkylene.

L^{3A}-L^{3B}-L^{3C}-L^{3D}-L^{3E} is not a bond or substituted or unsubstituted alkylene or a substituted or unsubstituted heteroalkylene with a terminal carbon atom at least one of L^{3A}, L^{3B}, L^{3C}, L^{3D} or L^{3E} is not a bond or substituted or unsubstituted 2 to 25 membered heteroalkylene.

R¹, R² and R³ are independently unsubstituted C₁-C₂₅ alkyl.

t is an integer from 1 to 5.

In an aspect, provided are methods including contacting a cell with compounds as described herein.

In an aspect, provided are methods including administering to a subject compounds as described herein.

In an aspect, provided are compounds as described herein for use in the preparation of a medicament.

In an aspect, provided are methods of introducing a nucleic acid into a cell within a subject, the method including administering to said subject compounds as described herein.

In an aspect, provided are cells including compounds as described herein.

In an aspect, provided are pharmaceutical compositions including a pharmaceutically acceptable excipient and the compounds as described herein.

Other aspects of the inventions are disclosed infra.

### DETAILED DESCRIPTION

### Overview

Provided are, inter alia, compounds including a nucleic acid (A) that is covalently bonded to one or more uptake motifs (UMs) including fatty acids (FA).

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where substituent groups are specified by their conventional chemical formulae, written from left to right, they equally encompass the chemically identical substituents that would result from writing the structure from right to left, e.g., —CH₂O— is equivalent to —OCH₂—.

“Compound” means a molecule comprising linked monomeric nucleotides. A compound may have one or more modified nucleotides. In embodiments, a compound comprises a double-stranded nucleic acid. In embodiments, a compound comprises a single-stranded nucleic acid. A compound may be provided as a pharmaceutical salt. A compound may be provided as a pharmaceutical composition.

“Oligonucleotide” means a polymer of linked monomeric nucleotides. One or more nucleotides of an oligonucleotide may be a modified nucleotide.

“Double-stranded nucleic acid” means a first nucleotide sequence hybridized to a second nucleotide sequence to form a duplex structure. Double-stranded nucleic acids include structures formed from annealing a first oligonucleotide to a second, complementary oligonucleotide, as in an siRNA. Such double-stranded nucleic acids may have a short nucleotide overhang at one or both ends of the duplex structure. Double-stranded nucleic acids also include structures formed from a single oligonucleotide with sufficient length and self-complementarity to form a duplex structure, as in an shRNA. Such double-stranded nucleic acids include stem-loop structures. A double-stranded nucleic acid may include one or more modifications relative to a naturally occurring terminus, sugar, nucleobase, and/or phosphate group.

“Double-stranded region” means the portion of a double-stranded nucleic acid where nucleotides of the first nucleotide sequence are hybridized to nucleotides of the second nucleotide sequence. A double-stranded region can be a defined portion within a double-stranded nucleic acid that is shorter than (e.g. encompassed by) the full double-stranded nucleic acid. Alternatively, a double-stranded region can be the same length as the full double-stranded nucleic acid. A double-stranded region may contain one or more mismatches between the first and second nucleotide sequences, and retain the ability hybridize with each other. Double-stranded regions do not include nucleotide overhangs.

“Antisense strand” means an oligonucleotide that is complementary to a target RNA (e.g. a mRNA) and is incorporated into the RNA-induced silencing complex (RISC) to direct gene silencing in a sequence-specific manner through the RNA interference pathway. The antisense strand may also be referred to as the “guide strand.”

“Sense strand” means an oligonucleotide that is complementary to the antisense strand of a double-stranded nucleic acid. The sense strand is typically degraded following incorporation of the antisense strand into RISC. The sense strand may also be referred to as the “passenger strand.”

“Nucleotide overhang” means an extension of one or more unpaired nucleotides from the double-stranded region of a double-stranded nucleic acid. For example, when the 3′ terminus of an antisense strand extends beyond the 5′ terminus of a sense strand, the 3′ terminus of the antisense strand has a nucleotide overhang. A nucleotide overhang can be one, two, three, four or five nucleotides. One or more nucleotides of a nucleotide overhang may be a modified nucleotide. A nucleotide overhang may be on the antisense strand, the sense strand, or both the antisense and sense strands.

“Blunt end” means a given terminus of a double-stranded nucleic acid with no unpaired nucleotides extending from the double-stranded region, i.e. there is no nucleotide overhang. A double-stranded nucleic acid may have a blunt end at one or both termini.

“siRNA” means a double-stranded nucleic acid formed from separate antisense and sense strands, which directs gene silencing in a sequence-specific manner by facilitating mRNA degradation before translation through the RNA interference pathway. The antisense and sense strands of an siRNA are not covalently linked.

“shRNA” means a double-stranded nucleic acid containing a loop structure that is processed in a cell to an siRNA which directs gene silencing in a sequence-specific manner, by facilitating mRNA degradation before translation through the RNA interference pathway.

“Single-stranded nucleic acid” means an antisense strand that is not hybridized to a complementary strand. A single-stranded nucleic acid is incorporated into RISC to direct gene silencing in a sequence-specific manner by facilitating mRNA degradation before translation through the RNA interference pathway.

“Hybridize” means the annealing of one nucleotide sequence to another nucleotide sequence based at least in part on nucleotide sequence complementarity. In embodiments, an antisense strand is hybridized to a sense strand. In embodiments, an antisense strand hybridizes to a target mRNA sequence.

“Complementary” means nucleobases having the capacity to pair non-covalently via hydrogen bonding.

“Fully complementary” means each nucleobase of a first nucleotide sequence is complementary to each nucleobase of a second nucleotide sequence. In embodiments, an antisense strand is fully complementary to its target mRNA. In embodiments, a sense strand and an antisense strand of double-stranded nucleic acid are fully complementary over their entire lengths. In embodiments, a sense strand and an antisense strand of double-stranded nucleic acid are fully complementary over the entire length of the double-stranded region of the siRNA, and one or both termini of either strand comprises single-stranded nucleotides.

“Identical” in the context of nucleotide sequences, means having the same nucleotide sequence, independent of sugar, linkage, and/or nucleobase modifications and independent of the methylation state of any pyrimidines present.

“Percent identity” means the number of nucleobases in a first nucleotide sequence that are identical to nucleobases at corresponding positions in a second nucleotide sequence, divided by the total number of nucleobases in the first nucleotide sequence.

“Mismatch” means a nucleobase of a first nucleotide sequence that is not capable of Watson-Crick pairing with a nucleobase at a corresponding position of a second nucleotide sequence.

“Nucleoside” means a monomer of a nucleobase and a pentofuranosyl sugar (e.g., either ribose or deoxyribose). Nucleosides may comprise bases such as A, C, G, T, or U, or modifications thereof. Nucleosides may be modified at the base and/or and the sugar. In embodiments, a nucleoside is a deoxyribonucleoside. In embodiments, the nucleoside is a ribonucleoside.

“Nucleotide” means a nucleoside covalently linked to a phosphate group at the 5′ carbon of the pentafuranosyl sugar. Nucleotides may be modified at one or more of the nucleobase, sugar moiety, internucleotide linkage and/or phosphate group.

“Nucleobase” means a heterocyclic base moiety capable of non-covalently pairing. Nucleobases include pyrimidines and purines.

Unless stated otherwise, numbering of nucleotide atoms is according to standard numbering convention, with the carbons of the pentafuranosyl sugar numbered 1′ through 5′, and the nucleobase atoms numbered 1 through 9 for purines and 1 through 6 for pyrimidines.

“Modified nucleoside” means a nucleoside having one or more modifications relative to a naturally occurring nucleoside. Such alterations may be present in a nucleobase and/or sugar moiety of the nucleoside. A modified nucleoside may have a modified sugar moiety and an unmodified nucleobase. A modified nucleoside may have a modified sugar moiety and a modified nucleobase.

“Modified nucleotide” means a nucleotide having one or more alterations relative to a naturally occurring nucleotide. An alteration may be present in an internucleoside linkage, a nucleobase, and/or a sugar moiety of the nucleotide. A modified nucleotide may have a modified sugar moiety and an unmodified phosphate group. A modified nucleotide may have an unmodified sugar moiety and a modified phosphate group. A modified nucleotide may have a modified sugar moiety and an unmodified nucleobase. A modified nucleotide may have a modified sugar moiety and a modified phosphate group.

“Modified nucleobase” means a nucleobase having one or more alterations relative to a naturally occurring nucleobase.

“Modified phosphate group” means any change from a naturally occurring phosphate group of a nucleotide.

“Modified internucleotide linkage” means any change from a naturally occurring phosphodiester linkage between two nucleotides.

“Phosphorothioate internucleotide linkage” means a substituted phosphodiester internucleotide linkage where one of the non-bridging atoms is a sulfur atom.

“Modified sugar moiety” means a sugar of a nucleotide having any change and/or substitution from a naturally occurring sugar moiety.

“beta-D-deoxyribonucleoside” means a naturally occurring nucleoside monomer of DNA.

“beta-D-ribonucleoside” means a naturally occurring nucleoside monomer of RNA.

“2′-O-methyl sugar” or “2′-OMe sugar” means a sugar having an O—CH₃ substitution at the 2′ position of the pentofuranosyl sugar.

“2′-O-methoxyethyl sugar” or “2′-MOE sugar” means a sugar having an OCH₂CH₂OCH₃ substitution at the 2′ position of the pentofuranosyl sugar.

“2′-fluoro sugar” or “2′-F sugar” means a sugar having a fluoro substitution at the 2′ position of the pentofuranosyl sugar.

“Bicyclic sugar” means a modified sugar moiety comprising a linkage connecting the 2′-carbon and 4′-carbon of the pentafuranosyl sugar, resulting in a bicyclic structure. Nonlimiting exemplary bicyclic sugar moieties include LNA, ENA, cEt, S-cEt, and R-cEt.

“Locked nucleic acid (LNA) sugar” means a substituted sugar moiety comprising a —CH₂—O— linkage between the 4′ and 2′ furanose ring atoms.

“ENA sugar” means a substituted sugar moiety comprising a —(CH₂)₂—O-linkage between the 4′ and 2′ furanose ring atoms.

“2′-O-methyl nucleotide” means a nucleotide having an O-methyl substitution at the 2′ position of the pentofuranosyl sugar. A 2′-O-methyl nucleotide may have a further modification in addition to the modified sugar moiety, for example a modified nucleobase and/or phosphate group.

“2′-fluoro nucleotide” means a nucleotide having a fluoro substitution at the 2′ position of the pentofuranosyl sugar. A 2′-O-fluoro nucleotide may have a further modification in addition to the modified sugar moiety, for example a modified nucleobase and/or phosphate group.

“Bicyclic nucleotide” means a nucleotide having a linkage connecting the 2′-carbon and 4′-carbon of the pentafuranosyl sugar. A bicyclic nucleotide may have a further modification in addition to the modified sugar moiety, for example a modified nucleobase and/or phosphate group

“Pharmaceutical salt” means a salt form of a compound that retains the biological effectiveness and properties of a compound and does not have undesired effects when administered to a subject.

“Therapeutically effective amount” means an amount sufficient for a compound to provide a therapeutic benefit to a subject.

“Subject” used herein means a human or non-human animal selected for treatment or therapy. In embodiments, a subject is a human.

“Administration” means providing a pharmaceutical agent or composition to a subject, and includes administration performed by a medical professional and self-administration. In embodiments, administration is intraocular administration. In embodiments, administration is topical administration.

“Treating” or “treatment” means the administration of one or more pharmaceutical agents to a subject to achieve a desired clinical result, including but not limited to the alleviation, amelioration, or slowing of the progression of at least one indicator or symptom of a disease in a subject.

“Prevent the onset of” means the prevention of the appearance of at least one indicator or symptom of a disease in a subject at risk for developing the disease.

“Effective amount” means an amount sufficient for a compound that, when administered to a subject, is sufficient to effect treatment of a disease in the subject. An effective amount may vary depending on the one or more of the compound, its mode of administration, the severity of the disease in the subject, concomitant pharmaceutical agents the subject is receiving, and characteristics of the subject such as the subject's medical history, age, and weight.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from an heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

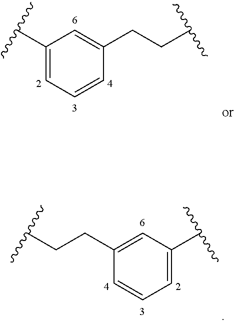 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)—NR″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R″, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula (CRR′)ₛ—X′—(C″R″R′″)ₐ—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur(S), phosphorus (P), and silicon (Si).

A “substituent group,” as used herein, means a group selected from the following moieties:
(A) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O) H, —NHC(O) OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O) H, —NHC(O) OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O) H, —NHC(O) OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O) H, —NHC(O) OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments of the compounds herein, each substituted or unsubstituted alkyl may be a substituted (e.g., substituted with a substituent group, a size-limited substituent group, or lower substituent group) or unsubstituted C₁-C₂₀ alkyl, each substituted or unsubstituted heteroalkyl is a substituted (e.g., substituted with a substituent group, a size-limited substituent group, or lower substituent group) or unsubstituted 2 to 20 membered heteroalkyl, each substituted or unsubstituted cycloalkyl is a substituted (e.g., substituted with a substituent group, a size-limited substituent group, or lower substituent group) or unsubstituted C₃-C₈ cycloalkyl, each substituted or unsubstituted heterocycloalkyl is a substituted (e.g., substituted with a substituent group, a size-limited substituent group, or lower substituent group) or unsubstituted 3 to 8 membered heterocycloalkyl, each or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 10 membered heteroaryl. In embodiments herein, each substituted or unsubstituted alkylene is a substituted (e.g., substituted with a substituent group, a size-limited substituent group, or lower substituent group) or unsubstituted C₁-C₂₀ alkylene, each substituted or unsubstituted heteroalkylene is a substituted (e.g., substituted with a substituent group, a size-limited substituent group, or lower substituent group) or unsubstituted 2 to 20 membered heteroalkylene, each substituted or unsubstituted cycloalkylene is a substituted (e.g., substituted with a substituent group, a size-limited substituent group, or lower substituent group) or unsubstituted C₃-C₈ cycloalkylene, each substituted or unsubstituted heterocycloalkylene is a substituted (e.g., substituted with a substituent group, a size-limited substituent group, or lower substituent group) or unsubstituted 3 to 8 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 10 membered heteroarylene.

In embodiments, each substituted or unsubstituted alkyl is a substituted (e.g., substituted with a substituent group, a size-limited substituent group, or lower substituent group) or unsubstituted C₁-C₈ alkyl, each substituted or unsubstituted heteroalkyl is a substituted (e.g., substituted with a substituent group, a size-limited substituent group, or lower substituent group) or unsubstituted 2 to 8 membered heteroalkyl, each substituted or unsubstituted cycloalkyl is a substituted (e.g., substituted with a substituent group, a size-limited substituent group, or lower substituent group) or unsubstituted C₃-C₇ cycloalkyl, each substituted or unsubstituted heterocycloalkyl is a substituted (e.g., substituted with a substituent group, a size-limited substituent group, or lower substituent group) or unsubstituted 3 to 7 membered heterocycloalkyl, each substituted or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 9 membered heteroaryl. In embodiments, each substituted or unsubstituted alkylene is a substituted (e.g., substituted with a substituent group, a size-limited substituent group, or lower substituent group) or unsubstituted C₁-C₈ alkylene, each substituted or unsubstituted heteroalkylene is a substituted (e.g., substituted with a substituent group, a size-limited substituent group, or lower substituent group) or unsubstituted 2 to 8 membered heteroalkylene, each substituted or unsubstituted cycloalkylene is a substituted (e.g., substituted with a substituent group, a size-limited substituent group, or lower substituent group) or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 9 membered heteroarylene. In embodiments, the compound is a chemical species set forth in the Examples section, figures, or tables below.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the present disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Description of compounds of provided herein is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

### Conjugated Compounds

Embodiments of the present disclosure relate to nucleic acid compounds covalently linked to an uptake motif comprising long chain fatty acids (LCFA). The nucleic acid compounds are targeted to an mRNA, and include double-stranded nucleic acids and single-stranded nucleic acids that act through an antisense mechanism to inhibit the expression of the mRNA.

In embodiments, a compound including an uptake motif has the structure (I)

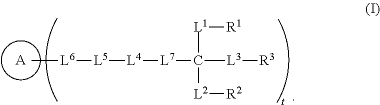

A is nucleic acid.

L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁵ is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁷ is independently a bond, substituted or unsubstituted alkylene or substituted or unsubstituted heteroalkylene.

L¹ is independently a bond, substituted or unsubstituted 2 to 50 membered heteroalkylene or L^{1A}-L^{1B}-L^{1C}-L^{1D}-L^{1E}.

L² is independently a bond, substituted or unsubstituted 2 to 50 membered heteroalkylene or L^{2A}-L^{2B}-L^{2C}-L^{2D}-L^{2E}.

L³ is independently a bond, substituted or unsubstituted 2 to 50 membered heteroalkylene or L^{3A}-L^{3B}-L^{3C}-L^{3D}-L^{3E}.

L^{1A}, L^{1B}, L^{1C}, L^{1D}, L^{1E}, L^{2A}, L^{2B}, L^{2C}, L^{2D}, L^{2E}, L^{3A}, L^{3B}, L^{3C}, L^{3D} and L^{3E} are independently a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted or unsubstituted C₁-C₂₅ alkylene, substituted or unsubstituted 2 to 25 membered heteroalkylene, substituted or unsubstituted cycloalkylene, substituted or unsubstituted heterocycloalkylene, substituted or unsubstituted arylene or substituted or unsubstituted heteroarylene.

L^{1A}-L^{1B}-L^{1C}-L^{1D}-L^{1E} is not a bond, substituted or unsubstituted alkylene or a substituted or unsubstituted heteroalkylene with a terminal carbon atom and at least one of L^{1A}, L^{1B}, L^{1C}, L^{1D} or L^{1E} is not a bond or substituted or unsubstituted 2 to 25 membered heteroalkylene.

L^{2A}-L^{2B}-L^{2C}-L^{2D}-L^{2E} is not a bond, substituted or unsubstituted alkylene or a substituted or unsubstituted heteroalkylene with a terminal carbon atom and at least one of L^{2A}, L^{2B}, L^{2C}, L^{2D} or L^{2E} is not a bond or substituted or unsubstituted 2 to 25 membered heteroalkylene.

L^{3A}-L^{3B}-L^{3C}-L^{3D}-L^{3E} is not a bond or substituted or unsubstituted alkylene or a substituted or unsubstituted heteroalkylene with a terminal carbon atom at least one of L^{3A}, L^{3B}, L^{3C}, L^{3D} or L^{3E} is not a bond or substituted or unsubstituted 2 to 25 membered heteroalkylene.

R¹, R² and R³ are independently unsubstituted C₁-C₂₅ alkyl.

t is an integer from 1 to 5.

In embodiments, a compound including an uptake motif has the structure (I)

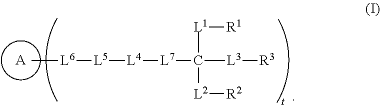

A is nucleic acid.

L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁵ is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁷ is independently a bond, substituted or unsubstituted alkylene or substituted or unsubstituted heteroalkylene.

L¹ is independently a bond, substituted or unsubstituted 2 to 50 membered heteroalkylene or L^{1A}-L^{1B}-L^{1C}-L^{1D}-L^{1E}.

L² is independently a bond, substituted or unsubstituted 2 to 50 membered heteroalkylene or L^{2A}-L^{2B}-L^{2C}-L^{2D}-L^{2E}.

L³ is independently a bond, substituted or unsubstituted 2 to 50 membered heteroalkylene or L^{3A}-L^{3B}-L^{3C}-L^{3D}-L^{3E}.

L^{1A}, L^{1B}, L^{1C}, L^{1D}, L^{1E}, L^{2A}, L^{2B}, L^{2C}, L^{2D}, L^{2E}, L^{3A}, L^{3B}, L^{3C}, L^{3D} and L^{3E} are independently a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted or unsubstituted C₁-C₂₅ alkylene, substituted or unsubstituted 2 to 25 membered heteroalkylene, substituted or unsubstituted cycloalkylene, substituted or unsubstituted heterocycloalkylene, substituted or unsubstituted arylene or substituted or unsubstituted heteroarylene.

L^{1A}-L^{1B}-L^{1C}-L^{1D}-L^{1E} is not a bond, substituted or unsubstituted alkylene or a substituted or unsubstituted heteroalkylene with a terminal carbon atom and at least one of L^{1A}, L^{1B}, L^{1C}, L^{1D} or L^{1E} is not a bond or substituted or unsubstituted 2 to 25 membered heteroalkylene.

L^{2A}-L^{2B}-L^{2C}-L^{2D}-L^{2E} is not a bond, substituted or unsubstituted alkylene or a substituted or unsubstituted heteroalkylene with a terminal carbon atom and at least one of L^{2A}, L^{2B}, L^{2C}, L^{2D} or L^{2E} is not a bond or substituted or unsubstituted 2 to 25 membered heteroalkylene.

L^{3A}-L^{3B}-L^{3C}-L^{3D}-L^{3E} is not a bond or substituted or unsubstituted alkylene or a substituted or unsubstituted heteroalkylene with a terminal carbon atom at least one of L^{3A}, L^{3B}, L^{3C}, L^{3D} or L^{3E} is not a bond or substituted or unsubstituted 2 to 25 membered heteroalkylene.

R¹, R² and R³ are independently unsubstituted C₈-C₂₀ alkyl.

t is an integer from 1 to 5.

In embodiments, L^{1A}-L^{1B}-L^{1C}-L^{1D}-L^{1E} is not a bond, substituted or unsubstituted alkylene or a substituted or unsubstituted heteroalkylene with a terminal carbon atom. In embodiments, L^{1A}-L^{1B}-L^{1C}-L^{1D}-L^{1E} is not a bond. In embodiments, L^{1A}-L^{1B}-L^{1C}-L^{1D}-L^{1E} is not a substituted or unsubstituted alkylene with a terminal carbon atom. In embodiments, L^{1A}-L^{1B}-L^{1C}-L^{1D}-L^{1E} is not a substituted alkylene with a terminal carbon atom. In embodiments, L^{1A}-L^{1B}-L^{1C}-L^{1D}-L^{1E} is not an unsubstituted alkylene with a terminal carbon atom. In embodiments, L^{1A}-L^{1B}-L^{1C}-L^{1D}-L^{1E} is not a substituted or unsubstituted heteroalkylene with a terminal carbon atom. In embodiments, L^{1A}-L^{1B}-L^{1C}-L^{1D}-L^{1E} is not a substituted heteroalkylene with a terminal carbon atom. In embodiments, L^{1A}-L^{1B}-L^{1C}-L^{1D}-L^{1E} is not an unsubstituted heteroalkylene with a terminal carbon atom.

In embodiments, at least one of L^{1A}, L^{1B}, L^{1C}, L^{1D} or L^{1E} is not a bond or substituted or unsubstituted 2 to 25 membered heteroalkylene. In embodiments, L^{1A} is not a bond. In embodiments, L^{1A} is not a substituted or unsubstituted 2 to 25 membered heteroalkylene. In embodiments, L^{1A} is not a substituted 2 to 25 membered heteroalkylene. In embodiments, L^{1A} is not an unsubstituted 2 to 25 membered heteroalkylene. In embodiments, L^{1B} is not a bond. In embodiments, L^{1B} is not a substituted or unsubstituted 2 to 25 membered heteroalkylene. In embodiments, L^{1B} is not a substituted 2 to 25 membered heteroalkylene. In embodiments, L^{1B} is not an unsubstituted 2 to 25 membered heteroalkylene. In embodiments, L^{1C} is not a bond. In embodiments, L^{1C} is not a substituted or unsubstituted 2 to 25 membered heteroalkylene. In embodiments, L^{1C} is not a substituted 2 to 25 membered heteroalkylene. In embodiments, L^{1C} is not an unsubstituted 2 to 25 membered heteroalkylene. In embodiments, L^{1D} is not a bond. In embodiments, L^{1D} is not a substituted or unsubstituted 2 to 25 membered heteroalkylene. In embodiments, L^{1D} is not a substituted 2 to 25 membered heteroalkylene. In embodiments, L^{1D} is not an unsubstituted 2 to 25 membered heteroalkylene. In embodiments, L^{1E} is not a bond. In embodiments, L^{1E} is not a substituted or unsubstituted 2 to 25 membered heteroalkylene. In embodiments, L^{1E} is not a substituted 2 to 25 membered heteroalkylene. In embodiments, L^{1E} is not an unsubstituted 2 to 25 membered heteroalkylene.

In embodiments, L^{2A}-L^{2B}-L^{2C}-L^{2D}-L^{2E} is not a bond, substituted or unsubstituted alkylene or a substituted or unsubstituted heteroalkylene with a terminal carbon atom. In embodiments, L^{2A}-L^{2B}-L^{2C}-L^{2D}-L^{2E} is not a bond. In embodiments, L^{2A}-L^{2B}-L^{2C}-L^{2D}-L^{2E} is not a substituted or unsubstituted alkylene with a terminal carbon atom. In embodiments, L^{2A}-L^{2B}-L^{2C}-L^{2D}-L^{2E} is not a substituted alkylene with a terminal carbon atom. In embodiments, L^{2A}-L^{2B}-L^{2C}-L^{2D}-L^{2E} is not an unsubstituted alkylene with a terminal carbon atom. In embodiments, L^{2A}-L^{2B}-L^{2C}-L^{2D}-L^{2E} is not a substituted or unsubstituted heteroalkylene with a terminal carbon atom. In embodiments, L^{2A}-L^{2B}-L^{2C}-L^{2D}-L^{2E} is not a substituted heteroalkylene with a terminal carbon atom. In embodiments, L^{2A}-L^{2B}-L^{2C}-L^{2D}-L^{2E} is not an unsubstituted heteroalkylene with a terminal carbon atom.

In embodiments, at least one of L^{2A}, L^{2B}, L^{2C}, L^{2D} or L^{2E} is not a bond or substituted or unsubstituted 2 to 25 membered heteroalkylene. In embodiments, L^{2A} is not a bond. In embodiments, L^{2A} is not a substituted or unsubstituted 2 to 25 membered heteroalkylene. In embodiments, L^{2A} is not a substituted 2 to 25 membered heteroalkylene. In embodiments, L^{2A} is not an unsubstituted 2 to 25 membered heteroalkylene. In embodiments, L^{2B} is not a bond. In embodiments, L^{2B} is not a substituted or unsubstituted 2 to 25 membered heteroalkylene. In embodiments, L^{2B} is not a substituted 2 to 25 membered heteroalkylene. In embodiments, L^{2B} is not an unsubstituted 2 to 25 membered heteroalkylene. In embodiments, L^{2C} is not a bond. In embodiments, L^{2C} is not a substituted or unsubstituted 2 to 25 membered heteroalkylene. In embodiments, L^{2C} is not a substituted 2 to 25 membered heteroalkylene. In embodiments, L^{2C} is not an unsubstituted 2 to 25 membered heteroalkylene. In embodiments, L^{2D} is not a bond. In embodiments, L^{2D} is not a substituted or unsubstituted 2 to 25 membered heteroalkylene. In embodiments, L^{2D} is not a substituted 2 to 25 membered heteroalkylene. In embodiments, L^{2D} is not an unsubstituted 2 to 25 membered heteroalkylene. In embodiments, L^{2E} is not a bond. In embodiments, L^{2E} is not a substituted or unsubstituted 2 to 25 membered heteroalkylene. In embodiments, L^{2E} is not a substituted 2 to 25 membered heteroalkylene. In embodiments, L^{2E} is not an unsubstituted 2 to 25 membered heteroalkylene.

In embodiments, L^{3A}-L^{3B}-L^{3C}-L^{3D}-L^{3E} is not a bond, substituted or unsubstituted alkylene or a substituted or unsubstituted heteroalkylene with a terminal carbon atom. In embodiments, L^{3A}-L^{3B}-L^{3C}-L^{3D}-L^{3E} is not a bond. In embodiments, L^{3A}-L^{3B}-L^{3C}-L^{3D}-L^{3E} is not a substituted or unsubstituted alkylene with a terminal carbon atom. In embodiments, L^{3A}-L^{3B}-L^{3C}-L^{3D}-L^{3E} is not a substituted alkylene with a terminal carbon atom. In embodiments, L^{3A}-L^{3B}-L^{3C}-L^{3D}-L^{3E} is not an unsubstituted alkylene with a terminal carbon atom. In embodiments, L^{3A}-L^{3B}-L^{3C}-L^{3D}-L^{3E} is not a substituted or unsubstituted heteroalkylene with a terminal carbon atom. In embodiments, L^{3A}-L^{3B}-L^{3C}-L^{3D}-L^{3E} is not a substituted heteroalkylene with a terminal carbon atom. In embodiments, L^{3A}-L^{3B}-L^{3C}-L^{3D}-L^{3E} is not an unsubstituted heteroalkylene with a terminal carbon atom.

In embodiments, at least one of L^{3A}, L^{3B}, L^{3C}, L^{3D} or L^{3E} is not a bond or substituted or unsubstituted 2 to 25 membered heteroalkylene. In embodiments, L^{3A} is not a bond. In embodiments, L^{3A} is not a substituted or unsubstituted 2 to 25 membered heteroalkylene. In embodiments, L^{3A} is not a substituted 2 to 25 membered heteroalkylene. In embodiments, L^{3A} is not an unsubstituted 2 to 25 membered heteroalkylene. In embodiments, L^{3B} is not a bond. In embodiments, L^{3B} is not a substituted or unsubstituted 2 to 25 membered heteroalkylene. In embodiments, L^{3B} is not a substituted 2 to 25 membered heteroalkylene. In embodiments, L^{3B} is not an unsubstituted 2 to 25 membered heteroalkylene. In embodiments, L^{3C} is not a bond. In embodiments, L^{3C} is not a substituted or unsubstituted 2 to 25 membered heteroalkylene. In embodiments, L^{3C} is not a substituted 2 to 25 membered heteroalkylene. In embodiments, L^{3C} is not an unsubstituted 2 to 25 membered heteroalkylene. In embodiments, L^{3D} is not a bond. In embodiments, L^{3D} is not a substituted or unsubstituted 2 to 25 membered heteroalkylene. In embodiments, L^{3D} is not a substituted 2 to 25 membered heteroalkylene. In embodiments, L^{3D} is not an unsubstituted 2 to 25 membered heteroalkylene. In embodiments, L^{3E} is not a bond. In embodiments, L^{3E} is not a substituted or unsubstituted 2 to 25 membered heteroalkylene. In embodiments, L^{3E} is not a substituted 2 to 25 membered heteroalkylene. In embodiments, L^{3E} is not an unsubstituted 2 to 25 membered heteroalkylene.

In embodiments, L¹, L² and L³ are independently substituted or unsubstituted 2 to 50 membered heteroalkylene. In embodiments, L¹ is substituted or unsubstituted 2 to 50 membered heteroalkylene. In embodiments, L¹ is substituted or unsubstituted 2 to 45 membered heteroalkylene. In embodiments, L¹ is substituted or unsubstituted 2 to 40 membered heteroalkylene. In embodiments, L¹ is substituted or unsubstituted 2 to 35 membered heteroalkylene. In embodiments, L¹ is substituted or unsubstituted 2 to 30 membered heteroalkylene. In embodiments, L¹ is substituted or unsubstituted 2 to 25 membered heteroalkylene. In embodiments, L¹ is substituted or unsubstituted 2 to 20 membered heteroalkylene. In embodiments, L¹ is substituted or unsubstituted 2 to 15 membered heteroalkylene. In embodiments, L¹ is substituted or unsubstituted 2 to 10 membered heteroalkylene. In embodiments, L¹ is substituted or unsubstituted 2 to 5 membered heteroalkylene. In embodiments, L² is substituted or unsubstituted 2 to 50 membered heteroalkylene. In embodiments, L² is substituted or unsubstituted 2 to 45 membered heteroalkylene. In embodiments, L² is substituted or unsubstituted 2 to 40 membered heteroalkylene. In embodiments, L² is substituted or unsubstituted 2 to 35 membered heteroalkylene. In embodiments, L² is substituted or unsubstituted 2 to 30 membered heteroalkylene. In embodiments, L² is substituted or unsubstituted 2 to 25 membered heteroalkylene. In embodiments, L² is substituted or unsubstituted 2 to 20 membered heteroalkylene. In embodiments, L² is substituted or unsubstituted 2 to 15 membered heteroalkylene. In embodiments, L² is substituted or unsubstituted 2 to 10 membered heteroalkylene. In embodiments, L² is substituted or unsubstituted 2 to 5 membered heteroalkylene. In embodiments, L³ is substituted or unsubstituted 2 to 50 membered heteroalkylene. In embodiments, L³ is substituted or unsubstituted 2 to 45 membered heteroalkylene. In embodiments, L³ is substituted or unsubstituted 2 to 40 membered heteroalkylene. In embodiments, L³ is substituted or unsubstituted 2 to 35 membered heteroalkylene. In embodiments, L³ is substituted or unsubstituted 2 to 30 membered heteroalkylene. In embodiments, L³ is substituted or unsubstituted 2 to 25 membered heteroalkylene. In embodiments, L³ is substituted or unsubstituted 2 to 20 membered heteroalkylene. In embodiments, L³ is substituted or unsubstituted 2 to 15 membered heteroalkylene. In embodiments, L³ is substituted or unsubstituted 2 to 10 membered heteroalkylene. In embodiments, L³ is substituted or unsubstituted 2 to 5 membered heteroalkylene.

In embodiments, L¹ is substituted 2 to 50 membered heteroalkylene. In embodiments, L¹ is substituted 2 to 45 membered heteroalkylene. In embodiments, L¹ is substituted 2 to 40 membered heteroalkylene. In embodiments, L¹ is substituted 2 to 35 membered heteroalkylene. In embodiments, L¹ is substituted 2 to 30 membered heteroalkylene. In embodiments, L¹ is substituted 2 to 25 membered heteroalkylene. In embodiments, L¹ is substituted 2 to 20 membered heteroalkylene. In embodiments, L¹ is substituted 2 to 15 membered heteroalkylene. In embodiments, L¹ is substituted 2 to 10 membered heteroalkylene. In embodiments, L¹ is substituted 2 to 5 membered heteroalkylene.

In embodiments, L² is substituted 2 to 50 membered heteroalkylene. In embodiments, L² is substituted 2 to 45 membered heteroalkylene. In embodiments, L² is substituted 2 to 40 membered heteroalkylene. In embodiments, L² is substituted 2 to 35 membered heteroalkylene. In embodiments, L² is substituted 2 to 30 membered heteroalkylene. In embodiments, L² is substituted 2 to 25 membered heteroalkylene. In embodiments, L² is substituted 2 to 20 membered heteroalkylene. In embodiments, L² is substituted 2 to 15 membered heteroalkylene. In embodiments, L² is substituted 2 to 10 membered heteroalkylene. In embodiments, L² is substituted 2 to 5 membered heteroalkylene.

In embodiments, L³ is substituted 2 to 50 membered heteroalkylene. In embodiments, L³ is substituted 2 to 45 membered heteroalkylene. In embodiments, L³ is substituted 2 to 40 membered heteroalkylene. In embodiments, L³ is substituted 2 to 35 membered heteroalkylene. In embodiments, L³ is substituted 2 to 30 membered heteroalkylene. In embodiments, L³ is substituted 2 to 25 membered heteroalkylene. In embodiments, L³ is substituted 2 to 20 membered heteroalkylene. In embodiments, L³ is substituted 2 to 15 membered heteroalkylene. In embodiments, L³ is substituted 2 to 10 membered heteroalkylene. In embodiments, L³ is substituted 2 to 5 membered heteroalkylene.

In embodiments, L¹ is unsubstituted 2 to 50 membered heteroalkylene. In embodiments, L¹ is unsubstituted 2 to 45 membered heteroalkylene. In embodiments, L¹ is unsubstituted 2 to 40 membered heteroalkylene. In embodiments, L¹ is unsubstituted 2 to 35 membered heteroalkylene. In embodiments, L¹ is unsubstituted 2 to 30 membered heteroalkylene. In embodiments, L¹ is unsubstituted 2 to 25 membered heteroalkylene. In embodiments, L¹ is unsubstituted 2 to 20 membered heteroalkylene. In embodiments, L¹ is unsubstituted 2 to 15 membered heteroalkylene. In embodiments, L¹ is unsubstituted 2 to 10 membered heteroalkylene. In embodiments, L¹ is unsubstituted 2 to 5 membered heteroalkylene.

In embodiments, L² is unsubstituted 2 to 50 membered heteroalkylene. In embodiments, L² is unsubstituted 2 to 45 membered heteroalkylene. In embodiments, L² is unsubstituted 2 to 40 membered heteroalkylene. In embodiments, L² is unsubstituted 2 to 35 membered heteroalkylene. In embodiments, L² is unsubstituted 2 to 30 membered heteroalkylene. In embodiments, L² is unsubstituted 2 to 25 membered heteroalkylene. In embodiments, L² is unsubstituted 2 to 20 membered heteroalkylene. In embodiments, L³ is unsubstituted 2 to 50 membered heteroalkylene. In embodiments, L² is unsubstituted 2 to 15 membered heteroalkylene. In embodiments, L² is unsubstituted 2 to 10 membered heteroalkylene. In embodiments, L² is unsubstituted 2 to 5 membered heteroalkylene. In embodiments, L³ is unsubstituted 2 to 45 membered heteroalkylene. In embodiments, L³ is unsubstituted 2 to 40 membered heteroalkylene. In embodiments, L³ is unsubstituted 2 to 35 membered heteroalkylene. In embodiments, L³ is unsubstituted 2 to 30 membered heteroalkylene. In embodiments, L³ is unsubstituted 2 to 25 membered heteroalkylene. In embodiments, L³ is unsubstituted 2 to 20 membered heteroalkylene. In embodiments, L³ is unsubstituted 2 to 15 membered heteroalkylene. In embodiments, L³ is unsubstituted 2 to 10 membered heteroalkylene. In embodiments, L³ is unsubstituted 2 to 5 membered heteroalkylene.

In embodiments, L¹, L² and L³ are independently R¹⁰-substituted or unsubstituted 2 to 50 membered heteroalkylene, and R¹⁰ is independently oxo, hydroxyl, or unsubstituted C₁-C₄ alkyl. In embodiments, R¹⁰ is independently oxo. In embodiments, R¹⁰ is independently hydroxyl. In embodiments, R¹⁰ is independently unsubstituted C₁-C₄ alkyl. In embodiments, R¹⁰ is independently methyl. In embodiments, R¹⁰ is independently ethyl. In embodiments, R¹⁰ is independently propyl. In embodiments, R¹⁰ is independently isopropyl. In embodiments, R¹⁰ is independently butyl. In embodiments, R¹⁰ is independently isobutyl. In embodiments, R¹⁰ is independently t-butyl.

In embodiments, L¹ is R¹⁰-substituted 2 to 50 membered heteroalkylene. In embodiments, L¹ is R¹⁰-substituted 2 to 45 membered heteroalkylene. In embodiments, L¹ is R¹⁰-substituted 2 to 40 membered heteroalkylene. In embodiments, L¹ is R¹⁰-substituted 2 to 35 membered heteroalkylene. In embodiments, L¹ is R¹⁰-substituted 2 to 30 membered heteroalkylene. In embodiments, L¹ is R¹⁰-substituted 2 to 25 membered heteroalkylene. In embodiments, L¹ is R¹⁰-substituted 2 to 20 membered heteroalkylene. In embodiments, L¹ is R¹⁰-substituted 2 to 15 membered heteroalkylene. In embodiments, L¹ is R¹⁰-substituted 2 to 10 membered heteroalkylene. In embodiments, L¹ is R¹⁰-substituted 2 to 5 membered heteroalkylene.

In embodiments, L² is R¹⁰-substituted 2 to 50 membered heteroalkylene. In embodiments, L² is R¹⁰-substituted 2 to 45 membered heteroalkylene. In embodiments, L² is R¹⁰-substituted 2 to 40 membered heteroalkylene. In embodiments, L² is R¹⁰-substituted 2 to 35 membered heteroalkylene. In embodiments, L² is R¹⁰-substituted 2 to 30 membered heteroalkylene. In embodiments, L² is R¹⁰-substituted 2 to 25 membered heteroalkylene. In embodiments, L² is R¹⁰-substituted 2 to 20 membered heteroalkylene. In embodiments, L² is R¹⁰-substituted 2 to 15 membered heteroalkylene. In embodiments, L² is R¹⁰-substituted 2 to 10 membered heteroalkylene. In embodiments, L² is R¹⁰-substituted 2 to 5 membered heteroalkylene.

In embodiments, L³ is R¹⁰-substituted 2 to 50 membered heteroalkylene. In embodiments, L³ is R¹⁰-substituted 2 to 45 membered heteroalkylene. In embodiments, L³ is R¹⁰-substituted 2 to 40 membered heteroalkylene. In embodiments, L³ is R¹⁰-substituted 2 to 35 membered heteroalkylene. In embodiments, L³ is R¹⁰-substituted 2 to 30 membered heteroalkylene. In embodiments, L³ is R¹⁰-substituted 2 to 25 membered heteroalkylene. In embodiments, L³ is R¹⁰-substituted 2 to 20 membered heteroalkylene. In embodiments, L³ is R¹⁰-substituted 2 to 15 membered heteroalkylene. In embodiments, L³ is R¹⁰-substituted 2 to 10 membered heteroalkylene. In embodiments, L³ is R¹⁰-substituted 2 to 5 membered heteroalkylene.

In embodiments, t is 1. In embodiments, t is 2. In embodiments, t is 3. In embodiments, t is 4. In embodiments, t is 5.

In embodiments, the nucleic acid (A) is an oligonucleotide. In embodiments, the nucleic acid (A) is a double-stranded nucleic acid, or a single-stranded nucleic acid.

In embodiments, one L⁶ is attached to a 3′ carbon of the oligonucleotide. In embodiments, the 3′ carbon is the 3′ carbon of a 3′ terminal nucleotide.

In embodiments, one L⁶ is attached to a 5′ carbon of the double-stranded nucleic acid or single-stranded nucleic acid. In embodiments, the 5′ carbon is the 5′ carbon of a 5′ terminal nucleotide.

In embodiments, one L⁶ is attached to a 2′ carbon of the double-stranded nucleic acid or single-stranded nucleic acid. In embodiments, one L⁶ is attached to a 2′ carbon of 3′ terminal nucleic acid.

In embodiments, one L⁶ is attached to an oxygen of 2′ hydroxy of the double-stranded nucleic acid or single-stranded nucleic acid. In embodiments, one L⁶ is attached to an oxygen of 2′ hydroxy of 3′ terminal nucleic acid.

In embodiments, one L⁶ is attached to a nucleobase of the double-stranded nucleic acid or single-stranded nucleic acid. In embodiments, one L⁶ is attached to a carbon atom of a nucleobase of the double-stranded nucleic acid or single-stranded nucleic acid. In embodiments, one L⁶ is attached to a nitrogen atom of a nucleobase of the double-stranded nucleic acid or single-stranded nucleic acid. In embodiments, one L⁶ is attached to an oxygen atom of a nucleobase of the double-stranded nucleic acid or single-stranded nucleic acid.

In embodiments, one L⁶ is attached to a 3′ nitrogen of the oligonucleotide (e.g., the 3′ nitrogen of a morpholino moiety) at its 3′ end. In embodiments, at least one L⁶ is attached to a 6′ carbon of the oligonucleotide (e.g., the 6′ carbon of a morpholino moiety) at its 5′ end. In embodiments, the double-stranded nucleic acid or single-stranded nucleic acid comprises a morpholino moiety. In embodiments, when the double-stranded nucleic acid or single-stranded nucleic acid comprises a morpholino moiety, one L⁶ is attached to a 3′ nitrogen of morpholino moiety. In embodiments, when the double-stranded nucleic acid or single-stranded nucleic acid comprises a morpholino moiety, one L⁶ is attached to a 6′ carbon of the morpholino moiety.

In embodiments, the nucleic acid (A) is a double-stranded oligonucleotide. In embodiments, one L⁶ is attached to a 3′ carbon of the double-stranded oligonucleotide. In embodiments, one L⁶ is attached to a 3′ carbon of the double-stranded oligonucleotide at either of its 3′ ends. In embodiments, one L⁶ is attached to a 3′ carbon of the double-stranded oligonucleotide at the 3′end of its antisense strand. In embodiments, one L⁶ is attached to a 3′ carbon of the double-stranded oligonucleotide at the 3′end of its sense strand.

In embodiments, one L⁶ is attached to a 3′ nitrogen of the double-stranded oligonucleotide (e.g., the 3′ nitrogen of a morpholino moiety) at either of its 3′ ends. In embodiments, one L⁶ is attached to a 3′ nitrogen of the double-stranded oligonucleotide (e.g., the 3′ nitrogen of a morpholino moiety) at the 3′end of its antisense strand. In embodiments, one L⁶ is attached to a 3′ nitrogen of the double-stranded oligonucleotide (e.g., the 3′ nitrogen of a morpholino moiety) at the 3′end of its sense strand.

In embodiments, one L⁶ is attached to a 5′ carbon of the double-stranded oligonucleotide. In embodiments, one L⁶ is attached to a 5′ carbon of the double-stranded oligonucleotide at either of its 5′ ends. In embodiments, one L⁶ is attached to a 5′ carbon of the double-stranded oligonucleotide at the 5′ end of its antisense strand. In embodiments, one L⁶ is attached to a 5′ carbon of the double-stranded oligonucleotide at the 5′ end of its sense strand.

In embodiments, one L⁶ is attached to a 6′ carbon of the double-stranded oligonucleotide (e.g., the 6′ carbon of a morpholino moiety) at either of its 5′ ends. In embodiments, one L⁶ is attached to a 6′ carbon of the double-stranded oligonucleotide (e.g., the 6′ carbon of a morpholino moiety) at the 5′ end of its antisense strand. In embodiments, one L⁶ is attached to a 6′ carbon of the double-stranded oligonucleotide (e.g., the 6′ carbon of a morpholino moiety) at the 5′ end of its sense strand.

In embodiments, one L⁶ is attached to a 2′ carbon of the double-stranded oligonucleotide. In embodiments, one L⁶ is attached to a 2′ carbon of the double-stranded oligonucleotide at either of its 3′ ends. In embodiments, one L⁶ is attached to a 2′ carbon at the 3′ end of the sense strand. In embodiments, one L⁶ is attached to a 2′ carbon at the 3′ end of the antisense strand.

In embodiments, one L⁶ is attached to an oxygen of 2′ hydroxy of the double-stranded nucleic acid or single-stranded nucleic acid. In embodiments, one L⁶ is attached to an oxygen of 2′ hydroxy of 3′ terminal nucleic acid.

In embodiments, one L⁶ is attached to an oxygen of 2′ hydroxy of the double-stranded oligonucleotide. In embodiments, one L⁶ is attached to an oxygen of 2′ hydroxy of the double-stranded oligonucleotide at either of its 3′ ends. In embodiments, one L⁶ is attached to an oxygen of 2′ hydroxy at the 3′ end of the sense strand. In embodiments, one L⁶ is attached to an oxygen of 2′ hydroxy at the 3′ end of the antisense strand.

In embodiments, one L⁶ is attached to a nucleobase of the double-stranded oligonucleotide. In embodiments, one L⁶ is attached to a nucleobase of the sense strand of the double-stranded oligonucleotide. In embodiments, one L⁶ is attached to a nucleobase of the antisense strand of the double-stranded oligonucleotide. In embodiments, one L⁶ is attached to a nucleobase of the double-stranded oligonucleotide at either of its 3′ ends. In embodiments, one L⁶ is attached to a nucleobase of the double-stranded oligonucleotide at the 3′end of its antisense strand. In embodiments, one L⁶ is attached to a nucleobase of the double-stranded oligonucleotide at the 3′end of its sense strand. In embodiments, one L⁶ is attached to a nucleobase of the double-stranded oligonucleotide at either of its 5′ ends. In embodiments, one L⁶ is attached to a nucleobase of the double-stranded oligonucleotide at the 5′ end of its antisense strand. In embodiments, one L⁶ is attached to a nucleobase of the double-stranded oligonucleotide at the 5′ end of its sense strand.

In embodiments, the nucleic acid (A) is a single-stranded oligonucleotide. In embodiments, one L⁶ is attached to a 3′ carbon of the single-stranded oligonucleotide at the 3′ end.

In embodiments, one L⁶ is attached to a 3′ nitrogen of the single-stranded oligonucleotide (e.g., the 3′ nitrogen of a morpholino moiety) at the 3′ end of the single-stranded oligonucleotide.

In embodiments, one L⁶ is attached to a 5′ carbon of the single-stranded oligonucleotide at the 5′ end.

In embodiments, one L⁶ is attached to a 6′ carbon of the single-stranded oligonucleotide (e.g., the 6′ carbon of a morpholino moiety) at the 5′ end.

In embodiments, one L⁶ is attached to a 2′ carbon of the single-stranded oligonucleotide. In embodiments, one L⁶ is attached to a 2′ carbon of the single-stranded oligonucleotide at its 5′ end. In embodiments, one L⁶ is attached to a 2′ carbon of the single-stranded oligonucleotide at its 3′ end.

In embodiments, one L⁶ is attached to an oxygen of 2′ hydroxy of the double-stranded nucleic acid or single-stranded nucleic acid. In embodiments, one L⁶ is attached to an oxygen of 2′ hydroxy of the double-stranded nucleic acid at its 3′ end. In embodiments, one L⁶ is attached to an oxygen of 2′ hydroxy of the single-stranded nucleic acid at its 3′ end.

In embodiments, one L⁶ is attached to a nucleobase of the single-stranded oligonucleotide. In embodiments, one L⁶ is attached to a nucleobase of the single-stranded oligonucleotide at the of 3′ end. In embodiments, one L⁶ is attached to a nucleobase of the single-stranded oligonucleotide at the 5′ end.

In embodiments, L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene (e.g., C₁-C₂₀, C₁-C₁₂, C₁-C₈, C₁-C₆, C₁-C₄, or C₁-C₂) or substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted C₁-C₈ alkylene or substituted or unsubstituted 2 to 8 membered heteroalkylene.

In embodiments, L⁴ is independently a bond. In embodiments, L⁴ is independently —NH—. In embodiments, L⁴ is independently —O—. In embodiments, L⁴ is independently —S—. In embodiments, L⁴ is independently —C(O)—. In embodiments, L⁴ is independently —NHC(O)—. In embodiments, L⁴ is independently —NHC(O)NH—. In embodiments, L⁴ is independently —C(O)O—. In embodiments, L⁴ is independently —OC(O)—. In embodiments, L⁴ is independently —C(O)NH—. In embodiments, L⁴ is independently —OPO₂—O—. In embodiments, L⁴ is independently —OP(S)(O)—O—. In embodiments, L⁴ is independently —OP(S)₂—O—. In embodiments, L⁴ is independently —S(O)₂NH—.

In embodiments, L⁴ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently unsubstituted alkylene (e.g., C₁-C₂₀, C₁-C₁₂, C₁-C₈, C₁-C₆, C₁-C₄, or C₁-C₂). In embodiments, L⁴ is independently substituted or unsubstituted C₁-C₂₀ alkylene. In embodiments, L⁴ is independently substituted C₁-C₂₀ alkylene. In embodiments, L⁴ is independently unsubstituted C₁-C₂₀ alkylene. In embodiments, L⁴ is independently substituted or unsubstituted C₁-C₁₂ alkylene. In embodiments, L⁴ is independently substituted C₁-C₁₂ alkylene. In embodiments, L⁴ is independently unsubstituted C₁-C₁₂ alkylene. In embodiments, L⁴ is independently substituted or unsubstituted C₁-C₈ alkylene. In embodiments, L⁴ is independently substituted C₁-C₈ alkylene. In embodiments, L⁴ is independently unsubstituted C₁-C₈ alkylene. In embodiments, L⁴ is independently substituted or unsubstituted C₁-C₆ alkylene. In embodiments, L⁴ is independently substituted C₁-C₆ alkylene. In embodiments, L⁴ is independently unsubstituted C₁-C₆ alkylene. In embodiments, L⁴ is independently substituted or unsubstituted C₁-C₄ alkylene. In embodiments, L⁴ is independently substituted C₁-C₄ alkylene. In embodiments, L⁴ is independently unsubstituted C₁-C₄ alkylene. In embodiments, L⁴ is independently substituted or unsubstituted ethylene. In embodiments, L⁴ is independently substituted ethylene. In embodiments, L⁴ is independently unsubstituted ethylene. In embodiments, L⁴ is independently substituted or unsubstituted methylene. In embodiments, L⁴ is independently substituted methylene. In embodiments, L⁴ is independently unsubstituted methylene.

In embodiments, L⁴ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ is independently substituted or unsubstituted 2 to 20 membered heteroalkylene. In embodiments, L⁴ is independently substituted 2 to 20 membered heteroalkylene. In embodiments, L⁴ is independently unsubstituted 2 to 20 membered heteroalkylene. In embodiments, L⁴ is independently substituted or unsubstituted 2 to 12 membered heteroalkylene. In embodiments, L⁴ is independently substituted 2 to 12 membered heteroalkylene. In embodiments, L⁴ is independently unsubstituted 2 to 12 membered heteroalkylene. In embodiments, L⁴ is independently substituted or unsubstituted 2 to 8 membered heteroalkylene. In embodiments, L⁴ is independently substituted 2 to 8 membered heteroalkylene. In embodiments, L⁴ is independently unsubstituted 2 to 8 membered heteroalkylene. In embodiments, L⁴ is independently substituted or unsubstituted 2 to 6 membered heteroalkylene. In embodiments, L⁴ is independently substituted 2 to 6 membered heteroalkylene. In embodiments, L⁴ is independently unsubstituted 2 to 6 membered heteroalkylene. In embodiments, L⁴ is independently substituted or unsubstituted 4 to 6 membered heteroalkylene. In embodiments, L⁴ is independently substituted 4 to 6 membered heteroalkylene. In embodiments, L⁴ is independently unsubstituted 4 to 6 membered heteroalkylene. In embodiments, L⁴ is independently substituted or unsubstituted 2 to 3 membered heteroalkylene. In embodiments, L⁴ is independently substituted 2 to 3 membered heteroalkylene. In embodiments, L⁴ is independently unsubstituted 2 to 3 membered heteroalkylene. In embodiments, L⁴ is independently substituted or unsubstituted 4 to 5 membered heteroalkylene. In embodiments, L⁴ is independently substituted 4 to 5 membered heteroalkylene. In embodiments, L⁴ is independently unsubstituted 4 to 5 membered heteroalkylene.

In embodiments, L⁶ is independently a bond. In embodiments, L⁶ is independently —NH—. In embodiments, L⁶ is independently —O—. In embodiments, L⁶ is independently —S—. In embodiments, L⁶ is independently —C(O)—. In embodiments, L⁶ is independently —NHC(O)—. In embodiments, L⁶ is independently —NHC(O)NH—. In embodiments, L⁶ is independently —C(O)O—. In embodiments, L⁶ is independently —OC(O)—. In embodiments, L⁶ is independently —C(O)NH—. In embodiments, L⁶ is independently —OPO₂—O—. In embodiments, L⁶ is independently —OP(S)(O)—O—. In embodiments, L⁶ is independently —OP(S)₂—O—. In embodiments, L⁶ is independently —S(O)₂NH—.

In embodiments, L⁶ is independently substituted or unsubstituted alkylene (e.g., C₁-C₂₀, C₁-C₁₂, C₁-C₈, C₁-C₆, C₁-C₄, or C₁-C₂). In embodiments, L⁶ is independently substituted alkylene (e.g., C₁-C₂₀, C₁-C₁₂, C₁-C₈, C₁-C₆, C₁-C₄, or C₁-C₂). In embodiments, L⁶ is independently unsubstituted alkylene (e.g., C₁-C₂₀, C₁-C₁₂, C₁-C₈, C₁-C₆, C₁-C₄, or C₁-C₂). In embodiments, L⁶ is independently substituted or unsubstituted C₁-C₂₀ alkylene. In embodiments, L⁶ is independently substituted C₁-C₂₀ alkylene. In embodiments, L⁶ is independently unsubstituted C₁-C₂₀ alkylene. In embodiments, L⁶ is independently substituted or unsubstituted C₁-C₁₂ alkylene. In embodiments, L⁶ is independently substituted C₁-C₁₂ alkylene. In embodiments, L⁶ is independently unsubstituted C₁-C₁₂ alkylene. In embodiments, L⁶ is independently substituted or unsubstituted C₁-C₈ alkylene. In embodiments, L⁶ is independently substituted C₁-C₈ alkylene. In embodiments, L⁶ is independently unsubstituted C₁-C₈ alkylene. In embodiments, L⁶ is independently substituted or unsubstituted C₁-C₆ alkylene. In embodiments, L⁶ is independently substituted C₁-C₆ alkylene. In embodiments, L⁶ is independently unsubstituted C₁-C₆ alkylene. In embodiments, L⁶ is independently substituted or unsubstituted C₁-C₄ alkylene. In embodiments, L⁶ is independently substituted C₁-C₄ alkylene. In embodiments, L⁶ is independently unsubstituted C₁-C₄ alkylene. In embodiments, L⁶ is independently substituted or unsubstituted ethylene. In embodiments, L⁶ is independently substituted ethylene. In embodiments, L⁶ is independently unsubstituted ethylene. In embodiments, L⁶ is independently substituted or unsubstituted methylene. In embodiments, L⁶ is independently substituted methylene. In embodiments, L⁶ is independently unsubstituted methylene.

In embodiments, L⁶ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted or unsubstituted 2 to 20 membered heteroalkylene. In embodiments, L⁶ is independently substituted 2 to 20 membered heteroalkylene. In embodiments, L⁶ is independently unsubstituted 2 to 20 membered heteroalkylene. In embodiments, L⁶ is independently substituted or unsubstituted 2 to 12 membered heteroalkylene. In embodiments, L⁶ is independently substituted 2 to 12 membered heteroalkylene. In embodiments, L⁶ is independently unsubstituted 2 to 12 membered heteroalkylene. In embodiments, L⁶ is independently substituted or unsubstituted 2 to 8 membered heteroalkylene. In embodiments, L⁶ is independently substituted 2 to 8 membered heteroalkylene. In embodiments, L⁶ is independently unsubstituted 2 to 8 membered heteroalkylene. In embodiments, L⁶ is independently substituted or unsubstituted 2 to 6 membered heteroalkylene. In embodiments, L⁶ is independently substituted 2 to 6 membered heteroalkylene. In embodiments, L⁶ is independently unsubstituted 2 to 6 membered heteroalkylene. In embodiments, L⁶ is independently substituted or unsubstituted 4 to 6 membered heteroalkylene. In embodiments, L⁶ is independently substituted 4 to 6 membered heteroalkylene. In embodiments, L⁶ is independently unsubstituted 4 to 6 membered heteroalkylene. In embodiments, L⁶ is independently substituted or unsubstituted 2 to 3 membered heteroalkylene. In embodiments, L⁶ is independently substituted 2 to 3 membered heteroalkylene. In embodiments, L⁶ is independently unsubstituted 2 to 3 membered heteroalkylene. In embodiments, L⁶ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene.

In embodiments, L⁴ and L⁶ are independently —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, R²⁰-substituted or unsubstituted C₁-C₈ alkylene, or R²⁰-substituted or unsubstituted 2 to 8 membered heteroalkylene, and R²⁰ is independently oxo, hydroxyl, or substituted or unsubstituted C₁-C₄ alkyl. In embodiments, L⁴ and L⁶ are independently R²⁰-substituted or unsubstituted C₁-C₈ alkylene. In embodiments, L⁴ and L⁶ are independently R²⁰-substituted C₁-C₈ alkylene. In embodiments, L⁴ and L⁶ are independently unsubstituted C₁-C₈alkylene. In embodiments, L⁴ and L⁶ are independently R²⁰-substituted or unsubstituted 2 to 8 membered heteroalkylene. In embodiments, L⁴ and L⁶ are independently R²⁰-substituted 2 to 8 membered heteroalkylene. In embodiments, L⁴ and L⁶ are independently unsubstituted 2 to 8 membered heteroalkylene.

In embodiments, R²⁰ is independently oxo, hydroxyl, or unsubstituted C₁-C₄ alkyl. In embodiments, R²⁰ is independently oxo. In embodiments, R²⁰ is independently hydroxyl. In embodiments, R²⁰ is independently unsubstituted C₁-C₄ alkyl. In embodiments, R²⁰ is independently methyl. In embodiments, R²⁰ is independently ethyl. In embodiments, R²⁰ is independently propyl. In embodiments, R²⁰ is independently isopropyl. In embodiments, R²⁰ is independently butyl. In embodiments, R²⁰ is independently isobutyl. In embodiments, R²⁰ is independently t-butyl.

In embodiments, L⁴ is independently R²⁰-substituted alkylene (e.g., C₁-C₂₀, C₁-C₁₂, C₁-C₈, C₁-C₆, C₁-C₄, or C₁-C₂). In embodiments, L⁴ is independently R²⁰-substituted C₁-C₂₀ alkylene. In embodiments, L⁴ is independently R²⁰-substituted C₁-C₁₂ alkylene. In embodiments, L⁴ is independently R²⁰-substituted C₁-C₈ alkylene. In embodiments, L⁴ is independently R²⁰-substituted C₁-C₆ alkylene. In embodiments, L⁴ is independently R²⁰-substituted C₁-C₄ alkylene. In embodiments, L⁴ is independently R²⁰-substituted ethylene. In embodiments, L⁴ is independently R²⁰-substituted methylene.

In embodiments, L⁴ is independently R²⁰-substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ is independently R²⁰-substituted 2 to 20 membered heteroalkylene. In embodiments, L⁴ is independently R²⁰-substituted 2 to 8 membered heteroalkylene. In embodiments, L⁴ is independently R²⁰-substituted 2 to 6 membered heteroalkylene. In embodiments, L⁴ is independently R²⁰-substituted 4 to 6 membered heteroalkylene. In embodiments, L⁴ is independently R²⁰-substituted 2 to 3 membered heteroalkylene. In embodiments, L⁴ is independently R²⁰-substituted 4 to 5 membered heteroalkylene.

In embodiments, L⁶ is independently R²⁰-substituted alkylene (e.g., C₁-C₂₀, C₁-C₁₂, C₁-C₈, C₁-C₆, C₁-C₄, or C₁-C₂). In embodiments, L⁶ is independently R²⁰-substituted C₁-C₂₀ alkylene. In embodiments, L⁶ is independently R²⁰-substituted C₁-C₁₂ alkylene. In embodiments, L⁶ is independently R²⁰-substituted C₁-C₈ alkylene. In embodiments, L⁶ is independently R²⁰-substituted C₁-C₆ alkylene. In embodiments, L⁶ is independently R²⁰-substituted C₁-C₄ alkylene. In embodiments, L⁶ is independently R²⁰-substituted ethylene. In embodiments, L⁶ is independently R²⁰-substituted methylene.

In embodiments, L⁶ is independently R²⁰-substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently R²⁰-substituted 2 to 20 membered heteroalkylene. In embodiments, L⁶ is independently R²⁰-substituted 2 to 8 membered heteroalkylene. In embodiments, L⁶ is independently R²⁰-substituted 2 to 6 membered heteroalkylene. In embodiments, L⁶ is independently R²⁰-substituted 4 to 6 membered heteroalkylene. In embodiments, L⁶ is independently R²⁰-substituted 2 to 3 membered heteroalkylene. In embodiments, L⁶ is independently R²⁰-substituted 4 to 5 membered heteroalkylene.

In embodiments, L⁶ is independently

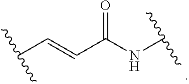 [SMILES: */C=C/C(=O)N*]

In embodiments, L⁶ is independently

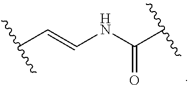 [SMILES: */C=C/NC(*)=O]

In embodiments, L⁴ is independently -L¹⁴-NH—C(O)— or -L¹⁴-C(O)—NH—, and L¹⁴ is independently substituted or unsubstituted C₁-C₂₀ alkylene, substituted or unsubstituted 2-20 membered heteroalkylene, or substituted or unsubstituted 2-20 membered heteroalkenylene.

In embodiments, L¹⁴ is independently substituted or unsubstituted alkylene (e.g., C₁-C₂₀, C₁-C₁₂, C₁-C₈, C₁-C₆, C₁-C₄, or C₁-C₂). In embodiments, L¹⁴ is independently substituted alkylene (e.g., C₁-C₂₀, C₁-C₁₂, C₁-C₈, C₁-C₆, C₁-C₄, or C₁-C₂). In embodiments, L¹⁴ is independently unsubstituted alkylene (e.g., C₁-C₂₀, C₁-C₁₂, C₁-C₈, C₁-C₆, C₁-C₄, or C₁-C₂). In embodiments, L¹⁴ is independently substituted or unsubstituted C₁-C₂₀ alkylene. In embodiments, L¹⁴ is independently substituted C₁-C₂₀ alkylene. In embodiments, L¹⁴ is independently unsubstituted C₁-C₂₀ alkylene. In embodiments, L¹⁴ is independently substituted or unsubstituted C₁-C₁₂ alkylene. In embodiments, L¹⁴ is independently substituted C₁-C₁₂ alkylene. In embodiments, L¹⁴ is independently unsubstituted C₁-C₁₂ alkylene. In embodiments, L¹⁴ is independently substituted or unsubstituted C₁-C₈ alkylene. In embodiments, L¹⁴ is independently substituted C₁-C₈alkylene. In embodiments, L¹⁴ is independently unsubstituted C₁-C₈ alkylene. In embodiments, L¹⁴ is independently substituted or unsubstituted C₁-C₆ alkylene. In embodiments, L¹⁴ is independently substituted C₁-C₆ alkylene. In embodiments, L¹⁴ is independently unsubstituted C₁-C₆ alkylene. In embodiments, L¹⁴ is independently substituted or unsubstituted C₁-C₄ alkylene. In embodiments, L¹⁴ is independently substituted C₁-C₄ alkylene. In embodiments, L¹⁴ is independently unsubstituted C₁-C₄ alkylene. In embodiments, L¹⁴ is independently substituted or unsubstituted ethylene. In embodiments, L¹⁴ is independently substituted ethylene. In embodiments, L¹⁴ is independently unsubstituted ethylene. In embodiments, L¹⁴ is independently substituted or unsubstituted methylene. In embodiments, L¹⁴ is independently substituted methylene. In embodiments, L¹⁴ is independently unsubstituted methylene.

In embodiments, L¹⁴ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁴ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁴ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁴ is independently substituted or unsubstituted 2 to 20 membered heteroalkylene. In embodiments, L¹⁴ is independently substituted 2 to 20 membered heteroalkylene. In embodiments, L¹⁴ is independently unsubstituted 2 to 20 membered heteroalkylene. In embodiments, L¹⁴ is independently substituted or unsubstituted 2 to 12 membered heteroalkylene. In embodiments, L¹⁴ is independently substituted 2 to 12 membered heteroalkylene. In embodiments, L¹⁴ is independently unsubstituted 2 to 12 membered heteroalkylene. In embodiments, L¹⁴ is independently substituted or unsubstituted 2 to 8 membered heteroalkylene. In embodiments, L¹⁴ is independently substituted 2 to 8 membered heteroalkylene. In embodiments, L¹⁴ is independently unsubstituted 2 to 8 membered heteroalkylene. In embodiments, L¹⁴ is independently substituted or unsubstituted 2 to 6 membered heteroalkylene. In embodiments, L¹⁴ is independently substituted 2 to 6 membered heteroalkylene. In embodiments, L¹⁴ is independently unsubstituted 2 to 6 membered heteroalkylene. In embodiments, L¹⁴ is independently substituted or unsubstituted 4 to 6 membered heteroalkylene. In embodiments, L¹⁴ is independently substituted 4 to 6 membered heteroalkylene. In embodiments, L¹⁴ is independently unsubstituted 4 to 6 membered heteroalkylene. In embodiments, L¹⁴ is independently substituted or unsubstituted 2 to 3 membered heteroalkylene. In embodiments, L¹⁴ is independently substituted 2 to 3 membered heteroalkylene. In embodiments, L¹⁴ is independently unsubstituted 2 to 3 membered heteroalkylene. In embodiments, L¹⁴ is independently substituted or unsubstituted 4 to 5 membered heteroalkylene. In embodiments, L¹⁴ is independently substituted 4 to 5 membered heteroalkylene. In embodiments, L¹⁴ is independently unsubstituted 4 to 5 membered heteroalkylene.

In embodiments, L¹⁴ is independently substituted or unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁴ is independently substituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁴ is independently unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁴ is independently substituted or unsubstituted 2 to 20 membered heteroalkenylene. In embodiments, L¹⁴ is independently substituted 2 to 20 membered heteroalkenylene. In embodiments, L¹⁴ is independently unsubstituted 2 to 20 membered heteroalkenylene. In embodiments, L¹⁴ is independently substituted or unsubstituted 2 to 12 membered heteroalkenylene. In embodiments, L¹⁴ is independently substituted 2 to 12 membered heteroalkenylene. In embodiments, L¹⁴ is independently unsubstituted 2 to 12 membered heteroalkenylene. In embodiments, L¹⁴ is independently substituted or unsubstituted 2 to 8 membered heteroalkenylene. In embodiments, L¹⁴ is independently substituted 2 to 8 membered heteroalkenylene. In embodiments, L¹⁴ is independently unsubstituted 2 to 8 membered heteroalkenylene. In embodiments, L¹⁴ is independently substituted or unsubstituted 2 to 6 membered heteroalkenylene. In embodiments, L¹⁴ is independently substituted 2 to 6 membered heteroalkenylene. In embodiments, L¹⁴ is independently unsubstituted 2 to 6 membered heteroalkenylene. In embodiments, L¹⁴ is independently substituted or unsubstituted 4 to 6 membered heteroalkenylene. In embodiments, L¹⁴ is independently substituted 4 to 6 membered heteroalkenylene. In embodiments, L¹⁴ is independently unsubstituted 4 to 6 membered heteroalkenylene. In embodiments, L¹⁴ is independently substituted or unsubstituted 2 to 3 membered heteroalkenylene. In embodiments, L¹⁴ is independently substituted 2 to 3 membered heteroalkenylene. In embodiments, L¹⁴ is independently unsubstituted 2 to 3 membered heteroalkenylene. In embodiments, L¹⁴ is independently substituted or unsubstituted 4 to 5 membered heteroalkenylene. In embodiments, L¹⁴ is independently substituted 4 to 5 membered heteroalkenylene. In embodiments, L¹⁴ is independently unsubstituted 4 to 5 membered heteroalkenylene.

In embodiments, L⁴ is independently -L¹⁴-NH—C(O)— or -L¹⁴-C(O)—NH—. In embodiments, L¹⁴ is substituted or unsubstituted C₁-C₈ alkylene. In embodiments, L¹⁴ is substituted C₁-C₈ alkylene. In embodiments, L¹⁴ unsubstituted C₁-C₈ alkylene.

In embodiments, L⁴ is independently

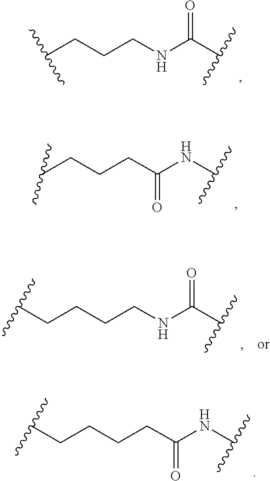 [SMILES: *CCCNC(*)=O ; *CCCC(=O)N* ; *CCCCNC(*)=O ; *CCCCC(=O)N*]

In embodiments, L⁴ is independently

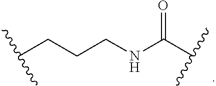 [SMILES: *CCCNC(*)=O]

In embodiments, L⁴ is independently

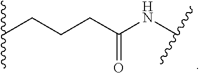 [SMILES: *CCCC(=O)N*]

In embodiments, L⁴ is independently

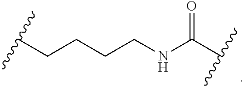 [SMILES: *CCCCNC(*)=O]

In embodiments, L⁴ is independently

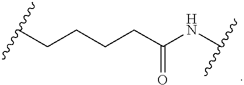 [SMILES: *CCCCC(=O)N*]

In embodiments, L⁵ is independently substituted or unsubstituted alkylene (e.g., C₁-C₂₀, C₁-C₁₂, C₁-C₈, C₁-C₆, C₁-C₄, or C₁-C₂). In embodiments, L⁵ is independently substituted alkylene (e.g., C₁-C₂₀, C₁-C₁₂, C₁-C₈, C₁-C₆, C₁-C₄, or C₁-C₂). In embodiments, L⁵ is independently unsubstituted alkylene (e.g., C₁-C₂₀, C₁-C₁₂, C₁-C₈, C₁-C₆, C₁-C₄, or C₁-C₂). In embodiments, L⁵ is independently substituted or unsubstituted C₁-C₂₀ alkylene. In embodiments, L⁵ is independently substituted C₁-C₂₀ alkylene. In embodiments, L⁵ is independently unsubstituted C₁-C₂₀ alkylene. In embodiments, L⁵ is independently substituted or unsubstituted C₁-C₁₂ alkylene. In embodiments, L⁵ is independently substituted C₁-C₁₂ alkylene. In embodiments, L⁵ is independently unsubstituted C₁-C₁₂ alkylene. In embodiments, L⁵ is independently substituted or unsubstituted C₁-C₈ alkylene. In embodiments, L⁵ is independently substituted C₁-C₈ alkylene. In embodiments, L⁵ is independently unsubstituted C₁-C₈ alkylene. In embodiments, L⁵ is independently substituted or unsubstituted C₁-C₆ alkylene. In embodiments, L⁵ is independently substituted C₁-C₆ alkylene. In embodiments, L⁵ is independently unsubstituted C₁-C₆ alkylene. In embodiments, L⁵ is independently substituted or unsubstituted C₁-C₄ alkylene. In embodiments, L⁵ is independently substituted C₁-C₄ alkylene. In embodiments, L⁵ is independently unsubstituted C₁-C₄ alkylene. In embodiments, L⁵ is independently substituted or unsubstituted ethylene. In embodiments, L⁵ is independently substituted ethylene. In embodiments, L⁵ is independently unsubstituted ethylene. In embodiments, L⁵ is independently substituted or unsubstituted methylene. In embodiments, L⁵ is independently substituted methylene. In embodiments, L⁵ is independently unsubstituted methylene.

In embodiments, L⁵ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted or unsubstituted 2 to 20 membered heteroalkylene. In embodiments, L⁵ is independently substituted 2 to 20 membered heteroalkylene. In embodiments, L⁵ is independently unsubstituted 2 to 20 membered heteroalkylene. In embodiments, L⁵ is independently substituted or unsubstituted 2 to 12 membered heteroalkylene. In embodiments, L⁵ is independently substituted 2 to 12 membered heteroalkylene. In embodiments, L⁵ is independently unsubstituted 2 to 12 membered heteroalkylene. In embodiments, L⁵ is independently substituted or unsubstituted 2 to 8 membered heteroalkylene. In embodiments, L⁵ is independently substituted 2 to 8 membered heteroalkylene. In embodiments, L⁵ is independently unsubstituted 2 to 8 membered heteroalkylene. In embodiments, L⁵ is independently substituted or unsubstituted 2 to 6 membered heteroalkylene. In embodiments, L⁵ is independently substituted 2 to 6 membered heteroalkylene. In embodiments, L⁵ is independently unsubstituted 2 to 6 membered heteroalkylene. In embodiments, L⁵ is independently substituted or unsubstituted 4 to 6 membered heteroalkylene. In embodiments, L⁵ is independently substituted 4 to 6 membered heteroalkylene. In embodiments, L⁵ is independently unsubstituted 4 to 6 membered heteroalkylene. In embodiments, L⁵ is independently substituted or unsubstituted 2 to 3 membered heteroalkylene. In embodiments, L⁵ is independently substituted 2 to 3 membered heteroalkylene. In embodiments, L⁵ is independently unsubstituted 2 to 3 membered heteroalkylene. In embodiments, L⁵ is independently substituted or unsubstituted 4 to 5 membered heteroalkylene. In embodiments, L⁵ is independently substituted 4 to 5 membered heteroalkylene. In embodiments, L⁵ is independently unsubstituted 4 to 5 membered heteroalkylene.

In embodiments, L⁵ is independently substituted or unsubstituted cycloalkylene (e.g., C₃-C₁₀, C₃-C₈, C₃-C₆, C₄-C₆, or C₅-C₆). In embodiments, L⁵ is independently substituted cycloalkylene (e.g., C₃-C₁₀, C₃-C₈, C₃-C₆, C₄-C₆, or C₅-C₆). In embodiments, L⁵ is independently unsubstituted cycloalkylene (e.g., C₃-C₁₀, C₃-C₈, C₃-C₆, C₄-C₆, or C₅-C₆). In embodiments, L⁵ is independently substituted or unsubstituted C₃-C₁₀ cycloalkylene. In embodiments, L⁵ is independently substituted C₃-C₁₀ cycloalkylene. In embodiments, L⁵ is independently unsubstituted C₃-C₁₀ cycloalkylene. In embodiments, L⁵ is independently substituted or unsubstituted C₃-C₈ cycloalkylene. In embodiments, L⁵ is independently substituted C₃-C₈ cycloalkylene. In embodiments, L⁵ is independently unsubstituted C₃-C₈ cycloalkylene. In embodiments, L⁵ is independently substituted or unsubstituted C₃-C₆ cycloalkylene. In embodiments, L⁵ is independently substituted C₃-C₆ cycloalkylene. In embodiments, L⁵ is independently unsubstituted C₃-C₆ cycloalkylene. In embodiments, L⁵ is independently substituted or unsubstituted C₄-C₆ cycloalkylene. In embodiments, L⁵ is independently substituted C₄-C₆ cycloalkylene. In embodiments, L⁵ is independently unsubstituted C₄-C₆ cycloalkylene. In embodiments, L⁵ is independently substituted or unsubstituted C₅-C₆ cycloalkylene. In embodiments, L⁵ is independently substituted C₅-C₆ cycloalkylene. In embodiments, L⁵ is independently unsubstituted C₅-C₆ cycloalkylene. In embodiments, L⁵ is independently substituted or unsubstituted hexylene. In embodiments, L⁵ is independently substituted hexylene. In embodiments, L⁵ is independently unsubstituted hexylene.

In embodiments, L⁵ is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L⁵ is independently substituted heterocycloalkylene (e.g., 3 to 10 membered, 3 to 8 membered, 3 to 6 membered, 4 to 6 membered, 4 to 5 membered, or 5 to 6 membered). In embodiments, L⁵ is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L⁵ is independently substituted or unsubstituted 3 to 10 membered heterocycloalkylene. In embodiments, L⁵ is independently substituted 3 to 10 membered heterocycloalkylene. In embodiments, L⁵ is independently unsubstituted 3 to 10 membered heterocycloalkylene. In embodiments, L⁵ is independently substituted or unsubstituted 3 to 8 membered heterocycloalkylene. In embodiments, L⁵ is independently substituted 3 to 8 membered heterocycloalkylene. In embodiments, L⁵ is independently unsubstituted 3 to 8 membered heterocycloalkylene. In embodiments, L⁵ is independently substituted or unsubstituted 3 to 6 membered heterocycloalkylene. In embodiments, L⁵ is independently substituted 3 to 6 membered heterocycloalkylene. In embodiments, L⁵ is independently unsubstituted 3 to 6 membered heterocycloalkylene. In embodiments, L⁵ is independently substituted or unsubstituted 4 to 6 membered heterocycloalkylene. In embodiments, L⁵ is independently substituted 4 to 6 membered heterocycloalkylene. In embodiments, L⁵ is independently unsubstituted 4 to 6 membered heterocycloalkylene. In embodiments, L⁵ is independently substituted or unsubstituted 4 to 5 membered heterocycloalkylene. In embodiments, L⁵ is independently substituted 4 to 5 membered heterocycloalkylene. In embodiments, L⁵ is independently unsubstituted 4 to 5 membered heterocycloalkylene. In embodiments, L⁵ is independently substituted or unsubstituted 5 to 6 membered heterocycloalkylene. In embodiments, L⁵ is independently substituted 5 to 6 membered heterocycloalkylene. In embodiments, L⁵ is independently unsubstituted 5 to 6 membered heterocycloalkylene.

In embodiments, L⁵ is independently substituted or unsubstituted arylene (e.g., C₆-C₁₂, C₆-C₁₀, or phenyl). In embodiments, L⁵ is independently substituted arylene (e.g., C₆-C₁₂, C₆-C₁₀, or phenyl). In embodiments, L⁵ is independently unsubstituted arylene (e.g., C₆-C₁₂, C₆-C₁₀, or phenyl). In embodiments, L⁵ is independently substituted or unsubstituted C₆-C₁₂ arylene. In embodiments, L⁵ is independently substituted C₆-C₁₂ arylene. In embodiments, L⁵ is independently unsubstituted C₆-C₁₂ arylene. In embodiments, L⁵ is independently substituted or unsubstituted C₆-C₁₀ arylene. In embodiments, L⁵ is independently substituted C₆-C₁₀ arylene. In embodiments, L⁵ is independently unsubstituted C₆-C₁₀ arylene. In embodiments, L⁵ is independently substituted or unsubstituted phenylene. In embodiments, L⁵ is independently substituted phenylene. In embodiments, L⁵ is independently unsubstituted phenylene. In embodiments, L⁵ is independently substituted or unsubstituted biphenylene. In embodiments, L⁵ is independently substituted biphenylene. In embodiments, L⁵ is independently unsubstituted biphenylene. In embodiments, L⁵ is independently substituted or unsubstituted naphthylene. In embodiments, L⁵ is independently substituted naphthylene. In embodiments, L⁵ is independently unsubstituted naphthylene.

In embodiments, L⁵ is independently substituted or unsubstituted heteroarylene. In embodiments, L⁵ is independently substituted heteroarylene. In embodiments, L⁵ is independently unsubstituted heteroarylene. In embodiments, L⁵ is independently substituted or unsubstituted heteroarylene (e.g., 5 to 12 membered, 5 to 10 membered, 5 to 9 membered, 4 to 6 membered, or 5 to 6 membered). In embodiments, L⁵ is independently substituted heteroarylene (e.g., 5 to 12 membered, 5 to 10 membered, 5 to 9 membered, 4 to 6 membered, or 5 to 6 membered). In embodiments, L⁵ is independently unsubstituted heteroarylene (e.g., 5 to 12 membered, 5 to 10 membered, 5 to 9 membered, 4 to 6 membered, or 5 to 6 membered). In embodiments, L⁵ is independently substituted or unsubstituted 5 to 12 membered heteroarylene. In embodiments, L⁵ is independently substituted 5 to 12 membered heteroarylene. In embodiments, L⁵ is independently unsubstituted 5 to 12 membered heteroarylene. In embodiments, L⁵ is independently substituted or unsubstituted 5 to 10 membered heteroarylene. In embodiments, L⁵ is independently substituted 5 to 10 membered heteroarylene. In embodiments, L⁵ is independently unsubstituted 5 to 10 membered heteroarylene. In embodiments, L⁵ is independently substituted or unsubstituted 5 to 9 membered heteroarylene. In embodiments, L⁵ is independently substituted 5 to 9 membered heteroarylene. In embodiments, L⁵ is independently unsubstituted 5 to 9 membered heteroarylene. In embodiments, L⁵ is independently substituted or unsubstituted 4 to 6 membered heteroarylene. In embodiments, L⁵ is independently substituted 4 to 6 membered heteroarylene. In embodiments, L⁵ is independently unsubstituted 4 to 6 membered heteroarylene. In embodiments, L⁵ is independently substituted or unsubstituted 5 to 6 membered heteroarylene. In embodiments, L⁵ is independently substituted 5 to 6 membered heteroarylene. In embodiments, L⁵ is independently unsubstituted 5 to 6 membered heteroarylene.

In embodiments, L⁵ is independently a bond.

In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁶ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂). In embodiments, L⁷ is independently substituted or unsubstituted C₁-C₂₀ alkylene. In embodiments, L⁶ is independently substituted C₁-C₂₀ alkylene. In embodiments, L⁷ is independently unsubstituted C₁-C₂₀ alkylene. In embodiments, L⁷ is independently substituted or unsubstituted C₁-C₁₂ alkylene. In embodiments, L⁷ is independently substituted C₁-C₁₂ alkylene. In embodiments, L⁷ is independently unsubstituted C₁-C₁₂ alkylene. In embodiments, L⁶ is independently substituted or unsubstituted C₁-C₈alkylene. In embodiments, L⁶ is independently substituted C₁-C₈ alkylene. In embodiments, L⁷ is independently unsubstituted C₁-C₈ alkylene. In embodiments, L⁷ is independently substituted or unsubstituted C₁-C₆ alkylene. In embodiments, L⁷ is independently substituted C₁-C₆ alkylene. In embodiments, L⁶ is independently unsubstituted C₁-C₆ alkylene. In embodiments, L⁷ is independently substituted or unsubstituted C₁-C₄ alkylene. In embodiments, L⁷ is independently substituted C₁-C₄ alkylene. In embodiments, L⁷ is independently unsubstituted C₁-C₄ alkylene. In embodiments, L⁷ is independently substituted or unsubstituted ethylene. In embodiments, L⁷ is independently substituted ethylene. In embodiments, L⁷ is independently unsubstituted ethylene. In embodiments, L⁷ is independently substituted or unsubstituted methylene. In embodiments, L⁷ is independently substituted methylene. In embodiments, L⁷ is independently unsubstituted methylene.

In embodiments, L⁷ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁷ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁷ is independently substituted or unsubstituted 2 to 20 membered heteroalkylene. In embodiments, L⁷ is independently substituted 2 to 20 membered heteroalkylene. In embodiments, L⁶ is independently unsubstituted 2 to 20 membered heteroalkylene. In embodiments, L⁶ is independently substituted or unsubstituted 2 to 12 membered heteroalkylene. In embodiments, L⁶ is independently substituted 2 to 12 membered heteroalkylene. In embodiments, L⁷ is independently unsubstituted 2 to 12 membered heteroalkylene. In embodiments, L⁷ is independently substituted or unsubstituted 2 to 8 membered heteroalkylene. In embodiments, L⁷ is independently substituted 2 to 8 membered heteroalkylene. In embodiments, L⁶ is independently unsubstituted 2 to 8 membered heteroalkylene. In embodiments, L⁷ is independently substituted or unsubstituted 2 to 6 membered heteroalkylene. In embodiments, L⁷ is independently substituted 2 to 6 membered heteroalkylene. In embodiments, L⁷ is independently unsubstituted 2 to 6 membered heteroalkylene. In embodiments, L⁷ is independently substituted or unsubstituted 4 to 6 membered heteroalkylene. In embodiments, L⁷ is independently substituted 4 to 6 membered heteroalkylene. In embodiments, L⁷ is independently unsubstituted 4 to 6 membered heteroalkylene. In embodiments, L⁶ is independently substituted or unsubstituted 2 to 3 membered heteroalkylene. In embodiments, L⁶ is independently substituted 2 to 3 membered heteroalkylene. In embodiments, L⁶ is independently unsubstituted 2 to 3 membered heteroalkylene. In embodiments, L⁷ is independently substituted or unsubstituted 4 to 5 membered heteroalkylene. In embodiments, L⁶ is independently substituted 4 to 5 membered heteroalkylene. In embodiments, L⁷ is independently unsubstituted 4 to 5 membered heteroalkylene.

In embodiments, L⁷ is independently a bond.

In embodiments, L⁷ is independently -L¹⁷-NH—C(O)— or -L¹⁷-C(O)—NH—, and L¹⁷ is independently substituted or unsubstituted C₁-C₂₀ alkylene, substituted or unsubstituted 2-20 membered heteroalkylene, or substituted or unsubstituted 2-20 membered heteroalkenylene.

In embodiments, L¹⁷ is independently substituted or unsubstituted alkylene (e.g., C₁-C₂₀, C₁-C₁₂, C₁-C₈, C₁-C₆, C₁-C₄, or C₁-C₂). In embodiments, L¹⁷ is independently substituted alkylene (e.g., C₁-C₂₀, C₁-C₁₂, C₁-C₈, C₁-C₆, C₁-C₄, or C₁-C₂). In embodiments, L¹⁷ is independently unsubstituted alkylene (e.g., C₁-C₂₀, C₁-C₁₂, C₁-C₈, C₁-C₆, C₁-C₄, or C₁-C₂). In embodiments, L¹⁷ is independently substituted or unsubstituted C₁-C₂₀ alkylene. In embodiments, L¹⁷ is independently substituted C₁-C₂₀ alkylene. In embodiments, L¹⁷ is independently unsubstituted C₁-C₂₀ alkylene. In embodiments, L¹⁷ is independently substituted or unsubstituted C₁-C₁₂ alkylene. In embodiments, L¹⁷ is independently substituted C₁-C₁₂ alkylene. In embodiments, L¹⁷ is independently unsubstituted C₁-C₁₂ alkylene. In embodiments, L¹⁷ is independently substituted or unsubstituted C₁-C₈ alkylene. In embodiments, L¹⁷ is independently substituted C₁-C₈ alkylene. In embodiments, L¹⁷ is independently unsubstituted C₁-C₈ alkylene. In embodiments, L¹⁷ is independently substituted or unsubstituted C₁-C₆ alkylene. In embodiments, L¹⁷ is independently substituted C₁-C₆ alkylene. In embodiments, L¹⁷ is independently unsubstituted C₁-C₆ alkylene. In embodiments, L¹⁷ is independently substituted or unsubstituted C₁-C₄ alkylene. In embodiments, L¹⁷ is independently substituted C₁-C₄ alkylene. In embodiments, L¹⁷ is independently unsubstituted C₁-C₄ alkylene. In embodiments, L¹⁷ is independently substituted or unsubstituted ethylene. In embodiments, L¹⁷ is independently substituted ethylene. In embodiments, L¹⁷ is independently unsubstituted ethylene. In embodiments, L¹⁷ is independently substituted or unsubstituted methylene. In embodiments, L¹⁷ is independently substituted methylene. In embodiments, L¹⁷ is independently unsubstituted methylene.

In embodiments, L¹⁷ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁷ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁷ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁷ is independently substituted or unsubstituted 2 to 20 membered heteroalkylene. In embodiments, L¹⁷ is independently substituted 2 to 20 membered heteroalkylene. In embodiments, L¹⁷ is independently unsubstituted 2 to 20 membered heteroalkylene. In embodiments, L¹⁷ is independently substituted or unsubstituted 2 to 12 membered heteroalkylene. In embodiments, L¹⁷ is independently substituted 2 to 12 membered heteroalkylene. In embodiments, L¹⁷ is independently unsubstituted 2 to 12 membered heteroalkylene. In embodiments, L¹⁷ is independently substituted or unsubstituted 2 to 8 membered heteroalkylene. In embodiments, L¹⁷ is independently substituted 2 to 8 membered heteroalkylene. In embodiments, L¹⁷ is independently unsubstituted 2 to 8 membered heteroalkylene. In embodiments, L¹⁷ is independently substituted or unsubstituted 2 to 6 membered heteroalkylene. In embodiments, L¹⁷ is independently substituted 2 to 6 membered heteroalkylene. In embodiments, L¹⁷ is independently unsubstituted 2 to 6 membered heteroalkylene. In embodiments, L¹⁷ is independently substituted or unsubstituted 4 to 6 membered heteroalkylene. In embodiments, L¹⁷ is independently substituted 4 to 6 membered heteroalkylene. In embodiments, L¹⁷ is independently unsubstituted 4 to 6 membered heteroalkylene. In embodiments, L¹⁷ is independently substituted or unsubstituted 2 to 3 membered heteroalkylene. In embodiments, L¹⁷ is independently substituted 2 to 3 membered heteroalkylene. In embodiments, L¹⁷ is independently unsubstituted 2 to 3 membered heteroalkylene. In embodiments, L¹⁷ is independently substituted or unsubstituted 4 to 5 membered heteroalkylene. In embodiments, L¹⁷ is independently substituted 4 to 5 membered heteroalkylene. In embodiments, L¹⁷ is independently unsubstituted 4 to 5 membered heteroalkylene.

In embodiments, L¹⁷ is independently substituted or unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁷ is independently substituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁷ is independently unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁷ is independently substituted or unsubstituted 2 to 20 membered heteroalkenylene. In embodiments, L¹⁷ is independently substituted 2 to 20 membered heteroalkenylene. In embodiments, L¹⁷ is independently unsubstituted 2 to 20 membered heteroalkenylene. In embodiments, L¹⁷ is independently substituted or unsubstituted 2 to 12 membered heteroalkenylene. In embodiments, L¹⁷ is independently substituted 2 to 12 membered heteroalkenylene. In embodiments, L¹⁷ is independently unsubstituted 2 to 12 membered heteroalkenylene. In embodiments, L¹⁷ is independently substituted or unsubstituted 2 to 8 membered heteroalkenylene. In embodiments, L¹⁷ is independently substituted 2 to 8 membered heteroalkenylene. In embodiments, L¹⁷ is independently unsubstituted 2 to 8 membered heteroalkenylene. In embodiments, L¹⁷ is independently substituted or unsubstituted 2 to 6 membered heteroalkenylene. In embodiments, L¹⁷ is independently substituted 2 to 6 membered heteroalkenylene. In embodiments, L¹⁷ is independently unsubstituted 2 to 6 membered heteroalkenylene. In embodiments, L¹⁷ is independently substituted or unsubstituted 4 to 6 membered heteroalkenylene. In embodiments, L¹⁷ is independently substituted 4 to 6 membered heteroalkenylene. In embodiments, L¹⁷ is independently unsubstituted 4 to 6 membered heteroalkenylene. In embodiments, L¹⁷ is independently substituted or unsubstituted 2 to 3 membered heteroalkenylene. In embodiments, L¹⁷ is independently substituted 2 to 3 membered heteroalkenylene. In embodiments, L¹⁷ is independently unsubstituted 2 to 3 membered heteroalkenylene. In embodiments, L¹⁷ is independently substituted or unsubstituted 4 to 5 membered heteroalkenylene. In embodiments, L¹⁷ is independently substituted 4 to 5 membered heteroalkenylene. In embodiments, L¹⁷ is independently unsubstituted 4 to 5 membered heteroalkenylene.

In embodiments, L⁷ is independently -L¹⁷-NH—C(O)—. In embodiments, L⁷ is independently -L¹⁷-C(O)—NH—. In embodiments, L¹⁷ is substituted or unsubstituted C₁-C₈ alkylene. In embodiments, L¹⁷ is substituted C₁-C₈ alkylene. In embodiments, L¹⁷ is unsubstituted C₁-C₈ alkylene.

In embodiments, L⁷ is independently —NHC(O)—.

In embodiments, L⁷ is independently

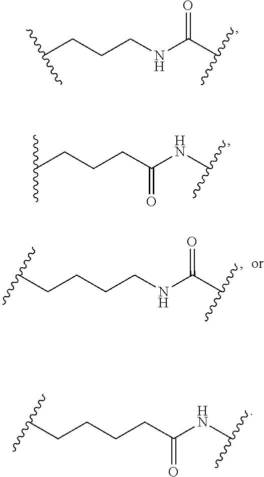 [SMILES: *CCCNC(*)=O ; *CCCC(=O)N* ; *CCCCNC(*)=O ; *CCCCC(=O)N*]

In embodiments, L⁷ is independently

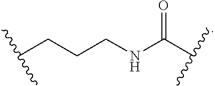 [SMILES: *CCCNC(*)=O]

In embodiments, L⁷ is independently

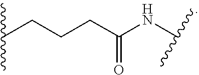 [SMILES: *CCCC(=O)N*]

In embodiments, L⁷ is independently

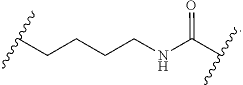 [SMILES: *CCCCNC(*)=O]

In embodiments, L⁷ is independently

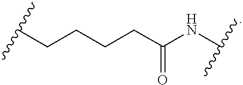 [SMILES: *CCCCC(=O)N*]

In embodiments, -L⁶-L⁵-L⁴- is independently a bond, or substituted or unsubstituted 2 to 50 membered heteroalkylene. In embodiments, -L⁶-L⁵-L⁴- is independently a bond.

In embodiments, -L⁶-L⁵-L⁴- is substituted or unsubstituted 2 to 50 membered heteroalkylene. In embodiments, -L⁶-L⁵-L⁴- is substituted or unsubstituted 2 to 45 membered heteroalkylene. In embodiments, -L⁶-L⁵-L⁴- is substituted or unsubstituted 2 to 40 membered heteroalkylene. In embodiments, -L⁶-L⁵-L⁴- is substituted or unsubstituted 2 to 35 membered heteroalkylene. In embodiments, -L⁶-L⁵-L⁴- is substituted or unsubstituted 2 to 30 membered heteroalkylene. In embodiments, -L⁶-L⁵-L⁴- is substituted or unsubstituted 2 to 25 membered heteroalkylene. In embodiments, -L⁶-L⁵-L⁴- is substituted or unsubstituted 2 to 20 membered heteroalkylene. In embodiments, -L⁶-L⁵-L⁴- is substituted or unsubstituted 2 to 15 membered heteroalkylene. In embodiments, -L⁶-L⁵-L⁴- is substituted or unsubstituted 2 to 10 membered heteroalkylene. In embodiments, -L⁶-L⁵-L⁴- is substituted or unsubstituted 2 to 5 membered heteroalkylene.

In embodiments, -L⁶-L⁵-L⁴- is substituted 2 to 50 membered heteroalkylene. In embodiments, -L⁶-L⁵-L⁴- is substituted 2 to 45 membered heteroalkylene. In embodiments, -L⁶-L⁵-L⁴- is substituted 2 to 40 membered heteroalkylene. In embodiments, -L⁶-L⁵-L⁴- is substituted 2 to 35 membered heteroalkylene. In embodiments, -L⁶-L⁵-L⁴- is substituted 2 to 30 membered heteroalkylene. In embodiments, -L⁶-L⁵-L⁴- is substituted 2 to 25 membered heteroalkylene. In embodiments, -L⁶-L⁵-L⁴- is substituted 2 to 20 membered heteroalkylene. In embodiments, L³ is substituted 2 to 50 membered heteroalkylene. In embodiments, -L⁶-L⁵-L⁴- is unsubstituted 2 to 15 membered heteroalkylene. In embodiments, -L⁶-L⁵-L⁴- is unsubstituted 2 to 10 membered heteroalkylene. In embodiments, -L⁶-L⁵-L⁴- is unsubstituted 2 to 5 membered heteroalkylene.

In embodiments, -L⁶-L⁵-L⁴- is unsubstituted 2 to 50 membered heteroalkylene. In embodiments, -L⁶-L⁵-L⁴- is unsubstituted 2 to 45 membered heteroalkylene. In embodiments, -L⁶-L⁵-L⁴- is unsubstituted 2 to 40 membered heteroalkylene. In embodiments, -L⁶-L⁵-L⁴- is unsubstituted 2 to 35 membered heteroalkylene. In embodiments, -L⁶-L⁵-L⁴- is unsubstituted 2 to 30 membered heteroalkylene. In embodiments, -L⁶-L⁵-L⁴- is unsubstituted 2 to 25 membered heteroalkylene. In embodiments, -L⁶-L⁵-L⁴- is unsubstituted 2 to 20 membered heteroalkylene. In embodiments, L³ is unsubstituted 2 to 50 membered heteroalkylene. In embodiments, -L⁶-L⁵-L⁴- is unsubstituted 2 to 15 membered heteroalkylene. In embodiments, -L⁶-L⁵-L⁴- is unsubstituted 2 to 10 membered heteroalkylene. In embodiments, -L⁶-L⁵-L⁴- is unsubstituted 2 to 5 membered heteroalkylene.

In embodiments, -L⁶-L⁵-L⁴- is independently R¹¹-substituted or unsubstituted 2 to 50 membered heteroalkylene. In embodiments, R¹¹ is oxo, hydroxyl, or unsubstituted C₁-C₄ alkyl. In embodiments, R¹¹ is oxo. In embodiments, R¹¹ is hydroxyl. In embodiments, R¹¹ is unsubstituted C₁-C₄ alkyl. In embodiments, R¹¹ is methyl. In embodiments, R¹¹ is ethyl. In embodiments, R¹¹ is propyl. In embodiments, R¹¹ is isopropyl. In embodiments, R¹¹ is butyl. In embodiments, R¹¹ is isobutyl. In embodiments, R¹¹ is t-butyl.

In embodiments, -L⁶-L⁵-L⁴- is independently R¹¹-substituted 2 to 50 membered heteroalkylene. In embodiments, -L⁶-L⁵-L⁴- is R¹¹-substituted 2 to 45 membered heteroalkylene. In embodiments, -L⁶-L⁵-L⁴- is R¹¹-substituted 2 to 40 membered heteroalkylene. In embodiments, -L⁶-L⁵-L⁴- is R¹¹-substituted 2 to 35 membered heteroalkylene. In embodiments, -L⁶-L⁵-L⁴- is R¹¹-substituted 2 to 30 membered heteroalkylene. In embodiments, -L⁶-L⁵-L⁴- is R¹¹-substituted 2 to 25 membered heteroalkylene. In embodiments, -L⁶-L⁵-L⁴- is R¹¹-substituted 2 to 20 membered heteroalkylene. In embodiments, -L⁶-L⁵-L⁴- is R¹¹-substituted 2 to 15 membered heteroalkylene. In embodiments, -L⁶-L⁵-L⁴- is R¹¹-substituted 2 to 11 membered heteroalkylene. In embodiments, -L⁶-L⁵-L⁴- is R¹¹-substituted 2 to 5 membered heteroalkylene.

In embodiments, -L⁶-L⁵-L⁴- is independently -L¹⁰-NH—C(O), or -L¹⁰-C(O)—NH—, and L¹⁰ is independently substituted or unsubstituted C₁-C₂₀ alkylene, substituted or unsubstituted 2-20 membered heteroalkylene, or substituted or unsubstituted 2-20 membered heteroalkenylene.

In embodiments, L¹⁰ is independently substituted or unsubstituted alkylene (e.g., C₁-C₂₀, C₁-C₁₂, C₁-C₈, C₁-C₆, C₁-C₄, or C₁-C₂). In embodiments, L¹⁰ is independently substituted alkylene (e.g., C₁-C₂₀, C₁-C₁₂, C₁-C₈, C₁-C₆, C₁-C₄, or C₁-C₂). In embodiments, L¹⁰ is independently unsubstituted alkylene (e.g., C₁-C₂₀, C₁-C₁₂, C₁-C₈, C₁-C₆, C₁-C₄, or C₁-C₂). In embodiments, L¹⁰ is independently substituted or unsubstituted C₁-C₂₀ alkylene. In embodiments, L¹⁰ is independently substituted C₁-C₂₀ alkylene. In embodiments, L¹⁰ is independently unsubstituted C₁-C₂₀ alkylene. In embodiments, L¹⁰ is independently substituted or unsubstituted C₁-C₁₂ alkylene. In embodiments, L¹⁰ is independently substituted C₁-C₁₂ alkylene. In embodiments, L¹⁰ is independently unsubstituted C₁-C₁₂ alkylene. In embodiments, L¹⁰ is independently substituted or unsubstituted C₁-C₈ alkylene. In embodiments, L¹⁰ is independently substituted C₁-C₈ alkylene. In embodiments, L¹⁰ is independently unsubstituted C₁-C₈ alkylene. In embodiments, L¹⁰ is independently substituted or unsubstituted C₁-C₆ alkylene. In embodiments, L¹⁰ is independently substituted C₁-C₆ alkylene. In embodiments, L¹⁰ is independently unsubstituted C₁-C₆ alkylene. In embodiments, L¹⁰ is independently substituted or unsubstituted C₁-C₄ alkylene. In embodiments, L¹⁰ is independently substituted C₁-C₄ alkylene. In embodiments, L¹⁰ is independently unsubstituted C₁-C₄ alkylene. In embodiments, L¹⁰ is independently substituted or unsubstituted ethylene. In embodiments, L¹⁰ is independently substituted ethylene. In embodiments, L¹⁰ is independently unsubstituted ethylene. In embodiments, L¹⁰ is independently substituted or unsubstituted methylene. In embodiments, L¹⁰ is independently substituted methylene. In embodiments, L¹⁰ is independently unsubstituted methylene.

In embodiments, L¹⁰ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁰ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁰ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹⁰ is independently substituted or unsubstituted 2 to 20 membered heteroalkylene. In embodiments, L¹⁰ is independently substituted 2 to 20 membered heteroalkylene. In embodiments, L¹⁰ is independently unsubstituted 2 to 20 membered heteroalkylene. In embodiments, L¹⁰ is independently substituted or unsubstituted 2 to 12 membered heteroalkylene. In embodiments, L¹⁰ is independently substituted 2 to 12 membered heteroalkylene. In embodiments, L¹⁰ is independently unsubstituted 2 to 12 membered heteroalkylene. In embodiments, L¹⁰ is independently substituted or unsubstituted 2 to 8 membered heteroalkylene. In embodiments, L¹⁰ is independently substituted 2 to 8 membered heteroalkylene. In embodiments, L¹⁰ is independently unsubstituted 2 to 8 membered heteroalkylene. In embodiments, L¹⁰ is independently substituted or unsubstituted 2 to 6 membered heteroalkylene. In embodiments, L¹⁰ is independently substituted 2 to 6 membered heteroalkylene. In embodiments, L¹⁰ is independently unsubstituted 2 to 6 membered heteroalkylene. In embodiments, L¹⁰ is independently substituted or unsubstituted 4 to 6 membered heteroalkylene. In embodiments, L¹⁰ is independently substituted 4 to 6 membered heteroalkylene. In embodiments, L¹⁰ is independently unsubstituted 4 to 6 membered heteroalkylene. In embodiments, L¹⁰ is independently substituted or unsubstituted 2 to 3 membered heteroalkylene. In embodiments, L¹⁰ is independently substituted 2 to 3 membered heteroalkylene. In embodiments, L¹⁰ is independently unsubstituted 2 to 3 membered heteroalkylene. In embodiments, L¹⁰ is independently substituted or unsubstituted 4 to 5 membered heteroalkylene. In embodiments, L¹⁰ is independently substituted 4 to 5 membered heteroalkylene. In embodiments, L¹⁰ is independently unsubstituted 4 to 5 membered heteroalkylene.

In embodiments, L¹⁰ is independently substituted or unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁰ is independently substituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁰ is independently unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹⁰ is independently substituted or unsubstituted 2 to 20 membered heteroalkenylene. In embodiments, L¹⁰ is independently substituted 2 to 20 membered heteroalkenylene. In embodiments, L¹⁰ is independently unsubstituted 2 to 20 membered heteroalkenylene. In embodiments, L¹⁰ is independently substituted or unsubstituted 2 to 12 membered heteroalkenylene. In embodiments, L¹⁰ is independently substituted 2 to 12 membered heteroalkenylene. In embodiments, L¹⁰ is independently unsubstituted 2 to 12 membered heteroalkenylene. In embodiments, L¹⁰ is independently substituted or unsubstituted 2 to 8 membered heteroalkenylene. In embodiments, L¹⁰ is independently substituted 2 to 8 membered heteroalkenylene. In embodiments, L¹⁰ is independently unsubstituted 2 to 8 membered heteroalkenylene. In embodiments, L¹⁰ is independently substituted or unsubstituted 2 to 6 membered heteroalkenylene. In embodiments, L¹⁰ is independently substituted 2 to 6 membered heteroalkenylene. In embodiments, L¹⁰ is independently unsubstituted 2 to 6 membered heteroalkenylene. In embodiments, L¹⁰ is independently substituted or unsubstituted 4 to 6 membered heteroalkenylene. In embodiments, L¹⁰ is independently substituted 4 to 6 membered heteroalkenylene. In embodiments, L¹⁰ is independently unsubstituted 4 to 6 membered heteroalkenylene. In embodiments, L¹⁰ is independently substituted or unsubstituted 2 to 3 membered heteroalkenylene. In embodiments, L¹⁰ is independently substituted 2 to 3 membered heteroalkenylene. In embodiments, L¹⁰ is independently unsubstituted 2 to 3 membered heteroalkenylene. In embodiments, L¹⁰ is independently substituted or unsubstituted 4 to 5 membered heteroalkenylene. In embodiments, L¹⁰ is independently substituted 4 to 5 membered heteroalkenylene. In embodiments, L¹⁰ is independently unsubstituted 4 to 5 membered heteroalkenylene.

In embodiments, -L⁶-L⁵-L⁴- is independently -L¹⁰-NH—C(O)—. In embodiments, -L⁶-L⁵-L⁴- is independently -L¹⁰-C(O)—NH—. In embodiments, L¹⁰ is substituted or unsubstituted C₁-C₈ alkylene. In embodiments, L¹⁰ is substituted C₁-C₈ alkylene. In embodiments, L¹⁰ is unsubstituted C₁-C₈ alkylene.

In embodiments, -L⁶-L⁵-L⁴- is independently

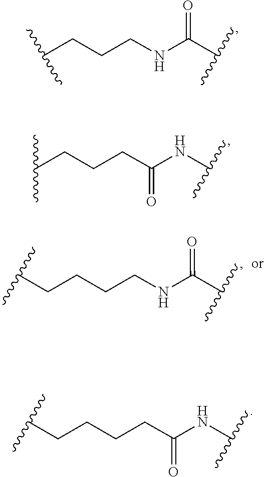 [SMILES: *CCCNC(*)=O ; *CCCC(=O)N* ; *CCCCNC(*)=O ; *CCCCC(=O)N*]

In embodiments, -L⁶-L⁵-L⁴- is independently

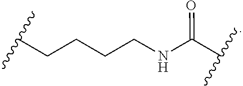 [SMILES: *CCCCNC(*)=O]

In embodiments, -L⁶-L⁵-L⁴- is independently

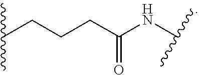 [SMILES: *CCCC(=O)N*]

In embodiments, -L⁶-L⁵-L⁴- is independently

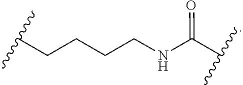 [SMILES: *CCCCNC(*)=O]

In embodiments, -L⁶-L⁵-L⁴- is independently

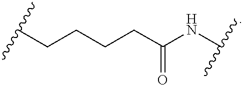 [SMILES: *CCCCC(=O)N*]

In embodiments, -L⁶-L⁵-L⁴- is independently —O-L¹⁰-NH—C(O)— or —O-L¹⁰-C(O)—NH—, and L¹⁰ is independently substituted or unsubstituted C₁-C₂₀ alkylene, substituted or unsubstituted 2-20 membered heteroalkylene, or substituted or unsubstituted 2-20 membered heteroalkenylene.

In embodiments, -L⁶-L⁵-L⁴- is independently —O-L¹⁰-NH—C(O)—. In embodiments, -L⁶-L⁵-L⁴- is —O-L¹⁰-C(O)—NH—. In embodiments, L¹⁰ is substituted or unsubstituted C₁-C₈ alkylene. In embodiments, L¹⁰ is substituted C₁-C₈ alkylene. In embodiments, L¹⁰ is hydroxyalkyl-substituted C₁-C₈ alkylene. In embodiments, L¹⁰ is hydroxymethyl-substituted C₁-C₈ alkylene. In embodiments, L¹⁰ is unsubstituted C₁-C₈ alkylene.

In embodiments, -L⁶-L⁵-L⁴- is independently

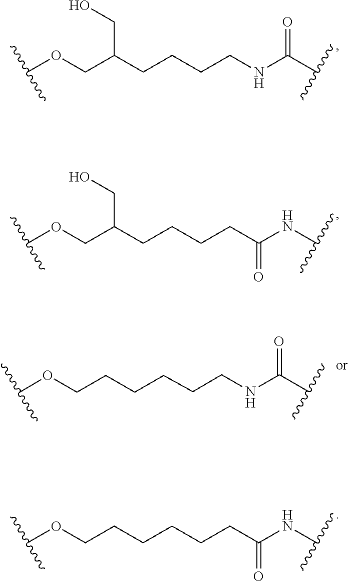 [SMILES: *OCC(CO)CCCCNC(*)=O ; *NC(=O)CCCCC(CO)CO* ; *OCCCCCCNC(*)=O ; *NC(=O)CCCCCCO*]

In embodiments, -L⁶-L⁵-L⁴- is independently

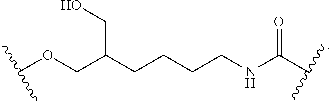 [SMILES: *OCC(CO)CCCCNC(*)=O]

In embodiments, -L⁶-L⁵-L⁴- is independently

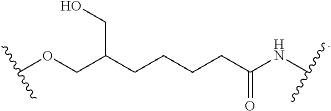 [SMILES: *NC(=O)CCCCC(CO)CO*]

In embodiments, -L⁶-L⁵-L⁴- is independently

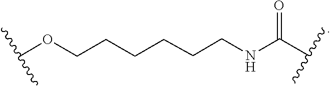 [SMILES: *OCCCCCCNC(*)=O]

In embodiments, -L⁶-L⁵-L⁴- is independently

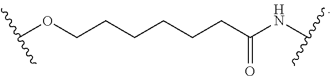 [SMILES: *NC(=O)CCCCCCO*]

In embodiments, -L⁶-L⁵-L⁴- is independently —OPO₂—O-L¹⁰-NH—C(O)— or —OPO₂—O-L¹⁰-C(O)—NH—, and L¹⁰ is independently substituted or unsubstituted C₁-C₂₀ alkylene, or substituted or unsubstituted 2-20 membered heteroalkylene.

In embodiments, -L⁶-L⁵-L⁴- is independently —OPO₂—O-L¹⁰-NH—C(O)—. In embodiments, -L⁶-L⁵-L⁴- is independently —OPO₂—O-L¹⁰-C(O)—NH—. In embodiments, L¹⁰ is substituted or unsubstituted C₁-C₈ alkylene. In embodiments, L¹⁰ is substituted C₁-C₈ alkylene. In embodiments, L¹⁰ is hydroxyalkyl-substituted C₁-C₈ alkylene. In embodiments, L¹⁰ is hydroxymethyl-substituted C₁-C₈ alkylene. In embodiments, L¹⁰ is unsubstituted C₁-C₈ alkylene.

In embodiments, -L⁶-L⁵-L⁴- is independently

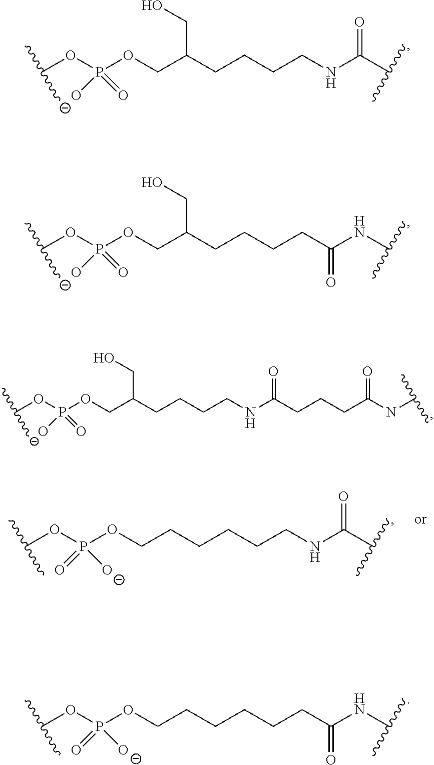 [SMILES: *OP(=O)([O-])OCC(CO)CCCCNC(*)=O ; *NC(=O)CCCCC(CO)COP(=O)([O-])O* ; *NC(=O)CCCC(=O)NCCCCC(CO)COP(=O)([O-])O* ; *OP(=O)([O-])OCCCCCCNC(*)=O ; *NC(=O)CCCCCCOP(=O)([O-])O*]

In embodiments, -L⁶-L⁵-L⁴- is independently

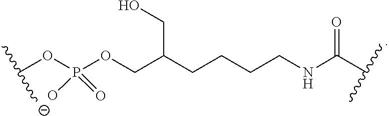 [SMILES: *OP(=O)([O-])OCC(CO)CCCCNC(*)=O]

In embodiments, -L⁶-L⁵-L⁴- is independently

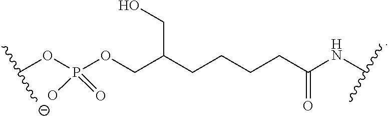 [SMILES: *NC(=O)CCCCC(CO)COP(=O)([O-])O*]

In embodiments, -L⁶-L⁵-L⁴- is independently

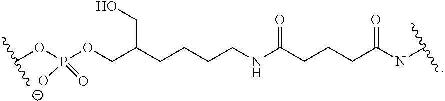

In embodiments, -L⁶-L⁵-L⁴- is independently

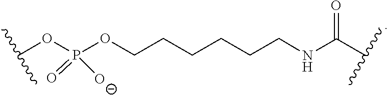 [SMILES: *OP(=O)([O-])OCCCCCCNC(*)=O]

In embodiments, -L⁶-L⁵-L⁴- is independently

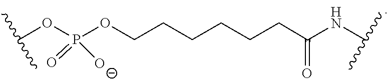 [SMILES: *NC(=O)CCCCCCOP(=O)([O-])O*]

In embodiments, -L⁶-L⁵-L⁴- is independently

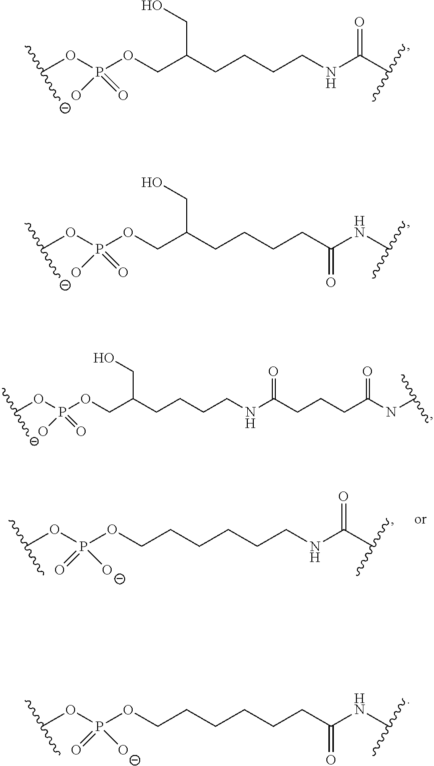 [SMILES: *OP(=O)([O-])OCC(CO)CCCCNC(*)=O ; *NC(=O)CCCCC(CO)COP(=O)([O-])O* ; *NC(=O)CCCC(=O)NCCCCC(CO)COP(=O)([O-])O* ; *OP(=O)([O-])OCCCCCCNC(*)=O ; *NC(=O)CCCCCCOP(=O)([O-])O*]

In embodiments, -L⁶-L⁵-L⁴- is independently

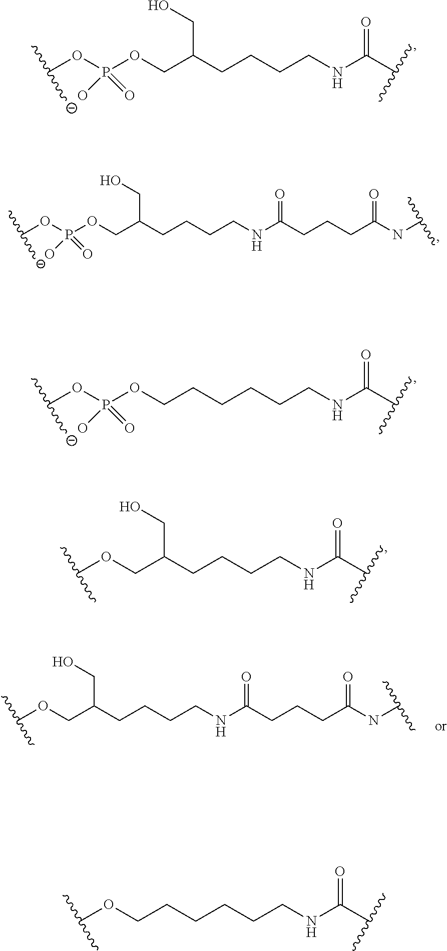 [SMILES: *OP(=O)([O-])OCC(CO)CCCCNC(*)=O ; *NC(=O)CCCC(=O)NCCCCC(CO)COP(=O)([O-])O* ; *OP(=O)([O-])OCCCCCCNC(*)=O ; *OCC(CO)CCCCNC(*)=O ; *NC(=O)CCCC(=O)NCCCCC(CO)CO* ; *OCCCCCCNC(*)=O]

and is attached to a 3′ carbon of the double-stranded nucleic acid or single-stranded nucleic acid. In embodiments, the 3′ carbon is the 3′ carbon of a 3′ terminal nucleotide. In embodiments, the 3′ carbon is the 3′ carbon of a 3′ terminal nucleotide. In embodiments, -L⁶-L⁵-L⁴- is independently

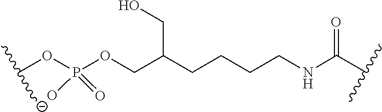 [SMILES: *OP(=O)([O-])OCC(CO)CCCCNC(*)=O]

and is attached to a 3′ carbon of the double-stranded nucleic acid or single-stranded nucleic acid. In embodiments, the 3′ carbon is the 3′ carbon of a 3′ terminal nucleotide. In embodiments, -L⁶-L⁵-L⁴- is independently

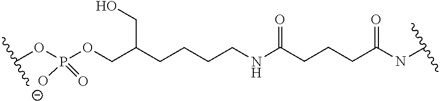

and is attached to a 3′ carbon of the double-stranded nucleic acid or single-stranded nucleic acid. In embodiments, the 3′ carbon is the 3′ carbon of a 3′ terminal nucleotide. In embodiments, -L⁶-L⁵-L⁴- is independently

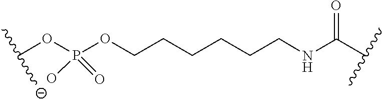

and is attached to a 3′ carbon of the double-stranded nucleic acid or single-stranded nucleic acid. In embodiments, the 3′ carbon is the 3′ carbon of a 3′ terminal nucleotide. In embodiments, -L⁶-L⁵-L⁴- is independently

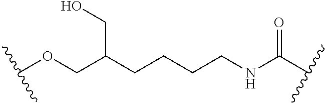 [SMILES: *OCC(CO)CCCCNC(*)=O]

and is attached to a 3′ carbon of the double-stranded nucleic acid or single-stranded nucleic acid. In embodiments, -L⁶-L⁵-L⁴- is independently

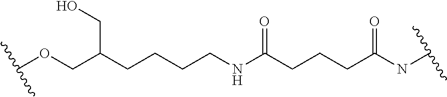 [SMILES: *NC(=O)CCCC(=O)NCCCCC(CO)CO*]

and is attached to a 3′ carbon of the double-stranded nucleic acid or single-stranded nucleic acid. In embodiments, the 3′ carbon is the 3′ carbon of a 3′ terminal nucleotide. In embodiments, -L⁶-L⁵-L⁴- is independently

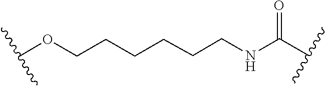 [SMILES: *OCCCCCCNC(*)=O]

and is attached to a 3′ carbon of the double-stranded nucleic acid or single-stranded nucleic acid.

In embodiments, -L⁶-L⁵-L⁴- is independently

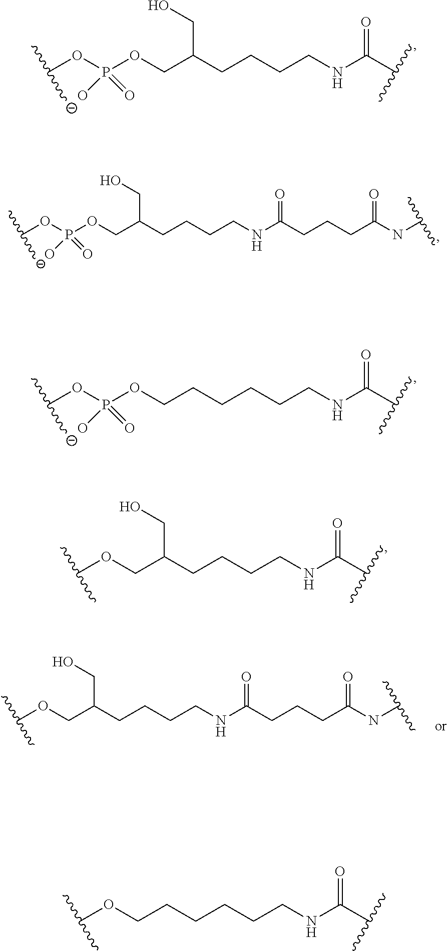 [SMILES: *OP(=O)([O-])OCC(CO)CCCCNC(*)=O ; *NC(=O)CCCC(=O)NCCCCC(CO)COP(=O)([O-])O* ; *OP(=O)([O-])OCCCCCCNC(*)=O ; *OCC(CO)CCCCNC(*)=O ; *NC(=O)CCCC(=O)NCCCCC(CO)CO* ; *OCCCCCCNC(*)=O]

and is attached to a 5′ carbon of the double-stranded nucleic acid or single-stranded nucleic acid. In embodiments, the 5′ carbon is the 5′ carbon of a 5′ terminal nucleotide. In embodiments, -L⁶-L⁵-L⁴- is independently

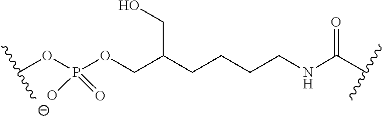 [SMILES: *OP(=O)([O-])OCC(CO)CCCCNC(*)=O]

and is attached to a 5′ carbon of the double-stranded nucleic acid or single-stranded nucleic acid. In embodiments, the 5′ carbon is the 5′ carbon of a 5′ terminal nucleotide. In embodiments, -L⁶-L⁵-L⁴- is independently

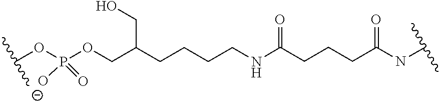

and is attached to a 5′ carbon of the double-stranded nucleic acid or single-stranded nucleic acid. In embodiments, -L⁶-L⁵-L⁴- is independently

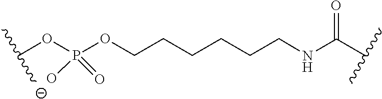

and is attached to a 5′ carbon of the double-stranded nucleic acid or single-stranded nucleic acid. In embodiments, -L⁶-L⁵-L⁴- is independently

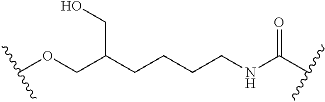 [SMILES: *OCC(CO)CCCCNC(*)=O]

and is attached to a 5′ carbon of the double-stranded nucleic acid or single-stranded nucleic acid. In embodiments, -L⁶-L⁵-L⁴- is independently

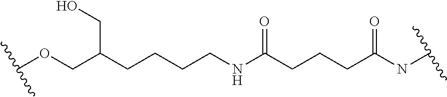 [SMILES: *NC(=O)CCCC(=O)NCCCCC(CO)CO*]

and is attached to a 5′ carbon of the double-stranded nucleic acid or single-stranded nucleic acid. In embodiments, -L⁶-L⁵-L⁴- is independently

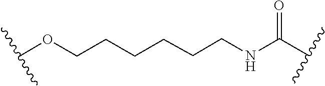 [SMILES: *OCCCCCCNC(*)=O]

and is attached to a 5′ carbon of the double-stranded nucleic acid or single-stranded nucleic acid.

In embodiments, -L⁶-L⁵-L⁴- is independently

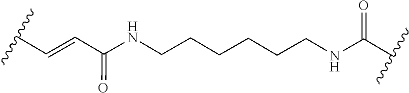 [SMILES: */C=C/C(=O)NCCCCCCNC(*)=O]

and is attached to a nucleotide base of the double-stranded nucleic acid or single-stranded nucleic acid.

In embodiments, L¹ is independently —NHC(O)—, —C(O)NH—, substituted or unsubstituted alkylene, or substituted or unsubstituted heteroalkylene. In embodiments, L¹ is independently —NHC(O)—. In embodiments, L¹ is independently —C(O)NH—. In embodiments, L¹ is substituted or unsubstituted alkylene. In embodiments, L¹ is substituted or unsubstituted heteroalkylene.

In embodiments, L¹ is independently substituted or unsubstituted alkylene (e.g., C₁-C₂₀, C₁-C₁₂, C₁-C₈, C₁-C₆, C₁-C₄, or C₁-C₂). In embodiments, L¹ is independently substituted alkylene (e.g., C₁-C₂₀, C₁-C₁₂, C₁-C₈, C₁-C₆, C₁-C₄, or C₁-C₂). In embodiments, L¹ is independently unsubstituted alkylene (e.g., C₁-C₂₀, C₁-C₁₂, C₁-C₈, C₁-C₆, C₁-C₄, or C₁-C₂). In embodiments, L¹ is independently substituted or unsubstituted C₁-C₂₀ alkylene. In embodiments, L¹ is independently substituted C₁-C₂₀ alkylene. In embodiments, L¹ is independently unsubstituted C₁-C₂₀ alkylene. In embodiments, L¹ is independently substituted or unsubstituted C₁-C₁₂ alkylene. In embodiments, L¹ is independently substituted C₁-C₁₂ alkylene. In embodiments, L¹ is independently unsubstituted C₁-C₁₂ alkylene. In embodiments, L¹ is independently substituted or unsubstituted C₁-C₈ alkylene. In embodiments, L¹ is independently substituted C₁-C₈ alkylene. In embodiments, L¹ is independently unsubstituted C₁-C₈ alkylene. In embodiments, L¹ is independently substituted or unsubstituted C₁-C₆ alkylene. In embodiments, L¹ is independently substituted C₁-C₆ alkylene. In embodiments, L¹ is independently unsubstituted C₁-C₆ alkylene. In embodiments, L¹ is independently substituted or unsubstituted C₁-C₄ alkylene. In embodiments, L¹ is independently substituted C₁-C₄ alkylene. In embodiments, L¹ is independently unsubstituted C₁-C₄ alkylene. In embodiments, L¹ is independently substituted or unsubstituted ethylene. In embodiments, L¹ is independently substituted ethylene. In embodiments, L¹ is independently unsubstituted ethylene. In embodiments, L¹ is independently substituted or unsubstituted methylene. In embodiments, L¹ is independently substituted methylene. In embodiments, L¹ is independently unsubstituted methylene.

In embodiments, L¹ is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L¹ is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L¹ is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L¹ is independently substituted or unsubstituted 2 to 25 membered heteroalkylene. In embodiments, L¹ is independently substituted 2 to 25 membered heteroalkylene. In embodiments, L¹ is independently unsubstituted 2 to 25 membered heteroalkylene. In embodiments, L¹ is independently substituted or unsubstituted 2 to 20 membered heteroalkylene. In embodiments, L¹ is independently substituted 2 to 20 membered heteroalkylene. In embodiments, L¹ is independently unsubstituted 2 to 20 membered heteroalkylene. In embodiments, L¹ is independently substituted or unsubstituted 2 to 12 membered heteroalkylene. In embodiments, L¹ is independently substituted 2 to 12 membered heteroalkylene. In embodiments, L¹ is independently unsubstituted 2 to 12 membered heteroalkylene. In embodiments, L¹ is independently substituted or unsubstituted 2 to 8 membered heteroalkylene. In embodiments, L¹ is independently substituted 2 to 8 membered heteroalkylene. In embodiments, L¹ is independently unsubstituted 2 to 8 membered heteroalkylene. In embodiments, L¹ is independently substituted or unsubstituted 2 to 6 membered heteroalkylene. In embodiments, L¹ is independently substituted 2 to 6 membered heteroalkylene. In embodiments, L¹ is independently unsubstituted 2 to 6 membered heteroalkylene. In embodiments, L¹ is independently substituted or unsubstituted 4 to 6 membered heteroalkylene. In embodiments, L¹ is independently substituted 4 to 6 membered heteroalkylene. In embodiments, L¹ is independently unsubstituted 4 to 6 membered heteroalkylene. In embodiments, L¹ is independently substituted or unsubstituted 2 to 3 membered heteroalkylene. In embodiments, L¹ is independently substituted 2 to 3 membered heteroalkylene. In embodiments, L¹ is independently unsubstituted 2 to 3 membered heteroalkylene. In embodiments, L¹ is independently substituted or unsubstituted 4 to 5 membered heteroalkylene. In embodiments, L¹ is independently substituted 4 to 5 membered heteroalkylene. In embodiments, L¹ is independently unsubstituted 4 to 5 membered heteroalkylene.

In embodiments, L¹ is L^{1A}-L^{1B}-L^{1C}-L^{1D}-L^{1E}. In embodiments, L^{1A} is independently a bond, —NHC(O)—, substituted or unsubstituted alkylene, or substituted or unsubstituted heteroalkylene; L^{1B} is independently a bond, —O—, —NHC(O)—, substituted or unsubstituted alkylene, substituted or unsubstituted heteroalkylene, or substituted or unsubstituted arylene; L^{1C} is independently a bond, —O—, —NHC(O)—, substituted or unsubstituted alkylene, substituted or substituted or unsubstituted heteroalkylene, or substituted or unsubstituted arylene; L^{1D} is independently a bond, —O—, —NHC(O)—, substituted or unsubstituted alkylene, substituted or unsubstituted heteroalkylene, or substituted or unsubstituted arylene; and L^{1E} is independently a bond, —NHC(O)—, substituted or unsubstituted alkylene, or substituted or unsubstituted heteroalkylene.

In embodiments, L^{1A} is independently a bond, —NHC(O)—, substituted or unsubstituted alkylene, or substituted or unsubstituted heteroalkylene. In embodiments, L^{1B} is independently a bond, —O—, —NHC(O)—, substituted or unsubstituted alkylene, substituted or unsubstituted heteroalkylene, or substituted or unsubstituted arylene. In embodiments, L^{1C} is independently a bond, —O—, —NHC(O)—, substituted or unsubstituted alkylene, substituted or substituted or unsubstituted heteroalkylene, or substituted or unsubstituted arylene. In embodiments, L^{1D} is independently a bond, —O—, —NHC(O)—, substituted or unsubstituted alkylene, substituted or unsubstituted heteroalkylene, or substituted or unsubstituted arylene. In embodiments, L^{1E} is independently a bond, —NHC(O)—, substituted or unsubstituted alkylene, or substituted or unsubstituted heteroalkylene.

In embodiments, L^{1A} is independently a bond, —NHC(O)—, substituted or unsubstituted C₁-C₈alkylene, or substituted or unsubstituted 2 to 8 membered heteroalkylene; L^{1B} is independently a bond, —O—, —NHC(O)—, substituted or unsubstituted C₁-C₈ alkylene, substituted or unsubstituted 2 to 8 membered heteroalkylene, or substituted or unsubstituted phenylene; L^{1C} is independently a bond, —O—, —NHC(O)—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene; L^{1D} is independently a bond, —O—, —NHC(O)—, substituted or unsubstituted C₁-C₈ alkylene, substituted or unsubstituted 2 to 8 membered heteroalkylene, or substituted or unsubstituted phenylene; and L^{1E} is independently a bond, —NHC(O)—, substituted or unsubstituted C₁-C₈ alkylene, or substituted or unsubstituted 2 to 8 membered heteroalkylene. In embodiments, L^{1A} is independently a bond, —NHC(O)—, substituted or unsubstituted C₁-C₈ alkylene, or substituted or unsubstituted 2 to 8 membered heteroalkylene. In embodiments, L^{1B} is independently a bond, —O—, —NHC(O)—, substituted or unsubstituted C₁-C₈ alkylene, substituted or unsubstituted 2 to 8 membered heteroalkylene, or substituted or unsubstituted phenylene. In embodiments, L^{1C} is independently a bond, —O—, —NHC(O)—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene. In embodiments, L^{1D} is independently a bond, —O—, —NHC(O)—, substituted or unsubstituted C₁-C₈ alkylene, substituted or unsubstituted 2 to 8 membered heteroalkylene, or substituted or unsubstituted phenylene. In embodiments, L^{1E} is independently a bond, —NHC(O)—, substituted or unsubstituted C₁-C₈ alkylene, or substituted or unsubstituted 2 to 8 membered heteroalkylene.

In embodiments, L^{1A} is independently unsubstituted C₁-C₈ alkylene, or unsubstituted 2 to 8 membered heteroalkylene; L^{1B} is independently a bond, —O—, —NHC(O)—, unsubstituted C₁-C₈ alkylene, unsubstituted C₂-C₈ alkynylene, unsubstituted 2 to 8 membered heteroalkylene, or unsubstituted phenylene; L^{1C} is independently a bond, —O—, —NHC(O)—, unsubstituted C₁-C₈ alkylene, unsubstituted 2 to 8 membered heteroalkylene, or unsubstituted phenylene; L^{1D} is independently a bond, —O—, —NHC(O)—, unsubstituted C₁-C₈ alkylene, or unsubstituted 2 to 8 membered heteroalkylene; and L^{1E} is independently —NHC(O)—.

In embodiments, L^{1A} is independently unsubstituted C₁-C₈ alkylene, or unsubstituted 2 to 8 membered heteroalkylene. In embodiments, L^{1B} is independently a bond, —O—, —NHC(O)—, unsubstituted C₁-C₈ alkylene, unsubstituted C₂-C₈ alkynylene, unsubstituted 2 to 8 membered heteroalkylene, or unsubstituted phenylene. In embodiments, L^{1C} is independently a bond, —O—, —NHC(O)—, unsubstituted C₁-C₈ alkylene, unsubstituted 2 to 8 membered heteroalkylene, or unsubstituted phenylene. In embodiments, L^{1D} is independently a bond, —O—, —NHC(O)—, unsubstituted C₁-C₈ alkylene, or unsubstituted 2 to 8 membered heteroalkylene. In embodiments, L^{1E} is independently —NHC(O)—.

In embodiments, L^{1A} is independently a bond. In embodiments, L^{1A} is independently —NH—. In embodiments, L^{1A} is independently —O—. In embodiments, L^{1A} is independently —S—. In embodiments, L^{1A} is independently —C(O)—. In embodiments, L^{1A} is independently —NHC(O)—. In embodiments, L^{1A} is independently —S(O)₂C(O)—. In embodiments, L^{1A} is independently —OPO₂—O—. In embodiments, L^{1A} is independently —OP(S)(O)—O—. In embodiments, L^{1A} is independently —OP(S)₂—O—. In embodiments, L^{1A} is independently —S(O)NH—. In embodiments, L^{1A} is independently —NHC(O)NH—. In embodiments, L^{1A} is independently —C(O)O—. In embodiments, L^{1A} is independently —OC(O)—. In embodiments, L^{1A} is independently —C(O)NH—. In embodiments, L^{1A} is not a bond.

In embodiments, L^{1A} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{1A} is independently substituted alkylene (e.g., C₁-C₂₅, C₁-C₂₀, C₁-C₁₂, C₁-C₈, C₁-C₆, C₁-C₄, or C₁-C₂). In embodiments, L^{1A} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{1A} is independently substituted or unsubstituted C₁-C₂₅ alkylene. In embodiments, L^{1A} is independently substituted C₁-C₂₅ alkylene. In embodiments, L^{1A} is independently unsubstituted C₁-C₂₅ alkylene. In embodiments, L^{1A} is independently substituted or unsubstituted C₁-C₂₀ alkylene. In embodiments, L^{1A} is independently substituted C₁-C₂₀ alkylene. In embodiments, L^{1A} is independently unsubstituted C₁-C₂₀ alkylene. In embodiments, L^{1A} is independently substituted or unsubstituted C₁-C₁₂ alkylene. In embodiments, L^{1A} is independently substituted C₁-C₁₂ alkylene. In embodiments, L^{1A} is independently unsubstituted C₁-C₁₂ alkylene. In embodiments, L^{1A} is independently substituted or unsubstituted C₁-C₈ alkylene. In embodiments, L^{1A} is independently substituted C₁-C₈ alkylene. In embodiments, L^{1A} is independently unsubstituted C₁-C₈alkylene. In embodiments, L^{1A} is independently substituted or unsubstituted C₁-C₆ alkylene. In embodiments, L^{1A} is independently substituted C₁-C₆ alkylene. In embodiments, L^{1A} is independently unsubstituted C₁-C₆ alkylene. In embodiments, L^{1A} is independently substituted or unsubstituted C₁-C₄ alkylene. In embodiments, L^{1A} is independently substituted C₁-C₄ alkylene. In embodiments, L^{1A} is independently unsubstituted C₁-C₄ alkylene. In embodiments, L^{1A} is independently substituted or unsubstituted ethylene. In embodiments, L^{1A} is independently substituted ethylene. In embodiments, L^{1A} is independently unsubstituted ethylene. In embodiments, L^{1A} is independently substituted or unsubstituted methylene. In embodiments, L^{1A} is independently substituted methylene. In embodiments, L^{1A} is independently unsubstituted methylene.

In embodiments, L^{1A} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1A} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1A} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1A} is independently substituted or unsubstituted 2 to 25 membered heteroalkylene. In embodiments, L^{1A} is independently substituted 2 to 25 membered heteroalkylene. In embodiments, L^{1A} is independently unsubstituted 2 to 25 membered heteroalkylene. In embodiments, L^{1A} is independently substituted or unsubstituted 2 to 20 membered heteroalkylene. In embodiments, L^{1A} is independently substituted 2 to 20 membered heteroalkylene. In embodiments, L^{1A} is independently unsubstituted 2 to 20 membered heteroalkylene. In embodiments, L^{1A} is independently substituted or unsubstituted 2 to 12 membered heteroalkylene. In embodiments, L^{1A} is independently substituted 2 to 12 membered heteroalkylene. In embodiments, L^{1A} is independently unsubstituted 2 to 12 membered heteroalkylene. In embodiments, L^{1A} is independently substituted or unsubstituted 2 to 8 membered heteroalkylene. In embodiments, L^{1A} is independently substituted 2 to 8 membered heteroalkylene. In embodiments, L^{1A} is independently unsubstituted 2 to 8 membered heteroalkylene. In embodiments, L^{1A} is independently substituted or unsubstituted 2 to 6 membered heteroalkylene. In embodiments, L^{1A} is independently substituted 2 to 6 membered heteroalkylene. In embodiments, L^{1A} is independently unsubstituted 2 to 6 membered heteroalkylene. In embodiments, L^{1A} is independently substituted or unsubstituted 4 to 6 membered heteroalkylene. In embodiments, L^{1A} is independently substituted 4 to 6 membered heteroalkylene. In embodiments, L^{1A} is independently unsubstituted 4 to 6 membered heteroalkylene. In embodiments, L^{1A} is independently substituted or unsubstituted 2 to 3 membered heteroalkylene. In embodiments, L^{1A} is independently substituted 2 to 3 membered heteroalkylene. In embodiments, L^{1A} is independently unsubstituted 2 to 3 membered heteroalkylene. In embodiments, L^{1A} is independently substituted or unsubstituted 4 to 5 membered heteroalkylene. In embodiments, L^{1A} is independently substituted 4 to 5 membered heteroalkylene. In embodiments, L^{1A} is independently unsubstituted 4 to 5 membered heteroalkylene.

In embodiments, L^{1A} is independently substituted or unsubstituted cyclocycloalkylene (e.g., C₃-C₁₀, C₃-C₈, C₃-C₆, C₄-C₆, or C₅-C₆). In embodiments, L^{1A} is independently substituted cyclocycloalkylene (e.g., C₃-C₁₀, C₃-C₈, C₃-C₆, C₄-C₆, or C₅-C₆). In embodiments, L^{1A} is independently unsubstituted cyclocycloalkylene (e.g., C₃-C₁₀, C₃-C₈, C₃-C₆, C₄-C₆, or C₅-C₆). In embodiments, L^{1A} is independently substituted or unsubstituted C₃-C₁₀ cycloalkylene. In embodiments, L^{1A} is independently substituted C₃-C₁₀ cycloalkylene. In embodiments, L^{1A} is independently unsubstituted C₃-C₁₀ cycloalkylene. In embodiments, L^{1A} is independently substituted or unsubstituted C₃-C₈ cycloalkylene. In embodiments, L^{1A} is independently substituted C₃-C₈ cycloalkylene. In embodiments, L^{1A} is independently unsubstituted C₃-C₈ cycloalkylene. In embodiments, L^{1A} is independently substituted or unsubstituted C₃-C₆ cycloalkylene. In embodiments, L^{1A} is independently substituted C₃-C₆ cycloalkylene. In embodiments, L^{1A} is independently unsubstituted C₃-C₆ cycloalkylene. In embodiments, L^{1A} is independently substituted or unsubstituted C₄-C₆ cycloalkylene. In embodiments, L^{1A} is independently substituted C₄-C₆ cycloalkylene. In embodiments, L^{1A} is independently unsubstituted C₄-C₆ cycloalkylene. In embodiments, L^{1A} is independently substituted or unsubstituted C₅-C₆ cycloalkylene. In embodiments, L^{1A} is independently substituted C₅-C₆ cycloalkylene. In embodiments, L^{1A} is independently unsubstituted C₅-C₆ cycloalkylene. In embodiments, L^{1A} is independently substituted or unsubstituted hexylene. In embodiments, L^{1A} is independently substituted hexylene. In embodiments, L^{1A} is independently unsubstituted hexylene.

In embodiments, L^{1A} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, LA is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{1A} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1A} is independently substituted or unsubstituted 3 to 10 membered heterocycloalkylene. In embodiments, L^{1A} is independently substituted 3 to 10 membered heterocycloalkylene. In embodiments, L^{1A} is independently unsubstituted 3 to 10 membered heterocycloalkylene. In embodiments, L^{1A} is independently substituted or unsubstituted 3 to 8 membered heterocycloalkylene. In embodiments, L^{1A} is independently substituted 3 to 8 membered heterocycloalkylene. In embodiments, L^{1A} is independently unsubstituted 3 to 8 membered heterocycloalkylene. In embodiments, L^{1A} is independently substituted or unsubstituted 3 to 6 membered heterocycloalkylene. In embodiments, L^{1A} is independently substituted 3 to 6 membered heterocycloalkylene. In embodiments, L^{1A} is independently unsubstituted 3 to 6 membered heterocycloalkylene. In embodiments, L^{1A} is independently substituted or unsubstituted 4 to 6 membered heterocycloalkylene. In embodiments, L^{1A} is independently substituted 4 to 6 membered heterocycloalkylene. In embodiments, L^{1A} is independently unsubstituted 4 to 6 membered heterocycloalkylene. In embodiments, L^{1A} is independently substituted or unsubstituted 4 to 5 membered heterocycloalkylene. In embodiments, L^{1A} is independently substituted 4 to 5 membered heterocycloalkylene. In embodiments, L^{1A} is independently unsubstituted 4 to 5 membered heterocycloalkylene. In embodiments, L^{1A} is independently substituted or unsubstituted 5 to 6 membered heterocycloalkylene. In embodiments, L^{1A} is independently substituted 5 to 6 membered heterocycloalkylene. In embodiments, L^{1A} is independently unsubstituted 5 to 6 membered heterocycloalkylene.

In embodiments, L^{1A} is independently substituted or unsubstituted arylene. In embodiments, L^{1A} is independently substituted or unsubstituted arylene (e.g., C₆-C₁₂, C₆-C₁₀, or phenyl). In embodiments, L^{1A} is independently substituted arylene (e.g., C₆-C₁₂, C₆-C₁₀, or phenyl). In embodiments, L^{1A} is independently unsubstituted arylene (e.g., C₆-C₁₂, C₆-C₁₀, or phenyl). In embodiments, L^{1A} is independently substituted or unsubstituted C₆-C₁₂ arylene. In embodiments, L^{1A} is independently substituted C₆-C₁₂ arylene. In embodiments, L^{1A} is independently unsubstituted C₆-C₁₂ arylene. In embodiments, L^{1A} is independently substituted or unsubstituted C₆-C₁₀ arylene. In embodiments, L^{1A} is independently substituted C₆-C₁₀ arylene. In embodiments, L^{1A} is independently unsubstituted C₆-C₁₀ arylene. In embodiments, L^{1A} is independently substituted or unsubstituted phenylene. In embodiments, L^{1A} is independently substituted phenylene. In embodiments, L^{1A} is independently unsubstituted phenylene. In embodiments, L^{1A} is independently substituted or unsubstituted biphenylene. In embodiments, L^{1A} is independently substituted biphenylene. In embodiments, L^{1A} is independently unsubstituted biphenylene. In embodiments, L^{1A} is independently substituted or unsubstituted naphthylene. In embodiments, L^{1A} is independently substituted naphthylene. In embodiments, LA is independently unsubstituted naphthylene.

In embodiments, L^{1A} is independently substituted or unsubstituted heteroarylene. In embodiments, L^{1A} is independently substituted heteroarylene. In embodiments, L^{1A} is independently unsubstituted heteroarylene. In embodiments, L^{1A} is independently substituted or unsubstituted heteroarylene (e.g., 5 to 12 membered, 5 to 10 membered, 5 to 9 membered, or 5 to 6 membered). In embodiments, L^{1A} is independently substituted heteroarylene (e.g., 5 to 12 membered, 5 to 10 membered, 5 to 9 membered, or 5 to 6 membered). In embodiments, L^{1A} is independently unsubstituted heteroarylene (e.g., 5 to 12 membered, 5 to 10 membered, 5 to 9 membered, or 5 to 6 membered). In embodiments, L^{1A} is independently substituted or unsubstituted 5 to 12 membered heteroarylene. In embodiments, L^{1A} is independently substituted 5 to 12 membered heteroarylene. In embodiments, L^{1A} is independently unsubstituted 5 to 12 membered heteroarylene. In embodiments, L^{1A} is independently substituted or unsubstituted 5 to 10 membered heteroarylene. In embodiments, L^{1A} is independently substituted 5 to 10 membered heteroarylene. In embodiments, L^{1A} is independently unsubstituted 5 to 10 membered heteroarylene. In embodiments, L^{1A} is independently substituted or unsubstituted 5 to 9 membered heteroarylene. In embodiments, L^{1A} is independently substituted 5 to 9 membered heteroarylene. In embodiments, L^{1A} is independently unsubstituted 5 to 9 membered heteroarylene. In embodiments, L^{1A} is independently substituted or unsubstituted 5 to 6 membered heteroarylene. In embodiments, L^{1A} is independently substituted 5 to 6 membered heteroarylene. In embodiments, L^{1A} is independently unsubstituted 5 to 6 membered heteroarylene.

In embodiments, L^{1B} is independently a bond. In embodiments, L^{1B} is independently —NH—. In embodiments, L^{1B} is independently —O—. In embodiments, L^{1B} is independently —S—. In embodiments, L^{1B} is independently —C(O)—. In embodiments, L^{1B} is independently —NHC(O)—. In embodiments, L^{1B} is independently —S(O)₂C(O)—. In embodiments, L^{1B} is independently —OPO₂—O—. In embodiments, L^{1B} is independently —OP(S)(O)—O—. In embodiments, L^{1B} is independently —OP(S)₂—O—. In embodiments, L^{1B} is independently —S(O)NH—. In embodiments, L^{1B} is independently —NHC(O)NH—. In embodiments, L^{1B} is independently —C(O)O—. In embodiments, L^{1B} is independently —OC(O)—. In embodiments, L^{1B} is independently —C(O)NH—. In embodiments, L^{1B} is not a bond.

In embodiments, L^{1B} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{1B} is independently substituted alkylene (e.g., C₁-C₂₅, C₁-C₂₀, C₁-C₁₂, C₁-C₈, C₁-C₆, C₁-C₄, or C₁-C₂). In embodiments, L^{1B} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{1B} is independently substituted or unsubstituted C₁-C₂₅ alkylene. In embodiments, L^{1B} is independently substituted C₁-C₂₅ alkylene. In embodiments, L^{1B} is independently unsubstituted C₁-C₂₅ alkylene. In embodiments, L^{1B} is independently substituted or unsubstituted C₁-C₂₀ alkylene. In embodiments, L^{1B} is independently substituted C₁-C₂₀ alkylene. In embodiments, L^{1B} is independently unsubstituted C₁-C₂₀ alkylene. In embodiments, L^{1B} is independently substituted or unsubstituted C₁-C₁₂ alkylene. In embodiments, L^{1B} is independently substituted C₁-C₁₂ alkylene. In embodiments, L^{1B} is independently unsubstituted C₁-C₁₂ alkylene. In embodiments, L^{1B} is independently substituted or unsubstituted C₁-C₈ alkylene. In embodiments, L^{1B} is independently substituted C₁-C₈ alkylene. In embodiments, L^{1B} is independently unsubstituted C₁-C₈ alkylene. In embodiments, L^{1B} is independently substituted or unsubstituted C₁-C₆ alkylene. In embodiments, L^{1B} is independently substituted C₁-C₆ alkylene. In embodiments, L^{1B} is independently unsubstituted C₁-C₆ alkylene. In embodiments, L^{1B} is independently substituted or unsubstituted C₁-C₄ alkylene. In embodiments, L^{1B} is independently substituted C₁-C₄ alkylene. In embodiments, L^{1B} is independently unsubstituted C₁-C₄ alkylene. In embodiments, L^{1B} is independently substituted or unsubstituted ethylene. In embodiments, L^{1B} is independently substituted ethylene. In embodiments, L^{1B} is independently unsubstituted ethylene. In embodiments, L^{1B} is independently substituted or unsubstituted methylene. In embodiments, L^{1B} is independently substituted methylene. In embodiments, L^{1B} is independently unsubstituted methylene.

In embodiments, L^{1B} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1B} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1B} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1B} is independently substituted or unsubstituted 2 to 25 membered heteroalkylene. In embodiments, L^{1B} is independently substituted 2 to 25 membered heteroalkylene. In embodiments, L^{1B} is independently unsubstituted 2 to 25 membered heteroalkylene. In embodiments, L^{1B} is independently substituted or unsubstituted 2 to 20 membered heteroalkylene. In embodiments, L^{1B} is independently substituted 2 to 20 membered heteroalkylene. In embodiments, L^{1B} is independently unsubstituted 2 to 20 membered heteroalkylene. In embodiments, L^{1B} is independently substituted or unsubstituted 2 to 12 membered heteroalkylene. In embodiments, L^{1B} is independently substituted 2 to 12 membered heteroalkylene. In embodiments, L^{1B} is independently unsubstituted 2 to 12 membered heteroalkylene. In embodiments, L^{1B} is independently substituted or unsubstituted 2 to 8 membered heteroalkylene. In embodiments, L^{1B} is independently substituted 2 to 8 membered heteroalkylene. In embodiments, L^{1B} is independently unsubstituted 2 to 8 membered heteroalkylene. In embodiments, L^{1B} is independently substituted or unsubstituted 2 to 6 membered heteroalkylene. In embodiments, L^{1B} is independently substituted 2 to 6 membered heteroalkylene. In embodiments, L^{1B} is independently unsubstituted 2 to 6 membered heteroalkylene. In embodiments, L^{1B} is independently substituted or unsubstituted 4 to 6 membered heteroalkylene. In embodiments, L^{1B} is independently substituted 4 to 6 membered heteroalkylene. In embodiments, L^{1B} is independently unsubstituted 4 to 6 membered heteroalkylene. In embodiments, L^{1B} is independently substituted or unsubstituted 2 to 3 membered heteroalkylene. In embodiments, L^{1B} is independently substituted 2 to 3 membered heteroalkylene. In embodiments, L^{1B} is independently unsubstituted 2 to 3 membered heteroalkylene. In embodiments, L^{1B} is independently substituted or unsubstituted 4 to 5 membered heteroalkylene. In embodiments, L^{1B} is independently substituted 4 to 5 membered heteroalkylene. In embodiments, L^{1B} is independently unsubstituted 4 to 5 membered heteroalkylene.

In embodiments, L^{1B} is independently substituted or unsubstituted cyclocycloalkylene (e.g., C₃-C₁₀, C₃-C₈, C₃-C₆, C₄-C₆, or C₅-C₆). In embodiments, L^{1B} is independently substituted cyclocycloalkylene (e.g., C₃-C₁₀, C₃-C₈, C₃-C₆, C₄-C₆, or C₅-C₆). In embodiments, L^{1B} is independently unsubstituted cyclocycloalkylene (e.g., C₃-C₁₀, C₃-C₈, C₃-C₆, C₄-C₆, or C₅-C₆). In embodiments, L^{1B} is independently substituted or unsubstituted C₃-C₁₀ cycloalkylene. In embodiments, L^{1B} is independently substituted C₃-C₁₀ cycloalkylene. In embodiments, L^{1B} is independently unsubstituted C₃-C₁₀ cycloalkylene. In embodiments, L^{1B} is independently substituted or unsubstituted C₃-C₈ cycloalkylene. In embodiments, L^{1B} is independently substituted C₃-C₈ cycloalkylene. In embodiments, L^{1B} is independently unsubstituted C₃-C₈ cycloalkylene. In embodiments, L^{1B} is independently substituted or unsubstituted C₃-C₆ cycloalkylene. In embodiments, L^{1B} is independently substituted C₃-C₆ cycloalkylene. In embodiments, L^{1B} is independently unsubstituted C₃-C₆ cycloalkylene. In embodiments, L^{1B} is independently substituted or unsubstituted C₄-C₆ cycloalkylene. In embodiments, L^{1B} is independently substituted C₄-C₆ cycloalkylene. In embodiments, L^{1B} is independently unsubstituted C₄-C₆ cycloalkylene. In embodiments, L^{1B} is independently substituted or unsubstituted C₅-C₆ cycloalkylene. In embodiments, L^{1B} is independently substituted C₅-C₆ cycloalkylene. In embodiments, L^{1B} is independently unsubstituted C₅-C₆ cycloalkylene. In embodiments, L^{1B} is independently substituted or unsubstituted hexylene. In embodiments, L^{1B} is independently substituted hexylene. In embodiments, L^{1B} is independently unsubstituted hexylene.

In embodiments, L^{1B} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1B} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{1B} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1B} is independently substituted or unsubstituted 3 to 10 membered heterocycloalkylene. In embodiments, L^{1B} is independently substituted 3 to 10 membered heterocycloalkylene. In embodiments, L^{1B} is independently unsubstituted 3 to 10 membered heterocycloalkylene. In embodiments, L^{1B} is independently substituted or unsubstituted 3 to 8 membered heterocycloalkylene. In embodiments, L^{1B} is independently substituted 3 to 8 membered heterocycloalkylene. In embodiments, L^{1B} is independently unsubstituted 3 to 8 membered heterocycloalkylene. In embodiments, L^{1B} is independently substituted or unsubstituted 3 to 6 membered heterocycloalkylene. In embodiments, L^{1B} is independently substituted 3 to 6 membered heterocycloalkylene. In embodiments, L^{1B} is independently unsubstituted 3 to 6 membered heterocycloalkylene. In embodiments, L^{1B} is independently substituted or unsubstituted 4 to 6 membered heterocycloalkylene. In embodiments, L^{1B} is independently substituted 4 to 6 membered heterocycloalkylene. In embodiments, L^{1B} is independently unsubstituted 4 to 6 membered heterocycloalkylene. In embodiments, L^{1B} is independently substituted or unsubstituted 4 to 5 membered heterocycloalkylene. In embodiments, L^{1B} is independently substituted 4 to 5 membered heterocycloalkylene. In embodiments, L^{1B} is independently unsubstituted 4 to 5 membered heterocycloalkylene. In embodiments, L^{1B} is independently substituted or unsubstituted 5 to 6 membered heterocycloalkylene. In embodiments, L^{1B} is independently substituted 5 to 6 membered heterocycloalkylene. In embodiments, L^{1B} is independently unsubstituted 5 to 6 membered heterocycloalkylene.

In embodiments, L^{1B} is independently substituted or unsubstituted arylene. In embodiments, L^{1B} is independently substituted or unsubstituted arylene (e.g., C₆-C₁₂, C₆-C₁₀, or phenyl). In embodiments, L^{1B} is independently substituted arylene (e.g., C₆-C₁₂, C₆-C₁₀, or phenyl). In embodiments, L^{1B} is independently unsubstituted arylene (e.g., C₆-C₁₂, C₆-C₁₀, or phenyl). In embodiments, L^{1B} is independently substituted or unsubstituted C₆-C₁₂ arylene. In embodiments, L^{1B} is independently substituted C₆-C₁₂ arylene. In embodiments, L^{1B} is independently unsubstituted C₆-C₁₂ arylene. In embodiments, L^{1B} is independently substituted or unsubstituted C₆-C₁₀ arylene. In embodiments, L^{1B} is independently substituted C₆-C₁₀ arylene. In embodiments, L^{1B} is independently unsubstituted C₆-C₁₀ arylene. In embodiments, L^{1B} is independently substituted or unsubstituted phenylene. In embodiments, L^{1B} is independently substituted phenylene. In embodiments, L^{1B} is independently unsubstituted phenylene. In embodiments, L^{1B} is independently substituted or unsubstituted biphenylene. In embodiments, L^{1B} is independently substituted biphenylene. In embodiments, L^{1B} is independently unsubstituted biphenylene. In embodiments, L^{1B} is independently substituted or unsubstituted naphthylene. In embodiments, L^{1B} is independently substituted naphthylene. In embodiments, L^{1B} is independently unsubstituted naphthylene.

In embodiments, L^{1B} is independently substituted or unsubstituted heteroarylene. In embodiments, L^{1B} is independently substituted heteroarylene. In embodiments, L^{1B} is independently unsubstituted heteroarylene. In embodiments, L^{1B} is independently substituted or unsubstituted heteroarylene (e.g., 5 to 12 membered, 5 to 10 membered, 5 to 9 membered, or 5 to 6 membered). In embodiments, L^{1B} is independently substituted heteroarylene (e.g., 5 to 12 membered, 5 to 10 membered, 5 to 9 membered, or 5 to 6 membered). In embodiments, L^{1B} is independently unsubstituted heteroarylene (e.g., 5 to 12 membered, 5 to 10 membered, 5 to 9 membered, or 5 to 6 membered). In embodiments, L^{1B} is independently substituted or unsubstituted 5 to 12 membered heteroarylene. In embodiments, L^{1B} is independently substituted 5 to 12 membered heteroarylene. In embodiments, L^{1B} is independently unsubstituted 5 to 12 membered heteroarylene. In embodiments, L^{1B} is independently substituted or unsubstituted 5 to 10 membered heteroarylene. In embodiments, L^{1B} is independently substituted 5 to 10 membered heteroarylene. In embodiments, L^{1B} is independently unsubstituted 5 to 10 membered heteroarylene. In embodiments, L^{1B} is independently substituted or unsubstituted 5 to 9 membered heteroarylene. In embodiments, L^{1B} is independently substituted 5 to 9 membered heteroarylene. In embodiments, L^{1B} is independently unsubstituted 5 to 9 membered heteroarylene. In embodiments, L^{1B} is independently substituted or unsubstituted 5 to 6 membered heteroarylene. In embodiments, L^{1B} is independently substituted 5 to 6 membered heteroarylene. In embodiments, L^{1B} is independently unsubstituted 5 to 6 membered heteroarylene.

In embodiments, L^{1C} is independently a bond. In embodiments, L^{1C} is independently —NH—. In embodiments, L^{1C} is independently —O—. In embodiments, L^{1C} is independently —S—. In embodiments, L^{1C} is independently —C(O)—. In embodiments, L^{1C} is independently —NHC(O)—. In embodiments, L^{1C} is independently —S(O)₂C(O)—. In embodiments, L^{1C} is independently —OPO₂—O—. In embodiments, L^{1C} is independently —OP(S)(O)—O—. In embodiments, L^{1C} is independently —OP(S)₂—O—. In embodiments, L^{1C} is independently —S(O)NH—. In embodiments, L^{1C} is independently —NHC(O)NH—. In embodiments, L^{1C} is independently —C(O)O—. In embodiments, L^{1C} is independently —OC(O)—. In embodiments, L^{1C} is independently —C(O)NH—. In embodiments, L^{1C} is not a bond.

In embodiments, L^{1C} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{1C} is independently substituted alkylene (e.g., C₁-C₂₅, C₁-C₂₀, C₁-C₁₂, C₁-C₈, C₁-C₆, C₁-C₄, or C₁-C₂). In embodiments, L^{1C} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{1C} is independently substituted or unsubstituted C₁-C₂₅ alkylene. In embodiments, L^{1C} is independently substituted C₁-C₂₅ alkylene. In embodiments, L^{1C} is independently unsubstituted C₁-C₂₅ alkylene. In embodiments, L^{1C} is independently substituted or unsubstituted C₁-C₂₀ alkylene. In embodiments, L^{1C} is independently substituted C₁-C₂₀ alkylene. In embodiments, L^{1C} is independently unsubstituted C₁-C₂₀ alkylene. In embodiments, L^{1C} is independently substituted or unsubstituted C₁-C₁₂ alkylene. In embodiments, L^{1C} is independently substituted C₁-C₁₂ alkylene. In embodiments, L^{1C} is independently unsubstituted C₁-C₁₂ alkylene. In embodiments, L^{1C} is independently substituted or unsubstituted C₁-C₈ alkylene. In embodiments, L^{1C} is independently substituted C₁-C₈ alkylene. In embodiments, L^{1C} is independently unsubstituted C₁-C₈ alkylene. In embodiments, L^{1C} is independently substituted or unsubstituted C₁-C₆ alkylene. In embodiments, L^{1C} is independently substituted C₁-C₆ alkylene. In embodiments, L^{1C} is independently unsubstituted C₁-C₆ alkylene. In embodiments, L^{1C} is independently substituted or unsubstituted C₁-C₄ alkylene. In embodiments, L^{1C} is independently substituted C₁-C₄ alkylene. In embodiments, L^{1C} is independently unsubstituted C₁-C₄ alkylene. In embodiments, L^{1C} is independently substituted or unsubstituted ethylene. In embodiments, L^{1C} is independently substituted ethylene. In embodiments, L^{1C} is independently unsubstituted ethylene. In embodiments, L^{1C} is independently substituted or unsubstituted methylene. In embodiments, L^{1C} is independently substituted methylene. In embodiments, L^{1C} is independently unsubstituted methylene.

In embodiments, L^{1C} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1C} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1C} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1C} is independently substituted or unsubstituted 2 to 25 membered heteroalkylene. In embodiments, L^{1C} is independently substituted 2 to 25 membered heteroalkylene. In embodiments, L^{1C} is independently unsubstituted 2 to 25 membered heteroalkylene. In embodiments, L^{1C} is independently substituted or unsubstituted 2 to 20 membered heteroalkylene. In embodiments, L^{1C} is independently substituted 2 to 20 membered heteroalkylene. In embodiments, L^{1C} is independently unsubstituted 2 to 20 membered heteroalkylene. In embodiments, L^{1C} is independently substituted or unsubstituted 2 to 12 membered heteroalkylene. In embodiments, L^{1C} is independently substituted 2 to 12 membered heteroalkylene. In embodiments, L^{1C} is independently unsubstituted 2 to 12 membered heteroalkylene. In embodiments, L^{1C} is independently substituted or unsubstituted 2 to 8 membered heteroalkylene. In embodiments, L^{1C} is independently substituted 2 to 8 membered heteroalkylene. In embodiments, L^{1C} is independently unsubstituted 2 to 8 membered heteroalkylene. In embodiments, L^{1C} is independently substituted or unsubstituted 2 to 6 membered heteroalkylene. In embodiments, L^{1C} is independently substituted 2 to 6 membered heteroalkylene. In embodiments, L^{1C} is independently unsubstituted 2 to 6 membered heteroalkylene. In embodiments, L^{1C} is independently substituted or unsubstituted 4 to 6 membered heteroalkylene. In embodiments, L^{1C} is independently substituted 4 to 6 membered heteroalkylene. In embodiments, L^{1C} is independently unsubstituted 4 to 6 membered heteroalkylene. In embodiments, L^{1C} is independently substituted or unsubstituted 2 to 3 membered heteroalkylene. In embodiments, L^{1C} is independently substituted 2 to 3 membered heteroalkylene. In embodiments, L^{1C} is independently unsubstituted 2 to 3 membered heteroalkylene. In embodiments, L^{1C} is independently substituted or unsubstituted 4 to 5 membered heteroalkylene. In embodiments, L^{1C} is independently substituted 4 to 5 membered heteroalkylene. In embodiments, L^{1C} is independently unsubstituted 4 to 5 membered heteroalkylene.

In embodiments, L^{1C} is independently substituted or unsubstituted cyclocycloalkylene (e.g., C₃-C₁₀, C₃-C₈, C₃-C₆, C₄-C₆, or C₅-C₆). In embodiments, L^{1C} is independently substituted cyclocycloalkylene (e.g., C₃-C₁₀, C₃-C₈, C₃-C₆, C₄-C₆, or C₅-C₆). In embodiments, L^{1C} is independently unsubstituted cyclocycloalkylene (e.g., C₃-C₁₀, C₃-C₈, C₃-C₆, C₄-C₆, or C₅-C₆). In embodiments, L^{1C} is independently substituted or unsubstituted C₃-C₁₀ cycloalkylene. In embodiments, L^{1C} is independently substituted C₃-C₁₀ cycloalkylene. In embodiments, L^{1C} is independently unsubstituted C₃-C₁₀ cycloalkylene. In embodiments, L^{1C} is independently substituted or unsubstituted C₃-C₈ cycloalkylene. In embodiments, L^{1C} is independently substituted C₃-C₈ cycloalkylene. In embodiments, L^{1C} is independently unsubstituted C₃-C₈ cycloalkylene. In embodiments, L^{1C} is independently substituted or unsubstituted C₃-C₆ cycloalkylene. In embodiments, L^{1C} is independently substituted C₃-C₆ cycloalkylene. In embodiments, L^{1C} is independently unsubstituted C₃-C₆ cycloalkylene. In embodiments, L^{1C} is independently substituted or unsubstituted C₄-C₆ cycloalkylene. In embodiments, L^{1C} is independently substituted C₄-C₆ cycloalkylene. In embodiments, L^{1C} is independently unsubstituted C₄-C₆ cycloalkylene. In embodiments, L^{1C} is independently substituted or unsubstituted C₅-C₆ cycloalkylene. In embodiments, L^{1C} is independently substituted C₅-C₆ cycloalkylene. In embodiments, L^{1C} is independently unsubstituted C₅-C₆ cycloalkylene. In embodiments, L^{1C} is independently substituted or unsubstituted hexylene. In embodiments, L^{1C} is independently substituted hexylene. In embodiments, L^{1C} is independently unsubstituted hexylene.

In embodiments, L^{1C} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1C} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{1C} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1C} is independently substituted or unsubstituted 3 to 10 membered heterocycloalkylene. In embodiments, L^{1C} is independently substituted 3 to 10 membered heterocycloalkylene. In embodiments, L^{1C} is independently unsubstituted 3 to 10 membered heterocycloalkylene. In embodiments, L^{1C} is independently substituted or unsubstituted 3 to 8 membered heterocycloalkylene. In embodiments, L^{1C} is independently substituted 3 to 8 membered heterocycloalkylene. In embodiments, L^{1C} is independently unsubstituted 3 to 8 membered heterocycloalkylene. In embodiments, L^{1C} is independently substituted or unsubstituted 3 to 6 membered heterocycloalkylene. In embodiments, L^{1C} is independently substituted 3 to 6 membered heterocycloalkylene. In embodiments, L^{1C} is independently unsubstituted 3 to 6 membered heterocycloalkylene. In embodiments, L^{1C} is independently substituted or unsubstituted 4 to 6 membered heterocycloalkylene. In embodiments, L^{1C} is independently substituted 4 to 6 membered heterocycloalkylene. In embodiments, L^{1C} is independently unsubstituted 4 to 6 membered heterocycloalkylene. In embodiments, L^{1C} is independently substituted or unsubstituted 4 to 5 membered heterocycloalkylene. In embodiments, L^{1C} is independently substituted 4 to 5 membered heterocycloalkylene. In embodiments, L^{1C} is independently unsubstituted 4 to 5 membered heterocycloalkylene. In embodiments, L^{1C} is independently substituted or unsubstituted 5 to 6 membered heterocycloalkylene. In embodiments, L^{1C} is independently substituted 5 to 6 membered heterocycloalkylene. In embodiments, L^{1C} is independently unsubstituted 5 to 6 membered heterocycloalkylene.

In embodiments, L^{1C} is independently substituted or unsubstituted arylene. In embodiments, L^{1C} is independently substituted or unsubstituted arylene (e.g., C₆-C₁₂, C₆-C₁₀, or phenyl). In embodiments, L^{1C} is independently substituted arylene (e.g., C₆-C₁₂, C₆-C₁₀, or phenyl). In embodiments, L^{1C} is independently unsubstituted arylene (e.g., C₆-C₁₂, C₆-C₁₀, or phenyl). In embodiments, L^{1C} is independently substituted or unsubstituted C₆-C₁₂ arylene. In embodiments, L^{1C} is independently substituted C₆-C₁₂ arylene. In embodiments, L^{1C} is independently unsubstituted C₆-C₁₂ arylene. In embodiments, L^{1C} is independently substituted or unsubstituted C₆-C₁₀ arylene. In embodiments, L^{1C} is independently substituted C₆-C₁₀ arylene. In embodiments, L^{1C} is independently unsubstituted C₆-C₁₀ arylene. In embodiments, L^{1C} is independently substituted or unsubstituted phenylene. In embodiments, L^{1C} is independently substituted phenylene. In embodiments, L^{1C} is independently unsubstituted phenylene. In embodiments, L^{1C} is independently substituted or unsubstituted biphenylene. In embodiments, L^{1C} is independently substituted biphenylene. In embodiments, L^{1C} is independently unsubstituted biphenylene. In embodiments, L^{1C} is independently substituted or unsubstituted naphthylene. In embodiments, L^{1C} is independently substituted naphthylene. In embodiments, L^{1C} is independently unsubstituted naphthylene.

In embodiments, L^{1C} is independently substituted or unsubstituted heteroarylene. In embodiments, L^{1C} is independently substituted heteroarylene. In embodiments, L^{1C} is independently unsubstituted heteroarylene. In embodiments, L^{1C} is independently substituted or unsubstituted heteroarylene (e.g., 5 to 12 membered, 5 to 10 membered, 5 to 9 membered, or 5 to 6 membered). In embodiments, L^{1C} is independently substituted heteroarylene (e.g., 5 to 12 membered, 5 to 10 membered, 5 to 9 membered, or 5 to 6 membered). In embodiments, L^{1C} is independently unsubstituted heteroarylene (e.g., 5 to 12 membered, 5 to 10 membered, 5 to 9 membered, or 5 to 6 membered). In embodiments, L^{1C} is independently substituted or unsubstituted 5 to 12 membered heteroarylene. In embodiments, L^{1C} is independently substituted 5 to 12 membered heteroarylene. In embodiments, L^{1C} is independently unsubstituted 5 to 12 membered heteroarylene. In embodiments, L^{1C} is independently substituted or unsubstituted 5 to 10 membered heteroarylene. In embodiments, L^{1C} is independently substituted 5 to 10 membered heteroarylene. In embodiments, L^{1C} is independently unsubstituted 5 to 10 membered heteroarylene. In embodiments, L^{1C} is independently substituted or unsubstituted 5 to 9 membered heteroarylene. In embodiments, L^{1C} is independently substituted 5 to 9 membered heteroarylene. In embodiments, L^{1C} is independently unsubstituted 5 to 9 membered heteroarylene. In embodiments, L^{1C} is independently substituted or unsubstituted 5 to 6 membered heteroarylene. In embodiments, L^{1C} is independently substituted 5 to 6 membered heteroarylene. In embodiments, L^{1C} is independently unsubstituted 5 to 6 membered heteroarylene.

In embodiments, L¹⁰ is independently a bond. In embodiments, L^{1D} is independently —NH—. In embodiments, L^{1D} is independently —O—. In embodiments, L^{1D} is independently —S—. In embodiments, L^{1D} is independently —C(O)—. In embodiments, L^{1D} is independently —NHC(O)—. In embodiments, L^{1D} is independently —S(O)₂C(O)—. In embodiments, L^{1D} is independently —OPO₂—O—. In embodiments, L^{1D} is independently —OP(S)(O)—O—. In embodiments, L^{1D} is independently —OP(S)₂—O—. In embodiments, L^{1D} is independently —S(O)NH—. In embodiments, L^{1D} is independently —NHC(O)NH—. In embodiments, L^{1D} is independently —C(O)O—. In embodiments, L^{1D} is independently —OC(O)—. In embodiments, L^{1D} is independently —C(O)NH—. In embodiments, L^{1D} is not a bond.

In embodiments, L^{1D} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{1D} is independently substituted alkylene (e.g., C₁-C₂₅, C₁-C₂₀, C₁-C₁₂, C₁-C₈, C₁-C₆, C₁-C₄, or C₁-C₂). In embodiments, L^{1D} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{1D} is independently substituted or unsubstituted C₁-C₂₅ alkylene. In embodiments, L^{1D} is independently substituted C₁-C₂₅ alkylene. In embodiments, L^{1D} is independently unsubstituted C₁-C₂₅ alkylene. In embodiments, L^{1D} is independently substituted or unsubstituted C₁-C₂₀ alkylene. In embodiments, L^{1D} is independently substituted C₁-C₂₀ alkylene. In embodiments, L^{1D} is independently unsubstituted C₁-C₂₀ alkylene. In embodiments, L^{1D} is independently substituted or unsubstituted C₁-C₁₂ alkylene. In embodiments, L^{1D} is independently substituted C₁-C₁₂ alkylene. In embodiments, L^{1D} is independently unsubstituted C₁-C₁₂ alkylene. In embodiments, L^{1D} is independently substituted or unsubstituted C₁-C₈ alkylene. In embodiments, L^{1D} is independently substituted C₁-C₈ alkylene. In embodiments, L^{1D} is independently unsubstituted C₁-C₈ alkylene. In embodiments, L^{1D} is independently substituted or unsubstituted C₁-C₆ alkylene. In embodiments, L¹⁰ is independently substituted C₁-C₆ alkylene. In embodiments, L^{1D} is independently unsubstituted C₁-C₆ alkylene. In embodiments, L^{1D} is independently substituted or unsubstituted C₁-C₄ alkylene. In embodiments, L¹⁰ is independently substituted C₁-C₄ alkylene. In embodiments, L^{1D} is independently unsubstituted C₁-C₄ alkylene. In embodiments, L^{1D} is independently substituted or unsubstituted ethylene. In embodiments, L^{1D} is independently substituted ethylene. In embodiments, L^{1D} is independently unsubstituted ethylene. In embodiments, L^{1D} is independently substituted or unsubstituted methylene. In embodiments, L^{1D} is independently substituted methylene. In embodiments, L^{1D} is independently unsubstituted methylene.

In embodiments, L^{1D} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1D} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1D} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1D} is independently substituted or unsubstituted 2 to 25 membered heteroalkylene. In embodiments, L^{1D} is independently substituted 2 to 25 membered heteroalkylene. In embodiments, L^{1D} is independently unsubstituted 2 to 25 membered heteroalkylene. In embodiments, L^{1D} is independently substituted or unsubstituted 2 to 20 membered heteroalkylene. In embodiments, L^{1D} is independently substituted 2 to 20 membered heteroalkylene. In embodiments, L^{1D} is independently unsubstituted 2 to 20 membered heteroalkylene. In embodiments, L^{1D} is independently substituted or unsubstituted 2 to 12 membered heteroalkylene. In embodiments, L^{1D} is independently substituted 2 to 12 membered heteroalkylene. In embodiments, L¹⁰ is independently unsubstituted 2 to 12 membered heteroalkylene. In embodiments, L^{1D} is independently substituted or unsubstituted 2 to 8 membered heteroalkylene. In embodiments, L^{1D} is independently substituted 2 to 8 membered heteroalkylene. In embodiments, L^{1D} is independently unsubstituted 2 to 8 membered heteroalkylene. In embodiments, L^{1D} is independently substituted or unsubstituted 2 to 6 membered heteroalkylene. In embodiments, L^{1D} is independently substituted 2 to 6 membered heteroalkylene. In embodiments, L^{1D} is independently unsubstituted 2 to 6 membered heteroalkylene. In embodiments, L^{1D} is independently substituted or unsubstituted 4 to 6 membered heteroalkylene. In embodiments, L^{1D} is independently substituted 4 to 6 membered heteroalkylene. In embodiments, L^{1D} is independently unsubstituted 4 to 6 membered heteroalkylene. In embodiments, L^{1D} is independently substituted or unsubstituted 2 to 3 membered heteroalkylene. In embodiments, L^{1D} is independently substituted 2 to 3 membered heteroalkylene. In embodiments, L^{1D} is independently unsubstituted 2 to 3 membered heteroalkylene. In embodiments, L^{1D} is independently substituted or unsubstituted 4 to 5 membered heteroalkylene. In embodiments, L¹⁰ is independently substituted 4 to 5 membered heteroalkylene. In embodiments, L^{1D} is independently unsubstituted 4 to 5 membered heteroalkylene.

In embodiments, L^{1D} is independently substituted or unsubstituted cyclocycloalkylene (e.g., C₃-C₁₀, C₃-C₈, C₃-C₆, C₄-C₆, or C₅-C₆). In embodiments, L^{1D} is independently substituted cyclocycloalkylene (e.g., C₃-C₁₀, C₃-C₈, C₃-C₆, C₄-C₆, or C₅-C₆). In embodiments, L^{1D} is independently unsubstituted cyclocycloalkylene (e.g., C₃-C₁₀, C₃-C₈, C₃-C₆, C₄-C₆, or C₅-C₆). In embodiments, L^{1D} is independently substituted or unsubstituted C₃-C₁₀ cycloalkylene. In embodiments, L^{1D} is independently substituted C₃-C₁₀ cycloalkylene. In embodiments, L^{1D} is independently unsubstituted C₃-C₁₀ cycloalkylene. In embodiments, L^{1D} is independently substituted or unsubstituted C₃-C₈ cycloalkylene. In embodiments, L^{1D} is independently substituted C₃-C₈ cycloalkylene. In embodiments, L^{1D} is independently unsubstituted C₃-C₈ cycloalkylene. In embodiments, L^{1D} is independently substituted or unsubstituted C₃-C₆ cycloalkylene. In embodiments, L^{1D} is independently substituted C₃-C₆ cycloalkylene. In embodiments, L^{1D} is independently unsubstituted C₃-C₆ cycloalkylene. In embodiments, L^{1D} is independently substituted or unsubstituted C₄-C₆ cycloalkylene. In embodiments, L^{1D} is independently substituted C₄-C₆ cycloalkylene. In embodiments, L^{1D} is independently unsubstituted C₄-C₆ cycloalkylene. In embodiments, L^{1D} is independently substituted or unsubstituted C₅-C₆ cycloalkylene. In embodiments, L^{1D} is independently substituted C₅-C₆ cycloalkylene. In embodiments, L¹⁰ is independently unsubstituted C₅-C₆ cycloalkylene. In embodiments, L^{1D} is independently substituted or unsubstituted hexylene. In embodiments, L^{1D} is independently substituted hexylene. In embodiments, L^{1D} is independently unsubstituted hexylene.

In embodiments, L^{1D} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1D} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{1D} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1D} is independently substituted or unsubstituted 3 to 10 membered heterocycloalkylene. In embodiments, L^{1D} is independently substituted 3 to 10 membered heterocycloalkylene. In embodiments, L^{1D} is independently unsubstituted 3 to 10 membered heterocycloalkylene. In embodiments, L^{1D} is independently substituted or unsubstituted 3 to 8 membered heterocycloalkylene. In embodiments, L^{1D} is independently substituted 3 to 8 membered heterocycloalkylene. In embodiments, L^{1D} is independently unsubstituted 3 to 8 membered heterocycloalkylene. In embodiments, L^{1D} is independently substituted or unsubstituted 3 to 6 membered heterocycloalkylene. In embodiments, L^{1D} is independently substituted 3 to 6 membered heterocycloalkylene. In embodiments, L^{1D} is independently unsubstituted 3 to 6 membered heterocycloalkylene. In embodiments, L^{1D} is independently substituted or unsubstituted 4 to 6 membered heterocycloalkylene. In embodiments, L^{1D} is independently substituted 4 to 6 membered heterocycloalkylene. In embodiments, L^{1D} is independently unsubstituted 4 to 6 membered heterocycloalkylene. In embodiments, L^{1D} is independently substituted or unsubstituted 4 to 5 membered heterocycloalkylene. In embodiments, L^{1D} is independently substituted 4 to 5 membered heterocycloalkylene. In embodiments, L^{1D} is independently unsubstituted 4 to 5 membered heterocycloalkylene. In embodiments, L^{1D} is independently substituted or unsubstituted 5 to 6 membered heterocycloalkylene. In embodiments, L^{1D} is independently substituted 5 to 6 membered heterocycloalkylene. In embodiments, L^{1D} is independently unsubstituted 5 to 6 membered heterocycloalkylene.

In embodiments, L^{1D} is independently substituted or unsubstituted arylene. In embodiments, L^{1D} is independently substituted or unsubstituted arylene (e.g., C₆-C₁₂, C₆-C₁₀, or phenyl). In embodiments, L^{1D} is independently substituted arylene (e.g., C₆-C₁₂, C₆-C₁₀, or phenyl). In embodiments, L^{1D} is independently unsubstituted arylene (e.g., C₆-C₁₂, C₆-C₁₀, or phenyl). In embodiments, L^{1D} is independently substituted or unsubstituted C₆-C₁₂ arylene. In embodiments, L^{1D} is independently substituted C₆-C₁₂ arylene. In embodiments, L^{1D} is independently unsubstituted C₆-C₁₂ arylene. In embodiments, L¹⁰ is independently substituted or unsubstituted C₆-C₁₀ arylene. In embodiments, L^{1D} is independently substituted C₆-C₁₀ arylene. In embodiments, L^{1D} is independently unsubstituted C₆-C₁₀ arylene. In embodiments, L^{1D} is independently substituted or unsubstituted phenylene. In embodiments, L^{1D} is independently substituted phenylene. In embodiments, L^{1D} is independently unsubstituted phenylene. In embodiments, L^{1D} is independently substituted or unsubstituted biphenylene. In embodiments, L¹⁰ is independently substituted biphenylene. In embodiments, L^{1D} is independently unsubstituted biphenylene. In embodiments, L^{1D} is independently substituted or unsubstituted naphthylene. In embodiments, L^{1D} is independently substituted naphthylene. In embodiments, L^{1D} is independently unsubstituted naphthylene.

In embodiments, L¹⁰ is independently substituted or unsubstituted heteroarylene. In embodiments, L^{1D} is independently substituted heteroarylene. In embodiments, L^{1D} is independently unsubstituted heteroarylene. In embodiments, L^{1D} is independently substituted or unsubstituted heteroarylene (e.g., 5 to 12 membered, 5 to 10 membered, 5 to 9 membered, or 5 to 6 membered). In embodiments, L¹⁰ is independently substituted heteroarylene (e.g., 5 to 12 membered, 5 to 10 membered, 5 to 9 membered, or 5 to 6 membered). In embodiments, L^{1D} is independently unsubstituted heteroarylene (e.g., 5 to 12 membered, 5 to 10 membered, 5 to 9 membered, or 5 to 6 membered). In embodiments, L^{1D} is independently substituted or unsubstituted 5 to 12 membered heteroarylene. In embodiments, L^{1D} is independently substituted 5 to 12 membered heteroarylene. In embodiments, L^{1D} is independently unsubstituted 5 to 12 membered heteroarylene. In embodiments, L^{1D} is independently substituted or unsubstituted 5 to 10 membered heteroarylene. In embodiments, L^{1D} is independently substituted 5 to 10 membered heteroarylene. In embodiments, L^{1D} is independently unsubstituted 5 to 10 membered heteroarylene. In embodiments, L^{1D} is independently substituted or unsubstituted 5 to 9 membered heteroarylene. In embodiments, L^{1D} is independently substituted 5 to 9 membered heteroarylene. In embodiments, L^{1D} is independently unsubstituted 5 to 9 membered heteroarylene. In embodiments, L^{1D} is independently substituted or unsubstituted 5 to 6 membered heteroarylene. In embodiments, L^{1D} is independently substituted 5 to 6 membered heteroarylene. In embodiments, L^{1D} is independently unsubstituted 5 to 6 membered heteroarylene.

In embodiments, L^{1E} is independently a bond. In embodiments, L^{1E} is independently —NH—. In embodiments, L^{1E} is independently —O—. In embodiments, L^{1E} is independently —S—. In embodiments, L^{1E} is independently —C(O)—. In embodiments, L^{1E} is independently —NHC(O)—. In embodiments, L^{1E} is independently —S(O)₂C(O)—. In embodiments, L^{1E} is independently —OPO₂—O—. In embodiments, L^{1E} is independently —OP(S)(O)—O—. In embodiments, L^{1E} is independently —OP(S)₂—O—. In embodiments, L^{1E} is independently —S(O)NH—. In embodiments, L^{1E} is independently —NHC(O)NH—. In embodiments, L^{1E} is independently —C(O)O—. In embodiments, L^{1E} is independently —OC(O)—. In embodiments, L^{1E} is independently —C(O)NH—. In embodiments, L^{1E} is not a bond.

In embodiments, L^{1E} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{1E} is independently substituted alkylene (e.g., C₁-C₂₅, C₁-C₂₀, C₁-C₁₂, C₁-C₈, C₁-C₆, C₁-C₄, or C₁-C₂). In embodiments, L^{1E} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{1E} is independently substituted or unsubstituted C₁-C₂₅ alkylene. In embodiments, L^{1E} is independently substituted C₁-C₂₅ alkylene. In embodiments, L^{1E} is independently unsubstituted C₁-C₂₅ alkylene. In embodiments, L^{1E} is independently substituted or unsubstituted C₁-C₂₀ alkylene. In embodiments, L^{1E} is independently substituted C₁-C₂₀ alkylene. In embodiments, L^{1E} is independently unsubstituted C₁-C₂₀ alkylene. In embodiments, L^{1E} is independently substituted or unsubstituted C₁-C₁₂ alkylene. In embodiments, L^{1E} is independently substituted C₁-C₁₂ alkylene. In embodiments, L^{1E} is independently unsubstituted C₁-C₁₂ alkylene. In embodiments, L^{1E} is independently substituted or unsubstituted C₁-C₈ alkylene. In embodiments, L^{1E} is independently substituted C₁-C₈ alkylene. In embodiments, L^{1E} is independently unsubstituted C₁-C₈alkylene. In embodiments, L^{1E} is independently substituted or unsubstituted C₁-C₆ alkylene. In embodiments, L^{1E} is independently substituted C₁-C₆ alkylene. In embodiments, L^{1E} is independently unsubstituted C₁-C₆ alkylene. In embodiments, L^{1E} is independently substituted or unsubstituted C₁-C₄ alkylene. In embodiments, L^{1E} is independently substituted C₁-C₄ alkylene. In embodiments, L^{1E} is independently unsubstituted C₁-C₄ alkylene. In embodiments, L^{1E} is independently substituted or unsubstituted ethylene. In embodiments, L^{1E} is independently substituted ethylene. In embodiments, L^{1E} is independently unsubstituted ethylene. In embodiments, L^{1E} is independently substituted or unsubstituted methylene. In embodiments, L^{1E} is independently substituted methylene. In embodiments, L^{1E} is independently unsubstituted methylene.

In embodiments, L^{1E} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1E} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1E} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{1E} is independently substituted or unsubstituted 2 to 25 membered heteroalkylene. In embodiments, L^{1E} is independently substituted 2 to 25 membered heteroalkylene. In embodiments, L^{1E} is independently unsubstituted 2 to 25 membered heteroalkylene. In embodiments, L^{1E} is independently substituted or unsubstituted 2 to 20 membered heteroalkylene. In embodiments, L^{1E} is independently substituted 2 to 20 membered heteroalkylene. In embodiments, L^{1E} is independently unsubstituted 2 to 20 membered heteroalkylene. In embodiments, L^{1E} is independently substituted or unsubstituted 2 to 12 membered heteroalkylene. In embodiments, L^{1E} is independently substituted 2 to 12 membered heteroalkylene. In embodiments, L^{1E} is independently unsubstituted 2 to 12 membered heteroalkylene. In embodiments, L^{1E} is independently substituted or unsubstituted 2 to 8 membered heteroalkylene. In embodiments, L^{1E} is independently substituted 2 to 8 membered heteroalkylene. In embodiments, L^{1E} is independently unsubstituted 2 to 8 membered heteroalkylene. In embodiments, L^{1E} is independently substituted or unsubstituted 2 to 6 membered heteroalkylene. In embodiments, L^{1E} is independently substituted 2 to 6 membered heteroalkylene. In embodiments, L^{1E} is independently unsubstituted 2 to 6 membered heteroalkylene. In embodiments, L^{1E} is independently substituted or unsubstituted 4 to 6 membered heteroalkylene. In embodiments, L^{1E} is independently substituted 4 to 6 membered heteroalkylene. In embodiments, L^{1E} is independently unsubstituted 4 to 6 membered heteroalkylene. In embodiments, L^{1E} is independently substituted or unsubstituted 2 to 3 membered heteroalkylene. In embodiments, L^{1E} is independently substituted 2 to 3 membered heteroalkylene. In embodiments, L^{1E} is independently unsubstituted 2 to 3 membered heteroalkylene. In embodiments, L^{1E} is independently substituted or unsubstituted 4 to 5 membered heteroalkylene. In embodiments, L^{1E} is independently substituted 4 to 5 membered heteroalkylene. In embodiments, L^{1E} is independently unsubstituted 4 to 5 membered heteroalkylene.

In embodiments, L^{1E} is independently substituted or unsubstituted cyclocycloalkylene (e.g., C₃-C₁₀, C₃-C₈, C₃-C₆, C₄-C₆, or C₅-C₆). In embodiments, L^{1E} is independently substituted cyclocycloalkylene (e.g., C₃-C₁₀, C₃-C₈, C₃-C₆, C₄-C₆, or C₅-C₆). In embodiments, L^{1E} is independently unsubstituted cyclocycloalkylene (e.g., C₃-C₁₀, C₃-C₈, C₃-C₆, C₄-C₆, or C₅-C₆). In embodiments, L^{1E} is independently substituted or unsubstituted C₃-C₁₀ cycloalkylene. In embodiments, L^{1E} is independently substituted C₃-C₁₀ cycloalkylene. In embodiments, L^{1E} is independently unsubstituted C₃-C₁₀ cycloalkylene. In embodiments, L^{1E} is independently substituted or unsubstituted C₃-C₈ cycloalkylene. In embodiments, L^{1E} is independently substituted C₃-C₈ cycloalkylene. In embodiments, L^{1E} is independently unsubstituted C₃-C₈ cycloalkylene. In embodiments, L^{1E} is independently substituted or unsubstituted C₃-C₆ cycloalkylene. In embodiments, L^{1E} is independently substituted C₃-C₆ cycloalkylene. In embodiments, L^{1E} is independently unsubstituted C₃-C₆ cycloalkylene. In embodiments, L^{1E} is independently substituted or unsubstituted C₄-C₆ cycloalkylene. In embodiments, L^{1E} is independently substituted C₄-C₆ cycloalkylene. In embodiments, L^{1E} is independently unsubstituted C₄-C₆ cycloalkylene. In embodiments, L^{1E} is independently substituted or unsubstituted C₅-C₆ cycloalkylene. In embodiments, L^{1E} is independently substituted C₅-C₆ cycloalkylene. In embodiments, L^{1E} is independently unsubstituted C₅-C₆ cycloalkylene. In embodiments, L^{1E} is independently substituted or unsubstituted hexylene. In embodiments, L^{1E} is independently substituted hexylene. In embodiments, L^{1E} is independently unsubstituted hexylene.

In embodiments, L^{1E} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1E} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{1E} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{1E} is independently substituted or unsubstituted 3 to 10 membered heterocycloalkylene. In embodiments, L^{1E} is independently substituted 3 to 10 membered heterocycloalkylene. In embodiments, L^{1E} is independently unsubstituted 3 to 10 membered heterocycloalkylene. In embodiments, L^{1E} is independently substituted or unsubstituted 3 to 8 membered heterocycloalkylene. In embodiments, L^{1E} is independently substituted 3 to 8 membered heterocycloalkylene. In embodiments, L^{1E} is independently unsubstituted 3 to 8 membered heterocycloalkylene. In embodiments, L^{1E} is independently substituted or unsubstituted 3 to 6 membered heterocycloalkylene. In embodiments, L^{1E} is independently substituted 3 to 6 membered heterocycloalkylene. In embodiments, L^{1E} is independently unsubstituted 3 to 6 membered heterocycloalkylene. In embodiments, L^{1E} is independently substituted or unsubstituted 4 to 6 membered heterocycloalkylene. In embodiments, L^{1E} is independently substituted 4 to 6 membered heterocycloalkylene. In embodiments, L^{1E} is independently unsubstituted 4 to 6 membered heterocycloalkylene. In embodiments, L^{1E} is independently substituted or unsubstituted 4 to 5 membered heterocycloalkylene. In embodiments, L^{1E} is independently substituted 4 to 5 membered heterocycloalkylene. In embodiments, L^{1E} is independently unsubstituted 4 to 5 membered heterocycloalkylene. In embodiments, L^{1E} is independently substituted or unsubstituted 5 to 6 membered heterocycloalkylene. In embodiments, L^{1E} is independently substituted 5 to 6 membered heterocycloalkylene. In embodiments, L^{1E} is independently unsubstituted 5 to 6 membered heterocycloalkylene.

In embodiments, L^{1E} is independently substituted or unsubstituted arylene. In embodiments, L^{1E} is independently substituted or unsubstituted arylene (e.g., C₆-C₁₂, C₆-C₁₀, or phenyl). In embodiments, L^{1E} is independently substituted arylene (e.g., C₆-C₁₂, C₆-C₁₀, or phenyl). In embodiments, L^{1E} is independently unsubstituted arylene (e.g., C₆-C₁₂, C₆-C₁₀, or phenyl). In embodiments, L^{1E} is independently substituted or unsubstituted C₆-C₁₂ arylene. In embodiments, L^{1E} is independently substituted C₆-C₁₂ arylene. In embodiments, L^{1E} is independently unsubstituted C₆-C₁₂ arylene. In embodiments, L^{1E} is independently substituted or unsubstituted C₆-C₁₀ arylene. In embodiments, L^{1E} is independently substituted C₆-C₁₀ arylene. In embodiments, L^{1E} is independently unsubstituted C₆-C₁₀ arylene. In embodiments, L^{1E} is independently substituted or unsubstituted phenylene. In embodiments, L^{1E} is independently substituted phenylene. In embodiments, L^{1E} is independently unsubstituted phenylene. In embodiments, L^{1E} is independently substituted or unsubstituted biphenylene. In embodiments, L^{1E} is independently substituted biphenylene. In embodiments, L^{1E} is independently unsubstituted biphenylene. In embodiments, L^{1E} is independently substituted or unsubstituted naphthylene. In embodiments, L^{1E} is independently substituted naphthylene. In embodiments, L^{1E} is independently unsubstituted naphthylene.

In embodiments, L^{1E} is independently substituted or unsubstituted heteroarylene. In embodiments, L^{1E} is independently substituted heteroarylene. In embodiments, L^{1E} is independently unsubstituted heteroarylene. In embodiments, L^{1E} is independently substituted or unsubstituted heteroarylene (e.g., 5 to 12 membered, 5 to 10 membered, 5 to 9 membered, or 5 to 6 membered). In embodiments, L^{1E} is independently substituted heteroarylene (e.g., 5 to 12 membered, 5 to 10 membered, 5 to 9 membered, or 5 to 6 membered). In embodiments, L^{1E} is independently unsubstituted heteroarylene (e.g., 5 to 12 membered, 5 to 10 membered, 5 to 9 membered, or 5 to 6 membered). In embodiments, L^{1E} is independently substituted or unsubstituted 5 to 12 membered heteroarylene. In embodiments, L^{1E} is independently substituted 5 to 12 membered heteroarylene. In embodiments, L^{1E} is independently unsubstituted 5 to 12 membered heteroarylene. In embodiments, L^{1E} is independently substituted or unsubstituted 5 to 10 membered heteroarylene. In embodiments, L^{1E} is independently substituted 5 to 10 membered heteroarylene. In embodiments, L^{1E} is independently unsubstituted 5 to 10 membered heteroarylene. In embodiments, L^{1E} is independently substituted or unsubstituted 5 to 9 membered heteroarylene. In embodiments, L^{1E} is independently substituted 5 to 9 membered heteroarylene. In embodiments, L^{1E} is independently unsubstituted 5 to 9 membered heteroarylene. In embodiments, L^{1E} is independently substituted or unsubstituted 5 to 6 membered heteroarylene. In embodiments, L^{1E} is independently substituted 5 to 6 membered heteroarylene. In embodiments, L^{1E} is independently unsubstituted 5 to 6 membered heteroarylene.

In embodiments, L¹ is independently -L¹¹-NH—C(O)— or -L¹¹-C(O)—NH—, and L¹¹ is independently substituted or unsubstituted C₁-C₂₀ alkylene, substituted or unsubstituted 2-20 membered heteroalkylene, or substituted or unsubstituted 2-20 membered heteroalkenylene.

In embodiments, L¹¹ is independently substituted or unsubstituted alkylene (e.g., C₁-C₂₀, C₁-C₁₂, C₁-C₈, C₁-C₆, C₁-C₄, or C₁-C₂). In embodiments, L¹¹ is independently substituted alkylene (e.g., C₁-C₂₀, C₁-C₁₂, C₁-C₈, C₁-C₆, C₁-C₄, or C₁-C₂). In embodiments, L¹¹ is independently unsubstituted alkylene (e.g., C₁-C₂₀, C₁-C₁₂, C₁-C₈, C₁-C₆, C₁-C₄, or C₁-C₂). In embodiments, L¹¹ is independently substituted or unsubstituted C₁-C₂₀ alkylene. In embodiments, L¹¹ is independently substituted C₁-C₂₀ alkylene. In embodiments, L¹¹ is independently unsubstituted C₁-C₂₀ alkylene. In embodiments, L¹¹ is independently substituted or unsubstituted C₁-C₁₂ alkylene. In embodiments, L¹¹ is independently substituted C₁-C₁₂ alkylene. In embodiments, L¹¹ is independently unsubstituted C₁-C₁₂ alkylene. In embodiments, L¹¹ is independently substituted or unsubstituted C₁-C₈ alkylene. In embodiments, L¹¹ is independently substituted C₁-C₈ alkylene. In embodiments, L¹¹ is independently unsubstituted C₁-C₈ alkylene. In embodiments, L¹¹ is independently substituted or unsubstituted C₁-C₆ alkylene. In embodiments, L¹¹ is independently substituted C₁-C₆ alkylene. In embodiments, L¹¹ is independently unsubstituted C₁-C₆ alkylene. In embodiments, L¹¹ is independently substituted or unsubstituted C₁-C₄ alkylene. In embodiments, L¹¹ is independently substituted C₁-C₄ alkylene. In embodiments, L¹¹ is independently unsubstituted C₁-C₄ alkylene. In embodiments, L¹¹ is independently substituted or unsubstituted ethylene. In embodiments, L¹¹ is independently substituted ethylene. In embodiments, L¹¹ is independently unsubstituted ethylene. In embodiments, L¹¹ is independently substituted or unsubstituted methylene. In embodiments, L¹¹ is independently substituted methylene. In embodiments, L¹¹ is independently unsubstituted methylene.

In embodiments, L¹¹ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹¹ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹¹ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹¹ is independently substituted or unsubstituted 2 to 20 membered heteroalkylene. In embodiments, L¹¹ is independently substituted 2 to 20 membered heteroalkylene. In embodiments, L¹¹ is independently unsubstituted 2 to 20 membered heteroalkylene. In embodiments, L¹¹ is independently substituted or unsubstituted 2 to 12 membered heteroalkylene. In embodiments, L¹¹ is independently substituted 2 to 12 membered heteroalkylene. In embodiments, L¹¹ is independently unsubstituted 2 to 12 membered heteroalkylene. In embodiments, L¹¹ is independently substituted or unsubstituted 2 to 8 membered heteroalkylene. In embodiments, L¹¹ is independently substituted 2 to 8 membered heteroalkylene. In embodiments, L¹¹ is independently unsubstituted 2 to 8 membered heteroalkylene. In embodiments, L¹¹ is independently substituted or unsubstituted 2 to 6 membered heteroalkylene. In embodiments, L¹¹ is independently substituted 2 to 6 membered heteroalkylene. In embodiments, L¹¹ is independently unsubstituted 2 to 6 membered heteroalkylene. In embodiments, L¹¹ is independently substituted or unsubstituted 4 to 6 membered heteroalkylene. In embodiments, L¹¹ is independently substituted 4 to 6 membered heteroalkylene. In embodiments, L¹¹ is independently unsubstituted 4 to 6 membered heteroalkylene. In embodiments, L¹¹ is independently substituted or unsubstituted 2 to 3 membered heteroalkylene. In embodiments, L¹¹ is independently substituted 2 to 3 membered heteroalkylene. In embodiments, L¹¹ is independently unsubstituted 2 to 3 membered heteroalkylene. In embodiments, L¹¹ is independently substituted or unsubstituted 4 to 5 membered heteroalkylene. In embodiments, L¹¹ is independently substituted 4 to 5 membered heteroalkylene. In embodiments, L¹¹ is independently unsubstituted 4 to 5 membered heteroalkylene.

In embodiments, L¹¹ is independently substituted or unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹¹ is independently substituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹¹ is independently unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹¹ is independently substituted or unsubstituted 2 to 20 membered heteroalkenylene. In embodiments, L¹¹ is independently substituted 2 to 20 membered heteroalkenylene. In embodiments, L¹¹ is independently unsubstituted 2 to 20 membered heteroalkenylene. In embodiments, L¹¹ is independently substituted or unsubstituted 2 to 12 membered heteroalkenylene. In embodiments, L¹¹ is independently substituted 2 to 12 membered heteroalkenylene. In embodiments, L¹¹ is independently unsubstituted 2 to 12 membered heteroalkenylene. In embodiments, L¹¹ is independently substituted or unsubstituted 2 to 8 membered heteroalkenylene. In embodiments, L¹¹ is independently substituted 2 to 8 membered heteroalkenylene. In embodiments, L¹¹ is independently unsubstituted 2 to 8 membered heteroalkenylene. In embodiments, L¹¹ is independently substituted or unsubstituted 2 to 6 membered heteroalkenylene. In embodiments, L¹¹ is independently substituted 2 to 6 membered heteroalkenylene. In embodiments, L¹¹ is independently unsubstituted 2 to 6 membered heteroalkenylene. In embodiments, L¹¹ is independently substituted or unsubstituted 4 to 6 membered heteroalkenylene. In embodiments, L¹¹ is independently substituted 4 to 6 membered heteroalkenylene. In embodiments, L¹¹ is independently unsubstituted 4 to 6 membered heteroalkenylene. In embodiments, L¹¹ is independently substituted or unsubstituted 2 to 3 membered heteroalkenylene. In embodiments, L¹¹ is independently substituted 2 to 3 membered heteroalkenylene. In embodiments, L¹¹ is independently unsubstituted 2 to 3 membered heteroalkenylene. In embodiments, L¹¹ is independently substituted or unsubstituted 4 to 5 membered heteroalkenylene. In embodiments, L¹¹ is independently substituted 4 to 5 membered heteroalkenylene. In embodiments, L¹¹ is independently unsubstituted 4 to 5 membered heteroalkenylene.

In embodiments, L¹ is independently -L¹¹-NH—C(O)—. In embodiments, L¹ is independently -L¹¹-C(O)—NH—. In embodiments, L¹¹ is substituted or unsubstituted C₁-C₈ alkylene. In embodiments, L¹¹ is substituted C₁-C₈ alkylene. In embodiments, L¹¹ is unsubstituted C₁-C₈ alkylene.

In embodiments, L¹ is independently

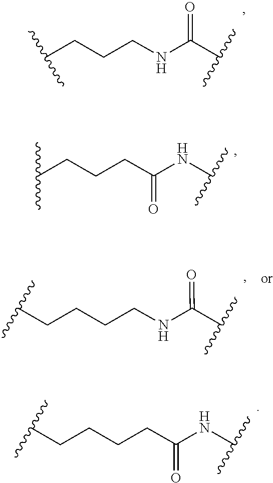 [SMILES: *CCCNC(*)=O ; *CCCC(=O)N* ; *CCCCNC(*)=O ; *CCCCC(=O)N*]

In embodiments, L¹ is independently

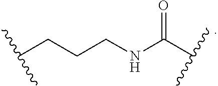 [SMILES: *CCCNC(*)=O]

In embodiments, L¹ is independently

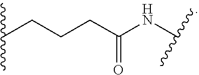 [SMILES: *CCCC(=O)N*]

In embodiments, L¹ is independently

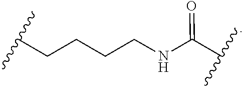 [SMILES: *CCCCNC(*)=O]

In embodiments, L¹ is independently

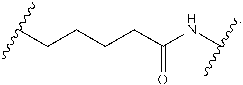 [SMILES: *CCCCC(=O)N*]

In embodiments, L¹ is independently a bond,

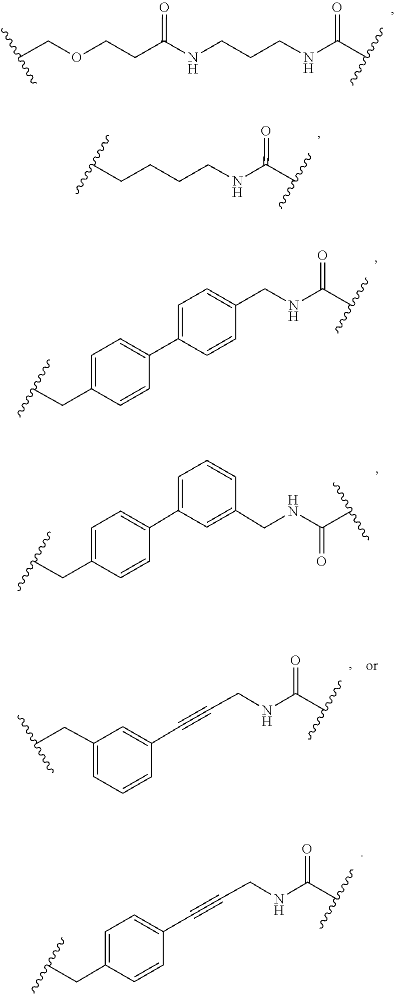 [SMILES: *COCCC(=O)NCCCNC(*)=O ; *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L¹ is independently a bond. In embodiments, L¹ is independently

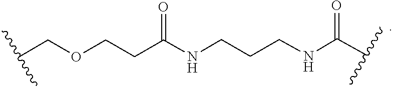 [SMILES: *COCCC(=O)NCCCNC(*)=O]

In embodiments, L¹ is independently

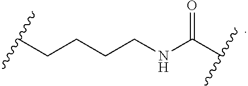 [SMILES: *CCCCNC(*)=O]

In embodiments, L¹ is independently

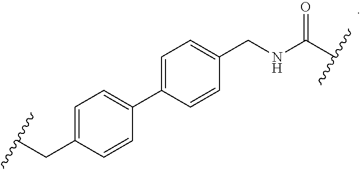 [SMILES: *Cc1ccc(-c2ccc(CNC(*)=O)cc2)cc1]

In embodiments, L¹ is independently

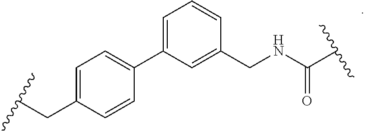 [SMILES: *Cc1ccc(-c2cccc(CNC(*)=O)c2)cc1]

In embodiments, L¹ is independently

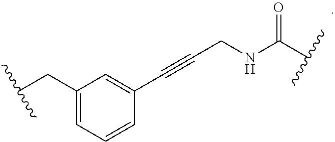 [SMILES: *Cc1cccc(C#CCNC(*)=O)c1]

In embodiments, L¹ is independently

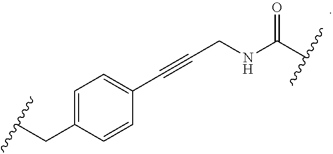 [SMILES: *Cc1ccc(C#CCNC(*)=O)cc1]

In embodiments, L^{1A} is independently substituted or unsubstituted 2 to 8 membered heteroalkylene; L^{1B} is independently —NHC(O)— or —C(O)NH—; L^{1C} is independently unsubstituted C₁-C₈alkylene; L^{1D} is independently a bond, —NHC(O)—, or —C(O)NH—; and L^{1E} is independently —NHC(O)—. In embodiments, L^{1A} is independently substituted or unsubstituted 2 to 5 membered heteroalkylene; L^{1B} is independently —NHC(O)— or —C(O)NH—; L^{1C} is independently unsubstituted C₁-C₄ alkylene; L^{1D} is independently a bond, —NHC(O)—, or —C(O)NH—; and L^{1E} is independently —NHC(O)—. In embodiments, L^{1A} is independently unsubstituted 4 to 5 membered heteroalkylene; L^{1B} is independently —NHC(O)— or —C(O)NH—; L^{1C} is independently unsubstituted C₁-C₄ alkylene; L^{1D} is independently a bond; and L^{1E} is independently —NHC(O)—.

In embodiments, L² is independently —NHC(O)—, —C(O)NH—, substituted or unsubstituted alkylene, or substituted or unsubstituted heteroalkylene. In embodiments, L² is independently —NHC(O)—. In embodiments, L² is independently —C(O)NH—. In embodiments, L² is substituted or unsubstituted alkylene. In embodiments, L² is substituted or unsubstituted heteroalkylene.

In embodiments, L² is independently substituted or unsubstituted alkylene (e.g., C₁-C₂₀, C₁-C₁₂, C₁-C₈, C₁-C₆, C₁-C₄, or C₁-C₂). In embodiments, L² is independently substituted alkylene (e.g., C₁-C₂₀, C₁-C₁₂, C₁-C₈, C₁-C₆, C₁-C₄, or C₁-C₂). In embodiments, L² is independently unsubstituted alkylene (e.g., C₁-C₂₀, C₁-C₁₂, C₁-C₈, C₁-C₆, C₁-C₄, or C₁-C₂). In embodiments, L² is independently substituted or unsubstituted C₁-C₂₀ alkylene. In embodiments, L² is independently substituted C₁-C₂₀ alkylene. In embodiments, L² is independently unsubstituted C₁-C₂₀ alkylene. In embodiments, L² is independently substituted or unsubstituted C₁-C₁₂ alkylene. In embodiments, L² is independently substituted C₁-C₁₂ alkylene. In embodiments, L² is independently unsubstituted C₁-C₁₂ alkylene. In embodiments, L² is independently substituted or unsubstituted C₁-C₈ alkylene. In embodiments, L² is independently substituted C₁-C₈ alkylene. In embodiments, L² is independently unsubstituted C₁-C₈alkylene. In embodiments, L² is independently substituted or unsubstituted C₁-C₆ alkylene. In embodiments, L² is independently substituted C₁-C₆ alkylene. In embodiments, L² is independently unsubstituted C₁-C₆ alkylene. In embodiments, L² is independently substituted or unsubstituted C₁-C₄ alkylene. In embodiments, L² is independently substituted C₁-C₄ alkylene. In embodiments, L² is independently unsubstituted C₁-C₄ alkylene. In embodiments, L² is independently substituted or unsubstituted ethylene. In embodiments, L² is independently substituted ethylene. In embodiments, L² is independently unsubstituted ethylene. In embodiments, L² is independently substituted or unsubstituted methylene. In embodiments, L² is independently substituted methylene. In embodiments, L² is independently unsubstituted methylene.

In embodiments, L² is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L² is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L² is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L² is independently substituted or unsubstituted 2 to 25 membered heteroalkylene. In embodiments, L² is independently substituted 2 to 25 membered heteroalkylene. In embodiments, L² is independently unsubstituted 2 to 25 membered heteroalkylene. In embodiments, L² is independently substituted or unsubstituted 2 to 20 membered heteroalkylene. In embodiments, L² is independently substituted 2 to 20 membered heteroalkylene. In embodiments, L² is independently unsubstituted 2 to 20 membered heteroalkylene. In embodiments, L² is independently substituted or unsubstituted 2 to 12 membered heteroalkylene. In embodiments, L² is independently substituted 2 to 12 membered heteroalkylene. In embodiments, L² is independently unsubstituted 2 to 12 membered heteroalkylene. In embodiments, L² is independently substituted or unsubstituted 2 to 8 membered heteroalkylene. In embodiments, L² is independently substituted 2 to 8 membered heteroalkylene. In embodiments, L² is independently unsubstituted 2 to 8 membered heteroalkylene. In embodiments, L² is independently substituted or unsubstituted 2 to 6 membered heteroalkylene. In embodiments, L² is independently substituted 2 to 6 membered heteroalkylene. In embodiments, L² is independently unsubstituted 2 to 6 membered heteroalkylene. In embodiments, L² is independently substituted or unsubstituted 4 to 6 membered heteroalkylene. In embodiments, L² is independently substituted 4 to 6 membered heteroalkylene. In embodiments, L² is independently unsubstituted 4 to 6 membered heteroalkylene. In embodiments, L² is independently substituted or unsubstituted 2 to 3 membered heteroalkylene. In embodiments, L² is independently substituted 2 to 3 membered heteroalkylene. In embodiments, L² is independently unsubstituted 2 to 3 membered heteroalkylene. In embodiments, L² is independently substituted or unsubstituted 4 to 5 membered heteroalkylene. In embodiments, L² is independently substituted 4 to 5 membered heteroalkylene. In embodiments, L² is independently unsubstituted 4 to 5 membered heteroalkylene.

In embodiments, L² is L^{2A}-L^{2B}-L^{2C}-L^{2D}-L^{2E}. In embodiments, L^{2A} is independently a bond, —NHC(O)—, substituted or unsubstituted alkylene, or substituted or unsubstituted heteroalkylene; L^{2B} is independently a bond, —O—, —NHC(O)—, substituted or unsubstituted alkylene, substituted or unsubstituted heteroalkylene, or substituted or unsubstituted arylene; L^{2C} is independently a bond, —O—, —NHC(O)—, substituted or unsubstituted alkylene, substituted or substituted or unsubstituted heteroalkylene, or substituted or unsubstituted arylene; L^{2D} is independently a bond, —O—, —NHC(O)—, substituted or unsubstituted alkylene, substituted or unsubstituted heteroalkylene, or substituted or unsubstituted arylene; and L^{2E} is independently a bond, —NHC(O)—, substituted or unsubstituted alkylene, or substituted or unsubstituted heteroalkylene.

In embodiments, L^{2A} is independently a bond, —NHC(O)—, substituted or unsubstituted alkylene, or substituted or unsubstituted heteroalkylene. In embodiments, L^{2B} is independently a bond, —O—, —NHC(O)—, substituted or unsubstituted alkylene, substituted or unsubstituted heteroalkylene, or substituted or unsubstituted arylene. In embodiments, L^{2C} is independently a bond, —O—, —NHC(O)—, substituted or unsubstituted alkylene, substituted or substituted or unsubstituted heteroalkylene, or substituted or unsubstituted arylene. In embodiments, L^{2D} is independently a bond, —O—, —NHC(O)—, substituted or unsubstituted alkylene, substituted or unsubstituted heteroalkylene, or substituted or unsubstituted arylene. In embodiments, L^{2E} is independently a bond, —NHC(O)—, substituted or unsubstituted alkylene, or substituted or unsubstituted heteroalkylene.

In embodiments, L^{2A} is independently a bond, —NHC(O)—, substituted or unsubstituted C₁-C₈ alkylene, or substituted or unsubstituted 2 to 8 membered heteroalkylene; L^{2B} is independently a bond, —O—, —NHC(O)—, substituted or unsubstituted C₁-C₈ alkylene, substituted or unsubstituted 2 to 8 membered heteroalkylene, or substituted or unsubstituted phenylene; L^{2C} is independently a bond, —O—, —NHC(O)—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene; L^{2D} is independently a bond, —O—, —NHC(O)—, substituted or unsubstituted C₁-C₈ alkylene, substituted or unsubstituted 2 to 8 membered heteroalkylene, or substituted or unsubstituted phenylene; and L^{2E} is independently a bond, —NHC(O)—, substituted or unsubstituted C₁-C₈ alkylene, or substituted or unsubstituted 2 to 8 membered heteroalkylene. In embodiments, L^{2A} is independently a bond, —NHC(O)—, substituted or unsubstituted C₁-C₈ alkylene, or substituted or unsubstituted 2 to 8 membered heteroalkylene. In embodiments, L^{2B} is independently a bond, —O—, —NHC(O)—, substituted or unsubstituted C₁-C₈ alkylene, substituted or unsubstituted 2 to 8 membered heteroalkylene, or substituted or unsubstituted phenylene. In embodiments, L^{2C} is independently a bond, —O—, —NHC(O)—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene. In embodiments, L^{2D} is independently a bond, —O—, —NHC(O)—, substituted or unsubstituted C₁-C₈ alkylene, substituted or unsubstituted 2 to 8 membered heteroalkylene, or substituted or unsubstituted phenylene. In embodiments, L^{2E} is independently a bond, —NHC(O)—, substituted or unsubstituted C₁-C₈ alkylene, or substituted or unsubstituted 2 to 8 membered heteroalkylene.

In embodiments, L^{2A} is independently unsubstituted C₁-C₈ alkylene, or unsubstituted 2 to 8 membered heteroalkylene; L^{2B} is independently a bond, —O—, —NHC(O)—, unsubstituted C₁-C₈ alkylene, unsubstituted C₂-C₈ alkynylene, unsubstituted 2 to 8 membered heteroalkylene, or unsubstituted phenylene; L^{2C} is independently a bond, —O—, —NHC(O)—, unsubstituted C₁-C₈ alkylene, unsubstituted 2 to 8 membered heteroalkylene, or unsubstituted phenylene; L^{2D} is independently a bond, —O—, —NHC(O)—, unsubstituted C₁-C₈ alkylene, or unsubstituted 2 to 8 membered heteroalkylene; and L^{2E} is independently —NHC(O)—.

In embodiments, L^{2A} is independently unsubstituted C₁-C₈ alkylene, or unsubstituted 2 to 8 membered heteroalkylene. In embodiments, L^{2B} is independently a bond, —O—, —NHC(O)—, unsubstituted C₁-C₈ alkylene, unsubstituted C₂-C₈ alkynylene, unsubstituted 2 to 8 membered heteroalkylene, or unsubstituted phenylene. In embodiments, L^{2C} is independently a bond, —O—, —NHC(O)—, unsubstituted C₁-C₈ alkylene, unsubstituted 2 to 8 membered heteroalkylene, or unsubstituted phenylene. In embodiments, L^{2D} is independently a bond, —O—, —NHC(O)—, unsubstituted C₁-C₈ alkylene, or unsubstituted 2 to 8 membered heteroalkylene. In embodiments, L^{2E} is independently —NHC(O)—.

In embodiments, L^{2A} is independently a bond. In embodiments, L^{2A} is independently —NH—. In embodiments, L^{2A} is independently —O—. In embodiments, L^{2A} is independently —S—. In embodiments, L^{2A} is independently —C(O)—. In embodiments, L^{2A} is independently —NHC(O)—. In embodiments, L^{2A} is independently —S(O)₂C(O)—. In embodiments, L^{2A} is independently —OPO₂—O—. In embodiments, L^{2A} is independently —OP(S)(O)—O—. In embodiments, L^{2A} is independently —OP(S)₂—O—. In embodiments, L^{2A} is independently —S(O)NH—. In embodiments, L^{2A} is independently —NHC(O)NH—. In embodiments, L^{2A} is independently —C(O)O—. In embodiments, L^{2A} is independently —OC(O)—. In embodiments, L^{2A} is independently —C(O)NH—. In embodiments, L^{2A} is not a bond.

In embodiments, L^{2A} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{2A} is independently substituted alkylene (e.g., C₁-C₂₅, C₁-C₂₀, C₁-C₁₂, C₁-C₈, C₁-C₆, C₁-C₄, or C₁-C₂). In embodiments, L^{2A} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{2A} is independently substituted or unsubstituted C₁-C₂₅ alkylene. In embodiments, L^{2A} is independently substituted C₁-C₂₅ alkylene. In embodiments, L^{2A} is independently unsubstituted C₁-C₂₅ alkylene. In embodiments, L^{2A} is independently substituted or unsubstituted C₁-C₂₀ alkylene. In embodiments, L^{2A} is independently substituted C₁-C₂₀ alkylene. In embodiments, L^{2A} is independently unsubstituted C₁-C₂₀ alkylene. In embodiments, L^{2A} is independently substituted or unsubstituted C₁-C₁₂ alkylene. In embodiments, L^{2A} is independently substituted C₁-C₁₂ alkylene. In embodiments, L^{2A} is independently unsubstituted C₁-C₁₂ alkylene. In embodiments, L^{2A} is independently substituted or unsubstituted C₁-C₈ alkylene. In embodiments, L^{2A} is independently substituted C₁-C₈ alkylene. In embodiments, L^{2A} is independently unsubstituted C₁-C₈ alkylene. In embodiments, L^{2A} is independently substituted or unsubstituted C₁-C₆ alkylene. In embodiments, L^{2A} is independently substituted C₁-C₆ alkylene. In embodiments, L^{2A} is independently unsubstituted C₁-C₆ alkylene. In embodiments, L^{2A} is independently substituted or unsubstituted C₁-C₄ alkylene. In embodiments, L^{2A} is independently substituted C₁-C₄ alkylene. In embodiments, L^{2A} is independently unsubstituted C₁-C₄ alkylene. In embodiments, L^{2A} is independently substituted or unsubstituted ethylene. In embodiments, L^{2A} is independently substituted ethylene. In embodiments, L^{2A} is independently unsubstituted ethylene. In embodiments, L^{2A} is independently substituted or unsubstituted methylene. In embodiments, L^{2A} is independently substituted methylene. In embodiments, L^{2A} is independently unsubstituted methylene.

In embodiments, L^{2A} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2A} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2A} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2A} is independently substituted or unsubstituted 2 to 25 membered heteroalkylene. In embodiments, L^{2A} is independently substituted 2 to 25 membered heteroalkylene. In embodiments, L^{2A} is independently unsubstituted 2 to 25 membered heteroalkylene. In embodiments, L^{2A} is independently substituted or unsubstituted 2 to 20 membered heteroalkylene. In embodiments, L^{2A} is independently substituted 2 to 20 membered heteroalkylene. In embodiments, L^{2A} is independently unsubstituted 2 to 20 membered heteroalkylene. In embodiments, L^{2A} is independently substituted or unsubstituted 2 to 12 membered heteroalkylene. In embodiments, L^{2A} is independently substituted 2 to 12 membered heteroalkylene. In embodiments, L^{2A} is independently unsubstituted 2 to 12 membered heteroalkylene. In embodiments, L^{2A} is independently substituted or unsubstituted 2 to 8 membered heteroalkylene. In embodiments, L^{2A} is independently substituted 2 to 8 membered heteroalkylene. In embodiments, L^{2A} is independently unsubstituted 2 to 8 membered heteroalkylene. In embodiments, L^{2A} is independently substituted or unsubstituted 2 to 6 membered heteroalkylene. In embodiments, L^{2A} is independently substituted 2 to 6 membered heteroalkylene. In embodiments, L^{2A} is independently unsubstituted 2 to 6 membered heteroalkylene. In embodiments, L^{2A} is independently substituted or unsubstituted 4 to 6 membered heteroalkylene. In embodiments, L^{2A} is independently substituted 4 to 6 membered heteroalkylene. In embodiments, L^{2A} is independently unsubstituted 4 to 6 membered heteroalkylene. In embodiments, L^{2A} is independently substituted or unsubstituted 2 to 3 membered heteroalkylene. In embodiments, L^{2A} is independently substituted 2 to 3 membered heteroalkylene. In embodiments, L^{2A} is independently unsubstituted 2 to 3 membered heteroalkylene. In embodiments, L^{2A} is independently substituted or unsubstituted 4 to 5 membered heteroalkylene. In embodiments, L^{2A} is independently substituted 4 to 5 membered heteroalkylene. In embodiments, L^{2A} is independently unsubstituted 4 to 5 membered heteroalkylene.

In embodiments, L^{2A} is independently substituted or unsubstituted cyclocycloalkylene (e.g., C₃-C₁₀, C₃-C₈, C₃-C₆, C₄-C₆, or C₅-C₆). In embodiments, L^{2A} is independently substituted cyclocycloalkylene (e.g., C₃-C₁₀, C₃-C₈, C₃-C₆, C₄-C₆, or C₅-C₆). In embodiments, L^{2A} is independently unsubstituted cyclocycloalkylene (e.g., C₃-C₁₀, C₃-C₈, C₃-C₆, C₄-C₆, or C₅-C₆). In embodiments, L^{2A} is independently substituted or unsubstituted C₃-C₁₀ cycloalkylene. In embodiments, L^{2A} is independently substituted C₃-C₁₀ cycloalkylene. In embodiments, L^{2A} is independently unsubstituted C₃-C₁₀ cycloalkylene. In embodiments, L^{2A} is independently substituted or unsubstituted C₃-C₈ cycloalkylene. In embodiments, L^{2A} is independently substituted C₃-C₈ cycloalkylene. In embodiments, L^{2A} is independently unsubstituted C₃-C₈ cycloalkylene. In embodiments, L^{2A} is independently substituted or unsubstituted C₃-C₆ cycloalkylene. In embodiments, L^{2A} is independently substituted C₃-C₆ cycloalkylene. In embodiments, L^{2A} is independently unsubstituted C₃-C₆ cycloalkylene. In embodiments, L^{2A} is independently substituted or unsubstituted C₄-C₆ cycloalkylene. In embodiments, L^{2A} is independently substituted C₄-C₆ cycloalkylene. In embodiments, L^{2A} is independently unsubstituted C₄-C₆ cycloalkylene. In embodiments, L^{2A} is independently substituted or unsubstituted C₅-C₆ cycloalkylene. In embodiments, L^{2A} is independently substituted C₅-C₆ cycloalkylene. In embodiments, L^{2A} is independently unsubstituted C₅-C₆ cycloalkylene. In embodiments, L^{2A} is independently substituted or unsubstituted hexylene. In embodiments, L^{2A} is independently substituted hexylene. In embodiments, L^{2A} is independently unsubstituted hexylene.

In embodiments, L^{2A} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2A} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{2A} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2A} is independently substituted or unsubstituted 3 to 10 membered heterocycloalkylene. In embodiments, L^{2A} is independently substituted 3 to 10 membered heterocycloalkylene. In embodiments, L^{2A} is independently unsubstituted 3 to 10 membered heterocycloalkylene. In embodiments, L^{2A} is independently substituted or unsubstituted 3 to 8 membered heterocycloalkylene. In embodiments, L^{2A} is independently substituted 3 to 8 membered heterocycloalkylene. In embodiments, L^{2A} is independently unsubstituted 3 to 8 membered heterocycloalkylene. In embodiments, L^{2A} is independently substituted or unsubstituted 3 to 6 membered heterocycloalkylene. In embodiments, L^{2A} is independently substituted 3 to 6 membered heterocycloalkylene. In embodiments, L^{2A} is independently unsubstituted 3 to 6 membered heterocycloalkylene. In embodiments, L^{2A} is independently substituted or unsubstituted 4 to 6 membered heterocycloalkylene. In embodiments, L^{2A} is independently substituted 4 to 6 membered heterocycloalkylene. In embodiments, L^{2A} is independently unsubstituted 4 to 6 membered heterocycloalkylene. In embodiments, L^{2A} is independently substituted or unsubstituted 4 to 5 membered heterocycloalkylene. In embodiments, L^{2A} is independently substituted 4 to 5 membered heterocycloalkylene. In embodiments, L^{2A} is independently unsubstituted 4 to 5 membered heterocycloalkylene. In embodiments, L^{2A} is independently substituted or unsubstituted 5 to 6 membered heterocycloalkylene. In embodiments, L^{2A} is independently substituted 5 to 6 membered heterocycloalkylene. In embodiments, L^{2A} is independently unsubstituted 5 to 6 membered heterocycloalkylene.

In embodiments, L^{2A} is independently substituted or unsubstituted arylene. In embodiments, L^{2A} is independently substituted or unsubstituted arylene (e.g., C₆-C₁₂, C₆-C₁₀, or phenyl). In embodiments, L^{2A} is independently substituted arylene (e.g., C₆-C₁₂, C₆-C₁₀, or phenyl). In embodiments, L^{2A} is independently unsubstituted arylene (e.g., C₆-C₁₂, C₆-C₁₀, or phenyl). In embodiments, L^{2A} is independently substituted or unsubstituted C₆-C₁₂ arylene. In embodiments, L^{2A} is independently substituted C₆-C₁₂ arylene. In embodiments, L^{2A} is independently unsubstituted C₆-C₁₂ arylene. In embodiments, L^{2A} is independently substituted or unsubstituted C₆-C₁₀ arylene. In embodiments, L^{2A} is independently substituted C₆-C₁₀ arylene. In embodiments, L^{2A} is independently unsubstituted C₆-C₁₀ arylene. In embodiments, L^{2A} is independently substituted or unsubstituted phenylene. In embodiments, L^{2A} is independently substituted phenylene. In embodiments, L^{2A} is independently unsubstituted phenylene. In embodiments, L^{2A} is independently substituted or unsubstituted biphenylene. In embodiments, L^{2A} is independently substituted biphenylene. In embodiments, L^{2A} is independently unsubstituted biphenylene. In embodiments, L^{2A} is independently substituted or unsubstituted naphthylene. In embodiments, L^{2A} is independently substituted naphthylene. In embodiments, L^{2A} is independently unsubstituted naphthylene.

In embodiments, L^{2A} is independently substituted or unsubstituted heteroarylene. In embodiments, L^{2A} is independently substituted heteroarylene. In embodiments, L^{2A} is independently unsubstituted heteroarylene. In embodiments, L^{2A} is independently substituted or unsubstituted heteroarylene (e.g., 5 to 12 membered, 5 to 10 membered, 5 to 9 membered, or 5 to 6 membered). In embodiments, L^{2A} is independently substituted heteroarylene (e.g., 5 to 12 membered, 5 to 10 membered, 5 to 9 membered, or 5 to 6 membered). In embodiments, L^{2A} is independently unsubstituted heteroarylene (e.g., 5 to 12 membered, 5 to 10 membered, 5 to 9 membered, or 5 to 6 membered). In embodiments, L^{2A} is independently substituted or unsubstituted 5 to 12 membered heteroarylene. In embodiments, L^{2A} is independently substituted 5 to 12 membered heteroarylene. In embodiments, L^{2A} is independently unsubstituted 5 to 12 membered heteroarylene. In embodiments, L^{2A} is independently substituted or unsubstituted 5 to 10 membered heteroarylene. In embodiments, L^{2A} is independently substituted 5 to 10 membered heteroarylene. In embodiments, L^{2A} is independently unsubstituted 5 to 10 membered heteroarylene. In embodiments, L^{2A} is independently substituted or unsubstituted 5 to 9 membered heteroarylene. In embodiments, L^{2A} is independently substituted 5 to 9 membered heteroarylene. In embodiments, L^{2A} is independently unsubstituted 5 to 9 membered heteroarylene. In embodiments, L^{2A} is independently substituted or unsubstituted 5 to 6 membered heteroarylene. In embodiments, L^{2A} is independently substituted 5 to 6 membered heteroarylene. In embodiments, L^{2A} is independently unsubstituted 5 to 6 membered heteroarylene.

In embodiments, L^{2B} is independently a bond. In embodiments, L^{2B} is independently —NH—. In embodiments, L^{2B} is independently —O—. In embodiments, L^{2B} is independently —S—. In embodiments, L^{2B} is independently —C(O)—. In embodiments, L^{2B} is independently —NHC(O)—. In embodiments, L^{2B} is independently —S(O)₂C(O)—. In embodiments, L^{2B} is independently —OPO₂—O—. In embodiments, L^{2B} is independently —OP(S)(O)—O—. In embodiments, L^{2B} is independently —OP(S)₂—O—. In embodiments, L^{2B} is independently —S(O)NH—. In embodiments, L^{2B} is independently —NHC(O)NH—. In embodiments, L^{2B} is independently —C(O)O—. In embodiments, L^{2B} is independently —OC(O)—. In embodiments, L^{2B} is independently —C(O)NH—. In embodiments, L^{2B} is not a bond.

In embodiments, L^{2B} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{2B} is independently substituted alkylene (e.g., C₁-C₂₅, C₁-C₂₀, C₁-C₁₂, C₁-C₈, C₁-C₆, C₁-C₄, or C₁-C₂). In embodiments, L^{2B} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{2B} is independently substituted or unsubstituted C₁-C₂₅ alkylene. In embodiments, L^{2B} is independently substituted C₁-C₂₅ alkylene. In embodiments, L^{2B} is independently unsubstituted C₁-C₂₅ alkylene. In embodiments, L^{2B} is independently substituted or unsubstituted C₁-C₂₀ alkylene. In embodiments, L^{2B} is independently substituted C₁-C₂₀ alkylene. In embodiments, L^{2B} is independently unsubstituted C₁-C₂₀ alkylene. In embodiments, L^{2B} is independently substituted or unsubstituted C₁-C₁₂ alkylene. In embodiments, L^{2B} is independently substituted C₁-C₁₂ alkylene. In embodiments, L^{2B} is independently unsubstituted C₁-C₁₂ alkylene. In embodiments, L^{2B} is independently substituted or unsubstituted C₁-C₈ alkylene. In embodiments, L^{2B} is independently substituted C₁-C₈ alkylene. In embodiments, L^{2B} is independently unsubstituted C₁-C₈ alkylene. In embodiments, L^{2B} is independently substituted or unsubstituted C₁-C₆ alkylene. In embodiments, L^{2B} is independently substituted C₁-C₆ alkylene. In embodiments, L^{2B} is independently unsubstituted C₁-C₆ alkylene. In embodiments, L^{2B} is independently substituted or unsubstituted C₁-C₄ alkylene. In embodiments, L^{2B} is independently substituted C₁-C₄ alkylene. In embodiments, L^{2B} is independently unsubstituted C₁-C₄ alkylene. In embodiments, L^{2B} is independently substituted or unsubstituted ethylene. In embodiments, L^{2B} is independently substituted ethylene. In embodiments, L^{2B} is independently unsubstituted ethylene. In embodiments, L^{2B} is independently substituted or unsubstituted methylene. In embodiments, L^{2B} is independently substituted methylene. In embodiments, L^{2B} is independently unsubstituted methylene.

In embodiments, L^{2B} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2B} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2B} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2B} is independently substituted or unsubstituted 2 to 25 membered heteroalkylene. In embodiments, L^{2B} is independently substituted 2 to 25 membered heteroalkylene. In embodiments, L^{2B} is independently unsubstituted 2 to 25 membered heteroalkylene. In embodiments, L^{2B} is independently substituted or unsubstituted 2 to 20 membered heteroalkylene. In embodiments, L^{2B} is independently substituted 2 to 20 membered heteroalkylene. In embodiments, L^{2B} is independently unsubstituted 2 to 20 membered heteroalkylene. In embodiments, L^{2B} is independently substituted or unsubstituted 2 to 12 membered heteroalkylene. In embodiments, L^{2B} is independently substituted 2 to 12 membered heteroalkylene. In embodiments, L^{2B} is independently unsubstituted 2 to 12 membered heteroalkylene. In embodiments, L^{2B} is independently substituted or unsubstituted 2 to 8 membered heteroalkylene. In embodiments, L^{2B} is independently substituted 2 to 8 membered heteroalkylene. In embodiments, L^{2B} is independently unsubstituted 2 to 8 membered heteroalkylene. In embodiments, L^{2B} is independently substituted or unsubstituted 2 to 6 membered heteroalkylene. In embodiments, L^{2B} is independently substituted 2 to 6 membered heteroalkylene. In embodiments, L^{2B} is independently unsubstituted 2 to 6 membered heteroalkylene. In embodiments, L^{2B} is independently substituted or unsubstituted 4 to 6 membered heteroalkylene. In embodiments, L^{2B} is independently substituted 4 to 6 membered heteroalkylene. In embodiments, L^{2B} is independently unsubstituted 4 to 6 membered heteroalkylene. In embodiments, L^{2B} is independently substituted or unsubstituted 2 to 3 membered heteroalkylene. In embodiments, L^{2B} is independently substituted 2 to 3 membered heteroalkylene. In embodiments, L^{2B} is independently unsubstituted 2 to 3 membered heteroalkylene. In embodiments, L^{2B} is independently substituted or unsubstituted 4 to 5 membered heteroalkylene. In embodiments, L^{2B} is independently substituted 4 to 5 membered heteroalkylene. In embodiments, L^{2B} is independently unsubstituted 4 to 5 membered heteroalkylene.

In embodiments, L^{2B} is independently substituted or unsubstituted cyclocycloalkylene (e.g., C₃-C₁₀, C₃-C₈, C₃-C₆, C₄-C₆, or C₅-C₆). In embodiments, L^{2B} is independently substituted cyclocycloalkylene (e.g., C₃-C₁₀, C₃-C₈, C₃-C₆, C₄-C₆, or C₅-C₆). In embodiments, L^{2B} is independently unsubstituted cyclocycloalkylene (e.g., C₃-C₁₀, C₃-C₈, C₃-C₆, C₄-C₆, or C₅-C₆). In embodiments, L^{2B} is independently substituted or unsubstituted C₃-C₁₀ cycloalkylene. In embodiments, L^{2B} is independently substituted C₃-C₁₀ cycloalkylene. In embodiments, L^{2B} is independently unsubstituted C₃-C₁₀ cycloalkylene. In embodiments, L^{2B} is independently substituted or unsubstituted C₃-C₈ cycloalkylene. In embodiments, L^{2B} is independently substituted C₃-C₈ cycloalkylene. In embodiments, L^{2B} is independently unsubstituted C₃-C₈ cycloalkylene. In embodiments, L^{2B} is independently substituted or unsubstituted C₃-C₆ cycloalkylene. In embodiments, L^{2B} is independently substituted C₃-C₆ cycloalkylene. In embodiments, L^{2B} is independently unsubstituted C₃-C₆ cycloalkylene. In embodiments, L^{2B} is independently substituted or unsubstituted C₄-C₆ cycloalkylene. In embodiments, L^{2B} is independently substituted C₄-C₆ cycloalkylene. In embodiments, L^{2B} is independently unsubstituted C₄-C₆ cycloalkylene. In embodiments, L^{2B} is independently substituted or unsubstituted C₅-C₆ cycloalkylene. In embodiments, L^{2B} is independently substituted C₅-C₆ cycloalkylene. In embodiments, L^{2B} is independently unsubstituted C₅-C₆ cycloalkylene. In embodiments, L^{2B} is independently substituted or unsubstituted hexylene. In embodiments, L^{2B} is independently substituted hexylene. In embodiments, L^{2B} is independently unsubstituted hexylene.

In embodiments, L^{2B} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2B} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{2B} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2B} is independently substituted or unsubstituted 3 to 10 membered heterocycloalkylene. In embodiments, L^{2B} is independently substituted 3 to 10 membered heterocycloalkylene. In embodiments, L^{2B} is independently unsubstituted 3 to 10 membered heterocycloalkylene. In embodiments, L^{2B} is independently substituted or unsubstituted 3 to 8 membered heterocycloalkylene. In embodiments, L^{2B} is independently substituted 3 to 8 membered heterocycloalkylene. In embodiments, L^{2B} is independently unsubstituted 3 to 8 membered heterocycloalkylene. In embodiments, L^{2B} is independently substituted or unsubstituted 3 to 6 membered heterocycloalkylene. In embodiments, L^{2B} is independently substituted 3 to 6 membered heterocycloalkylene. In embodiments, L^{2B} is independently unsubstituted 3 to 6 membered heterocycloalkylene. In embodiments, L^{2B} is independently substituted or unsubstituted 4 to 6 membered heterocycloalkylene. In embodiments, L^{2B} is independently substituted 4 to 6 membered heterocycloalkylene. In embodiments, L^{2B} is independently unsubstituted 4 to 6 membered heterocycloalkylene. In embodiments, L^{2B} is independently substituted or unsubstituted 4 to 5 membered heterocycloalkylene. In embodiments, L^{2B} is independently substituted 4 to 5 membered heterocycloalkylene. In embodiments, L^{2B} is independently unsubstituted 4 to 5 membered heterocycloalkylene. In embodiments, L^{2B} is independently substituted or unsubstituted 5 to 6 membered heterocycloalkylene. In embodiments, L^{2B} is independently substituted 5 to 6 membered heterocycloalkylene. In embodiments, L^{2B} is independently unsubstituted 5 to 6 membered heterocycloalkylene.

In embodiments, L^{2B} is independently substituted or unsubstituted arylene. In embodiments, L^{2B} is independently substituted or unsubstituted arylene (e.g., C₆-C₁₂, C₆-C₁₀, or phenyl). In embodiments, L^{2B} is independently substituted arylene (e.g., C₆-C₁₂, C₆-C₁₀, or phenyl). In embodiments, L^{2B} is independently unsubstituted arylene (e.g., C₆-C₁₂, C₆-C₁₀, or phenyl). In embodiments, L^{2B} is independently substituted or unsubstituted C₆-C₁₂ arylene. In embodiments, L^{2B} is independently substituted C₆-C₁₂ arylene. In embodiments, L^{2B} is independently unsubstituted C₆-C₁₂ arylene. In embodiments, L^{2B} is independently substituted or unsubstituted C₆-C₁₀ arylene. In embodiments, L^{2B} is independently substituted C₆-C₁₀ arylene. In embodiments, L^{2B} is independently unsubstituted C₆-C₁₀ arylene. In embodiments, L^{2B} is independently substituted or unsubstituted phenylene. In embodiments, L^{2B} is independently substituted phenylene. In embodiments, L^{2B} is independently unsubstituted phenylene. In embodiments, L^{2B} is independently substituted or unsubstituted biphenylene. In embodiments, L^{2B} is independently substituted biphenylene. In embodiments, L^{2B} is independently unsubstituted biphenylene. In embodiments, L^{2B} is independently substituted or unsubstituted naphthylene. In embodiments, L^{2B} is independently substituted naphthylene. In embodiments, L^{2B} is independently unsubstituted naphthylene.

In embodiments, L^{2B} is independently substituted or unsubstituted heteroarylene. In embodiments, L^{2B} is independently substituted heteroarylene. In embodiments, L^{2B} is independently unsubstituted heteroarylene. In embodiments, L^{2B} is independently substituted or unsubstituted heteroarylene (e.g., 5 to 12 membered, 5 to 10 membered, 5 to 9 membered, or 5 to 6 membered). In embodiments, L^{2B} is independently substituted heteroarylene (e.g., 5 to 12 membered, 5 to 10 membered, 5 to 9 membered, or 5 to 6 membered). In embodiments, L^{2B} is independently unsubstituted heteroarylene (e.g., 5 to 12 membered, 5 to 10 membered, 5 to 9 membered, or 5 to 6 membered). In embodiments, L^{2B} is independently substituted or unsubstituted 5 to 12 membered heteroarylene. In embodiments, L^{2B} is independently substituted 5 to 12 membered heteroarylene. In embodiments, L^{2B} is independently unsubstituted 5 to 12 membered heteroarylene. In embodiments, L^{2B} is independently substituted or unsubstituted 5 to 10 membered heteroarylene. In embodiments, L^{2B} is independently substituted 5 to 10 membered heteroarylene. In embodiments, L^{2B} is independently unsubstituted 5 to 10 membered heteroarylene. In embodiments, L^{2B} is independently substituted or unsubstituted 5 to 9 membered heteroarylene. In embodiments, L^{2B} is independently substituted 5 to 9 membered heteroarylene. In embodiments, L^{2B} is independently unsubstituted 5 to 9 membered heteroarylene. In embodiments, L^{2B} is independently substituted or unsubstituted 5 to 6 membered heteroarylene. In embodiments, L^{2B} is independently substituted 5 to 6 membered heteroarylene. In embodiments, L^{2B} is independently unsubstituted 5 to 6 membered heteroarylene.

In embodiments, L^{2C} is independently a bond. In embodiments, L^{2C} is independently —NH—. In embodiments, L^{2C} is independently —O—. In embodiments, L^{2C} is independently —S—. In embodiments, L^{2C} is independently —C(O)—. In embodiments, L^{2C} is independently —NHC(O)—. In embodiments, L^{2C} is independently —S(O)₂C(O)—. In embodiments, L^{2C} is independently —OPO₂—O—. In embodiments, L^{2C} is independently —OP(S)(O)—O—. In embodiments, L^{2C} is independently —OP(S)₂—O—. In embodiments, L^{2C} is independently —S(O)NH—. In embodiments, L^{2C} is independently —NHC(O)NH—. In embodiments, L^{2C} is independently —C(O)O—. In embodiments, L^{2C} is independently —OC(O)—. In embodiments, L^{2C} is independently —C(O)NH—. In embodiments, L^{2C} is not a bond.

In embodiments, L^{2C} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{2C} is independently substituted alkylene (e.g., C₁-C₂₅, C₁-C₂₀, C₁-C₁₂, C₁-C₈, C₁-C₆, C₁-C₄, or C₁-C₂). In embodiments, L^{2C} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{2C} is independently substituted or unsubstituted C₁-C₂₅ alkylene. In embodiments, L^{2C} is independently substituted C₁-C₂₅ alkylene. In embodiments, L^{2C} is independently unsubstituted C₁-C₂₅ alkylene. In embodiments, L^{2C} is independently substituted or unsubstituted C₁-C₂₀ alkylene. In embodiments, L^{2C} is independently substituted C₁-C₂₀ alkylene. In embodiments, L^{2C} is independently unsubstituted C₁-C₂₀ alkylene. In embodiments, L^{2C} is independently substituted or unsubstituted C₁-C₁₂ alkylene. In embodiments, L^{2C} is independently substituted C₁-C₁₂ alkylene. In embodiments, L^{2C} is independently unsubstituted C₁-C₁₂ alkylene. In embodiments, L^{2C} is independently substituted or unsubstituted C₁-C₈ alkylene. In embodiments, L^{2C} is independently substituted C₁-C₈ alkylene. In embodiments, L^{2C} is independently unsubstituted C₁-C₈ alkylene. In embodiments, L^{2C} is independently substituted or unsubstituted C₁-C₆ alkylene. In embodiments, L^{2C} is independently substituted C₁-C₆ alkylene. In embodiments, L^{2C} is independently unsubstituted C₁-C₆ alkylene. In embodiments, L^{2C} is independently substituted or unsubstituted C₁-C₄ alkylene. In embodiments, L^{2C} is independently substituted C₁-C₄ alkylene. In embodiments, L^{2C} is independently unsubstituted C₁-C₄ alkylene. In embodiments, L^{2C} is independently substituted or unsubstituted ethylene. In embodiments, L^{2C} is independently substituted ethylene. In embodiments, L^{2C} is independently unsubstituted ethylene. In embodiments, L^{2C} is independently substituted or unsubstituted methylene. In embodiments, L^{2C} is independently substituted methylene. In embodiments, L^{2C} is independently unsubstituted methylene.

In embodiments, L^{2C} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2C} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2C} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2C} is independently substituted or unsubstituted 2 to 25 membered heteroalkylene. In embodiments, L^{2C} is independently substituted 2 to 25 membered heteroalkylene. In embodiments, L^{2C} is independently unsubstituted 2 to 25 membered heteroalkylene. In embodiments, L^{2C} is independently substituted or unsubstituted 2 to 20 membered heteroalkylene. In embodiments, L^{2C} is independently substituted 2 to 20 membered heteroalkylene. In embodiments, L^{2C} is independently unsubstituted 2 to 20 membered heteroalkylene. In embodiments, L^{2C} is independently substituted or unsubstituted 2 to 12 membered heteroalkylene. In embodiments, L^{2C} is independently substituted 2 to 12 membered heteroalkylene. In embodiments, L^{2C} is independently unsubstituted 2 to 12 membered heteroalkylene. In embodiments, L^{2C} is independently substituted or unsubstituted 2 to 8 membered heteroalkylene. In embodiments, L^{2C} is independently substituted 2 to 8 membered heteroalkylene. In embodiments, L^{2C} is independently unsubstituted 2 to 8 membered heteroalkylene. In embodiments, L^{2C} is independently substituted or unsubstituted 2 to 6 membered heteroalkylene. In embodiments, L^{2C} is independently substituted 2 to 6 membered heteroalkylene. In embodiments, L^{2C} is independently unsubstituted 2 to 6 membered heteroalkylene. In embodiments, L^{2C} is independently substituted or unsubstituted 4 to 6 membered heteroalkylene. In embodiments, L^{2C} is independently substituted 4 to 6 membered heteroalkylene. In embodiments, L^{2C} is independently unsubstituted 4 to 6 membered heteroalkylene. In embodiments, L^{2C} is independently substituted or unsubstituted 2 to 3 membered heteroalkylene. In embodiments, L^{2C} is independently substituted 2 to 3 membered heteroalkylene. In embodiments, L^{2C} is independently unsubstituted 2 to 3 membered heteroalkylene. In embodiments, L^{2C} is independently substituted or unsubstituted 4 to 5 membered heteroalkylene. In embodiments, L^{2C} is independently substituted 4 to 5 membered heteroalkylene. In embodiments, L^{2C} is independently unsubstituted 4 to 5 membered heteroalkylene.

In embodiments, L^{2C} is independently substituted or unsubstituted cyclocycloalkylene (e.g., C₃-C₁₀, C₃-C₈, C₃-C₆, C₄-C₆, or C₅-C₆). In embodiments, L^{2C} is independently substituted cyclocycloalkylene (e.g., C₃-C₁₀, C₃-C₈, C₃-C₆, C₄-C₆, or C₅-C₆). In embodiments, L^{2C} is independently unsubstituted cyclocycloalkylene (e.g., C₃-C₁₀, C₃-C₈, C₃-C₆, C₄-C₆, or C₅-C₆). In embodiments, L^{2C} is independently substituted or unsubstituted C₃-C₁₀ cycloalkylene. In embodiments, L^{2C} is independently substituted C₃-C₁₀ cycloalkylene. In embodiments, L^{2C} is independently unsubstituted C₃-C₁₀ cycloalkylene. In embodiments, L^{2C} is independently substituted or unsubstituted C₃-C₈ cycloalkylene. In embodiments, L^{2C} is independently substituted C₃-C₈ cycloalkylene. In embodiments, L^{2C} is independently unsubstituted C₃-C₈ cycloalkylene. In embodiments, L^{2C} is independently substituted or unsubstituted C₃-C₆ cycloalkylene. In embodiments, L^{2C} is independently substituted C₃-C₆ cycloalkylene. In embodiments, L^{2C} is independently unsubstituted C₃-C₆ cycloalkylene. In embodiments, L^{2C} is independently substituted or unsubstituted C₄-C₆ cycloalkylene. In embodiments, L^{2C} is independently substituted C₄-C₆ cycloalkylene. In embodiments, L^{2C} is independently unsubstituted C₄-C₆ cycloalkylene. In embodiments, L^{2C} is independently substituted or unsubstituted C₅-C₆ cycloalkylene. In embodiments, L^{2C} is independently substituted C₅-C₆ cycloalkylene. In embodiments, L^{2C} is independently unsubstituted C₅-C₆ cycloalkylene. In embodiments, L^{2C} is independently substituted or unsubstituted hexylene. In embodiments, L^{2C} is independently substituted hexylene. In embodiments, L^{2C} is independently unsubstituted hexylene.

In embodiments, L^{2C} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2C} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{2C} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2C} is independently substituted or unsubstituted 3 to 10 membered heterocycloalkylene. In embodiments, L^{2C} is independently substituted 3 to 10 membered heterocycloalkylene. In embodiments, L^{2C} is independently unsubstituted 3 to 10 membered heterocycloalkylene. In embodiments, L^{2C} is independently substituted or unsubstituted 3 to 8 membered heterocycloalkylene. In embodiments, L^{2C} is independently substituted 3 to 8 membered heterocycloalkylene. In embodiments, L^{2C} is independently unsubstituted 3 to 8 membered heterocycloalkylene. In embodiments, L^{2C} is independently substituted or unsubstituted 3 to 6 membered heterocycloalkylene. In embodiments, L^{2C} is independently substituted 3 to 6 membered heterocycloalkylene. In embodiments, L^{2C} is independently unsubstituted 3 to 6 membered heterocycloalkylene. In embodiments, L^{2C} is independently substituted or unsubstituted 4 to 6 membered heterocycloalkylene. In embodiments, L^{2C} is independently substituted 4 to 6 membered heterocycloalkylene. In embodiments, L^{2C} is independently unsubstituted 4 to 6 membered heterocycloalkylene. In embodiments, L^{2C} is independently substituted or unsubstituted 4 to 5 membered heterocycloalkylene. In embodiments, L^{2C} is independently substituted 4 to 5 membered heterocycloalkylene. In embodiments, L^{2C} is independently unsubstituted 4 to 5 membered heterocycloalkylene. In embodiments, L^{2C} is independently substituted or unsubstituted 5 to 6 membered heterocycloalkylene. In embodiments, L^{2C} is independently substituted 5 to 6 membered heterocycloalkylene. In embodiments, L^{2C} is independently unsubstituted 5 to 6 membered heterocycloalkylene.

In embodiments, L^{2C} is independently substituted or unsubstituted arylene. In embodiments, L^{2C} is independently substituted or unsubstituted arylene (e.g., C₆-C₁₂, C₆-C₁₀, or phenyl). In embodiments, L^{2C} is independently substituted arylene (e.g., C₆-C₁₂, C₆-C₁₀, or phenyl). In embodiments, L^{2C} is independently unsubstituted arylene (e.g., C₆-C₁₂, C₆-C₁₀, or phenyl). In embodiments, L^{2C} is independently substituted or unsubstituted C₆-C₁₂ arylene. In embodiments, L^{2C} is independently substituted C₆-C₁₂ arylene. In embodiments, L^{2C} is independently unsubstituted C₆-C₁₂ arylene. In embodiments, L^{2C} is independently substituted or unsubstituted C₆-C₁₀ arylene. In embodiments, L^{2C} is independently substituted C₆-C₁₀ arylene. In embodiments, L^{2C} is independently unsubstituted C₆-C₁₀ arylene. In embodiments, L^{2C} is independently substituted or unsubstituted phenylene. In embodiments, L^{2C} is independently substituted phenylene. In embodiments, L^{2C} is independently unsubstituted phenylene. In embodiments, L^{2C} is independently substituted or unsubstituted biphenylene. In embodiments, L^{2C} is independently substituted biphenylene. In embodiments, L^{2C} is independently unsubstituted biphenylene. In embodiments, L^{2C} is independently substituted or unsubstituted naphthylene. In embodiments, L^{2C} is independently substituted naphthylene. In embodiments, L^{2C} is independently unsubstituted naphthylene.

In embodiments, L^{2C} is independently substituted or unsubstituted heteroarylene. In embodiments, L^{2C} is independently substituted heteroarylene. In embodiments, L^{2C} is independently unsubstituted heteroarylene. In embodiments, L^{2C} is independently substituted or unsubstituted heteroarylene (e.g., 5 to 12 membered, 5 to 10 membered, 5 to 9 membered, or 5 to 6 membered). In embodiments, L^{2C} is independently substituted heteroarylene (e.g., 5 to 12 membered, 5 to 10 membered, 5 to 9 membered, or 5 to 6 membered). In embodiments, L^{2C} is independently unsubstituted heteroarylene (e.g., 5 to 12 membered, 5 to 10 membered, 5 to 9 membered, or 5 to 6 membered). In embodiments, L^{2C} is independently substituted or unsubstituted 5 to 12 membered heteroarylene. In embodiments, L^{2C} is independently substituted 5 to 12 membered heteroarylene. In embodiments, L^{2C} is independently unsubstituted 5 to 12 membered heteroarylene. In embodiments, L^{2C} is independently substituted or unsubstituted 5 to 10 membered heteroarylene. In embodiments, L^{2C} is independently substituted 5 to 10 membered heteroarylene. In embodiments, L^{2C} is independently unsubstituted 5 to 10 membered heteroarylene. In embodiments, L^{2C} is independently substituted or unsubstituted 5 to 9 membered heteroarylene. In embodiments, L^{2C} is independently substituted 5 to 9 membered heteroarylene. In embodiments, L^{2C} is independently unsubstituted 5 to 9 membered heteroarylene. In embodiments, L^{2C} is independently substituted or unsubstituted 5 to 6 membered heteroarylene. In embodiments, L^{2C} is independently substituted 5 to 6 membered heteroarylene. In embodiments, L^{2C} is independently unsubstituted 5 to 6 membered heteroarylene.

In embodiments, L^{2D} is independently a bond. In embodiments, L^{2D} is independently —NH—. In embodiments, L^{2D} is independently —O—. In embodiments, L^{2D} is independently —S—. In embodiments, L^{2D} is independently —C(O)—. In embodiments, L^{2D} is independently —NHC(O)—. In embodiments, L^{2D} is independently —S(O)₂C(O)—. In embodiments, L^{2D} is independently —OPO₂—O—. In embodiments, L^{2D} is independently —OP(S)(O)—O—. In embodiments, L^{2D} is independently —OP(S)₂—O—. In embodiments, L^{2D} is independently —S(O)NH—. In embodiments, L^{2D} is independently —NHC(O)NH—. In embodiments, L^{2D} is independently —C(O)O—. In embodiments, L^{2D} is independently —OC(O)—. In embodiments, L^{2D} is independently —C(O)NH—. In embodiments, L^{2D} is not a bond.

In embodiments, L^{2D} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{2D} is independently substituted alkylene (e.g., C₁-C₂₅, C₁-C₂₀, C₁-C₁₂, C₁-C₈, C₁-C₆, C₁-C₄, or C₁-C₂). In embodiments, L^{2D} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{2D} is independently substituted or unsubstituted C₁-C₂₅ alkylene. In embodiments, L^{2D} is independently substituted C₁-C₂₅ alkylene. In embodiments, L^{2D} is independently unsubstituted C₁-C₂₅ alkylene. In embodiments, L^{2D} is independently substituted or unsubstituted C₁-C₂₀ alkylene. In embodiments, L^{2D} is independently substituted C₁-C₂₀ alkylene. In embodiments, L^{2D} is independently unsubstituted C₁-C₂₀ alkylene. In embodiments, L^{2D} is independently substituted or unsubstituted C₁-C₁₂ alkylene. In embodiments, L^{2D} is independently substituted C₁-C₁₂ alkylene. In embodiments, L^{2D} is independently unsubstituted C₁-C₁₂ alkylene. In embodiments, L^{2D} is independently substituted or unsubstituted C₁-C₈ alkylene. In embodiments, L^{2D} is independently substituted C₁-C₈alkylene. In embodiments, L^{2D} is independently unsubstituted C₁-C₈ alkylene. In embodiments, L^{2D} is independently substituted or unsubstituted C₁-C₆ alkylene. In embodiments, L^{2D} is independently substituted C₁-C₆ alkylene. In embodiments, L^{2D} is independently unsubstituted C₁-C₆ alkylene. In embodiments, L^{2D} is independently substituted or unsubstituted C₁-C₄ alkylene. In embodiments, L^{2D} is independently substituted C₁-C₄ alkylene. In embodiments, L^{2D} is independently unsubstituted C₁-C₄ alkylene. In embodiments, L^{2D} is independently substituted or unsubstituted ethylene. In embodiments, L^{2D} is independently substituted ethylene. In embodiments, L^{2D} is independently unsubstituted ethylene. In embodiments, L^{2D} is independently substituted or unsubstituted methylene. In embodiments, L^{2D} is independently substituted methylene. In embodiments, L^{2D} is independently unsubstituted methylene.

In embodiments, L^{2D} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2D} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2D} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2D} is independently substituted or unsubstituted 2 to 25 membered heteroalkylene. In embodiments, L^{2D} is independently substituted 2 to 25 membered heteroalkylene. In embodiments, L^{2D} is independently unsubstituted 2 to 25 membered heteroalkylene. In embodiments, L^{2D} is independently substituted or unsubstituted 2 to 20 membered heteroalkylene. In embodiments, L^{2D} is independently substituted 2 to 20 membered heteroalkylene. In embodiments, L^{2D} is independently unsubstituted 2 to 20 membered heteroalkylene. In embodiments, L^{2D} is independently substituted or unsubstituted 2 to 12 membered heteroalkylene. In embodiments, L^{2D} is independently substituted 2 to 12 membered heteroalkylene. In embodiments, L^{2D} is independently unsubstituted 2 to 12 membered heteroalkylene. In embodiments, L^{2D} is independently substituted or unsubstituted 2 to 8 membered heteroalkylene. In embodiments, L^{2D} is independently substituted 2 to 8 membered heteroalkylene. In embodiments, L^{2D} is independently unsubstituted 2 to 8 membered heteroalkylene. In embodiments, L^{2D} is independently substituted or unsubstituted 2 to 6 membered heteroalkylene. In embodiments, L^{2D} is independently substituted 2 to 6 membered heteroalkylene. In embodiments, L^{2D} is independently unsubstituted 2 to 6 membered heteroalkylene. In embodiments, L^{2D} is independently substituted or unsubstituted 4 to 6 membered heteroalkylene. In embodiments, L^{2D} is independently substituted 4 to 6 membered heteroalkylene. In embodiments, L^{2D} is independently unsubstituted 4 to 6 membered heteroalkylene. In embodiments, L^{2D} is independently substituted or unsubstituted 2 to 3 membered heteroalkylene. In embodiments, L^{2D} is independently substituted 2 to 3 membered heteroalkylene. In embodiments, L^{2D} is independently unsubstituted 2 to 3 membered heteroalkylene. In embodiments, L^{2D} is independently substituted or unsubstituted 4 to 5 membered heteroalkylene. In embodiments, L^{2D} is independently substituted 4 to 5 membered heteroalkylene. In embodiments, L^{2D} is independently unsubstituted 4 to 5 membered heteroalkylene.

In embodiments, L^{2D} is independently substituted or unsubstituted cyclocycloalkylene (e.g., C₃-C₁₀, C₃-C₈, C₃-C₆, C₄-C₆, or C₅-C₆). In embodiments, L^{2D} is independently substituted cyclocycloalkylene (e.g., C₃-C₁₀, C₃-C₈, C₃-C₆, C₄-C₆, or C₅-C₆). In embodiments, L^{2D} is independently unsubstituted cyclocycloalkylene (e.g., C₃-C₁₀, C₃-C₈, C₃-C₆, C₄-C₆, or C₅-C₆). In embodiments, L^{2D} is independently substituted or unsubstituted C₃-C₁₀ cycloalkylene. In embodiments, L^{2D} is independently substituted C₃-C₁₀ cycloalkylene. In embodiments, L^{2D} is independently unsubstituted C₃-C₁₀ cycloalkylene. In embodiments, L^{2D} is independently substituted or unsubstituted C₃-C₈ cycloalkylene. In embodiments, L^{2D} is independently substituted C₃-C₈ cycloalkylene. In embodiments, L^{2D} is independently unsubstituted C₃-C₈ cycloalkylene. In embodiments, L^{2D} is independently substituted or unsubstituted C₃-C₆ cycloalkylene. In embodiments, L^{2D} is independently substituted C₃-C₆ cycloalkylene. In embodiments, L^{2D} is independently unsubstituted C₃-C₆ cycloalkylene. In embodiments, L^{2D} is independently substituted or unsubstituted C₄-C₆ cycloalkylene. In embodiments, L^{2D} is independently substituted C₄-C₆ cycloalkylene. In embodiments, L^{2D} is independently unsubstituted C₄-C₆ cycloalkylene. In embodiments, L^{2D} is independently substituted or unsubstituted C₅-C₆ cycloalkylene. In embodiments, L^{2D} is independently substituted C₅-C₆ cycloalkylene. In embodiments, L^{2D} is independently unsubstituted C₅-C₆ cycloalkylene. In embodiments, L^{2D} is independently substituted or unsubstituted hexylene. In embodiments, L^{2D} is independently substituted hexylene. In embodiments, L^{2D} is independently unsubstituted hexylene.

In embodiments, L^{2D} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2D} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{2D} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2D} is independently substituted or unsubstituted 3 to 10 membered heterocycloalkylene. In embodiments, L^{2D} is independently substituted 3 to 10 membered heterocycloalkylene. In embodiments, L^{2D} is independently unsubstituted 3 to 10 membered heterocycloalkylene. In embodiments, L^{2D} is independently substituted or unsubstituted 3 to 8 membered heterocycloalkylene. In embodiments, L^{2D} is independently substituted 3 to 8 membered heterocycloalkylene. In embodiments, L^{2D} is independently unsubstituted 3 to 8 membered heterocycloalkylene. In embodiments, L^{2D} is independently substituted or unsubstituted 3 to 6 membered heterocycloalkylene. In embodiments, L^{2D} is independently substituted 3 to 6 membered heterocycloalkylene. In embodiments, L^{2D} is independently unsubstituted 3 to 6 membered heterocycloalkylene. In embodiments, L^{2D} is independently substituted or unsubstituted 4 to 6 membered heterocycloalkylene. In embodiments, L^{2D} is independently substituted 4 to 6 membered heterocycloalkylene. In embodiments, L^{2D} is independently unsubstituted 4 to 6 membered heterocycloalkylene. In embodiments, L^{2D} is independently substituted or unsubstituted 4 to 5 membered heterocycloalkylene. In embodiments, L^{2D} is independently substituted 4 to 5 membered heterocycloalkylene. In embodiments, L^{2D} is independently unsubstituted 4 to 5 membered heterocycloalkylene. In embodiments, L^{2D} is independently substituted or unsubstituted 5 to 6 membered heterocycloalkylene. In embodiments, L^{2D} is independently substituted 5 to 6 membered heterocycloalkylene. In embodiments, L^{2D} is independently unsubstituted 5 to 6 membered heterocycloalkylene.

In embodiments, L^{2D} is independently substituted or unsubstituted arylene. In embodiments, L^{2D} is independently substituted or unsubstituted arylene (e.g., C₆-C₁₂, C₆-C₁₀, or phenyl). In embodiments, L^{2D} is independently substituted arylene (e.g., C₆-C₁₂, C₆-C₁₀, or phenyl). In embodiments, L^{2D} is independently unsubstituted arylene (e.g., C₆-C₁₂, C₆-C₁₀, or phenyl). In embodiments, L^{2D} is independently substituted or unsubstituted C₆-C₁₂ arylene. In embodiments, L^{2D} is independently substituted C₆-C₁₂ arylene. In embodiments, L^{2D} is independently unsubstituted C₆-C₁₂ arylene. In embodiments, L^{2D} is independently substituted or unsubstituted C₆-C₁₀ arylene. In embodiments, L^{2D} is independently substituted C₆-C₁₀ arylene. In embodiments, L^{2D} is independently unsubstituted C₆-C₁₀ arylene. In embodiments, L^{2D} is independently substituted or unsubstituted phenylene. In embodiments, L^{2D} is independently substituted phenylene. In embodiments, L^{2D} is independently unsubstituted phenylene. In embodiments, L^{2D} is independently substituted or unsubstituted biphenylene. In embodiments, L^{2D} is independently substituted biphenylene. In embodiments, L^{2D} is independently unsubstituted biphenylene. In embodiments, L^{2D} is independently substituted or unsubstituted naphthylene. In embodiments, L^{2D} is independently substituted naphthylene. In embodiments, L^{2D} is independently unsubstituted naphthylene.

In embodiments, L^{2D} is independently substituted or unsubstituted heteroarylene. In embodiments, L^{2D} is independently substituted heteroarylene. In embodiments, L^{2D} is independently unsubstituted heteroarylene. In embodiments, L^{2D} is independently substituted or unsubstituted heteroarylene (e.g., 5 to 12 membered, 5 to 10 membered, 5 to 9 membered, or 5 to 6 membered). In embodiments, L^{2D} is independently substituted heteroarylene (e.g., 5 to 12 membered, 5 to 10 membered, 5 to 9 membered, or 5 to 6 membered). In embodiments, L^{2D} is independently unsubstituted heteroarylene (e.g., 5 to 12 membered, 5 to 10 membered, 5 to 9 membered, or 5 to 6 membered). In embodiments, L^{2D} is independently substituted or unsubstituted 5 to 12 membered heteroarylene. In embodiments, L^{2D} is independently substituted 5 to 12 membered heteroarylene. In embodiments, L^{2D} is independently unsubstituted 5 to 12 membered heteroarylene. In embodiments, L^{2D} is independently substituted or unsubstituted 5 to 10 membered heteroarylene. In embodiments, L^{2D} is independently substituted 5 to 10 membered heteroarylene. In embodiments, L^{2D} is independently unsubstituted 5 to 10 membered heteroarylene. In embodiments, L^{2D} is independently substituted or unsubstituted 5 to 9 membered heteroarylene. In embodiments, L^{2D} is independently substituted 5 to 9 membered heteroarylene. In embodiments, L^{2D} is independently unsubstituted 5 to 9 membered heteroarylene. In embodiments, L^{2D} is independently substituted or unsubstituted 5 to 6 membered heteroarylene. In embodiments, L^{2D} is independently substituted 5 to 6 membered heteroarylene. In embodiments, L^{2D} is independently unsubstituted 5 to 6 membered heteroarylene.

In embodiments, L^{2E} is independently a bond. In embodiments, L^{2E} is independently —NH—. In embodiments, L^{2E} is independently —O—. In embodiments, L^{2E} is independently —S—. In embodiments, L^{2E} is independently —C(O)—. In embodiments, L^{2E} is independently —NHC(O)—. In embodiments, L^{2E} is independently —S(O)₂C(O)—. In embodiments, L^{2E} is independently —OPO₂—O—. In embodiments, L^{2E} is independently —OP(S)(O)—O—. In embodiments, L^{2E} is independently —OP(S)₂—O—. In embodiments, L^{2E} is independently —S(O)NH—. In embodiments, L^{2E} is independently —NHC(O)NH—. In embodiments, L^{2E} is independently —C(O)O—. In embodiments, L^{2E} is independently —OC(O)—. In embodiments, L^{2E} is independently —C(O)NH—. In embodiments, L^{2E} is not a bond.

In embodiments, L^{2E} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{2E} is independently substituted alkylene (e.g., C₁-C₂₅, C₁-C₂₀, C₁-C₁₂, C₁-C₈, C₁-C₆, C₁-C₄, or C₁-C₂). In embodiments, L^{2E} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{2E} is independently substituted or unsubstituted C₁-C₂₅ alkylene. In embodiments, L^{2E} is independently substituted C₁-C₂₅ alkylene. In embodiments, L^{2E} is independently unsubstituted C₁-C₂₅ alkylene. In embodiments, L^{2E} is independently substituted or unsubstituted C₁-C₂₀ alkylene. In embodiments, L^{2E} is independently substituted C₁-C₂₀ alkylene. In embodiments, L^{2E} is independently unsubstituted C₁-C₂₀ alkylene. In embodiments, L^{2E} is independently substituted or unsubstituted C₁-C₁₂ alkylene. In embodiments, L^{2E} is independently substituted C₁-C₁₂ alkylene. In embodiments, L^{2E} is independently unsubstituted C₁-C₁₂ alkylene. In embodiments, L^{2E} is independently substituted or unsubstituted C₁-C₈ alkylene. In embodiments, L^{2E} is independently substituted C₁-C₈alkylene. In embodiments, L^{2E} is independently unsubstituted C₁-C₈ alkylene. In embodiments, L^{2E} is independently substituted or unsubstituted C₁-C₆ alkylene. In embodiments, L^{2E} is independently substituted C₁-C₆ alkylene. In embodiments, L^{2E} is independently unsubstituted C₁-C₆ alkylene. In embodiments, L^{2E} is independently substituted or unsubstituted C₁-C₄ alkylene. In embodiments, L^{2E} is independently substituted C₁-C₄ alkylene. In embodiments, L^{2E} is independently unsubstituted C₁-C₄ alkylene. In embodiments, L^{2E} is independently substituted or unsubstituted ethylene. In embodiments, L^{2E} is independently substituted ethylene. In embodiments, L^{2E} is independently unsubstituted ethylene. In embodiments, L^{2E} is independently substituted or unsubstituted methylene. In embodiments, L^{2E} is independently substituted methylene. In embodiments, L^{2E} is independently unsubstituted methylene.

In embodiments, L^{2E} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2E} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2E} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{2E} is independently substituted or unsubstituted 2 to 25 membered heteroalkylene. In embodiments, L^{2E} is independently substituted 2 to 25 membered heteroalkylene. In embodiments, L^{2E} is independently unsubstituted 2 to 25 membered heteroalkylene. In embodiments, L^{2E} is independently substituted or unsubstituted 2 to 20 membered heteroalkylene. In embodiments, L^{2E} is independently substituted 2 to 20 membered heteroalkylene. In embodiments, L^{2E} is independently unsubstituted 2 to 20 membered heteroalkylene. In embodiments, L^{2E} is independently substituted or unsubstituted 2 to 12 membered heteroalkylene. In embodiments, L^{2E} is independently substituted 2 to 12 membered heteroalkylene. In embodiments, L^{2E} is independently unsubstituted 2 to 12 membered heteroalkylene. In embodiments, L^{2E} is independently substituted or unsubstituted 2 to 8 membered heteroalkylene. In embodiments, L^{2E} is independently substituted 2 to 8 membered heteroalkylene. In embodiments, L^{2E} is independently unsubstituted 2 to 8 membered heteroalkylene. In embodiments, L^{2E} is independently substituted or unsubstituted 2 to 6 membered heteroalkylene. In embodiments, L^{2E} is independently substituted 2 to 6 membered heteroalkylene. In embodiments, L^{2E} is independently unsubstituted 2 to 6 membered heteroalkylene. In embodiments, L^{2E} is independently substituted or unsubstituted 4 to 6 membered heteroalkylene. In embodiments, L^{2E} is independently substituted 4 to 6 membered heteroalkylene. In embodiments, L^{2E} is independently unsubstituted 4 to 6 membered heteroalkylene. In embodiments, L^{2E} is independently substituted or unsubstituted 2 to 3 membered heteroalkylene. In embodiments, L^{2E} is independently substituted 2 to 3 membered heteroalkylene. In embodiments, L^{2E} is independently unsubstituted 2 to 3 membered heteroalkylene. In embodiments, L^{2E} is independently substituted or unsubstituted 4 to 5 membered heteroalkylene. In embodiments, L^{2E} is independently substituted 4 to 5 membered heteroalkylene. In embodiments, L^{2E} is independently unsubstituted 4 to 5 membered heteroalkylene.

In embodiments, L^{2E} is independently substituted or unsubstituted cyclocycloalkylene (e.g., C₃-C₁₀, C₃-C₈, C₃-C₆, C₄-C₆, or C₅-C₆). In embodiments, L^{2E} is independently substituted cyclocycloalkylene (e.g., C₃-C₁₀, C₃-C₈, C₃-C₆, C₄-C₆, or C₅-C₆). In embodiments, L^{2E} is independently unsubstituted cyclocycloalkylene (e.g., C₃-C₁₀, C₃-C₈, C₃-C₆, C₄-C₆, or C₅-C₆). In embodiments, L^{2E} is independently substituted or unsubstituted C₃-C₁₀ cycloalkylene. In embodiments, L^{2E} is independently substituted C₃-C₁₀ cycloalkylene. In embodiments, L^{2E} is independently unsubstituted C₃-C₁₀ cycloalkylene. In embodiments, L^{2E} is independently substituted or unsubstituted C₃-C₈ cycloalkylene. In embodiments, L^{2E} is independently substituted C₃-C₈ cycloalkylene. In embodiments, L^{2E} is independently unsubstituted C₃-C₈ cycloalkylene. In embodiments, L^{2E} is independently substituted or unsubstituted C₃-C₆ cycloalkylene. In embodiments, L^{2E} is independently substituted C₃-C₆ cycloalkylene. In embodiments, L^{2E} is independently unsubstituted C₃-C₆ cycloalkylene. In embodiments, L^{2E} is independently substituted or unsubstituted C₄-C₆ cycloalkylene. In embodiments, L^{2E} is independently substituted C₄-C₆ cycloalkylene. In embodiments, L^{2E} is independently unsubstituted C₄-C₆ cycloalkylene. In embodiments, L^{2E} is independently substituted or unsubstituted C₅-C₆ cycloalkylene. In embodiments, L^{2E} is independently substituted C₅-C₆ cycloalkylene. In embodiments, L^{2E} is independently unsubstituted C₅-C₆ cycloalkylene. In embodiments, L^{2E} is independently substituted or unsubstituted hexylene. In embodiments, L^{2E} is independently substituted hexylene. In embodiments, L^{2E} is independently unsubstituted hexylene.

In embodiments, L^{2E} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2E} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{2E} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{2E} is independently substituted or unsubstituted 3 to 10 membered heterocycloalkylene. In embodiments, L^{2E} is independently substituted 3 to 10 membered heterocycloalkylene. In embodiments, L^{2E} is independently unsubstituted 3 to 10 membered heterocycloalkylene. In embodiments, L^{2E} is independently substituted or unsubstituted 3 to 8 membered heterocycloalkylene. In embodiments, L^{2E} is independently substituted 3 to 8 membered heterocycloalkylene. In embodiments, L^{2E} is independently unsubstituted 3 to 8 membered heterocycloalkylene. In embodiments, L^{2E} is independently substituted or unsubstituted 3 to 6 membered heterocycloalkylene. In embodiments, L^{2E} is independently substituted 3 to 6 membered heterocycloalkylene. In embodiments, L^{2E} is independently unsubstituted 3 to 6 membered heterocycloalkylene. In embodiments, L^{2E} is independently substituted or unsubstituted 4 to 6 membered heterocycloalkylene. In embodiments, L^{2E} is independently substituted 4 to 6 membered heterocycloalkylene. In embodiments, L^{2E} is independently unsubstituted 4 to 6 membered heterocycloalkylene. In embodiments, L^{2E} is independently substituted or unsubstituted 4 to 5 membered heterocycloalkylene. In embodiments, L^{2E} is independently substituted 4 to 5 membered heterocycloalkylene. In embodiments, L^{2E} is independently unsubstituted 4 to 5 membered heterocycloalkylene. In embodiments, L^{2E} is independently substituted or unsubstituted 5 to 6 membered heterocycloalkylene. In embodiments, L^{2E} is independently substituted 5 to 6 membered heterocycloalkylene. In embodiments, L^{2E} is independently unsubstituted 5 to 6 membered heterocycloalkylene.

In embodiments, L^{2E} is independently substituted or unsubstituted arylene. In embodiments, L^{2E} is independently substituted or unsubstituted arylene (e.g., C₆-C₁₂, C₆-C₁₀, or phenyl). In embodiments, L^{2E} is independently substituted arylene (e.g., C₆-C₁₂, C₆-C₁₀, or phenyl). In embodiments, L^{2E} is independently unsubstituted arylene (e.g., C₆-C₁₂, C₆-C₁₀, or phenyl). In embodiments, L^{2E} is independently substituted or unsubstituted C₆-C₁₂ arylene. In embodiments, L^{2E} is independently substituted C₆-C₁₂ arylene. In embodiments, L^{2E} is independently unsubstituted C₆-C₁₂ arylene. In embodiments, L^{2E} is independently substituted or unsubstituted C₆-C₁₀ arylene. In embodiments, L^{2E} is independently substituted C₆-C₁₀ arylene. In embodiments, L^{2E} is independently unsubstituted C₆-C₁₀ arylene. In embodiments, L^{2E} is independently substituted or unsubstituted phenylene. In embodiments, L^{2E} is independently substituted phenylene. In embodiments, L^{2E} is independently unsubstituted phenylene. In embodiments, L^{2E} is independently substituted or unsubstituted biphenylene. In embodiments, L^{2E} is independently substituted biphenylene. In embodiments, L^{2E} is independently unsubstituted biphenylene. In embodiments, L^{2E} is independently substituted or unsubstituted naphthylene. In embodiments, L^{2E} is independently substituted naphthylene. In embodiments, L^{2E} is independently unsubstituted naphthylene.

In embodiments, L^{2E} is independently substituted or unsubstituted heteroarylene. In embodiments, L^{2E} is independently substituted heteroarylene. In embodiments, L^{2E} is independently unsubstituted heteroarylene. In embodiments, L^{2E} is independently substituted or unsubstituted heteroarylene (e.g., 5 to 12 membered, 5 to 10 membered, 5 to 9 membered, or 5 to 6 membered). In embodiments, L^{2E} is independently substituted heteroarylene (e.g., 5 to 12 membered, 5 to 10 membered, 5 to 9 membered, or 5 to 6 membered). In embodiments, L^{2E} is independently unsubstituted heteroarylene (e.g., 5 to 12 membered, 5 to 10 membered, 5 to 9 membered, or 5 to 6 membered). In embodiments, L^{2E} is independently substituted or unsubstituted 5 to 12 membered heteroarylene. In embodiments, L^{2E} is independently substituted 5 to 12 membered heteroarylene. In embodiments, L^{2E} is independently unsubstituted 5 to 12 membered heteroarylene. In embodiments, L^{2E} is independently substituted or unsubstituted 5 to 10 membered heteroarylene. In embodiments, L^{2E} is independently substituted 5 to 10 membered heteroarylene. In embodiments, L^{2E} is independently unsubstituted 5 to 10 membered heteroarylene. In embodiments, L^{2E} is independently substituted or unsubstituted 5 to 9 membered heteroarylene. In embodiments, L^{2E} is independently substituted 5 to 9 membered heteroarylene. In embodiments, L^{2E} is independently unsubstituted 5 to 9 membered heteroarylene. In embodiments, L^{2E} is independently substituted or unsubstituted 5 to 6 membered heteroarylene. In embodiments, L^{2E} is independently substituted 5 to 6 membered heteroarylene. In embodiments, L^{2E} is independently unsubstituted 5 to 6 membered heteroarylene.

In embodiments, L² is independently -L¹²-NH—C(O)— or -L¹²-C(O)—NH—, and L¹² is independently substituted or unsubstituted C₁-C₂₀ alkylene, substituted or unsubstituted 2-20 membered heteroalkylene, or substituted or unsubstituted 2-20 membered heteroalkenylene.

In embodiments, L¹² is independently substituted or unsubstituted alkylene (e.g., C₁-C₂₀, C₁-C₁₂, C₁-C₈, C₁-C₆, C₁-C₄, or C₁-C₂). In embodiments, L¹² is independently substituted alkylene (e.g., C₁-C₂₀, C₁-C₁₂, C₁-C₈, C₁-C₆, C₁-C₄, or C₁-C₂). In embodiments, L¹² is independently unsubstituted alkylene (e.g., C₁-C₂₀, C₁-C₁₂, C₁-C₈, C₁-C₆, C₁-C₄, or C₁-C₂). In embodiments, L¹² is independently substituted or unsubstituted C₁-C₂₀ alkylene. In embodiments, L¹² is independently substituted C₁-C₂₀ alkylene. In embodiments, L¹² is independently unsubstituted C₁-C₂₀ alkylene. In embodiments, L¹² is independently substituted or unsubstituted C₁-C₁₂ alkylene. In embodiments, L¹² is independently substituted C₁-C₁₂ alkylene. In embodiments, L¹² is independently unsubstituted C₁-C₁₂ alkylene. In embodiments, L¹² is independently substituted or unsubstituted C₁-C₈ alkylene. In embodiments, L¹² is independently substituted C₁-C₈ alkylene. In embodiments, L¹² is independently unsubstituted C₁-C₈ alkylene. In embodiments, L¹² is independently substituted or unsubstituted C₁-C₆ alkylene. In embodiments, L¹² is independently substituted C₁-C₆ alkylene. In embodiments, L¹² is independently unsubstituted C₁-C₆ alkylene. In embodiments, L¹² is independently substituted or unsubstituted C₁-C₄ alkylene. In embodiments, L¹² is independently substituted C₁-C₄ alkylene. In embodiments, L¹² is independently unsubstituted C₁-C₄ alkylene. In embodiments, L¹² is independently substituted or unsubstituted ethylene. In embodiments, L¹² is independently substituted ethylene. In embodiments, L¹² is independently unsubstituted ethylene. In embodiments, L¹² is independently substituted or unsubstituted methylene. In embodiments, L¹² is independently substituted methylene. In embodiments, L¹² is independently unsubstituted methylene.

In embodiments, L¹² is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹² is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹² is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹² is independently substituted or unsubstituted 2 to 20 membered heteroalkylene. In embodiments, L¹² is independently substituted 2 to 20 membered heteroalkylene. In embodiments, L¹² is independently unsubstituted 2 to 20 membered heteroalkylene. In embodiments, L¹² is independently substituted or unsubstituted 2 to 12 membered heteroalkylene. In embodiments, L¹² is independently substituted 2 to 12 membered heteroalkylene. In embodiments, L¹² is independently unsubstituted 2 to 12 membered heteroalkylene. In embodiments, L¹² is independently substituted or unsubstituted 2 to 8 membered heteroalkylene. In embodiments, L¹² is independently substituted 2 to 8 membered heteroalkylene. In embodiments, L¹² is independently unsubstituted 2 to 8 membered heteroalkylene. In embodiments, L¹² is independently substituted or unsubstituted 2 to 6 membered heteroalkylene. In embodiments, L¹² is independently substituted 2 to 6 membered heteroalkylene. In embodiments, L¹² is independently unsubstituted 2 to 6 membered heteroalkylene. In embodiments, L¹² is independently substituted or unsubstituted 4 to 6 membered heteroalkylene. In embodiments, L¹² is independently substituted 4 to 6 membered heteroalkylene. In embodiments, L¹² is independently unsubstituted 4 to 6 membered heteroalkylene. In embodiments, L¹² is independently substituted or unsubstituted 2 to 3 membered heteroalkylene. In embodiments, L¹² is independently substituted 2 to 3 membered heteroalkylene. In embodiments, L¹² is independently unsubstituted 2 to 3 membered heteroalkylene. In embodiments, L¹² is independently substituted or unsubstituted 4 to 5 membered heteroalkylene. In embodiments, L¹² is independently substituted 4 to 5 membered heteroalkylene. In embodiments, L¹² is independently unsubstituted 4 to 5 membered heteroalkylene.

In embodiments, L¹² is independently substituted or unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹² is independently substituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹² is independently unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹² is independently substituted or unsubstituted 2 to 20 membered heteroalkenylene. In embodiments, L¹² is independently substituted 2 to 20 membered heteroalkenylene. In embodiments, L¹² is independently unsubstituted 2 to 20 membered heteroalkenylene. In embodiments, L¹² is independently substituted or unsubstituted 2 to 12 membered heteroalkenylene. In embodiments, L¹² is independently substituted 2 to 12 membered heteroalkenylene. In embodiments, L¹² is independently unsubstituted 2 to 12 membered heteroalkenylene. In embodiments, L¹² is independently substituted or unsubstituted 2 to 8 membered heteroalkenylene. In embodiments, L¹² is independently substituted 2 to 8 membered heteroalkenylene. In embodiments, L¹² is independently unsubstituted 2 to 8 membered heteroalkenylene. In embodiments, L¹² is independently substituted or unsubstituted 2 to 6 membered heteroalkenylene. In embodiments, L¹² is independently substituted 2 to 6 membered heteroalkenylene. In embodiments, L¹² is independently unsubstituted 2 to 6 membered heteroalkenylene. In embodiments, L¹² is independently substituted or unsubstituted 4 to 6 membered heteroalkenylene. In embodiments, L¹² is independently substituted 4 to 6 membered heteroalkenylene. In embodiments, L¹² is independently unsubstituted 4 to 6 membered heteroalkenylene. In embodiments, L¹² is independently substituted or unsubstituted 2 to 3 membered heteroalkenylene. In embodiments, L¹² is independently substituted 2 to 3 membered heteroalkenylene. In embodiments, L¹² is independently unsubstituted 2 to 3 membered heteroalkenylene. In embodiments, L¹² is independently substituted or unsubstituted 4 to 5 membered heteroalkenylene. In embodiments, L¹² is independently substituted 4 to 5 membered heteroalkenylene. In embodiments, L¹² is independently unsubstituted 4 to 5 membered heteroalkenylene.

In embodiments, L² is independently -L¹²-NH—C(O)—. In embodiments, L² is independently -L¹²-C(O)—NH—. In embodiments, L¹² is substituted or unsubstituted C₁-C₈ alkylene. In embodiments, L¹² is substituted C₁-C₈ alkylene. In embodiments, L¹² is unsubstituted C₁-C₈ alkylene.

In embodiments, L² is independently

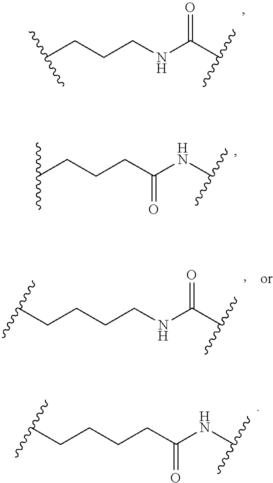 [SMILES: *CCCNC(*)=O ; *CCCC(=O)N* ; *CCCCNC(*)=O ; *CCCCC(=O)N*]

In embodiments, L² is independently

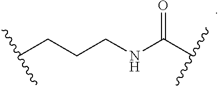 [SMILES: *CCCNC(*)=O]

In embodiments, L² is independently

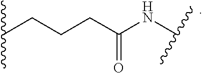 [SMILES: *CCCC(=O)N*]

In embodiments, L² is independently

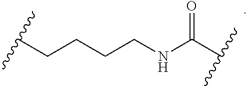 [SMILES: *CCCCNC(*)=O]

In embodiments, L² is independently

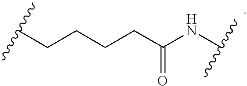 [SMILES: *CCCCC(=O)N*]

In embodiments, L² is independently a bond,

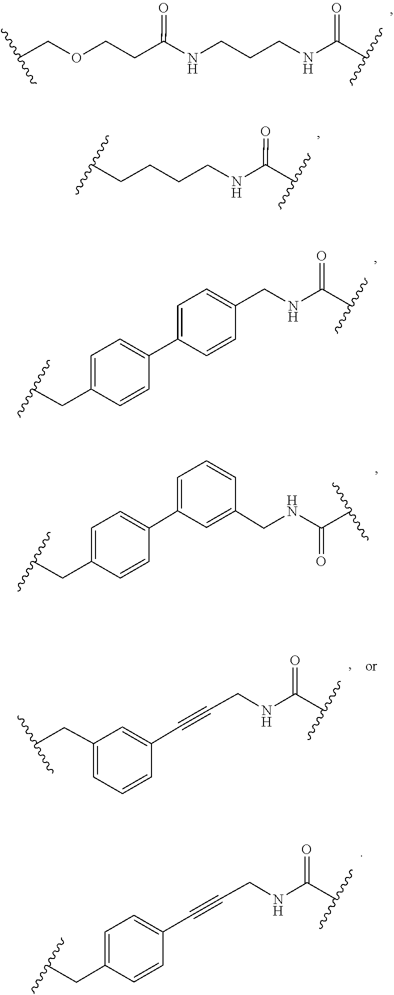 [SMILES: *COCCC(=O)NCCCNC(*)=O ; *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L² is independently a bond. In embodiments, L² is independently

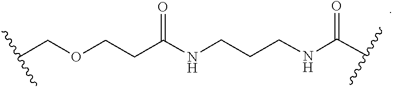 [SMILES: *COCCC(=O)NCCCNC(*)=O]

In embodiments, L² is independently

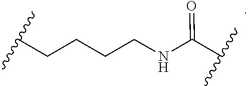 [SMILES: *CCCCNC(*)=O]

In embodiments, L² is independently

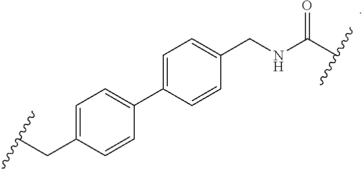 [SMILES: *Cc1ccc(-c2ccc(CNC(*)=O)cc2)cc1]

In embodiments, L² is independently

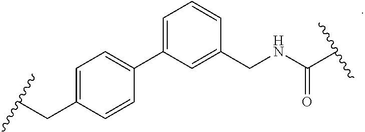 [SMILES: *Cc1ccc(-c2cccc(CNC(*)=O)c2)cc1]

In embodiments, L² is independently

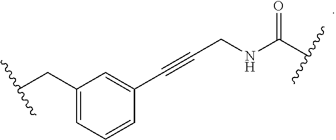 [SMILES: *Cc1cccc(C#CCNC(*)=O)c1]

In embodiments, L² is independently

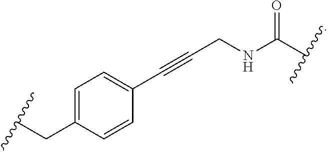 [SMILES: *Cc1ccc(C#CCNC(*)=O)cc1]

In embodiments, L^{2A} is independently substituted or unsubstituted 2 to 8 membered heteroalkylene; L^{2B} is independently —NHC(O)— or —C(O)NH—; L^{2C} is independently unsubstituted C₁-C₈ alkylene; L^{2D} is independently a bond, —NHC(O)—, or —C(O)NH—; and L^{2E} is independently —NHC(O)—. In embodiments, L^{2A} is independently substituted or unsubstituted 2 to 5 membered heteroalkylene; L^{2B} is independently —NHC(O)— or —C(O)NH—; L^{2C} is independently unsubstituted C₁-C₄ alkylene; L^{2D} is independently a bond, —NHC(O)—, or —C(O)NH—; and L^{2E} is independently —NHC(O)—. In embodiments, L^{2A} is independently unsubstituted 4 to 5 membered heteroalkylene; L^{2B} is independently —NHC(O)— or —C(O)NH—; L^{2C} is independently unsubstituted C₁-C₄ alkylene; L^{2D} is independently a bond; and L^{2E} is independently —NHC(O)—.

In embodiments, L³ is independently —NHC(O)—, —C(O)NH—, substituted or unsubstituted alkylene, or substituted or unsubstituted heteroalkylene. In embodiments, L³ is independently —NHC(O)—. In embodiments, L³ is independently —C(O)NH—. In embodiments, L³ is substituted or unsubstituted alkylene. In embodiments, L³ is substituted or unsubstituted heteroalkylene.

In embodiments, L³ is independently substituted or unsubstituted alkylene (e.g., C₁-C₂₀, C₁-C₁₂, C₁-C₈, C₁-C₆, C₁-C₄, or C₁-C₂). In embodiments, L³ is independently substituted alkylene (e.g., C₁-C₂₀, C₁-C₁₂, C₁-C₈, C₁-C₆, C₁-C₄, or C₁-C₂). In embodiments, L³ is independently unsubstituted alkylene (e.g., C₁-C₂₀, C₁-C₁₂, C₁-C₈, C₁-C₆, C₁-C₄, or C₁-C₂). In embodiments, L³ is independently substituted or unsubstituted C₁-C₂₀ alkylene. In embodiments, L³ is independently substituted C₁-C₂₀ alkylene. In embodiments, L³ is independently unsubstituted C₁-C₂₀ alkylene. In embodiments, L³ is independently substituted or unsubstituted C₁-C₁₂ alkylene. In embodiments, L³ is independently substituted C₁-C₁₂ alkylene. In embodiments, L³ is independently unsubstituted C₁-C₁₂ alkylene. In embodiments, L³ is independently substituted or unsubstituted C₁-C₈ alkylene. In embodiments, L³ is independently substituted C₁-C₈ alkylene. In embodiments, L³ is independently unsubstituted C₁-C₈ alkylene. In embodiments, L³ is independently substituted or unsubstituted C₁-C₆ alkylene. In embodiments, L³ is independently substituted C₁-C₆ alkylene. In embodiments, L³ is independently unsubstituted C₁-C₆ alkylene. In embodiments, L³ is independently substituted or unsubstituted C₁-C₄ alkylene. In embodiments, L³ is independently substituted C₁-C₄ alkylene. In embodiments, L³ is independently unsubstituted C₁-C₄ alkylene. In embodiments, L³ is independently substituted or unsubstituted ethylene. In embodiments, L³ is independently substituted ethylene. In embodiments, L³ is independently unsubstituted ethylene. In embodiments, L³ is independently substituted or unsubstituted methylene. In embodiments, L³ is independently substituted methylene. In embodiments, L³ is independently unsubstituted methylene.

In embodiments, L³ is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L³ is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L³ is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L³ is independently substituted or unsubstituted 2 to 25 membered heteroalkylene. In embodiments, L³ is independently substituted 2 to 25 membered heteroalkylene. In embodiments, L³ is independently unsubstituted 2 to 25 membered heteroalkylene. In embodiments, L³ is independently substituted or unsubstituted 2 to 20 membered heteroalkylene. In embodiments, L³ is independently substituted 2 to 20 membered heteroalkylene. In embodiments, L³ is independently unsubstituted 2 to 20 membered heteroalkylene. In embodiments, L³ is independently substituted or unsubstituted 2 to 12 membered heteroalkylene. In embodiments, L³ is independently substituted 2 to 12 membered heteroalkylene. In embodiments, L³ is independently unsubstituted 2 to 12 membered heteroalkylene. In embodiments, L³ is independently substituted or unsubstituted 2 to 8 membered heteroalkylene. In embodiments, L³ is independently substituted 2 to 8 membered heteroalkylene. In embodiments, L³ is independently unsubstituted 2 to 8 membered heteroalkylene. In embodiments, L³ is independently substituted or unsubstituted 2 to 6 membered heteroalkylene. In embodiments, L³ is independently substituted 2 to 6 membered heteroalkylene. In embodiments, L³ is independently unsubstituted 2 to 6 membered heteroalkylene. In embodiments, L³ is independently substituted or unsubstituted 4 to 6 membered heteroalkylene. In embodiments, L³ is independently substituted 4 to 6 membered heteroalkylene. In embodiments, L³ is independently unsubstituted 4 to 6 membered heteroalkylene. In embodiments, L³ is independently substituted or unsubstituted 2 to 3 membered heteroalkylene. In embodiments, L³ is independently substituted 2 to 3 membered heteroalkylene. In embodiments, L³ is independently unsubstituted 2 to 3 membered heteroalkylene. In embodiments, L³ is independently substituted or unsubstituted 4 to 5 membered heteroalkylene. In embodiments, L³ is independently substituted 4 to 5 membered heteroalkylene. In embodiments, L³ is independently unsubstituted 4 to 5 membered heteroalkylene.

In embodiments, L³ is L^{3A}-L^{3B}-L^{3C}-L^{3D}-L^{3E}. In embodiments, L^{3A} is independently a bond, —NHC(O)—, substituted or unsubstituted alkylene, or substituted or unsubstituted heteroalkylene; L^{3B} is independently a bond, —O—, —NHC(O)—, substituted or unsubstituted alkylene, substituted or unsubstituted heteroalkylene, or substituted or unsubstituted arylene; L^{3C} is independently a bond, —O—, —NHC(O)—, substituted or unsubstituted alkylene, substituted or substituted or unsubstituted heteroalkylene, or substituted or unsubstituted arylene; L^{3D} is independently a bond, —O—, —NHC(O)—, substituted or unsubstituted alkylene, substituted or unsubstituted heteroalkylene, or substituted or unsubstituted arylene; and L^{3E} is independently a bond, —NHC(O)—, substituted or unsubstituted alkylene, or substituted or unsubstituted heteroalkylene.

In embodiments, L^{3A} is independently a bond, —NHC(O)—, substituted or unsubstituted alkylene, or substituted or unsubstituted heteroalkylene. In embodiments, L^{3B} is independently a bond, —O—, —NHC(O)—, substituted or unsubstituted alkylene, substituted or unsubstituted heteroalkylene, or substituted or unsubstituted arylene. In embodiments, L^{3C} is independently a bond, —O—, —NHC(O)—, substituted or unsubstituted alkylene, substituted or substituted or unsubstituted heteroalkylene, or substituted or unsubstituted arylene. In embodiments, L^{3D} is independently a bond, —O—, —NHC(O)—, substituted or unsubstituted alkylene, substituted or unsubstituted heteroalkylene, or substituted or unsubstituted arylene.

In embodiments, L^{3E} is independently a bond, —NHC(O)—, substituted or unsubstituted alkylene, or substituted or unsubstituted heteroalkylene.

In embodiments, L^{3A} is independently a bond, —NHC(O)—, substituted or unsubstituted C₁-C₈ alkylene, or substituted or unsubstituted 3 to 8 membered heteroalkylene; L^{3B} is independently a bond, —O—, —NHC(O)—, substituted or unsubstituted C₁-C₈ alkylene, substituted or unsubstituted 3 to 8 membered heteroalkylene, or substituted or unsubstituted phenylene; L^{3C} is independently a bond, —O—, —NHC(O)—, substituted or unsubstituted C₁-C₈ alkylene, substituted or unsubstituted C₃-C₈ alkynylene, substituted or substituted or unsubstituted 3 to 8 membered heteroalkylene, or substituted or unsubstituted phenylene; L^{3D} is independently a bond, —O—, —NHC(O)—, substituted or unsubstituted C₁-C₈ alkylene, substituted or unsubstituted 3 to 8 membered heteroalkylene, or substituted or unsubstituted phenylene; and L^{3E} is independently a bond, —NHC(O)—, substituted or unsubstituted C₁-C₈ alkylene, or substituted or unsubstituted 3 to 8 membered heteroalkylene. In embodiments, L^{3A} is independently a bond, —NHC(O)—, substituted or unsubstituted C₁-C₈ alkylene, or substituted or unsubstituted 3 to 8 membered heteroalkylene. In embodiments, L^{3B} is independently a bond, —O—, —NHC(O)—, substituted or unsubstituted C₁-C₈ alkylene, substituted or unsubstituted 3 to 8 membered heteroalkylene, or substituted or unsubstituted phenylene. In embodiments, L^{3C} is independently a bond, —O—, —NHC(O)—, substituted or unsubstituted C₁-C₈ alkylene, substituted or unsubstituted C₃-C₈ alkynylene, substituted or substituted or unsubstituted 3 to 8 membered heteroalkylene, or substituted or unsubstituted phenylene. In embodiments, L^{3D} is independently a bond, —O—, —NHC(O)—, substituted or unsubstituted C₁-C₈ alkylene, substituted or unsubstituted 3 to 8 membered heteroalkylene, or substituted or unsubstituted phenylene. In embodiments, L^{3E} is independently a bond, —NHC(O)—, substituted or unsubstituted C₁-C₈ alkylene, or substituted or unsubstituted 3 to 8 membered heteroalkylene.

In embodiments, L^{3A} is independently unsubstituted C₁-C₈ alkylene, or unsubstituted 3 to 8 membered heteroalkylene; L^{3B} is independently a bond, —O—, —NHC(O)—, unsubstituted C₁-C₈ alkylene, unsubstituted C₃-C₈ alkynylene, unsubstituted 3 to 8 membered heteroalkylene, or unsubstituted phenylene; L^{3C} is independently a bond, —O—, —NHC(O)—, unsubstituted C₁-C₈ alkylene, unsubstituted 3 to 8 membered heteroalkylene, or unsubstituted phenylene; L^{3D} is independently a bond, —O—, —NHC(O)—, unsubstituted C₁-C₈ alkylene, or unsubstituted 3 to 8 membered heteroalkylene; and L^{3E} is independently —NHC(O)—.

In embodiments, L^{3A} is independently unsubstituted C₁-C₈ alkylene, or unsubstituted 3 to 8 membered heteroalkylene. In embodiments, L^{3B} is independently a bond, —O—, —NHC(O)—, unsubstituted C₁-C₈alkylene, unsubstituted C₃-C₈ alkynylene, unsubstituted 3 to 8 membered heteroalkylene, or unsubstituted phenylene. In embodiments, L^{3C} is independently a bond, —O—, —NHC(O)—, unsubstituted C₁-C₈alkylene, unsubstituted 3 to 8 membered heteroalkylene, or unsubstituted phenylene. In embodiments, L^{3D} is independently a bond, —O—, —NHC(O)—, unsubstituted C₁-C₈alkylene, or unsubstituted 3 to 8 membered heteroalkylene. In embodiments, L^{3E} is independently —NHC(O)—.

In embodiments, L^{3A} is independently a bond. In embodiments, L^{3A} is independently —NH—. In embodiments, L^{3A} is independently —O—. In embodiments, L^{3A} is independently —S—. In embodiments, L^{3A} is independently —C(O)—. In embodiments, L^{3A} is independently —NHC(O)—. In embodiments, L^{3A} is independently —S(O)₂C(O)—. In embodiments, L^{3A} is independently —OPO₂—O—. In embodiments, L^{3A} is independently —OP(S)(O)—O—. In embodiments, L^{3A} is independently —OP(S)₂—O—. In embodiments, L^{3A} is independently —S(O)NH—. In embodiments, L^{3A} is independently —NHC(O)NH—. In embodiments, L^{3A} is independently —C(O)O—. In embodiments, L^{3A} is independently —OC(O)—. In embodiments, L^{3A} is independently —C(O)NH—. In embodiments, L^{3A} is not a bond.

In embodiments, L^{3A} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{3A} is independently substituted alkylene (e.g., C₁-C₂₅, C₁-C₂₀, C₁-C₁₂, C₁-C₈, C₁-C₆, C₁-C₄, or C₁-C₂). In embodiments, L^{3A} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{3A} is independently substituted or unsubstituted C₁-C₂₅ alkylene. In embodiments, L^{3A} is independently substituted C₁-C₂₅ alkylene. In embodiments, L^{3A} is independently unsubstituted C₁-C₂₅ alkylene. In embodiments, L^{3A} is independently substituted or unsubstituted C₁-C₂₀ alkylene. In embodiments, L^{3A} is independently substituted C₁-C₂₀ alkylene. In embodiments, L^{3A} is independently unsubstituted C₁-C₂₀ alkylene. In embodiments, L^{3A} is independently substituted or unsubstituted C₁-C₁₂ alkylene. In embodiments, L^{3A} is independently substituted C₁-C₁₂ alkylene. In embodiments, L^{3A} is independently unsubstituted C₁-C₁₂ alkylene. In embodiments, L^{3A} is independently substituted or unsubstituted C₁-C₈ alkylene. In embodiments, L^{3A} is independently substituted C₁-C₈ alkylene. In embodiments, L^{3A} is independently unsubstituted C₁-C₈alkylene. In embodiments, L^{3A} is independently substituted or unsubstituted C₁-C₆ alkylene. In embodiments, L^{3A} is independently substituted C₁-C₆ alkylene. In embodiments, L^{3A} is independently unsubstituted C₁-C₆ alkylene. In embodiments, L^{3A} is independently substituted or unsubstituted C₁-C₄ alkylene. In embodiments, L^{3A} is independently substituted C₁-C₄ alkylene. In embodiments, L^{3A} is independently unsubstituted C₁-C₄ alkylene. In embodiments, L^{3A} is independently substituted or unsubstituted ethylene. In embodiments, L^{3A} is independently substituted ethylene. In embodiments, L^{3A} is independently unsubstituted ethylene. In embodiments, L^{3A} is independently substituted or unsubstituted methylene. In embodiments, L^{3A} is independently substituted methylene. In embodiments, L^{3A} is independently unsubstituted methylene.

In embodiments, L^{3A} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3A} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3A} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3A} is independently substituted or unsubstituted 2 to 25 membered heteroalkylene. In embodiments, L^{3A} is independently substituted 2 to 25 membered heteroalkylene. In embodiments, L^{3A} is independently unsubstituted 2 to 25 membered heteroalkylene. In embodiments, L^{3A} is independently substituted or unsubstituted 2 to 20 membered heteroalkylene. In embodiments, L^{3A} is independently substituted 2 to 20 membered heteroalkylene. In embodiments, L^{3A} is independently unsubstituted 2 to 20 membered heteroalkylene. In embodiments, L^{3A} is independently substituted or unsubstituted 2 to 12 membered heteroalkylene. In embodiments, L^{3A} is independently substituted 2 to 12 membered heteroalkylene. In embodiments, L^{3A} is independently unsubstituted 2 to 12 membered heteroalkylene. In embodiments, L^{3A} is independently substituted or unsubstituted 2 to 8 membered heteroalkylene. In embodiments, L^{3A} is independently substituted 2 to 8 membered heteroalkylene. In embodiments, L^{3A} is independently unsubstituted 2 to 8 membered heteroalkylene. In embodiments, L^{3A} is independently substituted or unsubstituted 2 to 6 membered heteroalkylene. In embodiments, L^{3A} is independently substituted 2 to 6 membered heteroalkylene. In embodiments, L^{3A} is independently unsubstituted 2 to 6 membered heteroalkylene. In embodiments, L^{3A} is independently substituted or unsubstituted 4 to 6 membered heteroalkylene. In embodiments, L^{3A} is independently substituted 4 to 6 membered heteroalkylene. In embodiments, L^{3A} is independently unsubstituted 4 to 6 membered heteroalkylene. In embodiments, L^{3A} is independently substituted or unsubstituted 2 to 3 membered heteroalkylene. In embodiments, L^{3A} is independently substituted 2 to 3 membered heteroalkylene. In embodiments, L^{3A} is independently unsubstituted 2 to 3 membered heteroalkylene. In embodiments, L^{3A} is independently substituted or unsubstituted 4 to 5 membered heteroalkylene. In embodiments, L^{3A} is independently substituted 4 to 5 membered heteroalkylene. In embodiments, L^{3A} is independently unsubstituted 4 to 5 membered heteroalkylene.

In embodiments, L^{3A} is independently substituted or unsubstituted cyclocycloalkylene (e.g., C₃-C₁₀, C₃-C₈, C₃-C₆, C₄-C₆, or C₅-C₆). In embodiments, L^{3A} is independently substituted cyclocycloalkylene (e.g., C₃-C₁₀, C₃-C₈, C₃-C₆, C₄-C₆, or C₅-C₆). In embodiments, L^{3A} is independently unsubstituted cyclocycloalkylene (e.g., C₃-C₁₀, C₃-C₈, C₃-C₆, C₄-C₆, or C₅-C₆). In embodiments, L^{3A} is independently substituted or unsubstituted C₃-C₁₀ cycloalkylene. In embodiments, L^{3A} is independently substituted C₃-C₁₀ cycloalkylene. In embodiments, L^{3A} is independently unsubstituted C₃-C₁₀ cycloalkylene. In embodiments, L^{3A} is independently substituted or unsubstituted C₃-C₈ cycloalkylene. In embodiments, L^{3A} is independently substituted C₃-C₈ cycloalkylene. In embodiments, L^{3A} is independently unsubstituted C₃-C₈ cycloalkylene. In embodiments, L^{3A} is independently substituted or unsubstituted C₃-C₆ cycloalkylene. In embodiments, L^{3A} is independently substituted C₃-C₆ cycloalkylene. In embodiments, L^{3A} is independently unsubstituted C₃-C₆ cycloalkylene. In embodiments, L^{3A} is independently substituted or unsubstituted C₄-C₆ cycloalkylene. In embodiments, L^{3A} is independently substituted C₄-C₆ cycloalkylene. In embodiments, L^{3A} is independently unsubstituted C₄-C₆ cycloalkylene. In embodiments, L^{3A} is independently substituted or unsubstituted C₅-C₆ cycloalkylene. In embodiments, L^{3A} is independently substituted C₅-C₆ cycloalkylene. In embodiments, L^{3A} is independently unsubstituted C₅-C₆ cycloalkylene. In embodiments, L^{3A} is independently substituted or unsubstituted hexylene. In embodiments, L^{3A} is independently substituted hexylene. In embodiments, L^{3A} is independently unsubstituted hexylene.

In embodiments, L^{3A} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3A} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{3A} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3A} is independently substituted or unsubstituted 3 to 10 membered heterocycloalkylene. In embodiments, L^{3A} is independently substituted 3 to 10 membered heterocycloalkylene. In embodiments, L^{3A} is independently unsubstituted 3 to 10 membered heterocycloalkylene. In embodiments, L^{3A} is independently substituted or unsubstituted 3 to 8 membered heterocycloalkylene. In embodiments, L^{3A} is independently substituted 3 to 8 membered heterocycloalkylene. In embodiments, L^{3A} is independently unsubstituted 3 to 8 membered heterocycloalkylene. In embodiments, L^{3A} is independently substituted or unsubstituted 3 to 6 membered heterocycloalkylene. In embodiments, L^{3A} is independently substituted 3 to 6 membered heterocycloalkylene. In embodiments, L^{3A} is independently unsubstituted 3 to 6 membered heterocycloalkylene. In embodiments, L^{3A} is independently substituted or unsubstituted 4 to 6 membered heterocycloalkylene. In embodiments, L^{3A} is independently substituted 4 to 6 membered heterocycloalkylene. In embodiments, L^{3A} is independently unsubstituted 4 to 6 membered heterocycloalkylene. In embodiments, L^{3A} is independently substituted or unsubstituted 4 to 5 membered heterocycloalkylene. In embodiments, L^{3A} is independently substituted 4 to 5 membered heterocycloalkylene. In embodiments, L^{3A} is independently unsubstituted 4 to 5 membered heterocycloalkylene. In embodiments, L^{3A} is independently substituted or unsubstituted 5 to 6 membered heterocycloalkylene. In embodiments, L^{3A} is independently substituted 5 to 6 membered heterocycloalkylene. In embodiments, L^{3A} is independently unsubstituted 5 to 6 membered heterocycloalkylene.

In embodiments, L^{3A} is independently substituted or unsubstituted arylene. In embodiments, L^{3A} is independently substituted or unsubstituted arylene (e.g., C₆-C₁₂, C₆-C₁₀, or phenyl). In embodiments, L^{3A} is independently substituted arylene (e.g., C₆-C₁₂, C₆-C₁₀, or phenyl). In embodiments, L^{3A} is independently unsubstituted arylene (e.g., C₆-C₁₂, C₆-C₁₀, or phenyl). In embodiments, L^{3A} is independently substituted or unsubstituted C₆-C₁₂ arylene. In embodiments, L^{3A} is independently substituted C₆-C₁₂ arylene. In embodiments, L^{3A} is independently unsubstituted C₆-C₁₂ arylene. In embodiments, L^{3A} is independently substituted or unsubstituted C₆-C₁₀ arylene. In embodiments, L^{3A} is independently substituted C₆-C₁₀ arylene. In embodiments, L^{3A} is independently unsubstituted C₆-C₁₀ arylene. In embodiments, L^{3A} is independently substituted or unsubstituted phenylene. In embodiments, L^{3A} is independently substituted phenylene. In embodiments, L^{3A} is independently unsubstituted phenylene. In embodiments, L^{3A} is independently substituted or unsubstituted biphenylene. In embodiments, L^{3A} is independently substituted biphenylene. In embodiments, L^{3A} is independently unsubstituted biphenylene. In embodiments, L^{3A} is independently substituted or unsubstituted naphthylene. In embodiments, L^{3A} is independently substituted naphthylene. In embodiments, L^{3A} is independently unsubstituted naphthylene.

In embodiments, L^{3A} is independently substituted or unsubstituted heteroarylene. In embodiments, L^{3A} is independently substituted heteroarylene. In embodiments, L^{3A} is independently unsubstituted heteroarylene. In embodiments, L^{3A} is independently substituted or unsubstituted heteroarylene (e.g., 5 to 12 membered, 5 to 10 membered, 5 to 9 membered, or 5 to 6 membered). In embodiments, L^{3A} is independently substituted heteroarylene (e.g., 5 to 12 membered, 5 to 10 membered, 5 to 9 membered, or 5 to 6 membered). In embodiments, L^{3A} is independently unsubstituted heteroarylene (e.g., 5 to 12 membered, 5 to 10 membered, 5 to 9 membered, or 5 to 6 membered). In embodiments, L^{3A} is independently substituted or unsubstituted 5 to 12 membered heteroarylene. In embodiments, L^{3A} is independently substituted 5 to 12 membered heteroarylene. In embodiments, L^{3A} is independently unsubstituted 5 to 12 membered heteroarylene. In embodiments, L^{3A} is independently substituted or unsubstituted 5 to 10 membered heteroarylene. In embodiments, L^{3A} is independently substituted 5 to 10 membered heteroarylene. In embodiments, L^{3A} is independently unsubstituted 5 to 10 membered heteroarylene. In embodiments, L^{3A} is independently substituted or unsubstituted 5 to 9 membered heteroarylene. In embodiments, L^{3A} is independently substituted 5 to 9 membered heteroarylene. In embodiments, L^{3A} is independently unsubstituted 5 to 9 membered heteroarylene. In embodiments, L^{3A} is independently substituted or unsubstituted 5 to 6 membered heteroarylene. In embodiments, L^{3A} is independently substituted 5 to 6 membered heteroarylene. In embodiments, L^{3A} is independently unsubstituted 5 to 6 membered heteroarylene.

In embodiments, L^{3B} is independently a bond. In embodiments, L^{3B} is independently —NH—. In embodiments, L^{3B} is independently —O—. In embodiments, L^{3B} is independently —S—. In embodiments, L^{3B} is independently —C(O)—. In embodiments, L^{3B} is independently —NHC(O)—. In embodiments, L^{3B} is independently —S(O)₂C(O)—. In embodiments, L^{3B} is independently —OPO₂—O—. In embodiments, L^{3B} is independently —OP(S)(O)—O—. In embodiments, L^{3B} is independently —OP(S)₂—O—. In embodiments, L^{3B} is independently —S(O)NH—. In embodiments, L^{3B} is independently —NHC(O)NH—. In embodiments, L^{3B} is independently —C(O)O—. In embodiments, L^{3B} is independently —OC(O)—. In embodiments, L^{3B} is independently —C(O)NH—. In embodiments, L^{3B} is not a bond.

In embodiments, L^{3B} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{3B} is independently substituted alkylene (e.g., C₁-C₂₅, C₁-C₂₀, C₁-C₁₂, C₁-C₈, C₁-C₆, C₁-C₄, or C₁-C₂). In embodiments, L^{3B} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{3B} is independently substituted or unsubstituted C₁-C₂₅ alkylene. In embodiments, L^{3B} is independently substituted C₁-C₂₅ alkylene. In embodiments, L^{3B} is independently unsubstituted C₁-C₂₅ alkylene. In embodiments, L^{3B} is independently substituted or unsubstituted C₁-C₂₀ alkylene. In embodiments, L^{3B} is independently substituted C₁-C₂₀ alkylene. In embodiments, L^{3B} is independently unsubstituted C₁-C₂₀ alkylene. In embodiments, L^{3B} is independently substituted or unsubstituted C₁-C₁₂ alkylene. In embodiments, L^{3B} is independently substituted C₁-C₁₂ alkylene. In embodiments, L^{3B} is independently unsubstituted C₁-C₁₂ alkylene. In embodiments, L^{3B} is independently substituted or unsubstituted C₁-C₈ alkylene. In embodiments, L^{3B} is independently substituted C₁-C₈ alkylene. In embodiments, L^{3B} is independently unsubstituted C₁-C₈ alkylene. In embodiments, L^{3B} is independently substituted or unsubstituted C₁-C₆ alkylene. In embodiments, L^{3B} is independently substituted C₁-C₆ alkylene. In embodiments, L^{3B} is independently unsubstituted C₁-C₆ alkylene. In embodiments, L^{3B} is independently substituted or unsubstituted C₁-C₄ alkylene. In embodiments, L^{3B} is independently substituted C₁-C₄ alkylene. In embodiments, L^{3B} is independently unsubstituted C₁-C₄ alkylene. In embodiments, L^{3B} is independently substituted or unsubstituted ethylene. In embodiments, L^{3B} is independently substituted ethylene. In embodiments, L^{3B} is independently unsubstituted ethylene. In embodiments, L^{3B} is independently substituted or unsubstituted methylene. In embodiments, L^{3B} is independently substituted methylene. In embodiments, L^{3B} is independently unsubstituted methylene.

In embodiments, L^{3B} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3B} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3B} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3B} is independently substituted or unsubstituted 2 to 25 membered heteroalkylene. In embodiments, L^{3B} is independently substituted 2 to 25 membered heteroalkylene. In embodiments, L^{3B} independently unsubstituted 2 to 25 membered heteroalkylene. In embodiments, L^{3B} is independently substituted or unsubstituted 2 to 20 membered heteroalkylene. In embodiments, L^{3B} is independently substituted 2 to 20 membered heteroalkylene. In embodiments, L^{3B} is independently unsubstituted 2 to 20 membered heteroalkylene. In embodiments, L^{3B} is independently substituted or unsubstituted 2 to 12 membered heteroalkylene. In embodiments, L^{3B} is independently substituted 2 to 12 membered heteroalkylene. In embodiments, L^{3B} is independently unsubstituted 2 to 12 membered heteroalkylene. In embodiments, L^{3B} is independently substituted or unsubstituted 2 to 8 membered heteroalkylene. In embodiments, L^{3B} is independently substituted 2 to 8 membered heteroalkylene. In embodiments, L^{3B} is independently unsubstituted 2 to 8 membered heteroalkylene. In embodiments, L^{3B} is independently substituted or unsubstituted 2 to 6 membered heteroalkylene. In embodiments, L^{3B} is independently substituted 2 to 6 membered heteroalkylene. In embodiments, L^{3B} is independently unsubstituted 2 to 6 membered heteroalkylene. In embodiments, L^{3B} is independently substituted or unsubstituted 4 to 6 membered heteroalkylene. In embodiments, L^{3B} is independently substituted 4 to 6 membered heteroalkylene. In embodiments, L^{3B} is independently unsubstituted 4 to 6 membered heteroalkylene. In embodiments, L^{3B} is independently substituted or unsubstituted 2 to 3 membered heteroalkylene. In embodiments, L^{3B} is independently substituted 2 to 3 membered heteroalkylene. In embodiments, L^{3B} is independently unsubstituted 2 to 3 membered heteroalkylene. In embodiments, L^{3B} is independently substituted or unsubstituted 4 to 5 membered heteroalkylene. In embodiments, L^{3B} is independently substituted 4 to 5 membered heteroalkylene. In embodiments, L^{3B} is independently unsubstituted 4 to 5 membered heteroalkylene.

In embodiments, L^{3B} is independently substituted or unsubstituted cyclocycloalkylene (e.g., C₃-C₁₀, C₃-C₈, C₃-C₆, C₄-C₆, or C₅-C₆). In embodiments, L^{3B} is independently substituted cyclocycloalkylene (e.g., C₃-C₁₀, C₃-C₈, C₃-C₆, C₄-C₆, or C₅-C₆). In embodiments, L^{3B} is independently unsubstituted cyclocycloalkylene (e.g., C₃-C₁₀, C₃-C₈, C₃-C₆, C₄-C₆, or C₅-C₆). In embodiments, L^{3B} is independently substituted or unsubstituted C₃-C₁₀ cycloalkylene. In embodiments, L^{3B} is independently substituted C₃-C₁₀ cycloalkylene. In embodiments, L^{3B} is independently unsubstituted C₃-C₁₀ cycloalkylene. In embodiments, L^{3B} is independently substituted or unsubstituted C₃-C₈ cycloalkylene. In embodiments, L^{3B} is independently substituted C₃-C₈ cycloalkylene. In embodiments, L^{3B} is independently unsubstituted C₃-C₈ cycloalkylene. In embodiments, L^{3B} is independently substituted or unsubstituted C₃-C₆ cycloalkylene. In embodiments, L^{3B} is independently substituted C₃-C₆ cycloalkylene. In embodiments, L^{3B} is independently unsubstituted C₃-C₆ cycloalkylene. In embodiments, L^{3B} is independently substituted or unsubstituted C₄-C₆ cycloalkylene. In embodiments, L^{3B} is independently substituted C₄-C₆ cycloalkylene. In embodiments, L^{3B} is independently unsubstituted C₄-C₆ cycloalkylene. In embodiments, L^{3B} is independently substituted or unsubstituted C₅-C₆ cycloalkylene. In embodiments, L^{3B} is independently substituted C₅-C₆ cycloalkylene. In embodiments, L^{3B} is independently unsubstituted C₅-C₆ cycloalkylene. In embodiments, L^{3B} is independently substituted or unsubstituted hexylene. In embodiments, L^{3B} is independently substituted hexylene. In embodiments, L^{3B} is independently unsubstituted hexylene.

In embodiments, L^{3B} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3B} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{3B} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3B} is independently substituted or unsubstituted 3 to 10 membered heterocycloalkylene. In embodiments, L^{3B} is independently substituted 3 to 10 membered heterocycloalkylene. In embodiments, L^{3B} is independently unsubstituted 3 to 10 membered heterocycloalkylene. In embodiments, L^{3B} is independently substituted or unsubstituted 3 to 8 membered heterocycloalkylene. In embodiments, L^{3B} is independently substituted 3 to 8 membered heterocycloalkylene. In embodiments, L^{3B} is independently unsubstituted 3 to 8 membered heterocycloalkylene. In embodiments, L^{3B} is independently substituted or unsubstituted 3 to 6 membered heterocycloalkylene. In embodiments, L^{3B} is independently substituted 3 to 6 membered heterocycloalkylene. In embodiments, L^{3B} is independently unsubstituted 3 to 6 membered heterocycloalkylene. In embodiments, L^{3B} is independently substituted or unsubstituted 4 to 6 membered heterocycloalkylene. In embodiments, L^{3B} is independently substituted 4 to 6 membered heterocycloalkylene. In embodiments, L^{3B} is independently unsubstituted 4 to 6 membered heterocycloalkylene. In embodiments, L^{3B} is independently substituted or unsubstituted 4 to 5 membered heterocycloalkylene. In embodiments, L^{3B} is independently substituted 4 to 5 membered heterocycloalkylene. In embodiments, L^{3B} is independently unsubstituted 4 to 5 membered heterocycloalkylene. In embodiments, L^{3B} is independently substituted or unsubstituted 5 to 6 membered heterocycloalkylene. In embodiments, L^{3B} is independently substituted 5 to 6 membered heterocycloalkylene. In embodiments, L^{3B} is independently unsubstituted 5 to 6 membered heterocycloalkylene.

In embodiments, L^{3B} is independently substituted or unsubstituted arylene. In embodiments, L^{3B} is independently substituted or unsubstituted arylene (e.g., C₆-C₁₂, C₆-C₁₀, or phenyl). In embodiments, L^{3B} is independently substituted arylene (e.g., C₆-C₁₂, C₆-C₁₀, or phenyl). In embodiments, L^{3B} is independently unsubstituted arylene (e.g., C₆-C₁₂, C₆-C₁₀, or phenyl). In embodiments, L^{3B} is independently substituted or unsubstituted C₆-C₁₂ arylene. In embodiments, L^{3B} is independently substituted C₆-C₁₂ arylene. In embodiments, L^{3B} is independently unsubstituted C₆-C₁₂ arylene. In embodiments, L^{3B} is independently substituted or unsubstituted C₆-C₁₀ arylene. In embodiments, L^{3B} is independently substituted C₆-C₁₀ arylene. In embodiments, L^{3B} is independently unsubstituted C₆-C₁₀ arylene. In embodiments, L^{3B} is independently substituted or unsubstituted phenylene. In embodiments, L^{3B} is independently substituted phenylene. In embodiments, L^{3B} is independently unsubstituted phenylene. In embodiments, L^{3B} is independently substituted or unsubstituted biphenylene. In embodiments, L^{3B} is independently substituted biphenylene. In embodiments, L^{3B} is independently unsubstituted biphenylene. In embodiments, L^{3B} is independently substituted or unsubstituted naphthylene. In embodiments, L^{3B} is independently substituted naphthylene. In embodiments, L^{3B} is independently unsubstituted naphthylene.

In embodiments, L^{3B} is independently substituted or unsubstituted heteroarylene. In embodiments, L^{3B} is independently substituted heteroarylene. In embodiments, L^{3B} is independently unsubstituted heteroarylene. In embodiments, L^{3B} is independently substituted or unsubstituted heteroarylene (e.g., 5 to 12 membered, 5 to 10 membered, 5 to 9 membered, or 5 to 6 membered). In embodiments, L^{3B} is independently substituted heteroarylene (e.g., 5 to 12 membered, 5 to 10 membered, 5 to 9 membered, or 5 to 6 membered). In embodiments, L^{3B} is independently unsubstituted heteroarylene (e.g., 5 to 12 membered, 5 to 10 membered, 5 to 9 membered, or 5 to 6 membered). In embodiments, L^{3B} is independently substituted or unsubstituted 5 to 12 membered heteroarylene. In embodiments, L^{3B} is independently substituted 5 to 12 membered heteroarylene. In embodiments, L^{3B} is independently unsubstituted 5 to 12 membered heteroarylene. In embodiments, L^{3B} is independently substituted or unsubstituted 5 to 10 membered heteroarylene. In embodiments, L^{3B} is independently substituted 5 to 10 membered heteroarylene. In embodiments, L^{3B} is independently unsubstituted 5 to 10 membered heteroarylene. In embodiments, L^{3B} is independently substituted or unsubstituted 5 to 9 membered heteroarylene. In embodiments, L^{3B} is independently substituted 5 to 9 membered heteroarylene. In embodiments, L^{3B} is independently unsubstituted 5 to 9 membered heteroarylene. In embodiments, L^{3B} is independently substituted or unsubstituted 5 to 6 membered heteroarylene. In embodiments, L^{3B} is independently substituted 5 to 6 membered heteroarylene. In embodiments, L^{3B} independently unsubstituted 5 to 6 membered heteroarylene.

In embodiments, L^{3C} is independently a bond. In embodiments, L^{3C} is independently —NH—. In embodiments, L^{3C} is independently —O—. In embodiments, L^{3C} is independently —S—. In embodiments, L^{3C} is independently —C(O)—. In embodiments, L^{3C} is independently —NHC(O)—. In embodiments, L^{3C} is independently —S(O)₂C(O)—. In embodiments, L^{3C} is independently —OPO₂—O—. In embodiments, L^{3C} is independently —OP(S)(O)—O—. In embodiments, L^{3C} is independently —OP(S)₂—O—. In embodiments, L^{3C} is independently —S(O)NH—. In embodiments, L^{3C} is independently —NHC(O)NH—. In embodiments, L^{3C} is independently —C(O)O—. In embodiments, L^{3C} is independently —OC(O)—. In embodiments, L^{3C} is independently —C(O)NH—. In embodiments, L^{3C} is not a bond.

In embodiments, L^{3C} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{3C} is independently substituted alkylene (e.g., C₁-C₂₅, C₁-C₂₀, C₁-C₁₂, C₁-C₈, C₁-C₆, C₁-C₄, or C₁-C₂). In embodiments, L^{3C} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{3C} is independently substituted or unsubstituted C₁-C₂₅ alkylene. In embodiments, L^{3C} is independently substituted C₁-C₂₅ alkylene. In embodiments, L^{3C} is independently unsubstituted C₁-C₂₅ alkylene. In embodiments, L^{3C} is independently substituted or unsubstituted C₁-C₂₀ alkylene. In embodiments, L^{3C} is independently substituted C₁-C₂₀ alkylene. In embodiments, L^{3C} is independently unsubstituted C₁-C₂₀ alkylene. In embodiments, L^{3C} is independently substituted or unsubstituted C₁-C₁₂ alkylene. In embodiments, L^{3C} is independently substituted C₁-C₁₂ alkylene. In embodiments, L^{3C} is independently unsubstituted C₁-C₁₂ alkylene. In embodiments, L^{3C} is independently substituted or unsubstituted C₁-C₈alkylene. In embodiments, L^{3C} is independently substituted C₁-C₈ alkylene. In embodiments, L^{3C} is independently unsubstituted C₁-C₈ alkylene. In embodiments, L^{3C} is independently substituted or unsubstituted C₁-C₆ alkylene. In embodiments, L^{3C} is independently substituted C₁-C₆ alkylene. In embodiments, L^{3C} is independently unsubstituted C₁-C₆ alkylene. In embodiments, L^{3C} is independently substituted or unsubstituted C₁-C₄ alkylene. In embodiments, L^{3C} is independently substituted C₁-C₄ alkylene. In embodiments, L^{3C} is independently unsubstituted C₁-C₄ alkylene. In embodiments, L^{3C} is independently substituted or unsubstituted ethylene. In embodiments, L^{3C} is independently substituted ethylene. In embodiments, L^{3C} is independently unsubstituted ethylene. In embodiments, L^{3C} is independently substituted or unsubstituted methylene. In embodiments, L^{3C} is independently substituted methylene. In embodiments, L^{3C} is independently unsubstituted methylene.

In embodiments, L^{3C} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3C} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3C} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3C} is independently substituted or unsubstituted 2 to 25 membered heteroalkylene. In embodiments, L^{3C} is independently substituted 2 to 25 membered heteroalkylene. In embodiments, L^{3C} is independently unsubstituted 2 to 25 membered heteroalkylene. In embodiments, L^{3C} is independently substituted or unsubstituted 2 to 20 membered heteroalkylene. In embodiments, L^{3C} is independently substituted 2 to 20 membered heteroalkylene. In embodiments, L^{3C} is independently unsubstituted 2 to 20 membered heteroalkylene. In embodiments, L^{3C} is independently substituted or unsubstituted 2 to 12 membered heteroalkylene. In embodiments, L^{3C} is independently substituted 2 to 12 membered heteroalkylene. In embodiments, L^{3C} is independently unsubstituted 2 to 12 membered heteroalkylene. In embodiments, L^{3C} is independently substituted or unsubstituted 2 to 8 membered heteroalkylene. In embodiments, L^{3C} is independently substituted 2 to 8 membered heteroalkylene. In embodiments, L^{3C} is independently unsubstituted 2 to 8 membered heteroalkylene. In embodiments, L^{3C} is independently substituted or unsubstituted 2 to 6 membered heteroalkylene. In embodiments, L^{3C} is independently substituted 2 to 6 membered heteroalkylene. In embodiments, L^{3C} is independently unsubstituted 2 to 6 membered heteroalkylene. In embodiments, L^{3C} is independently substituted or unsubstituted 4 to 6 membered heteroalkylene. In embodiments, L^{3C} is independently substituted 4 to 6 membered heteroalkylene. In embodiments, L^{3C} is independently unsubstituted 4 to 6 membered heteroalkylene. In embodiments, L^{3C} is independently substituted or unsubstituted 2 to 3 membered heteroalkylene. In embodiments, L^{3C} is independently substituted 2 to 3 membered heteroalkylene. In embodiments, L^{3C} is independently unsubstituted 2 to 3 membered heteroalkylene. In embodiments, L^{3C} is independently substituted or unsubstituted 4 to 5 membered heteroalkylene. In embodiments, L^{3C} is independently substituted 4 to 5 membered heteroalkylene. In embodiments, L^{3C} is independently unsubstituted 4 to 5 membered heteroalkylene.

In embodiments, L^{3C} is independently substituted or unsubstituted cyclocycloalkylene (e.g., C₃-C₁₀, C₃-C₈, C₃-C₆, C₄-C₆, or C₅-C₆). In embodiments, L^{3C} is independently substituted cyclocycloalkylene (e.g., C₃-C₁₀, C₃-C₈, C₃-C₆, C₄-C₆, or C₅-C₆). In embodiments, L^{3C} is independently unsubstituted cyclocycloalkylene (e.g., C₃-C₁₀, C₃-C₈, C₃-C₆, C₄-C₆, or C₅-C₆). In embodiments, L^{3C} is independently substituted or unsubstituted C₃-C₁₀ cycloalkylene. In embodiments, L^{3C} is independently substituted C₃-C₁₀ cycloalkylene. In embodiments, L^{3C} is independently unsubstituted C₃-C₁₀ cycloalkylene. In embodiments, L^{3C} is independently substituted or unsubstituted C₃-C₈ cycloalkylene. In embodiments, L^{3C} is independently substituted C₃-C₈ cycloalkylene. In embodiments, L^{3C} is independently unsubstituted C₃-C₈ cycloalkylene. In embodiments, L^{3C} is independently substituted or unsubstituted C₃-C₆ cycloalkylene. In embodiments, L^{3C} is independently substituted C₃-C₆ cycloalkylene. In embodiments, L^{3C} is independently unsubstituted C₃-C₆ cycloalkylene. In embodiments, L^{3C} is independently substituted or unsubstituted C₄-C₆ cycloalkylene. In embodiments, L^{3C} is independently substituted C₄-C₆ cycloalkylene. In embodiments, L^{3C} is independently unsubstituted C₄-C₆ cycloalkylene. In embodiments, L^{3C} is independently substituted or unsubstituted C₅-C₆ cycloalkylene. In embodiments, L^{3C} is independently substituted C₅-C₆ cycloalkylene. In embodiments, L^{3C} is independently unsubstituted C₅-C₆ cycloalkylene. In embodiments, L^{3C} is independently substituted or unsubstituted hexylene. In embodiments, L^{3C} is independently substituted hexylene. In embodiments, L^{3C} is independently unsubstituted hexylene.

In embodiments, L^{3C} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3C} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{3C} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3C} is independently substituted or unsubstituted 3 to 10 membered heterocycloalkylene. In embodiments, L^{3C} is independently substituted 3 to 10 membered heterocycloalkylene. In embodiments, L^{3C} is independently unsubstituted 3 to 10 membered heterocycloalkylene. In embodiments, L^{3C} is independently substituted or unsubstituted 3 to 8 membered heterocycloalkylene. In embodiments, L^{3C} is independently substituted 3 to 8 membered heterocycloalkylene. In embodiments, L^{3C} is independently unsubstituted 3 to 8 membered heterocycloalkylene. In embodiments, L^{3C} is independently substituted or unsubstituted 3 to 6 membered heterocycloalkylene. In embodiments, L^{3C} is independently substituted 3 to 6 membered heterocycloalkylene. In embodiments, L^{3C} is independently unsubstituted 3 to 6 membered heterocycloalkylene. In embodiments, L^{3C} is independently substituted or unsubstituted 4 to 6 membered heterocycloalkylene. In embodiments, L^{3C} is independently substituted 4 to 6 membered heterocycloalkylene. In embodiments, L^{3C} is independently unsubstituted 4 to 6 membered heterocycloalkylene. In embodiments, L^{3C} is independently substituted or unsubstituted 4 to 5 membered heterocycloalkylene. In embodiments, L^{3C} is independently substituted 4 to 5 membered heterocycloalkylene. In embodiments, L^{3C} is independently unsubstituted 4 to 5 membered heterocycloalkylene. In embodiments, L^{3C} is independently substituted or unsubstituted 5 to 6 membered heterocycloalkylene. In embodiments, L^{3C} is independently substituted 5 to 6 membered heterocycloalkylene. In embodiments, L^{3C} is independently unsubstituted 5 to 6 membered heterocycloalkylene.

In embodiments, L^{3C} is independently substituted or unsubstituted arylene. In embodiments, L^{3C} is independently substituted or unsubstituted arylene (e.g., C₆-C₁₂, C₆-C₁₀, or phenyl). In embodiments, L^{3C} is independently substituted arylene (e.g., C₆-C₁₂, C₆-C₁₀, or phenyl). In embodiments, L^{3C} is independently unsubstituted arylene (e.g., C₆-C₁₂, C₆-C₁₀, or phenyl). In embodiments, L^{3C} is independently substituted or unsubstituted C₆-C₁₂ arylene. In embodiments, L^{3C} is independently substituted C₆-C₁₂ arylene. In embodiments, L^{3C} is independently unsubstituted C₆-C₁₂ arylene. In embodiments, L^{3C} is independently substituted or unsubstituted C₆-C₁₀ arylene. In embodiments, L^{3C} is independently substituted C₆-C₁₀ arylene. In embodiments, L^{3C} is independently unsubstituted C₆-C₁₀ arylene. In embodiments, L^{3C} is independently substituted or unsubstituted phenylene. In embodiments, L^{3C} is independently substituted phenylene. In embodiments, L^{3C} is independently unsubstituted phenylene. In embodiments, L^{3C} is independently substituted or unsubstituted biphenylene. In embodiments, L^{3C} is independently substituted biphenylene. In embodiments, L^{3C} is independently unsubstituted biphenylene. In embodiments, L^{3C} is independently substituted or unsubstituted naphthylene. In embodiments, L^{3C} is independently substituted naphthylene. In embodiments, L^{3C} is independently unsubstituted naphthylene.

In embodiments, L^{3C} is independently substituted or unsubstituted heteroarylene. In embodiments, L^{3C} is independently substituted heteroarylene. In embodiments, L^{3C} is independently unsubstituted heteroarylene. In embodiments, L^{3C} is independently substituted or unsubstituted heteroarylene (e.g., 5 to 12 membered, 5 to 10 membered, 5 to 9 membered, or 5 to 6 membered). In embodiments, L^{3C} is independently substituted heteroarylene (e.g., 5 to 12 membered, 5 to 10 membered, 5 to 9 membered, or 5 to 6 membered). In embodiments, L^{3C} is independently unsubstituted heteroarylene (e.g., 5 to 12 membered, 5 to 10 membered, 5 to 9 membered, or 5 to 6 membered). In embodiments, L^{3C} is independently substituted or unsubstituted 5 to 12 membered heteroarylene. In embodiments, L^{3C} is independently substituted 5 to 12 membered heteroarylene. In embodiments, L^{3C} is independently unsubstituted 5 to 12 membered heteroarylene. In embodiments, L^{3C} is independently substituted or unsubstituted 5 to 10 membered heteroarylene. In embodiments, L^{3C} is independently substituted 5 to 10 membered heteroarylene. In embodiments, L^{3C} is independently unsubstituted 5 to 10 membered heteroarylene. In embodiments, L^{3C} is independently substituted or unsubstituted 5 to 9 membered heteroarylene. In embodiments, L^{3C} is independently substituted 5 to 9 membered heteroarylene. In embodiments, L^{3C} is independently unsubstituted 5 to 9 membered heteroarylene. In embodiments, L^{3C} is independently substituted or unsubstituted 5 to 6 membered heteroarylene. In embodiments, L^{3C} is independently substituted 5 to 6 membered heteroarylene. In embodiments, L^{3C} is independently unsubstituted 5 to 6 membered heteroarylene.

In embodiments, L^{3D} is independently a bond. In embodiments, L^{3D} is independently —NH—. In embodiments, L^{3D} is independently —O—. In embodiments, L^{3D} is independently —S—. In embodiments, L^{3D} is independently —C(O)—. In embodiments, L^{3D} is independently —NHC(O)—. In embodiments, L^{3D} is independently —S(O)₂C(O)—. In embodiments, L^{3D} is independently —OPO₂—O—. In embodiments, L^{3D} is independently —OP(S)(O)—O—. In embodiments, L^{3D} is independently —OP(S)₂—O—. In embodiments, L^{3D} is independently —S(O)NH—. In embodiments, L^{3D} is independently —NHC(O)NH—. In embodiments, L^{3D} is independently —C(O)O—. In embodiments, L^{3D} is independently —OC(O)—. In embodiments, L^{3D} is independently —C(O)NH—. In embodiments, L^{3D} is not a bond.

In embodiments, L^{3D} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{3D} is independently substituted alkylene (e.g., C₁-C₂₅, C₁-C₂₀, C₁-C₁₂, C₁-C₈, C₁-C₆, C₁-C₄, or C₁-C₂). In embodiments, L^{3D} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{3D} is independently substituted or unsubstituted C₁-C₂₅ alkylene. In embodiments, L^{3D} is independently substituted C₁-C₂₅ alkylene. In embodiments, L^{3D} is independently unsubstituted C₁-C₂₅ alkylene. In embodiments, L^{3D} is independently substituted or unsubstituted C₁-C₂₀ alkylene. In embodiments, L^{3D} is independently substituted C₁-C₂₀ alkylene. In embodiments, L^{3D} is independently unsubstituted C₁-C₂₀ alkylene. In embodiments, L^{3D} is independently substituted or unsubstituted C₁-C₁₂ alkylene. In embodiments, L^{3D} is independently substituted C₁-C₁₂ alkylene. In embodiments, L^{3D} is independently unsubstituted C₁-C₁₂ alkylene. In embodiments, L^{3D} is independently substituted or unsubstituted C₁-C₈ alkylene. In embodiments, L^{3D} is independently substituted C₁-C₈ alkylene. In embodiments, L^{3D} is independently unsubstituted C₁-C₈ alkylene. In embodiments, L^{3D} is independently substituted or unsubstituted C₁-C₆ alkylene. In embodiments, L^{3D} is independently substituted C₁-C₆ alkylene. In embodiments, L^{3D} is independently unsubstituted C₁-C₆ alkylene. In embodiments, L^{3D} is independently substituted or unsubstituted C₁-C₄ alkylene. In embodiments, L^{3D} is independently substituted C₁-C₄ alkylene. In embodiments, L^{3D} is independently unsubstituted C₁-C₄ alkylene. In embodiments, L^{3D} is independently substituted or unsubstituted ethylene. In embodiments, L^{3D} is independently substituted ethylene. In embodiments, L^{3D} is independently unsubstituted ethylene. In embodiments, L^{3D} is independently substituted or unsubstituted methylene. In embodiments, L^{3D} is independently substituted methylene. In embodiments, L^{3D} is independently unsubstituted methylene.

In embodiments, L^{3D} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3D} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3D} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3D} is independently substituted or unsubstituted 2 to 25 membered heteroalkylene. In embodiments, L^{3D} is independently substituted 2 to 25 membered heteroalkylene. In embodiments, L^{3D} is independently unsubstituted 2 to 25 membered heteroalkylene. In embodiments, L^{3D} is independently substituted or unsubstituted 2 to 20 membered heteroalkylene. In embodiments, L^{3D} is independently substituted 2 to 20 membered heteroalkylene. In embodiments, L^{3D} is independently unsubstituted 2 to 20 membered heteroalkylene. In embodiments, L^{3D} is independently substituted or unsubstituted 2 to 12 membered heteroalkylene. In embodiments, L^{3D} is independently substituted 2 to 12 membered heteroalkylene. In embodiments, L^{3D} is independently unsubstituted 2 to 12 membered heteroalkylene. In embodiments, L^{3D} is independently substituted or unsubstituted 2 to 8 membered heteroalkylene. In embodiments, L^{3D} is independently substituted 2 to 8 membered heteroalkylene. In embodiments, L^{3D} is independently unsubstituted 2 to 8 membered heteroalkylene. In embodiments, L^{3D} is independently substituted or unsubstituted 2 to 6 membered heteroalkylene. In embodiments, L^{3D} is independently substituted 2 to 6 membered heteroalkylene. In embodiments, L^{3D} is independently unsubstituted 2 to 6 membered heteroalkylene. In embodiments, L^{3D} is independently substituted or unsubstituted 4 to 6 membered heteroalkylene. In embodiments, L^{3D} is independently substituted 4 to 6 membered heteroalkylene. In embodiments, L^{3D} is independently unsubstituted 4 to 6 membered heteroalkylene. In embodiments, L^{3D} is independently substituted or unsubstituted 2 to 3 membered heteroalkylene. In embodiments, L^{3D} is independently substituted 2 to 3 membered heteroalkylene. In embodiments, L^{3D} is independently unsubstituted 2 to 3 membered heteroalkylene. In embodiments, L^{3D} is independently substituted or unsubstituted 4 to 5 membered heteroalkylene. In embodiments, L^{3D} is independently substituted 4 to 5 membered heteroalkylene. In embodiments, L^{3D} is independently unsubstituted 4 to 5 membered heteroalkylene.

In embodiments, L^{3D} is independently substituted or unsubstituted cyclocycloalkylene (e.g., C₃-C₁₀, C₃-C₈, C₃-C₆, C₄-C₆, or C₅-C₆). In embodiments, L^{3D} is independently substituted cyclocycloalkylene (e.g., C₃-C₁₀, C₃-C₈, C₃-C₆, C₄-C₆, or C₅-C₆). In embodiments, L^{3D} is independently unsubstituted cyclocycloalkylene (e.g., C₃-C₁₀, C₃-C₈, C₃-C₆, C₄-C₆, or C₅-C₆). In embodiments, L^{3D} is independently substituted or unsubstituted C₃-C₁₀ cycloalkylene. In embodiments, L^{3D} is independently substituted C₃-C₁₀ cycloalkylene. In embodiments, L^{3D} is independently unsubstituted C₃-C₁₀ cycloalkylene. In embodiments, L^{3D} is independently substituted or unsubstituted C₃-C₈ cycloalkylene. In embodiments, L^{3D} is independently substituted C₃-C₈ cycloalkylene. In embodiments, L^{3D} is independently unsubstituted C₃-C₈ cycloalkylene. In embodiments, L^{3D} is independently substituted or unsubstituted C₃-C₆ cycloalkylene. In embodiments, L^{3D} is independently substituted C₃-C₆ cycloalkylene. In embodiments, L^{3D} is independently unsubstituted C₃-C₆ cycloalkylene. In embodiments, L^{3D} is independently substituted or unsubstituted C₄-C₆ cycloalkylene. In embodiments, L^{3D} is independently substituted C₄-C₆ cycloalkylene. In embodiments, L^{3D} is independently unsubstituted C₄-C₆ cycloalkylene. In embodiments, L^{3D} is independently substituted or unsubstituted C₅-C₆ cycloalkylene. In embodiments, L^{3D} is independently substituted C₅-C₆ cycloalkylene. In embodiments, L^{3D} is independently unsubstituted C₅-C₆ cycloalkylene. In embodiments, L^{3D} is independently substituted or unsubstituted hexylene. In embodiments, L^{3D} is independently substituted hexylene. In embodiments, L^{3D} is independently unsubstituted hexylene.

In embodiments, L^{3D} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3D} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{3D} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3D} is independently substituted or unsubstituted 3 to 10 membered heterocycloalkylene. In embodiments, L^{3D} is independently substituted 3 to 10 membered heterocycloalkylene. In embodiments, L^{3D} is independently unsubstituted 3 to 10 membered heterocycloalkylene. In embodiments, L^{3D} is independently substituted or unsubstituted 3 to 8 membered heterocycloalkylene. In embodiments, L^{3D} is independently substituted 3 to 8 membered heterocycloalkylene. In embodiments, L^{3D} is independently unsubstituted 3 to 8 membered heterocycloalkylene. In embodiments, L^{3D} is independently substituted or unsubstituted 3 to 6 membered heterocycloalkylene. In embodiments, L^{3D} is independently substituted 3 to 6 membered heterocycloalkylene. In embodiments, L^{3D} is independently unsubstituted 3 to 6 membered heterocycloalkylene. In embodiments, L^{3D} is independently substituted or unsubstituted 4 to 6 membered heterocycloalkylene. In embodiments, L^{3D} is independently substituted 4 to 6 membered heterocycloalkylene. In embodiments, L^{3D} is independently unsubstituted 4 to 6 membered heterocycloalkylene. In embodiments, L^{3D} is independently substituted or unsubstituted 4 to 5 membered heterocycloalkylene. In embodiments, L^{3D} is independently substituted 4 to 5 membered heterocycloalkylene. In embodiments, L^{3D} is independently unsubstituted 4 to 5 membered heterocycloalkylene. In embodiments, L^{3D} is independently substituted or unsubstituted 5 to 6 membered heterocycloalkylene. In embodiments, L^{3D} is independently substituted 5 to 6 membered heterocycloalkylene. In embodiments, L^{3D} is independently unsubstituted 5 to 6 membered heterocycloalkylene.

In embodiments, L^{3D} is independently substituted or unsubstituted arylene. In embodiments, L^{3D} is independently substituted or unsubstituted arylene (e.g., C₆-C₁₂, C₆-C₁₀, or phenyl). In embodiments, L^{3D} is independently substituted arylene (e.g., C₆-C₁₂, C₆-C₁₀, or phenyl). In embodiments, L^{3D} is independently unsubstituted arylene (e.g., C₆-C₁₂, C₆-C₁₀, or phenyl). In embodiments, L^{3D} is independently substituted or unsubstituted C₆-C₁₂ arylene. In embodiments, L^{3D} is independently substituted C₆-C₁₂ arylene. In embodiments, L^{3D} is independently unsubstituted C₆-C₁₂ arylene. In embodiments, L^{3D} is independently substituted or unsubstituted C₆-C₁₀ arylene. In embodiments, L^{3D} is independently substituted C₆-C₁₀ arylene. In embodiments, L^{3D} is independently unsubstituted C₆-C₁₀ arylene. In embodiments, L^{3D} is independently substituted or unsubstituted phenylene. In embodiments, L^{3D} is independently substituted phenylene. In embodiments, L^{3D} is independently unsubstituted phenylene. In embodiments, L^{3D} is independently substituted or unsubstituted biphenylene. In embodiments, L^{3D} is independently substituted biphenylene. In embodiments, L^{3D} is independently unsubstituted biphenylene. In embodiments, L^{3D} is independently substituted or unsubstituted naphthylene. In embodiments, L^{3D} is independently substituted naphthylene. In embodiments, L^{3D} is independently unsubstituted naphthylene.

In embodiments, L^{3D} is independently substituted or unsubstituted heteroarylene. In embodiments, L^{3D} is independently substituted heteroarylene. In embodiments, L^{3D} is independently unsubstituted heteroarylene. In embodiments, L^{3D} is independently substituted or unsubstituted heteroarylene (e.g., 5 to 12 membered, 5 to 10 membered, 5 to 9 membered, or 5 to 6 membered). In embodiments, L^{3D} is independently substituted heteroarylene (e.g., 5 to 12 membered, 5 to 10 membered, 5 to 9 membered, or 5 to 6 membered). In embodiments, L^{3D} is independently unsubstituted heteroarylene (e.g., 5 to 12 membered, 5 to 10 membered, 5 to 9 membered, or 5 to 6 membered). In embodiments, L^{3D} is independently substituted or unsubstituted 5 to 12 membered heteroarylene. In embodiments, L^{3D} is independently substituted 5 to 12 membered heteroarylene. In embodiments, L^{3D} is independently unsubstituted 5 to 12 membered heteroarylene. In embodiments, L^{3D} is independently substituted or unsubstituted 5 to 10 membered heteroarylene. In embodiments, L^{3D} is independently substituted 5 to 10 membered heteroarylene. In embodiments, L^{3D} is independently unsubstituted 5 to 10 membered heteroarylene. In embodiments, L^{3D} is independently substituted or unsubstituted 5 to 9 membered heteroarylene. In embodiments, L^{3D} is independently substituted 5 to 9 membered heteroarylene. In embodiments, L^{3D} is independently unsubstituted 5 to 9 membered heteroarylene. In embodiments, L^{3D} is independently substituted or unsubstituted 5 to 6 membered heteroarylene. In embodiments, L^{3D} is independently substituted 5 to 6 membered heteroarylene. In embodiments, L^{3D} is independently unsubstituted 5 to 6 membered heteroarylene.

In embodiments, L^{3E} is independently a bond. In embodiments, L^{3E} is independently —NH—. In embodiments, L^{3E} is independently —O—. In embodiments, L^{3E} independently —S—. In embodiments, L^{3E} is independently —C(O)—. In embodiments, L^{3E} is independently —NHC(O)—. In embodiments, L^{3E} is independently —S(O)₂C(O)—. In embodiments, L^{3E} is independently —OPO₂—O—. In embodiments, L^{3E} is independently —OP(S)(O)—O—. In embodiments, L^{3E} is independently —OP(S)₂—O—. In embodiments, L^{3E} is independently —S(O)NH—. In embodiments, L^{3E} is independently —NHC(O)NH—. In embodiments, L^{3E} is independently —C(O)O—. In embodiments, L^{3E} is independently —OC(O)—. In embodiments, L^{3E} is independently —C(O)NH—. In embodiments, L^{3E} is not a bond.

In embodiments, L^{3E} is independently substituted or unsubstituted alkylene (e.g., C₁-C₂₅, C₁-C₂₀, C₁-C₁₂, C₁-C₈, C₁-C₆, C₁-C₄, or C₁-C₂). In embodiments, L^{3E} is independently substituted alkylene (e.g., C₁-C₂₅, C₁-C₂₀, C₁-C₁₂, C₁-C₈, C₁-C₆, C₁-C₄, or C₁-C₂). In embodiments, L^{3E} is independently unsubstituted alkylene (e.g., C₁-C₂₅, C₁-C₂₀, C₁-C₁₂, C₁-C₈, C₁-C₆, C₁-C₄, or C₁-C₂). In embodiments, L^{3E} is independently substituted or unsubstituted C₁-C₂₅ alkylene. In embodiments, L^{3E} is independently substituted C₁-C₂₅ alkylene. In embodiments, L^{3E} is independently unsubstituted C₁-C₂₅ alkylene. In embodiments, L^{3E} is independently substituted or unsubstituted C₁-C₂₀ alkylene. In embodiments, L^{3E} is independently substituted C₁-C₂₀ alkylene. In embodiments, L^{3E} is independently unsubstituted C₁-C₂₀ alkylene. In embodiments, L^{3E} is independently substituted or unsubstituted C₁-C₁₂ alkylene. In embodiments, L^{3E} is independently substituted C₁-C₁₂ alkylene. In embodiments, L^{3E} is independently unsubstituted C₁-C₁₂ alkylene. In embodiments, L^{3E} is independently substituted or unsubstituted C₁-C₈ alkylene. In embodiments, L^{3E} is independently substituted C₁-C₈ alkylene. In embodiments, L^{3E} is independently unsubstituted C₁-C₈ alkylene. In embodiments, L^{3E} is independently substituted or unsubstituted C₁-C₆ alkylene. In embodiments, L^{3E} is independently substituted C₁-C₆ alkylene. In embodiments, L^{3E} is independently unsubstituted C₁-C₆ alkylene. In embodiments, L^{3E} is independently substituted or unsubstituted C₁-C₄ alkylene. In embodiments, L^{3E} is independently substituted C₁-C₄ alkylene. In embodiments, L^{3E} is independently unsubstituted C₁-C₄ alkylene. In embodiments, L^{3E} is independently substituted or unsubstituted ethylene. In embodiments, L^{3E} is independently substituted ethylene. In embodiments, L^{3E} is independently unsubstituted ethylene. In embodiments, L^{3E} is independently substituted or unsubstituted methylene. In embodiments, L^{3E} is independently substituted methylene. In embodiments, L^{3E} is independently unsubstituted methylene.

In embodiments, L^{3E} is independently substituted or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3E} is independently substituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3E} is independently unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L^{3E} is independently substituted or unsubstituted 2 to 25 membered heteroalkylene. In embodiments, L^{3E} is independently substituted 2 to 25 membered heteroalkylene. In embodiments, L^{3E} is independently unsubstituted 2 to 25 membered heteroalkylene. In embodiments, L^{3E} is independently substituted or unsubstituted 2 to 20 membered heteroalkylene. In embodiments, L^{3E} is independently substituted 2 to 20 membered heteroalkylene. In embodiments, L^{3E} is independently unsubstituted 2 to 20 membered heteroalkylene. In embodiments, L^{3E} is independently substituted or unsubstituted 2 to 12 membered heteroalkylene. In embodiments, L^{3E} is independently substituted 2 to 12 membered heteroalkylene. In embodiments, L^{3E} is independently unsubstituted 2 to 12 membered heteroalkylene. In embodiments, L^{3E} is independently substituted or unsubstituted 2 to 8 membered heteroalkylene. In embodiments, L^{3E} is independently substituted 2 to 8 membered heteroalkylene. In embodiments, L^{3E} is independently unsubstituted 2 to 8 membered heteroalkylene. In embodiments, L^{3E} is independently substituted or unsubstituted 2 to 6 membered heteroalkylene. In embodiments, L^{3E} is independently substituted 2 to 6 membered heteroalkylene. In embodiments, L^{3E} is independently unsubstituted 2 to 6 membered heteroalkylene. In embodiments, L^{3E} is independently substituted or unsubstituted 4 to 6 membered heteroalkylene. In embodiments, L^{3E} is independently substituted 4 to 6 membered heteroalkylene. In embodiments, L^{3E} is independently unsubstituted 4 to 6 membered heteroalkylene. In embodiments, L^{3E} is independently substituted or unsubstituted 2 to 3 membered heteroalkylene. In embodiments, L^{3E} is independently substituted 2 to 3 membered heteroalkylene. In embodiments, L^{3E} is independently unsubstituted 2 to 3 membered heteroalkylene. In embodiments, L^{3E} is independently substituted or unsubstituted 4 to 5 membered heteroalkylene. In embodiments, L^{3E} is independently substituted 4 to 5 membered heteroalkylene. In embodiments, L^{3E} is independently unsubstituted 4 to 5 membered heteroalkylene.

In embodiments, L^{3E} is independently substituted or unsubstituted cyclocycloalkylene (e.g., C₃-C₁₀, C₃-C₈, C₃-C₆, C₄-C₆, or C₅-C₆). In embodiments, L^{3E} is independently substituted cyclocycloalkylene (e.g., C₃-C₁₀, C₃-C₈, C₃-C₆, C₄-C₆, or C₅-C₆). In embodiments, L^{3E} is independently unsubstituted cyclocycloalkylene (e.g., C₃-C₁₀, C₃-C₈, C₃-C₆, C₄-C₆, or C₅-C₆). In embodiments, L^{3E} is independently substituted or unsubstituted C₃-C₁₀ cycloalkylene. In embodiments, L^{3E} is independently substituted C₃-C₁₀ cycloalkylene. In embodiments, L^{3E} is independently unsubstituted C₃-C₁₀ cycloalkylene. In embodiments, L^{3E} is independently substituted or unsubstituted C₃-C₈ cycloalkylene. In embodiments, L^{3E} is independently substituted C₃-C₈ cycloalkylene. In embodiments, L^{3E} independently unsubstituted C₃-C₈ cycloalkylene. In embodiments, L^{3E} is independently substituted or unsubstituted C₃-C₆ cycloalkylene. In embodiments, L^{3E} is independently substituted C₃-C₆ cycloalkylene. In embodiments, L^{3E} is independently unsubstituted C₃-C₆ cycloalkylene. In embodiments, L^{3E} is independently substituted or unsubstituted C₄-C₆ cycloalkylene. In embodiments, L^{3E} is independently substituted C₄-C₆ cycloalkylene. In embodiments, L^{3E} is independently unsubstituted C₄-C₆ cycloalkylene. In embodiments, L^{3E} is independently substituted or unsubstituted C₅-C₆ cycloalkylene. In embodiments, L^{3E} is independently substituted C₅-C₆ cycloalkylene. In embodiments, L^{3E} is independently unsubstituted C₅-C₆ cycloalkylene. In embodiments, L^{3E} is independently substituted or unsubstituted hexylene. In embodiments, L^{3E} is independently substituted hexylene. In embodiments, L^{3E} is independently unsubstituted hexylene.

In embodiments, L^{3E} is independently substituted or unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3E} is independently substituted heterocycloalkylene (e.g., 3 to 10 membered, 3 to 8 membered, 3 to 6 membered, 4 to 6 membered, 4 to 5 membered, or 5 to 6 membered). In embodiments, L^{3E} is independently unsubstituted heterocycloalkylene (e.g., 3 to 10 membered, 3 to 8 membered, 3 to 6 membered, 4 to 6 membered, 4 to 5 membered, or 5 to 6 membered). In embodiments, L^{3E} is independently substituted or unsubstituted 3 to 10 membered heterocycloalkylene. In embodiments, L^{3E} is independently substituted 3 to 10 membered heterocycloalkylene. In embodiments, L^{3E} is independently unsubstituted 3 to 10 membered heterocycloalkylene. In embodiments, L^{3E} is independently substituted or unsubstituted 3 to 8 membered heterocycloalkylene. In embodiments, L^{3E} is independently substituted 3 to 8 membered heterocycloalkylene. In embodiments, L^{3E} is independently unsubstituted 3 to 8 membered heterocycloalkylene. In embodiments, L^{3E} is independently substituted or unsubstituted 3 to 6 membered heterocycloalkylene. In embodiments, L^{3E} is independently substituted 3 to 6 membered heterocycloalkylene. In embodiments, L^{3E} is independently unsubstituted 3 to 6 membered heterocycloalkylene. In embodiments, L^{3E} is independently substituted or unsubstituted 4 to 6 membered heterocycloalkylene. In embodiments, L^{3E} is independently substituted 4 to 6 membered heterocycloalkylene. In embodiments, L^{3E} is independently unsubstituted 4 to 6 membered heterocycloalkylene. In embodiments, L^{3E} is independently substituted or unsubstituted 4 to 5 membered heterocycloalkylene. In embodiments, L^{3E} is independently substituted 4 to 5 membered heterocycloalkylene. In embodiments, L^{3E} is independently unsubstituted 4 to 5 membered heterocycloalkylene. In embodiments, L^{3E} is independently substituted or unsubstituted 5 to 6 membered heterocycloalkylene. In embodiments, L^{3E} is independently substituted 5 to 6 membered heterocycloalkylene. In embodiments, L^{3E} is independently unsubstituted 5 to 6 membered heterocycloalkylene.

In embodiments, L^{3E} is independently substituted or unsubstituted arylene. In embodiments, L^{3E} is independently substituted or unsubstituted arylene (e.g., C₆-C₁₂, C₆-C₁₀, or phenyl). In embodiments, L^{3E} is independently substituted arylene (e.g., C₆-C₁₂, C₆-C₁₀, or phenyl). In embodiments, L^{3E} is independently unsubstituted arylene (e.g., C₆-C₁₂, C₆-C₁₀, or phenyl). In embodiments, L^{3E} is independently substituted or unsubstituted C₆-C₁₂ arylene. In embodiments, L^{3E} is independently substituted C₆-C₁₂ arylene. In embodiments, L^{3E} is independently unsubstituted C₆-C₁₂ arylene. In embodiments, L^{3E} is independently substituted or unsubstituted C₆-C₁₀ arylene. In embodiments, L^{3E} is independently substituted C₆-C₁₀ arylene. In embodiments, L^{3E} is independently unsubstituted C₆-C₁₀ arylene. In embodiments, L^{3E} is independently substituted or unsubstituted phenylene. In embodiments, L^{3E} is independently substituted phenylene. In embodiments, L^{3E} is independently unsubstituted phenylene. In embodiments, L^{3E} is independently substituted or unsubstituted biphenylene. In embodiments, L^{3E} is independently substituted biphenylene. In embodiments, L^{3E} is independently unsubstituted biphenylene. In embodiments, L^{3E} is independently substituted or unsubstituted naphthylene. In embodiments, L^{3E} is independently substituted naphthylene. In embodiments, L^{3E} is independently unsubstituted naphthylene.

In embodiments, L^{3E} is independently substituted or unsubstituted heteroarylene. In embodiments, L^{3E} is independently substituted heteroarylene. In embodiments, L^{3E} is independently unsubstituted heteroarylene. In embodiments, L^{3E} is independently substituted or unsubstituted heteroarylene (e.g., 5 to 12 membered, 5 to 10 membered, 5 to 9 membered, or 5 to 6 membered). In embodiments, L^{3E} is independently substituted heteroarylene (e.g., 5 to 12 membered, 5 to 10 membered, 5 to 9 membered, or 5 to 6 membered). In embodiments, L^{3E} is independently unsubstituted heteroarylene (e.g., 5 to 12 membered, 5 to 10 membered, 5 to 9 membered, or 5 to 6 membered). In embodiments, L^{3E} is independently substituted or unsubstituted 5 to 12 membered heteroarylene. In embodiments, L^{3E} is independently substituted 5 to 12 membered heteroarylene. In embodiments, L^{3E} is independently unsubstituted 5 to 12 membered heteroarylene. In embodiments, L^{3E} is independently substituted or unsubstituted 5 to 10 membered heteroarylene. In embodiments, L^{3E} is independently substituted 5 to 10 membered heteroarylene. In embodiments, L^{3E} is independently unsubstituted 5 to 10 membered heteroarylene. In embodiments, L^{3E} is independently substituted or unsubstituted 5 to 9 membered heteroarylene. In embodiments, L^{3E} is independently substituted 5 to 9 membered heteroarylene. In embodiments, L^{3E} is independently unsubstituted 5 to 9 membered heteroarylene. In embodiments, L^{3E} is independently substituted or unsubstituted 5 to 6 membered heteroarylene. In embodiments, L^{3E} is independently substituted 5 to 6 membered heteroarylene. In embodiments, L^{3E} is independently unsubstituted 5 to 6 membered heteroarylene.

In embodiments, L³ is independently -L¹³-NH—C(O)— or -L¹³-C(O)—NH—, and L¹³ is independently substituted or unsubstituted C₁-C₂₀ alkylene, substituted or unsubstituted 2-20 membered heteroalkylene, or substituted or unsubstituted 2-20 membered heteroalkenylene.

In embodiments, L¹³ is independently substituted or unsubstituted alkylene (e.g., C₁-C₂₀, C₁-C₁₂, C₁-C₈, C₁-C₆, C₁-C₄, or C₁-C₂). In embodiments, L¹³ is independently substituted alkylene (e.g., C₁-C₂₀, C₁-C₁₂, C₁-C₈, C₁-C₆, C₁-C₄, or C₁-C₂). In embodiments, L¹³ is independently unsubstituted alkylene (e.g., C₁-C₂₀, C₁-C₁₂, C₁-C₈, C₁-C₆, C₁-C₄, or C₁-C₂). In embodiments, L¹³ is independently substituted or unsubstituted C₁-C₂₀ alkylene. In embodiments, L¹³ is independently substituted C₁-C₂₀ alkylene. In embodiments, L¹³ is independently unsubstituted C₁-C₂₀ alkylene. In embodiments, L¹³ is independently substituted or unsubstituted C₁-C₁₂ alkylene. In embodiments, L¹³ is independently substituted C₁-C₁₂ alkylene. In embodiments, L¹³ is independently unsubstituted C₁-C₁₂ alkylene. In embodiments, L¹³ is independently substituted or unsubstituted C₁-C₈ alkylene. In embodiments, L¹³ is independently substituted C₁-C₈ alkylene. In embodiments, L¹³ is independently unsubstituted C₁-C₈ alkylene. In embodiments, L¹³ is independently substituted or unsubstituted C₁-C₆ alkylene. In embodiments, L¹³ is independently substituted C₁-C₆ alkylene. In embodiments, L¹³ is independently unsubstituted C₁-C₆ alkylene. In embodiments, L¹³ is independently substituted or unsubstituted C₁-C₄ alkylene. In embodiments, L¹³ is independently substituted C₁-C₄ alkylene. In embodiments, L¹³ is independently unsubstituted C₁-C₄ alkylene. In embodiments, L¹³ is independently substituted or unsubstituted ethylene. In embodiments, L¹³ is independently substituted ethylene. In embodiments, L¹³ is independently unsubstituted ethylene. In embodiments, L¹³ is independently substituted or unsubstituted methylene. In embodiments, L¹³ is independently substituted methylene. In embodiments, L¹³ is independently unsubstituted methylene.

In embodiments, L¹³ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹³ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹³ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L¹³ is independently substituted or unsubstituted 2 to 20 membered heteroalkylene. In embodiments, L¹³ is independently substituted 2 to 20 membered heteroalkylene. In embodiments, L¹³ is independently unsubstituted 2 to 20 membered heteroalkylene. In embodiments, L¹³ is independently substituted or unsubstituted 2 to 12 membered heteroalkylene. In embodiments, L¹³ is independently substituted 2 to 12 membered heteroalkylene. In embodiments, L¹³ is independently unsubstituted 2 to 12 membered heteroalkylene. In embodiments, L¹³ is independently substituted or unsubstituted 2 to 8 membered heteroalkylene. In embodiments, L¹³ is independently substituted 2 to 8 membered heteroalkylene. In embodiments, L¹³ is independently unsubstituted 2 to 8 membered heteroalkylene. In embodiments, L¹³ is independently substituted or unsubstituted 2 to 6 membered heteroalkylene. In embodiments, L¹³ is independently substituted 2 to 6 membered heteroalkylene. In embodiments, L¹³ is independently unsubstituted 2 to 6 membered heteroalkylene. In embodiments, L¹³ is independently substituted or unsubstituted 4 to 6 membered heteroalkylene. In embodiments, L¹³ is independently substituted 4 to 6 membered heteroalkylene. In embodiments, L¹³ is independently unsubstituted 4 to 6 membered heteroalkylene. In embodiments, L¹³ is independently substituted or unsubstituted 2 to 3 membered heteroalkylene. In embodiments, L¹³ is independently substituted 2 to 3 membered heteroalkylene. In embodiments, L¹³ is independently unsubstituted 2 to 3 membered heteroalkylene. In embodiments, L¹³ is independently substituted or unsubstituted 4 to 5 membered heteroalkylene. In embodiments, L¹³ is independently substituted 4 to 5 membered heteroalkylene. In embodiments, L¹³ is independently unsubstituted 4 to 5 membered heteroalkylene.

In embodiments, L¹³ is independently substituted or unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹³ is independently substituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹³ is independently unsubstituted heteroalkenylene (e.g., 2 to 20 membered, 2 to 12 membered, 2 to 8 membered, 2 to 6 membered, 4 to 6 membered, 2 to 3 membered, or 4 to 5 membered). In embodiments, L¹³ is independently substituted or unsubstituted 2 to 20 membered heteroalkenylene. In embodiments, L¹³ is independently substituted 2 to 20 membered heteroalkenylene. In embodiments, L¹³ is independently unsubstituted 2 to 20 membered heteroalkenylene. In embodiments, L¹³ is independently substituted or unsubstituted 2 to 12 membered heteroalkenylene. In embodiments, L¹³ is independently substituted 2 to 12 membered heteroalkenylene. In embodiments, L¹³ is independently unsubstituted 2 to 12 membered heteroalkenylene. In embodiments, L¹³ is independently substituted or unsubstituted 2 to 8 membered heteroalkenylene. In embodiments, L¹³ is independently substituted 2 to 8 membered heteroalkenylene. In embodiments, L¹³ is independently unsubstituted 2 to 8 membered heteroalkenylene. In embodiments, L¹³ is independently substituted or unsubstituted 2 to 6 membered heteroalkenylene. In embodiments, L¹³ is independently substituted 2 to 6 membered heteroalkenylene. In embodiments, L¹³ is independently unsubstituted 2 to 6 membered heteroalkenylene. In embodiments, L¹³ is independently substituted or unsubstituted 4 to 6 membered heteroalkenylene. In embodiments, L¹³ is independently substituted 4 to 6 membered heteroalkenylene. In embodiments, L¹³ is independently unsubstituted 4 to 6 membered heteroalkenylene. In embodiments, L¹³ is independently substituted or unsubstituted 2 to 3 membered heteroalkenylene. In embodiments, L¹³ is independently substituted 2 to 3 membered heteroalkenylene. In embodiments, L¹³ is independently unsubstituted 2 to 3 membered heteroalkenylene. In embodiments, L¹³ is independently substituted or unsubstituted 4 to 5 membered heteroalkenylene. In embodiments, L¹³ is independently substituted 4 to 5 membered heteroalkenylene. In embodiments, L¹³ is independently unsubstituted 4 to 5 membered heteroalkenylene.

In embodiments, L³ is independently -L¹³-NH—C(O)—. In embodiments, L³ is independently -L¹³-C(O)—NH—. In embodiments, L¹³ is substituted or unsubstituted C₁-C₈ alkylene. In embodiments, L¹³ is substituted C₁-C₈ alkylene. In embodiments, L¹³ is unsubstituted C₁-C₈ alkylene.

In embodiments, L³ is independently

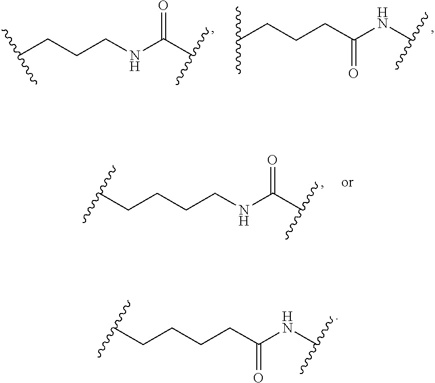 [SMILES: *CCCNC(*)=O ; *CCCC(=O)N* ; *CCCCNC(*)=O ; *CCCCC(=O)N*]

In embodiments, L³ is independently

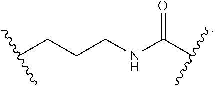 [SMILES: *CCCNC(*)=O]

In embodiments, L³ is independently

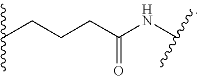 [SMILES: *CCCC(=O)N*]

In embodiments, L³ is independently

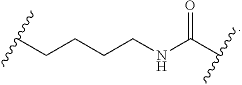 [SMILES: *CCCCNC(*)=O]

In embodiments, L³ is independently

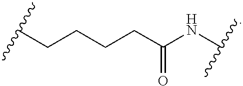 [SMILES: *CCCCC(=O)N*]

In embodiments, L³ is independently a bond,

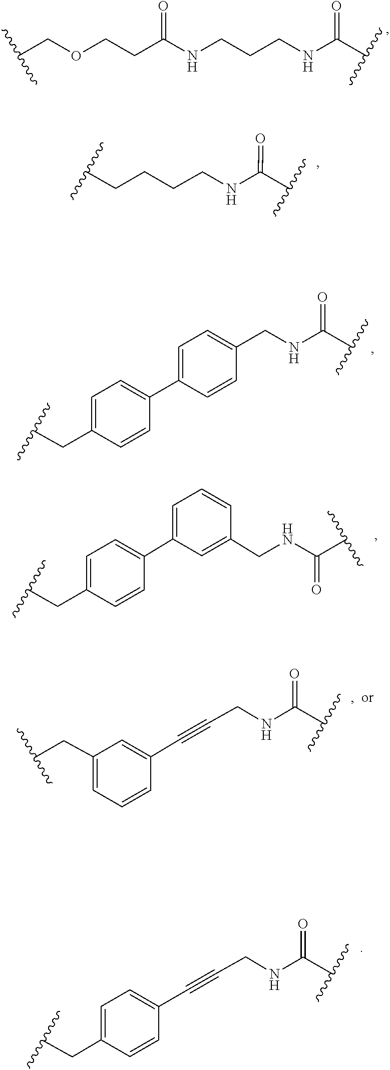 [SMILES: *COCCC(=O)NCCCNC(*)=O ; *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L³ is independently a bond. In embodiments, L³ is independently

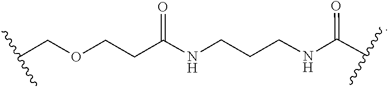 [SMILES: *COCCC(=O)NCCCNC(*)=O]

In embodiments, L³ is independently

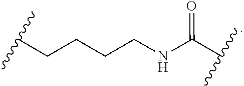 [SMILES: *CCCCNC(*)=O]

In embodiments, L³ is independently

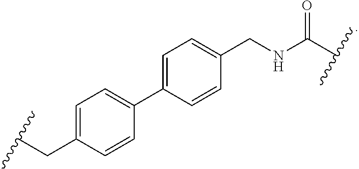 [SMILES: *Cc1ccc(-c2ccc(CNC(*)=O)cc2)cc1]

In embodiments, L³ is independently

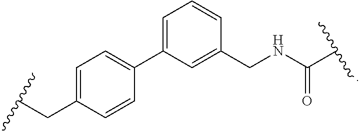 [SMILES: *Cc1ccc(-c2cccc(CNC(*)=O)c2)cc1]

In embodiments, L³ is independently

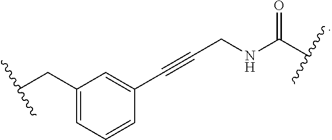 [SMILES: *Cc1cccc(C#CCNC(*)=O)c1]

In embodiments, L³ is independently

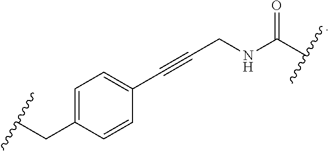 [SMILES: *Cc1ccc(C#CCNC(*)=O)cc1]

In embodiments, L^{3A} is independently substituted or unsubstituted 2 to 8 membered heteroalkylene; L^{3B} is independently —NHC(O)— or —C(O)NH—; L^{3C} is independently unsubstituted C₁-C₈ alkylene; L^{3D} is independently a bond, —NHC(O)—, or —C(O)NH—; and L^{3E} is independently —NHC(O)—. In embodiments, L^{3A} is independently substituted or unsubstituted 2 to 5 membered heteroalkylene; L^{3B} is independently —NHC(O)— or —C(O)NH—; L^{3C} is independently unsubstituted C₁-C₄ alkylene; L^{3D} is independently a bond, —NHC(O)—, or —C(O)NH—; and L^{3E} is independently —NHC(O)—. In embodiments, L^{3A} is independently unsubstituted 4 to 5 membered heteroalkylene; L^{3B} is independently —NHC(O)— or —C(O)NH—; L^{3C} is independently unsubstituted C₁-C₄ alkylene; L^{3D} is independently a bond; and L^{3E} is independently —NHC(O)—.

In embodiments, R¹ is independently unsubstituted alkyl (e.g., C₁-C₂₅, C₁₁-C₂₅, C₁₁-C₁₇, or C₁₄-C₁₅). In embodiments, R¹ is independently unsubstituted C₁-C₂₅ alkyl. In embodiments, R¹ is independently unsubstituted C₇-C₂₅ alkyl. In embodiments, R¹ is independently unsubstituted C₇-C₂₀ alkyl. In embodiments, R¹ is independently unsubstituted C₇-C₁₇ alkyl. In embodiments, R¹ is independently unsubstituted C₇-C₁₁ alkyl. In embodiments, R¹ is independently unsubstituted C₁₁-C₂₅ alkyl. In embodiments, R¹ is independently unsubstituted C₁₁-C₁₇ alkyl. In embodiments, R¹ is independently unsubstituted C₁₄-C₁₅ alkyl. In embodiments, R¹ is independently unsubstituted C₇ alkyl. In embodiments, R¹ is independently unsubstituted C₉ alkyl. In embodiments, R¹ is independently unsubstituted C₁₁ alkyl. In embodiments, R¹ is independently unsubstituted C₁₃ alkyl. In embodiments, R¹ is independently unsubstituted C₁₄ alkyl. In embodiments, R¹ is independently unsubstituted C₁₅ alkyl. In embodiments, R¹ is independently unsubstituted C₁₆ alkyl.

In embodiments, R¹ is independently unsubstituted branched alkyl (e.g., C₁-C₂₅, C₁₁-C₂₅, C₁₁-C₁₇, or C₁₄-C₁₅). In embodiments, R¹ is independently unsubstituted branched C₁-C₂₅ alkyl. In embodiments, R¹ is independently unsubstituted branched C₇-C₂₅ alkyl. In embodiments, R¹ is independently unsubstituted branched C₇-C₂₀ alkyl. In embodiments, R¹ is independently unsubstituted branched C₇-C₂₀ alkyl. In embodiments, R¹ is independently unsubstituted branched C₇-C₁₇ alkyl. In embodiments, R¹ is independently unsubstituted branched C₇-C₁₁ alkyl. In embodiments, R¹ is independently unsubstituted branched C₁₁-C₂₅ alkyl. In embodiments, R¹ is independently unsubstituted branched C₁₁-C₁₇ alkyl. In embodiments, R¹ is independently unsubstituted branched C₁₄-C₁₅ alkyl. In embodiments, R¹ is independently unsubstituted branched C₇ alkyl. In embodiments, R¹ is independently unsubstituted branched C₉ alkyl. In embodiments, R¹ is independently unsubstituted branched C₁₁ alkyl. In embodiments, R¹ is independently unsubstituted branched C₁₃ alkyl. In embodiments, R¹ is independently unsubstituted branched C₁₄ alkyl. In embodiments, R¹ is independently unsubstituted branched C₁₅ alkyl. In embodiments, R¹ is independently unsubstituted branched C₁₆ alkyl.

In embodiments, R¹ is independently unsubstituted unbranched alkyl (e.g., C₁-C₂₅, C₁₁-C₂₅, C₁₁-C₁₇, or C₁₄-C₁₅). In embodiments, R¹ is independently unsubstituted unbranched C₁-C₂₅ alkyl. In embodiments, R¹ is independently unsubstituted unbranched C₇-C₂₅ alkyl. In embodiments, R¹ is independently unsubstituted unbranched C₇-C₂₀ alkyl. In embodiments, R¹ is independently unsubstituted unbranched C₇-C₁₇ alkyl. In embodiments, R¹ is independently unsubstituted unbranched C₇-C₁₁ alkyl. In embodiments, R¹ is independently unsubstituted unbranched C₁₁-C₂₅ alkyl. In embodiments, R¹ is independently unsubstituted unbranched C₁₁-C₁₇ alkyl. In embodiments, R¹ is independently unsubstituted unbranched C₁₄-C₁₅ alkyl. In embodiments, R¹ is independently unsubstituted unbranched C₇ alkyl. In embodiments, R¹ is independently unsubstituted unbranched C₉ alkyl. In embodiments, R¹ is independently unsubstituted unbranched C₁₁ alkyl. In embodiments, R¹ is independently unsubstituted unbranched C₁₃ alkyl. In embodiments, R¹ is independently unsubstituted unbranched C₁₄ alkyl. In embodiments, R¹ is independently unsubstituted unbranched C₁₅ alkyl. In embodiments, R¹ is independently unsubstituted unbranched C₁₆ alkyl.

In embodiments, R¹ is independently unsubstituted branched saturated alkyl (e.g., C₁-C₂₅, C₁₁-C₂₅, C₁₁-C₁₇, or C₁₄-C₁₅). In embodiments, R¹ is independently unsubstituted branched saturated C₁-C₂₅ alkyl. In embodiments, R¹ is independently unsubstituted branched saturated C₇-C₂₅ alkyl. In embodiments, R¹ is independently unsubstituted branched saturated C₇-C₂₀ alkyl. In embodiments, R¹ is independently unsubstituted branched saturated C₇-C₁₇ alkyl. In embodiments, R¹ is independently unsubstituted branched saturated C₇-C₁₁ alkyl. In embodiments, R¹ is independently unsubstituted branched saturated C₁₁-C₂₅ alkyl. In embodiments, R¹ is independently unsubstituted branched saturated C₁₁-C₁₇ alkyl. In embodiments, R¹ is independently unsubstituted branched saturated C₁₄-C₁₅ alkyl. In embodiments, R¹ is independently unsubstituted branched saturated C₇ alkyl. In embodiments, R¹ is independently unsubstituted branched saturated C₉ alkyl. In embodiments, R¹ is independently unsubstituted branched saturated C₁₁ alkyl. In embodiments, R¹ is independently unsubstituted branched saturated C₁₃ alkyl. In embodiments, R¹ is independently unsubstituted branched saturated C₁₄ alkyl. In embodiments, R¹ is independently unsubstituted branched saturated C₁₅ alkyl. In embodiments, R¹ is independently unsubstituted branched saturated C₁₆ alkyl.

In embodiments, R¹ is independently unsubstituted branched unsaturated alkyl (e.g., C₁-C₂₅, C₁₁-C₂₅, C₁₁-C₁₇, or C₁₄-C₁₅). In embodiments, R¹ is independently unsubstituted branched unsaturated C₁-C₂₅ alkyl. In embodiments, R¹ is independently unsubstituted branched unsaturated C₇-C₂₅ alkyl. In embodiments, R¹ is independently unsubstituted branched unsaturated C₇-C₂₀ alkyl. In embodiments, R¹ is independently unsubstituted branched unsaturated C₇-C₁₇ alkyl. In embodiments, R¹ is independently unsubstituted branched unsaturated C₇-C₁₁ alkyl. In embodiments, R¹ is independently unsubstituted branched unsaturated C₁₁-C₂₅ alkyl. In embodiments, R¹ is independently unsubstituted branched unsaturated C₁₁-C₁₇ alkyl. In embodiments, R¹ is independently unsubstituted branched unsaturated C₁₄-C₁₅ alkyl. In embodiments, R¹ is independently unsubstituted branched unsaturated C₇ alkyl. In embodiments, R¹ is independently unsubstituted branched unsaturated C₉ alkyl. In embodiments, R¹ is independently unsubstituted branched unsaturated C₁₁ alkyl. In embodiments, R¹ is independently unsubstituted branched unsaturated C₁₃ alkyl. In embodiments, R¹ is independently unsubstituted branched unsaturated C₁₄ alkyl. In embodiments, R¹ is independently unsubstituted branched unsaturated C₁₅ alkyl. In embodiments, R¹ is independently unsubstituted branched saturated C₁₆ alkyl.

In embodiments, R¹ is independently unsubstituted unbranched saturated alkyl (e.g., C₁-C₂₅, C₁₁-C₂₅, C₁₁-C₁₇, or C₁₄-C₁₅). In embodiments, R¹ is independently unsubstituted unbranched saturated C₁-C₂₅ alkyl. In embodiments, R¹ is independently unsubstituted unbranched saturated C₇-C₂₅ alkyl. In embodiments, R¹ is independently unsubstituted unbranched saturated C₇-C₂₀ alkyl. In embodiments, R¹ is independently unsubstituted unbranched saturated C₇-C₁₇ alkyl. In embodiments, R¹ is independently unsubstituted unbranched saturated C₇-C₁₁ alkyl. In embodiments, R¹ is independently unsubstituted unbranched saturated C₁₁-C₂₅ alkyl. In embodiments, R¹ is independently unsubstituted unbranched saturated C₁₁-C₁₇ alkyl. In embodiments, R¹ is independently unsubstituted unbranched saturated C₁₄-C₁₅ alkyl. In embodiments, R¹ is independently unsubstituted unbranched saturated C₇ alkyl. In embodiments, R¹ is independently unsubstituted unbranched saturated C₉ alkyl. In embodiments, R¹ is independently unsubstituted unbranched saturated C₁₁ alkyl. In embodiments, R¹ is independently unsubstituted unbranched saturated C₁₃ alkyl. In embodiments, R¹ is independently unsubstituted unbranched saturated C₁₄ alkyl. In embodiments, R¹ is independently unsubstituted unbranched saturated C₁₅ alkyl. In embodiments, R¹ is independently unsubstituted unbranched saturated C₁₆ alkyl.

In embodiments, R¹ is independently unsubstituted unbranched unsaturated alkyl (e.g., C₁-C₂₅, C₁₁-C₂₅, C₁₁-C₁₇, or C₁₄-C₁₅). In embodiments, R¹ is independently unsubstituted unbranched unsaturated C₁-C₂₅ alkyl. In embodiments, R¹ is independently unsubstituted unbranched unsaturated C₇-C₂₅ alkyl. In embodiments, R¹ is independently unsubstituted unbranched unsaturated C₇-C₂₀ alkyl. In embodiments, R¹ is independently unsubstituted unbranched unsaturated C₇-C₁₇ alkyl. In embodiments, R¹ is independently unsubstituted unbranched unsaturated C₇-C₁₁ alkyl. In embodiments, R¹ is independently unsubstituted unbranched unsaturated C₁₁-C₂₅ alkyl. In embodiments, R¹ is independently unsubstituted unbranched unsaturated C₁₁-C₁₇ alkyl. In embodiments, R¹ is independently unsubstituted unbranched unsaturated C₁₄-C₁₅ alkyl. In embodiments, R¹ is independently unsubstituted unbranched unsaturated C₇ alkyl. In embodiments, R¹ is independently unsubstituted unbranched unsaturated C₉ alkyl. In embodiments, R¹ is independently unsubstituted unbranched unsaturated C₁₁ alkyl. In embodiments, R¹ is independently unsubstituted unbranched unsaturated C₁₃ alkyl. In embodiments, R¹ is independently unsubstituted unbranched unsaturated C₁₄ alkyl. In embodiments, R¹ is independently unsubstituted unbranched unsaturated C₁₅ alkyl. In embodiments, R¹ is independently unsubstituted unbranched unsaturated C₁₆ alkyl.

In embodiments, R¹ is independently unsubstituted C₉-C₁₉ alkyl. In embodiments, R¹ is independently unsubstituted branched C₉-C₁₉ alkyl. In embodiments, R¹ is independently unsubstituted unbranched C₉-C₁₉ alkyl. In embodiments, R¹ is independently unsubstituted branched saturated C₉-C₁₉ alkyl. In embodiments, R¹ is independently unsubstituted branched unsaturated C₉-C₁₉ alkyl. In embodiments, R¹ is independently unsubstituted unbranched saturated C₉-C₁₉ alkyl. In embodiments, R¹ is independently unsubstituted unbranched unsaturated C₉-C₁₉ alkyl.

In embodiments, R¹ is independently unsubstituted C₁₃₋₁₄ alkyl. In embodiments, R¹ is independently unsubstituted unbranched C₁₃₋₁₄ alkyl. In embodiments, R¹ is independently unsubstituted branched C₁₃₋₁₄ alkyl. In embodiments, R¹ is independently unsubstituted unbranched unsaturated C₁₃₋₁₄ alkyl. In embodiments, R¹ is independently unsubstituted unbranched saturated C₁₃₋₁₄ alkyl. In embodiments, R¹ is independently unsubstituted branched unsaturated C₁₃₋₁₄ alkyl. In embodiments, R¹ is independently unsubstituted branched saturated C₁₃₋₁₄ alkyl. In embodiments, R¹ is independently unsubstituted C₁₂₋₁₄ alkyl. In embodiments, R¹ is independently unsubstituted unbranched C₁₂₋₁₄ alkyl. In embodiments, R¹ is independently unsubstituted branched C₁₂₋₁₄ alkyl. In embodiments, R¹ is independently unsubstituted unbranched unsaturated C₁₂₋₁₄ alkyl. In embodiments, R¹ is independently unsubstituted unbranched saturated C₁₂₋₁₄ alkyl. In embodiments, R¹ is independently unsubstituted branched unsaturated C₁₂₋₁₄ alkyl. In embodiments, R¹ is independently unsubstituted branched saturated C₁₂₋₁₄ alkyl. In embodiments, R¹ is not unsubstituted C₁₅ alkyl.

In embodiments, R² is independently unsubstituted alkyl (e.g., C₁-C₂₅, C₁₁-C₂₅, C₁₁-C₁₇, or C₁₄-C₁₅). In embodiments, R² is independently unsubstituted C₁-C₂₅ alkyl. In embodiments, R² is independently unsubstituted C₇-C₂₅ alkyl. In embodiments, R² is independently unsubstituted C₇-C₂₀ alkyl. In embodiments, R² is independently unsubstituted C₇-C₁₇ alkyl. In embodiments, R² is independently unsubstituted C₇-C₁₁ alkyl. In embodiments, R² is independently unsubstituted C₁₁-C₂₅ alkyl. In embodiments, R² is independently unsubstituted C₁₁-C₁₇ alkyl. In embodiments, R² is independently unsubstituted C₁₄-C₁₅ alkyl. In embodiments, R² is independently unsubstituted C₇ alkyl. In embodiments, R² is independently unsubstituted C₉ alkyl. In embodiments, R² is independently unsubstituted C₁₁ alkyl. In embodiments, R² is independently unsubstituted C₁₃ alkyl. In embodiments, R² is independently unsubstituted C₁₄ alkyl. In embodiments, R² is independently unsubstituted C₁₅ alkyl. In embodiments, R² is independently unsubstituted C₁₆ alkyl.

In embodiments, R² is independently unsubstituted branched alkyl (e.g., C₁-C₂₅, C₁₁-C₂₅, C₁₁-C₁₇, or C₁₄-C₁₅). In embodiments, R² is independently unsubstituted branched C₁-C₂₅ alkyl. In embodiments, R² is independently unsubstituted branched C₇-C₂₅ alkyl. In embodiments, R² is independently unsubstituted branched C₇-C₂₀ alkyl. In embodiments, R² is independently unsubstituted branched C₇-C₁₇ alkyl. In embodiments, R² is independently unsubstituted branched C₇-C₁₁ alkyl. In embodiments, R² is independently unsubstituted branched C₁₁-C₂₅ alkyl. In embodiments, R² is independently unsubstituted branched C₁₁-C₁₇ alkyl. In embodiments, R² is independently unsubstituted branched C₁₄-C₁₅ alkyl. In embodiments, R² is independently unsubstituted branched C₇ alkyl. In embodiments, R² is independently unsubstituted branched C₉ alkyl. In embodiments, R² is independently unsubstituted branched C₁₁ alkyl. In embodiments, R² is independently unsubstituted branched C₁₃ alkyl. In embodiments, R² is independently unsubstituted branched C₁₄ alkyl. In embodiments, R² is independently unsubstituted branched C₁₅ alkyl. In embodiments, R² is independently unsubstituted branched C₁₆ alkyl.

In embodiments, R² is independently unsubstituted unbranched alkyl (e.g., C₁-C₂₅, C₁₁-C₂₅, C₁₁-C₁₇, or C₁₄-C₁₅). In embodiments, R² is independently unsubstituted unbranched C₁-C₂₅ alkyl. In embodiments, R² is independently unsubstituted unbranched C₇-C₂₅ alkyl. In embodiments, R² is independently unsubstituted unbranched C₇-C₂₀ alkyl. In embodiments, R² is independently unsubstituted unbranched C₇-C₁₇ alkyl. In embodiments, R² is independently unsubstituted unbranched C₇-C₁₁ alkyl. In embodiments, R² is independently unsubstituted unbranched C₁₁-C₂₅ alkyl. In embodiments, R² is independently unsubstituted unbranched C₁₁-C₁₇ alkyl. In embodiments, R² is independently unsubstituted unbranched C₁₄-C₁₅ alkyl. In embodiments, R² is independently unsubstituted unbranched C₇ alkyl. In embodiments, R² is independently unsubstituted unbranched C₉ alkyl. In embodiments, R² is independently unsubstituted unbranched C₁₁ alkyl. In embodiments, R² is independently unsubstituted unbranched C₁₃ alkyl. In embodiments, R² is independently unsubstituted unbranched C₁₄ alkyl. In embodiments, R² is independently unsubstituted unbranched C₁₅ alkyl. In embodiments, R² is independently unsubstituted unbranched C₁₆ alkyl.

In embodiments, R² is independently unsubstituted branched saturated alkyl (e.g., C₁-C₂₅, C₁₁-C₂₅, C₁₁-C₁₇, or C₁₄-C₁₅). In embodiments, R² is independently unsubstituted branched saturated C₁-C₂₅ alkyl. In embodiments, R² is independently unsubstituted branched saturated C₇-C₂₅ alkyl. In embodiments, R² is independently unsubstituted branched saturated C₇-C₂₀ alkyl. In embodiments, R² is independently unsubstituted branched saturated C₇-C₁₇ alkyl. In embodiments, R² is independently unsubstituted branched saturated C₇-C₁₁ alkyl. In embodiments, R² is independently unsubstituted branched saturated C₁₁-C₂₅ alkyl. In embodiments, R² is independently unsubstituted branched saturated C₁₁-C₁₇ alkyl. In embodiments, R² is independently unsubstituted branched saturated C₁₄-C₁₅ alkyl. In embodiments, R² is independently unsubstituted branched saturated C₇ alkyl. In embodiments, R² is independently unsubstituted branched saturated C₉ alkyl. In embodiments, R² is independently unsubstituted branched saturated C₁₁ alkyl. In embodiments, R² is independently unsubstituted branched saturated C₁₃ alkyl. In embodiments, R² is independently unsubstituted branched saturated C₁₄ alkyl. In embodiments, R² is independently unsubstituted branched saturated C₁₅ alkyl. In embodiments, R² is independently unsubstituted branched saturated C₁₆ alkyl.

In embodiments, R² is independently unsubstituted branched unsaturated alkyl (e.g., C₁-C₂₅, C₁₁-C₂₅, C₁₁-C₁₇, or C₁₄-C₁₅). In embodiments, R² is independently unsubstituted branched unsaturated C₁-C₂₅ alkyl. In embodiments, R² is independently unsubstituted branched unsaturated C₇-C₂₅ alkyl. In embodiments, R² is independently unsubstituted branched unsaturated C₇-C₂₀ alkyl. In embodiments, R² is independently unsubstituted branched unsaturated C₇-C₁₇ alkyl. In embodiments, R² is independently unsubstituted branched unsaturated C₇-C₁₁ alkyl. In embodiments, R² is independently unsubstituted branched unsaturated C₁₁-C₂₅ alkyl. In embodiments, R² is independently unsubstituted branched unsaturated C₁₁-C₁₇ alkyl. In embodiments, R² is independently unsubstituted branched unsaturated C₁₄-C₁₅ alkyl. In embodiments, R² is independently unsubstituted branched unsaturated C₇ alkyl. In embodiments, R² is independently unsubstituted branched unsaturated C₉ alkyl. In embodiments, R² is independently unsubstituted branched unsaturated C₁₁ alkyl. In embodiments, R² is independently unsubstituted branched unsaturated C₁₃ alkyl. In embodiments, R² is independently unsubstituted branched unsaturated C₁₄ alkyl. In embodiments, R² is independently unsubstituted branched unsaturated C₁₅ alkyl. In embodiments, R² is independently unsubstituted branched saturated C₁₆ alkyl.

In embodiments, R² is independently unsubstituted unbranched saturated alkyl (e.g., C₁-C₂₅, C₁₁-C₂₅, C₁₁-C₁₇, or C₁₄-C₁₅). In embodiments, R² is independently unsubstituted unbranched saturated C₁-C₂₅ alkyl. In embodiments, R² is independently unsubstituted unbranched saturated C₇-C₂₅ alkyl. In embodiments, R² is independently unsubstituted unbranched saturated C₇-C₂₀ alkyl. In embodiments, R² is independently unsubstituted unbranched saturated C₇-C₁₇ alkyl. In embodiments, R² is independently unsubstituted unbranched saturated C₇-C₁₁ alkyl. In embodiments, R² is independently unsubstituted unbranched saturated C₁₁-C₂₅ alkyl. In embodiments, R² is independently unsubstituted unbranched saturated C₁₁-C₁₇ alkyl. In embodiments, R² is independently unsubstituted unbranched saturated C₁₄-C₁₅ alkyl. In embodiments, R² is independently unsubstituted unbranched saturated C₇ alkyl. In embodiments, R² is independently unsubstituted unbranched saturated C₉ alkyl. In embodiments, R² is independently unsubstituted unbranched saturated C₁₁ alkyl. In embodiments, R² is independently unsubstituted unbranched saturated C₁₃ alkyl. In embodiments, R² is independently unsubstituted unbranched saturated C₁₄ alkyl. In embodiments, R² is independently unsubstituted unbranched saturated C₁₅ alkyl. In embodiments, R² is independently unsubstituted unbranched saturated C₁₆ alkyl.

In embodiments, R² is independently unsubstituted unbranched unsaturated alkyl (e.g., C₁-C₂₅, C₁₁-C₂₅, C₁₁-C₁₇, or C₁₄-C₁₅). In embodiments, R² is independently unsubstituted unbranched unsaturated C₁-C₂₅ alkyl. In embodiments, R² is independently unsubstituted unbranched unsaturated C₇-C₂₅ alkyl. In embodiments, R² is independently unsubstituted unbranched unsaturated C₇-C₂₀ alkyl. In embodiments, R² is independently unsubstituted unbranched unsaturated C₇-C₁₇ alkyl. In embodiments, R² is independently unsubstituted unbranched unsaturated C₇-C₁₁ alkyl. In embodiments, R² is independently unsubstituted unbranched unsaturated C₁₁-C₂₅ alkyl. In embodiments, R² is independently unsubstituted unbranched unsaturated C₁₁-C₁₇ alkyl. In embodiments, R² is independently unsubstituted unbranched unsaturated C₁₄-C₁₅ alkyl. In embodiments, R² is independently unsubstituted unbranched unsaturated C₇ alkyl. In embodiments, R² is independently unsubstituted unbranched unsaturated C₉ alkyl. In embodiments, R² is independently unsubstituted unbranched unsaturated C₁₁ alkyl. In embodiments, R² is independently unsubstituted unbranched unsaturated C₁₃ alkyl. In embodiments, R² is independently unsubstituted unbranched unsaturated C₁₄ alkyl. In embodiments, R² is independently unsubstituted unbranched unsaturated C₁₅ alkyl. In embodiments, R² is independently unsubstituted unbranched unsaturated C₁₆ alkyl.

In embodiments, R² is independently unsubstituted C₉-C₁₉ alkyl. In embodiments, R² is independently unsubstituted branched C₉-C₁₉ alkyl. In embodiments, R² is independently unsubstituted unbranched C₉-C₁₉ alkyl. In embodiments, R² is independently unsubstituted branched saturated C₉-C₁₉ alkyl. In embodiments, R² is independently unsubstituted branched unsaturated C₉-C₁₉ alkyl. In embodiments, R² is independently unsubstituted unbranched saturated C₉-C₁₉ alkyl. In embodiments, R² is independently unsubstituted unbranched unsaturated C₉-C₁₉ alkyl.

In embodiments, R² is independently unsubstituted C₁₃₋₁₄ alkyl. In embodiments, R² is independently unsubstituted unbranched C₁₃₋₁₄ alkyl. In embodiments, R² is independently unsubstituted branched C₁₃₋₁₄ alkyl. In embodiments, R² is independently unsubstituted unbranched unsaturated C₁₃₋₁₄ alkyl. In embodiments, R² is independently unsubstituted unbranched saturated C₁₃₋₁₄ alkyl. In embodiments, R² is independently unsubstituted branched unsaturated C₁₃₋₁₄ alkyl. In embodiments, R² is independently unsubstituted branched saturated C₁₃₋₁₄ alkyl. In embodiments, R² is independently unsubstituted C₁₂₋₁₄ alkyl. In embodiments, R² is independently unsubstituted unbranched C₁₂₋₁₄ alkyl. In embodiments, R² is independently unsubstituted branched C₁₂₋₁₄ alkyl. In embodiments, R² is independently unsubstituted unbranched unsaturated C₁₂₋₁₄ alkyl. In embodiments, R² is independently unsubstituted unbranched saturated C₁₂₋₁₄ alkyl. In embodiments, R² is independently unsubstituted branched unsaturated C₁₂₋₁₄ alkyl. In embodiments, R² is independently unsubstituted branched saturated C₁₂₋₁₄ alkyl. In embodiments, R² is not unsubstituted C₁₅ alkyl.

In embodiments, R³ is independently unsubstituted alkyl (e.g., C₁-C₂₅, C₁₁-C₂₅, C₁₁-C₁₇, or C₁₄-C₁₅). In embodiments, R³ is independently unsubstituted C₁-C₂₅ alkyl. In embodiments, R³ is independently unsubstituted C₇-C₂₅ alkyl. In embodiments, R³ is independently unsubstituted C₇-C₂₀ alkyl. In embodiments, R³ is independently unsubstituted C₇-C₁₇ alkyl. In embodiments, R³ is independently unsubstituted C₇-C₁₁ alkyl. In embodiments, R³ is independently unsubstituted C₁₁-C₂₅ alkyl. In embodiments, R³ is independently unsubstituted C₁₁-C₁₇ alkyl. In embodiments, R³ is independently unsubstituted C₁₄-C₁₅ alkyl. In embodiments, R³ is independently unsubstituted C₇ alkyl. In embodiments, R³ is independently unsubstituted C₉ alkyl. In embodiments, R³ is independently unsubstituted C₁₁ alkyl. In embodiments, R³ is independently unsubstituted C₁₃ alkyl. In embodiments, R³ is independently unsubstituted C₁₄ alkyl. In embodiments, R³ is independently unsubstituted C₁₅ alkyl. In embodiments, R³ is independently unsubstituted C₁₆ alkyl.

In embodiments, R³ is independently unsubstituted branched alkyl (e.g., C₁-C₂₅, C₁₁-C₂₅, C₁₁-C₁₇, or C₁₄-C₁₅). In embodiments, R³ is independently unsubstituted branched C₁-C₂₅ alkyl. In embodiments, R³ is independently unsubstituted branched C₇-C₂₅ alkyl. In embodiments, R³ is independently unsubstituted branched C₇-C₂₀ alkyl. In embodiments, R³ is independently unsubstituted branched C₇-C₁₇ alkyl. In embodiments, R³ is independently unsubstituted branched C₇-C₁₁ alkyl. In embodiments, R³ is independently unsubstituted branched C₁₁-C₂₅ alkyl. In embodiments, R³ is independently unsubstituted branched C₁₁-C₁₇ alkyl. In embodiments, R³ is independently unsubstituted branched C₁₄-C₁₅ alkyl. In embodiments, R³ is independently unsubstituted branched C₇ alkyl. In embodiments, R³ is independently unsubstituted branched C₉ alkyl. In embodiments, R³ is independently unsubstituted branched C₁₁ alkyl. In embodiments, R³ is independently unsubstituted branched C₁₃ alkyl. In embodiments, R³ is independently unsubstituted branched C₁₄ alkyl. In embodiments, R³ is independently unsubstituted branched C₁₅ alkyl. In embodiments, R³ is independently unsubstituted branched C₁₆ alkyl.

In embodiments, R³ is independently unsubstituted unbranched alkyl (e.g., C₁-C₂₅, C₁₁-C₂₅, C₁₁-C₁₇, or C₁₄-C₁₅). In embodiments, R³ is independently unsubstituted unbranched C₁-C₂₅ alkyl. In embodiments, R³ is independently unsubstituted unbranched C₇-C₂₅ alkyl. In embodiments, R³ is independently unsubstituted unbranched C₇-C₂₀ alkyl. In embodiments, R³ is independently unsubstituted unbranched C₇-C₁₇ alkyl. In embodiments, R³ is independently unsubstituted unbranched C₇-C₁₁ alkyl. In embodiments, R³ is independently unsubstituted unbranched C₁₁-C₂₅ alkyl. In embodiments, R³ is independently unsubstituted unbranched C₁₁-C₁₇ alkyl. In embodiments, R³ is independently unsubstituted unbranched C₁₄-C₁₅ alkyl. In embodiments, R³ is independently unsubstituted unbranched C₇ alkyl. In embodiments, R³ is independently unsubstituted unbranched C₉ alkyl. In embodiments, R³ is independently unsubstituted unbranched C₁₁ alkyl. In embodiments, R³ is independently unsubstituted unbranched C₁₃ alkyl. In embodiments, R³ is independently unsubstituted unbranched C₁₄ alkyl. In embodiments, R³ is independently unsubstituted unbranched C₁₅ alkyl. In embodiments, R³ is independently unsubstituted unbranched C₁₆ alkyl.

In embodiments, R³ is independently unsubstituted branched saturated alkyl (e.g., C₁-C₂₅, C₁₁-C₂₅, C₁₁-C₁₇, or C₁₄-C₁₅). In embodiments, R³ is independently unsubstituted branched saturated C₁-C₂₅ alkyl. In embodiments, R³ is independently unsubstituted branched saturated C₇-C₂₅ alkyl. In embodiments, R³ is independently unsubstituted branched saturated C₇-C₂₀ alkyl. In embodiments, R³ is independently unsubstituted branched saturated C₇-C₁₇ alkyl. In embodiments, R³ is independently unsubstituted branched saturated C₇-C₁₁ alkyl. In embodiments, R³ is independently unsubstituted branched saturated C₁₁-C₂₅ alkyl. In embodiments, R³ is independently unsubstituted branched saturated C₁₁-C₁₇ alkyl. In embodiments, R³ is independently unsubstituted branched saturated C₁₄-C₁₅ alkyl. In embodiments, R³ is independently unsubstituted branched saturated C₇ alkyl. In embodiments, R³ is independently unsubstituted branched saturated C₉ alkyl. In embodiments, R³ is independently unsubstituted branched saturated C₁₁ alkyl. In embodiments, R³ is independently unsubstituted branched saturated C₁₃ alkyl. In embodiments, R³ is independently unsubstituted branched saturated C₁₄ alkyl. In embodiments, R³ is independently unsubstituted branched saturated C₁₅ alkyl. In embodiments, R³ is independently unsubstituted branched saturated C₁₆ alkyl.

In embodiments, R³ is independently unsubstituted branched unsaturated alkyl (e.g., C₁-C₂₅, C₁₁-C₂₅, C₁₁-C₁₇, or C₁₄-C₁₅). In embodiments, R³ is independently unsubstituted branched unsaturated C₁-C₂₅ alkyl. In embodiments, R³ is independently unsubstituted branched unsaturated C₇-C₂₅ alkyl. In embodiments, R³ is independently unsubstituted branched unsaturated C₇-C₂₀ alkyl. In embodiments, R³ is independently unsubstituted branched unsaturated C₇-C₁₇ alkyl. In embodiments, R³ is independently unsubstituted branched unsaturated C₇-C₁₁ alkyl. In embodiments, R³ is independently unsubstituted branched unsaturated C₁₁-C₂₅ alkyl. In embodiments, R³ is independently unsubstituted branched unsaturated C₁₁-C₁₇ alkyl. In embodiments, R³ is independently unsubstituted branched unsaturated C₁₄-C₁₅ alkyl. In embodiments, R³ is independently unsubstituted branched unsaturated C₇ alkyl. In embodiments, R³ is independently unsubstituted branched unsaturated C₉ alkyl. In embodiments, R³ is independently unsubstituted branched unsaturated C₁₁ alkyl. In embodiments, R³ is independently unsubstituted branched unsaturated C₁₃ alkyl. In embodiments, R³ is independently unsubstituted branched unsaturated C₁₄ alkyl. In embodiments, R³ is independently unsubstituted branched unsaturated C₁₅ alkyl. In embodiments, R³ is independently unsubstituted branched saturated C₁₆ alkyl.

In embodiments, R³ is independently unsubstituted unbranched saturated alkyl (e.g., C₁-C₂₅, C₁₁-C₂₅, C₁₁-C₁₇, or C₁₄-C₁₅). In embodiments, R³ is independently unsubstituted unbranched saturated C₁-C₂₅ alkyl. In embodiments, R³ is independently unsubstituted unbranched saturated C₇-C₂₅ alkyl. In embodiments, R³ is independently unsubstituted unbranched saturated C₇-C₂₀ alkyl. In embodiments, R³ is independently unsubstituted unbranched saturated C₇-C₁₇ alkyl. In embodiments, R³ is independently unsubstituted unbranched saturated C₇-C₁₁ alkyl. In embodiments, R³ is independently unsubstituted unbranched saturated C₁₁-C₂₅ alkyl. In embodiments, R³ is independently unsubstituted unbranched saturated C₁₁-C₁₇ alkyl. In embodiments, R³ is independently unsubstituted unbranched saturated C₁₄-C₁₅ alkyl. In embodiments, R³ is independently unsubstituted unbranched saturated C₇ alkyl. In embodiments, R³ is independently unsubstituted unbranched saturated C₉ alkyl. In embodiments, R³ is independently unsubstituted unbranched saturated C₁₁ alkyl. In embodiments, R³ is independently unsubstituted unbranched saturated C₁₃ alkyl. In embodiments, R³ is independently unsubstituted unbranched saturated C₁₄ alkyl. In embodiments, R³ is independently unsubstituted unbranched saturated C₁₅ alkyl. In embodiments, R³ is independently unsubstituted unbranched saturated C₁₆ alkyl.

In embodiments, R³ is independently unsubstituted unbranched unsaturated alkyl (e.g., C₁-C₂₅, C₁₁-C₂₅, C₁₁-C₁₇, or C₁₄-C₁₅). In embodiments, R³ is independently unsubstituted unbranched unsaturated C₁-C₂₅ alkyl. In embodiments, R³ is independently unsubstituted unbranched unsaturated C₇-C₂₅ alkyl. In embodiments, R³ is independently unsubstituted unbranched unsaturated C₇-C₂₀ alkyl. In embodiments, R³ is independently unsubstituted unbranched unsaturated C₇-C₁₇ alkyl. In embodiments, R³ is independently unsubstituted unbranched unsaturated C₇-C₁₁ alkyl. In embodiments, R³ is independently unsubstituted unbranched unsaturated C₁₁-C₂₅ alkyl. In embodiments, R³ is independently unsubstituted unbranched unsaturated C₁₁-C₁₇ alkyl. In embodiments, R³ is independently unsubstituted unbranched unsaturated C₁₄-C₁₅ alkyl. In embodiments, R³ is independently unsubstituted unbranched unsaturated C₇ alkyl. In embodiments, R³ is independently unsubstituted unbranched unsaturated C₉ alkyl. In embodiments, R³ is independently unsubstituted unbranched unsaturated C₁₁ alkyl. In embodiments, R³ is independently unsubstituted unbranched unsaturated C₁₃ alkyl. In embodiments, R³ is independently unsubstituted unbranched unsaturated C₁₄ alkyl. In embodiments, R³ is independently unsubstituted unbranched unsaturated C₁₅ alkyl. In embodiments, R³ is independently unsubstituted unbranched unsaturated C₁₆ alkyl.

In embodiments, R³ is independently unsubstituted C₉-C₁₉ alkyl. In embodiments, R³ is independently unsubstituted branched C₉-C₁₉ alkyl. In embodiments, R³ is independently unsubstituted unbranched C₉-C₁₉ alkyl. In embodiments, R³ is independently unsubstituted branched saturated C₉-C₁₉ alkyl. In embodiments, R³ is independently unsubstituted branched unsaturated C₉-C₁₉ alkyl. In embodiments, R³ is independently unsubstituted unbranched saturated C₉-C₁₉ alkyl. In embodiments, R³ is independently unsubstituted unbranched unsaturated C₉-C₁₉ alkyl.

In embodiments, R³ is independently unsubstituted C₁₃₋₁₄ alkyl. In embodiments, R³ is independently unsubstituted unbranched C₁₃₋₁₄ alkyl. In embodiments, R³ is independently unsubstituted branched C₁₃₋₁₄ alkyl. In embodiments, R³ is independently unsubstituted unbranched unsaturated C₁₃₋₁₄ alkyl. In embodiments, R³ is independently unsubstituted unbranched saturated C₁₃₋₁₄ alkyl. In embodiments, R³ is independently unsubstituted branched unsaturated C₁₃₋₁₄ alkyl. In embodiments, R³ is independently unsubstituted branched saturated C₁₃₋₁₄ alkyl. In embodiments, R³ is independently unsubstituted C₁₂₋₁₄ alkyl. In embodiments, R³ is independently unsubstituted unbranched C₁₂₋₁₄ alkyl. In embodiments, R³ is independently unsubstituted branched C₁₂₋₁₄ alkyl. In embodiments, R³ is independently unsubstituted unbranched unsaturated C₁₂₋₁₄ alkyl. In embodiments, R³ is independently unsubstituted unbranched saturated C₁₂₋₁₄ alkyl. In embodiments, R³ is independently unsubstituted branched unsaturated C₁₂₋₁₄ alkyl. In embodiments, R³ is independently unsubstituted branched saturated C₁₂₋₁₄ alkyl. In embodiments, R³ is not unsubstituted C₁₅ alkyl.

In embodiments, the compound includes one or more uptake motifs and one or more linkers.

An “uptake motif” as used herein refers to a portion including one or more long chain fatty acids (LCFA). The uptake motif in Formula (I) is defined as

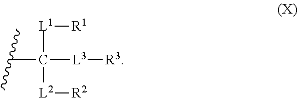

R¹, R², R³, L¹, L², and L³ are as defined herein.

In embodiments, the compound includes an uptake motif having the structure of

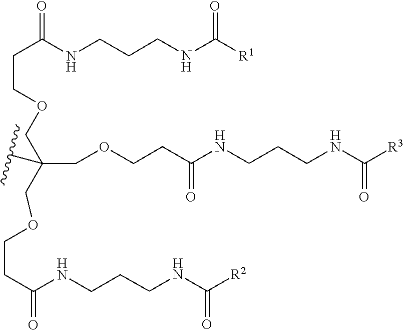

wherein the wavy line represents attachment point to -L⁶-L⁵-L⁴-L⁷-. R¹, R², and R³ are as described herein.

A “linker” refers to a portion of the compound that connects (or positioned between) the nucleic acids to the uptake motifs. In embodiments, a linker may be attached to an uptake motif directly or via one or more bridging groups (e.g., —C(O)—, or —C(O)NH—). In embodiments, a linker may be attached to an uptake motif in combination with one or more bridging groups (e.g., —C(O)—, or —C(O)NH—). In embodiments, a linker in combination with —C(O)— is attached to an uptake motif. In embodiments, a linker in combination with —C(O)NH— is attached to an uptake motif.

In embodiments, one or more linkers and one or more bridging groups (e.g., —C(O)—, or —C(O)NH—) constitute -L⁶-L⁵-L⁴-. In embodiments, one or more linkers and one or more bridging groups (e.g., —C(O)—, or —C(O)NH—) constitute -L⁶-L⁵-L⁴-L⁷-In embodiments, one or more linkers and one or more bridging groups (e.g., —C(O)—, or —C(O)NH—) constitute -L⁶-L⁵-. In embodiments, one or more linkers and one or more bridging groups (e.g., —C(O)—, or —C(O)NH—) constitute -L⁶-L⁵-L⁷-. In embodiments, one or more linkers and one or more bridging heteroatoms (e.g., —C(O)—, or —C(O)NH—) constitute -L⁶-L⁴-. In embodiments, one or more linkers and one or more bridging groups (e.g., —C(O)—, or —C(O)NH—) constitute -L⁶-L⁴-L⁷-. In embodiments, one or more linkers and one or more bridging groups (e.g., —C(O)—, or —C(O)NH—) constitute -L⁶-. In embodiments, one or more linkers and one or more bridging groups (e.g., —C(O)—, or —C(O)NH—) constitute -L⁶-L⁷-. In embodiments, one or more linkers and one or more bridging groups (e.g., —C(O)—, or —C(O)NH—) constitute -L⁷-.

In embodiments, the linker has one-atom length (e.g., substituted or unsubstituted methylene, —NH—, or —O—). In embodiments, the linker has two-atom length (e.g., substituted or unsubstituted ethylene, or substituted or unsubstituted 2-membered heteroalkylene). In embodiments, the linker has three-atom length (e.g., substituted or unsubstituted C3, or substituted or unsubstituted 3-membered heteroalkylene). In embodiments, the linker has four-atom length (e.g., substituted or unsubstituted C4, or substituted or unsubstituted 4-membered heteroalkylene). In embodiments, the linker has five-atom length (e.g., substituted or unsubstituted C5, or substituted or unsubstituted 5-membered heteroalkylene). In embodiments, the linker has six-atom length (e.g., substituted or unsubstituted C6, or substituted or unsubstituted 6-membered heteroalkylene). In embodiments, the linker has seven-atom length (e.g., substituted or unsubstituted C7, or substituted or unsubstituted 7-membered heteroalkylene). In embodiments, the linker has eight-atom length (e.g., substituted or unsubstituted C8, or substituted or unsubstituted 8-membered heteroalkylene). In embodiments, the linker has nine-atom length (e.g., substituted or unsubstituted C9, or substituted or unsubstituted 9-membered heteroalkylene). In embodiments, the linker has ten-atom length (e.g., substituted or unsubstituted C10, or substituted or unsubstituted 10-membered heteroalkylene). In embodiments, the linker has eleven-atom length (e.g., substituted or unsubstituted C11, or substituted or unsubstituted 11-membered heteroalkylene). In embodiments, the linker has twelve-atom length (e.g., substituted or unsubstituted C12, or substituted or unsubstituted 12-membered heteroalkylene). In embodiments, the linker has thirteen-atom length (e.g., substituted or unsubstituted C13, or substituted or unsubstituted 13-membered heteroalkylene). In embodiments, the linker has fourteen-atom length (e.g., substituted or unsubstituted C14, or substituted or unsubstituted 14-membered heteroalkylene). In embodiments, the linker has fifteen-atom length (e.g., substituted or unsubstituted C15, or substituted or unsubstituted 15-membered heteroalkylene). In embodiments, the linker has sixteen-atom length (e.g., substituted or unsubstituted C16, or substituted or unsubstituted 16-membered heteroalkylene). In embodiments, the linker has seventeen-atom length (e.g., substituted or unsubstituted C17, or substituted or unsubstituted 17-membered heteroalkylene). In embodiments, the linker has eighteen-atom length (e.g., substituted or unsubstituted C18, or substituted or unsubstituted 18-membered heteroalkylene). In embodiments, the linker has nineteen-atom length (e.g., substituted or unsubstituted C19, or substituted or unsubstituted 19-membered heteroalkylene). In embodiments, the linker has twenty-atom length (e.g., substituted or unsubstituted C20, or substituted or unsubstituted 20-membered heteroalkylene). In embodiments, the linker has twentyone-atom length (e.g., substituted or unsubstituted C21, or substituted or unsubstituted 21-membered heteroalkylene). In embodiments, the linker has twentytwo-atom length (e.g., substituted or unsubstituted C22, or substituted or unsubstituted 22-membered heteroalkylene). In embodiments, the linker has twentythree-atom length (e.g., substituted or unsubstituted C23, or substituted or unsubstituted 23-membered heteroalkylene). In embodiments, the linker has twentyfour-atom length (e.g., substituted or unsubstituted C24, or substituted or unsubstituted 23-membered heteroalkylene). In embodiments, the linker has twentyfive-atom length (e.g., substituted or unsubstituted C25 or substituted or unsubstituted 25-membered heteroalkylene).

In embodiments, the compound includes a linker having a structure shown in Table 1 below. In embodiments, the compound includes a linker C7 in Table 1. In embodiments, the compound includes a linker C6 in Table 1. In embodiments, the compound includes a linker C3 in Table 1. In embodiments, the compound includes a linker 6A-SER in Table 1. In embodiments, the compound includes a linker TEGN in Table 1. In embodiments, the compound includes a linker C12 in Table 1. In embodiments, the compound includes a linker AMC-N in Table 1. In embodiments, the compound includes a linker 5A-MEG in Table 1. In embodiments, the compound includes a linker AMC-P in Table 1. In embodiments, the compound includes a linker C6-P in Table 1. In embodiments, the compound includes a linker TEG-P in Table 1. In embodiments, the compound includes a linker PYR-C5-C3 in Table 1. In embodiments, the compound includes a linker PYR-DEG in Table 1. In embodiments, the compound includes a linker SP6-PO-C7 in Table 1. In embodiments, the compound includes a linker TEG9-PO-C7 in Table 1.

TABLE 1
Linker and —L⁶—L⁵—L⁴—Structures

| Linker | Linker Structure |
|---|---|
| Abbreviation | Example of —L⁶—L⁵—L⁴— |
| C7 | 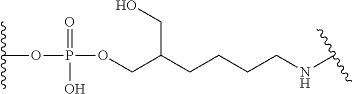 [SMILES: *NCCCCC(CO)COP(=O)(O)O*] |
| | 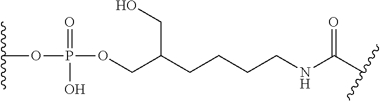 [SMILES: *OP(=O)(O)OCC(CO)CCCCNC(*)=O] |
| C6 | 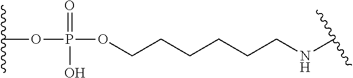 [SMILES: *NCCCCCCOP(=O)(O)O*] |
| | 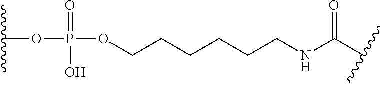 [SMILES: *OP(=O)(O)OCCCCCCNC(*)=O] |
| C3 | 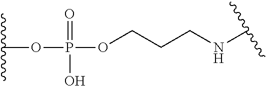 [SMILES: *NCCCOP(=O)(O)O*] |
| | 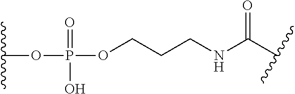 [SMILES: *OP(=O)(O)OCCCNC(*)=O] |
| 6A-SER | 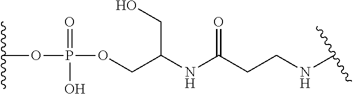 [SMILES: *NCCC(=O)NC(CO)COP(=O)(O)O*] |
| | 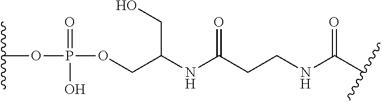 [SMILES: *OP(=O)(O)OCC(CO)NC(=O)CCNC(*)=O] |
| TEGN | 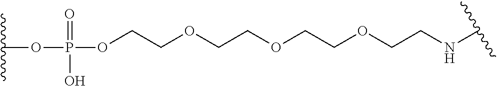 [SMILES: *NCCOCCOCCOCCOP(=O)(O)O*] |
| | 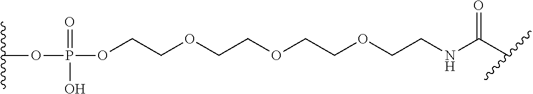 [SMILES: *OP(=O)(O)OCCOCCOCCOCCNC(*)=O] |
| C12 | 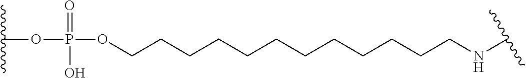 [SMILES: *NCCCCCCCCCCCCOP(=O)(O)O*] |
| | 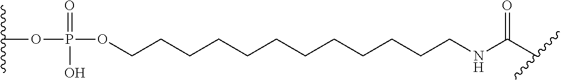 [SMILES: *OP(=O)(O)OCCCCCCCCCCCCNC(*)=O] |
| AMC-N | 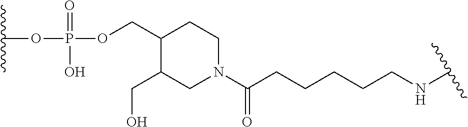 [SMILES: *NCCCCCC(=O)N1CCC(COP(=O)(O)O*)C(CO)C1] |
| | 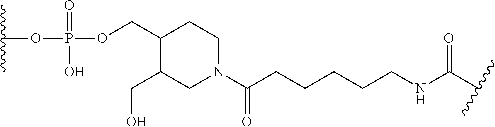 [SMILES: *OP(=O)(O)OCC1CCN(C(=O)CCCCCNC(*)=O)CC1CO] |
| 5A-MEG | 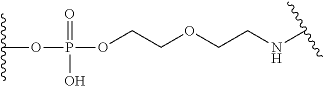 [SMILES: *NCCOCCOP(=O)(O)O*] |
| | 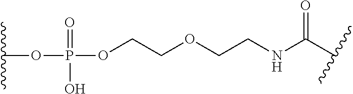 [SMILES: *OP(=O)(O)OCCOCCNC(*)=O] |
| AMC-P | 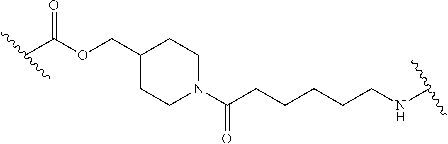 [SMILES: *NCCCCCC(=O)N1CCC(COC(*)=O)CC1] |
| | 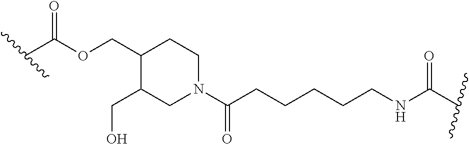 [SMILES: *C(=O)NCCCCCC(=O)N1CCC(COC(*)=O)C(CO)C1] |
| C6-P | 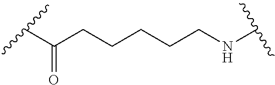 [SMILES: *NCCCCCC(*)=O] |
| | 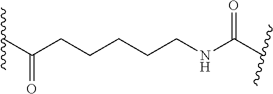 [SMILES: *C(=O)CCCCCNC(*)=O] |
| TEG-P | 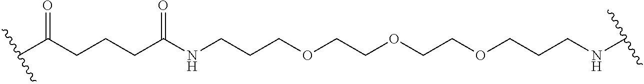 [SMILES: *NCCCOCCOCCOCCCNC(=O)CCCC(*)=O] |
| | 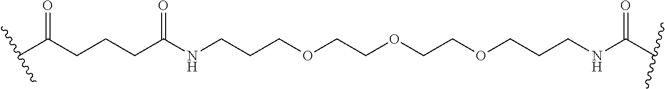 [SMILES: *C(=O)CCCC(=O)NCCCOCCOCCOCCCNC(*)=O] |
| PYR-C5-C3 | 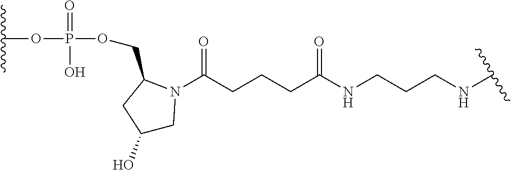 [SMILES: *NCCCNC(=O)CCCC(=O)N1C[C@H](O)C[C@H]1COP(=O)(O)O*] |
| | 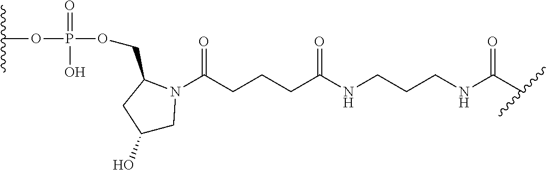 [SMILES: *OP(=O)(O)OC[C@@H]1C[C@@H](O)CN1C(=O)CCCC(=O)NCCCNC(*)=O] |
| PYR-DEG | 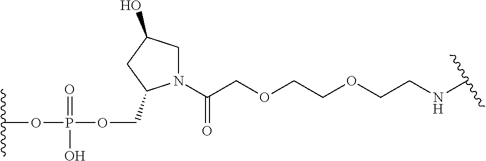 [SMILES: *NCCOCCOCC(=O)N1C[C@H](O)C[C@H]1COP(=O)(O)O*] |
| | 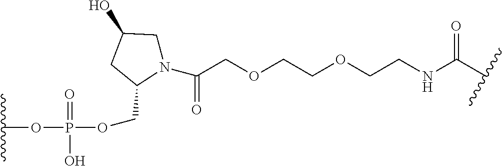 [SMILES: *OP(=O)(O)OC[C@@H]1C[C@@H](O)CN1C(=O)COCCOCCNC(*)=O] |
| SP6-PO-C7 | 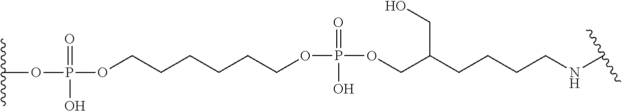 [SMILES: *NCCCCC(CO)COP(=O)(O)OCCCCCCOP(=O)(O)O*] |
| | 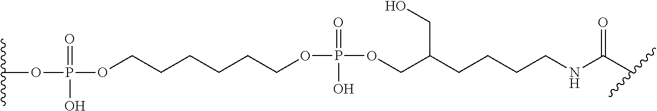 [SMILES: *OP(=O)(O)OCCCCCCOP(=O)(O)OCC(CO)CCCCNC(*)=O] |
| TEG9-PO-C7 | 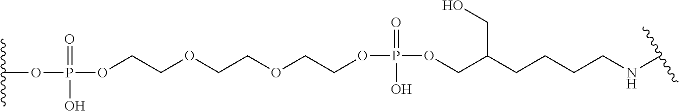 [SMILES: *NCCCCC(CO)COP(=O)(O)OCCOCCOCCOP(=O)(O)O*] |
| | 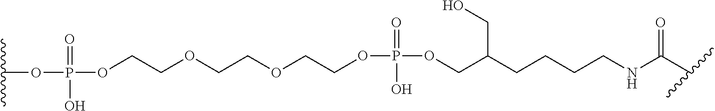 [SMILES: *OP(=O)(O)OCCOCCOCCOP(=O)(O)OCC(CO)CCCCNC(*)=O] |

L¹ is independently a bond, substituted (e.g., substituted with a substituent group, a size-limited substituent group, or lower substituent group) or unsubstituted heteroalkylene (e.g., 2 to 50 membered, 2 to 40 membered, 2 to 30 membered, 2 to 20 membered, 2 to 10 membered or 2 to 5 membered), or L^{1A}-L^{1B}-L^{1C}-L^{1D}-L^{1E}. In embodiments, L¹ is independently a bond, or substituted (e.g., substituted with a substituent group, a size-limited substituent group, or lower substituent group) heteroalkylene (e.g., 2 to 50 membered, 2 to 40 membered, 2 to 30 membered, 2 to 20 membered, 2 to 10 membered or 2 to 5 membered). In embodiments, L¹ is independently a bond, or unsubstituted heteroalkylene (e.g., 2 to 50 membered, 2 to 40 membered, 2 to 30 membered, 2 to 20 membered, 2 to 10 membered or 2 to 5 membered). In embodiments, L¹ is independently a bond. In embodiments, L¹ is not a bond. In embodiments, L¹ is independently L^{1A}-L^{1B}-L^{1C}-L^{1D}-L^{1E}.

In embodiments, when L¹ is substituted, L¹ is substituted with a substituent group. In embodiments, when L¹ is substituted, L¹ is substituted with a size-limited substituent group. In embodiments, when L¹ is substituted, L¹ is substituted with a lower substituent

L^{1A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1A} is substituted, L^{1A} is substituted with a substituent group. In embodiments, when L^{1A} is substituted, L^{1A} is substituted with a size-limited substituent group. In embodiments, when L^{1A} is substituted, L^{1A} is substituted with a lower substituent group.

L^{1B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1B} is substituted, L^{1B} is substituted with a substituent group. In embodiments, when L^{1B} is substituted, L^{1B} is substituted with a size-limited substituent group. In embodiments, when L^{1B} is substituted, L^{1B} is substituted with a lower substituent group.

L^{1C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1C} is substituted, L^{1C} is substituted with a substituent group. In embodiments, when L^{1C} is substituted, L^{1C} is substituted with a size-limited substituent group. In embodiments, when L^{1C} is substituted, L^{1C} is substituted with a lower substituent group.

L^{1D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1D} is substituted, L^{1D} is substituted with a substituent group. In embodiments, when L^{1D} is substituted, L^{1D} is substituted with a size-limited substituent group. In embodiments, when L^{1D} is substituted, L^{1D} is substituted with a lower substituent group.

L^{1E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1E} is substituted, L^{1E} is substituted with a substituent group. In embodiments, when L^{1E} is substituted, L^{1E} is substituted with a size-limited substituent group. In embodiments, when L^{1E} is substituted, L^{1E} is substituted with a lower substituent group.

L² is independently a bond, substituted (e.g., substituted with a substituent group, a size-limited substituent group, or lower substituent group) or unsubstituted heteroalkylene (e.g., 2 to 50 membered, 2 to 40 membered, 2 to 30 membered, 2 to 20 membered, 2 to 20 membered or 2 to 5 membered), or L^{2A}-L^{2B}-L^{2C}-L^{2D}-L^{2E}. In embodiments, L² is independently a bond, or substituted (e.g., substituted with a substituent group, a size-limited substituent group, or lower substituent group) heteroalkylene (e.g., 2 to 50 membered, 2 to 40 membered, 2 to 30 membered, 2 to 20 membered, 2 to 20 membered or 2 to 5 membered). In embodiments, L² is independently a bond, or unsubstituted heteroalkylene (e.g., 2 to 50 membered, 2 to 40 membered, 2 to 30 membered, 2 to 20 membered, 2 to 20 membered or 2 to 5 membered). In embodiments, L² is independently a bond. In embodiments, L² is not a bond. In embodiments, L² is independently L^{2A}-L^{2B}-L^{2C}-L^{2D}-L^{2E}.

In embodiments, when L² is substituted, L² is substituted with a substituent group. In embodiments, when L² is substituted, L² is substituted with a size-limited substituent group. In embodiments, when L² is substituted, L² is substituted with a lower substituent group.

L^{2A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{2A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{2A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{2A} is substituted, L^{2A} is substituted with a substituent group. In embodiments, when L^{2A} is substituted, L^{2A} is substituted with a size-limited substituent group. In embodiments, when L^{2A} is substituted, L^{2A} is substituted with a lower substituent

L^{2B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{2B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{2B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{2B} is substituted, L^{2B} is substituted with a substituent group. In embodiments, when L^{2B} is substituted, L^{2B} is substituted with a size-limited substituent group. In embodiments, when L^{2B} is substituted, L^{2B} is substituted with a lower substituent group.

L^{2C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{2C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{2C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{2C} is substituted, L^{2C} is substituted with a substituent group. In embodiments, when L^{2C} is substituted, L^{2C} is substituted with a size-limited substituent group. In embodiments, when L^{2C} is substituted, L^{2C} is substituted with a lower substituent group.

L^{2D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{2D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{2D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{2D} is substituted, L^{2D} is substituted with a substituent group. In embodiments, when L^{2D} is substituted, L^{2D} is substituted with a size-limited substituent group. In embodiments, when L^{2D} is substituted, L^{2D} is substituted with a lower substituent group.

L^{2E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{2E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{2E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{2E} is substituted, L^{2E} is substituted with a substituent group. In embodiments, when L^{2E} is substituted, L^{2E} is substituted with a size-limited substituent group. In embodiments, when L^{2E} is substituted, L^{2E} is substituted with a lower substituent group.

L³ is independently a bond, substituted (e.g., substituted with a substituent group, a size-limited substituent group, or lower substituent group) or unsubstituted heteroalkylene (e.g., 2 to 50 membered, 2 to 40 membered, 2 to 30 membered, 2 to 20 membered, 2 to 30 membered or 2 to 5 membered), or L^{3A}-L^{3B}-L^{3C}-L^{3D}-L^{3E}. In embodiments, L³ is independently a bond, or substituted (e.g., substituted with a substituent group, a size-limited substituent group, or lower substituent group) heteroalkylene (e.g., 2 to 50 membered, 2 to 40 membered, 2 to 30 membered, 2 to 20 membered, 2 to 30 membered or 2 to 5 membered). In embodiments, L³ is independently a bond, or unsubstituted heteroalkylene (e.g., 2 to 50 membered, 2 to 40 membered, 2 to 30 membered, 2 to 20 membered, 2 to 30 membered or 2 to 5 membered). In embodiments, L³ is independently a bond. In embodiments, L³ is not a bond. In embodiments, L³ is independently L^{3A}-L^{3B}-L^{3C}-L^{3D}-L^{3E}

In embodiments, when L³ is substituted, L³ is substituted with a substituent group. In embodiments, when L³ is substituted, L³ is substituted with a size-limited substituent group. In embodiments, when L³ is substituted, L³ is substituted with a lower substituent group.

L^{3A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{3A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{3A} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{3A} is substituted, L^{3A} is substituted with a substituent group. In embodiments, when L^{3A} is substituted, L^{3A} is substituted with a size-limited substituent group. In embodiments, when L^{3A} is substituted, L^{3A} is substituted with a lower substituent group.

L^{3B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{3B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{3B} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{3B} is substituted, L^{3B} is substituted with a substituent group. In embodiments, when L^{3B} is substituted, L^{3B} is substituted with a size-limited substituent group. In embodiments, when L^{3B} is substituted, L^{3B} is substituted with a lower substituent group.

L^{3C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{3C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{3C} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{3C} is substituted, L^{3C} is substituted with a substituent group. In embodiments, when L^{3C} is substituted, L^{3C} is substituted with a size-limited substituent group. In embodiments, when L^{3C} is substituted, L^{3C} is substituted with a lower substituent group.

L^{3D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{3D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{3D} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{3D} is substituted, L^{3D} is substituted with a substituent group. In embodiments, when L^{3D} is substituted, L^{3D} is substituted with a size-limited substituent group. In embodiments, when L^{3D} is substituted, L^{3D} is substituted with a lower substituent group.

L^{3E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{3E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{3E} is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{3E} is substituted, L^{3E} is substituted with a substituent group. In embodiments, when L^{3E} is substituted, L^{3E} is substituted with a size-limited substituent group. In embodiments, when L^{3E} is substituted, L^{3E} is substituted with a lower substituent group.

L⁴ is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁴ is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁴ is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁴ is substituted, L⁴ is substituted with a substituent group. In embodiments, when L⁴ is substituted, L⁴ is substituted with a size-limited substituent group. In embodiments, when L⁴ is substituted, L⁴ is substituted with a lower substituent group.

L⁵ is a bond, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁵ is a bond, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁵ is a bond, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁵ is substituted, L⁵ is substituted with a substituent group. In embodiments, when L⁵ is substituted, L⁵ is substituted with a size-limited substituent group. In embodiments, when L⁵ is substituted, L⁵ is substituted with a lower substituent group.

L⁶ is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶ is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁶ is a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁶ is substituted, L⁶ is substituted with a substituent group. In embodiments, when L⁶ is substituted, L⁶ is substituted with a size-limited substituent group. In embodiments, when L⁶ is substituted, L⁶ is substituted with a lower substituent group.

L⁷ is independently a bond, substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 8 membered, 2 to 6 membered, 4 to 6 membered, 2 to 3 membered, or 4 to 5 membered). In embodiments, L⁶ independently is a bond, substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L⁶ is independently a bond, unsubstituted alkylene (e.g., C₁-C₂₅, C₁-C₂₀, C₁-C₁₂, C₁-C₈, C₁-C₆, C₁-C₄, or C₁-C₂), or unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, when L⁷ is substituted, L⁶ is substituted with a substituent group. In embodiments, when L⁷ is substituted, L⁶ is substituted with a size-limited substituent group. In embodiments, when L⁷ is substituted, L⁷ is substituted with a lower substituent group.

L¹⁰ is independently substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹⁰ is independently substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹⁰ is independently unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, when L¹⁰ is substituted, L¹⁰ is substituted with a substituent group. In embodiments, when L¹⁰ is substituted, L¹⁰ is substituted with a size-limited substituent group. In embodiments, when L¹⁰ is substituted, L¹⁰ is substituted with a lower substituent group.

L¹¹ is independently substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹¹ is independently substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹¹ is independently unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, when L¹¹ is substituted, L¹¹ is substituted with a substituent group. In embodiments, when L¹¹ is substituted, L¹¹ is substituted with a size-limited substituent group. In embodiments, when L¹¹ is substituted, L¹¹ is substituted with a lower substituent group.

L¹² is independently substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹² is independently substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹² is independently unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, when L¹² is substituted, L¹² is substituted with a substituent group. In embodiments, when L¹² is substituted, L¹² is substituted with a size-limited substituent group. In embodiments, when L¹² is substituted, L¹² is substituted with a lower substituent group.

L¹³ is independently substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹³ is independently substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹³ is independently unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, when L¹³ is substituted, L¹³ is substituted with a substituent group. In embodiments, when L¹³ is substituted, L¹³ is substituted with a size-limited substituent group. In embodiments, when L¹³ is substituted, L¹³ is substituted with a lower substituent group.

L¹⁴ is independently substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹⁴ is independently substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹⁴ is independently unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, when L¹⁴ is substituted, L¹⁴ is substituted with a substituent group. In embodiments, when L¹⁴ is substituted, L¹⁴ is substituted with a size-limited substituent group. In embodiments, when L¹⁴ is substituted, L¹⁴ is substituted with a lower substituent group.

L¹⁷ is independently substituted (e.g., substituted with a substituent group, a size-limited substituent group, or lower substituent group) or unsubstituted alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹⁷ is independently substituted (e.g., substituted with a substituent group, a size-limited substituent group, or lower substituent group) alkylene (e.g., C₁-C₂₅, C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or substituted (e.g., substituted with a substituent group, a size-limited substituent group, or lower substituent group) membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, L¹⁷ is independently unsubstituted alkylene (e.g., C₁-C₂₅, C₁-C₂₀, C₁-C₁₂, C₁-C₈, C₁-C₆, C₁-C₄, or C₁-C₂), unsubstituted heteroalkylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered), or unsubstituted membered heteroalkenylene (e.g., 2 to 25 membered, 2 to 20 membered, 2 to 12 membered, 2 to 12 membered, 2 to 8 membered, 2 to 6 membered, 4 to 6 membered, 2 to 3 membered, or 4 to 5 membered). In embodiments, when L¹⁷ is substituted, L¹⁷ is substituted with a substituent group. In embodiments, when L¹⁷ is substituted, L¹⁷ is substituted with a size-limited substituent group. In embodiments, when L¹⁷ is substituted, L¹⁷ is substituted with a lower substituent group.

A is a nucleic acid. In embodiments, a nucleic acid is a double-stranded nucleic acid. In embodiments, a nucleic acid is a single-stranded nucleic acid. In embodiments, the double-stranded nucleic acid is a small interfering RNA, a short hairpin RNA or a microRNA mimic. In embodiments, the single-stranded nucleic acid is a single-strand small interfering RNA, an RNaseH oligonucleotide, an anti-microRNA oligonucleotide, a steric blocking oligonucleotide, exon-skipping oligonucleotide, a CRISPR guide RNA, or an aptamer.

In embodiments, a nucleic acid is a double-stranded nucleic acid comprising an antisense strand complementary to the mRNA and a sense strand complementary to the antisense strand. In embodiments, the antisense strand and sense strand of a compound are two separate strands and are not covalently linked and form a small interfering RNA (siRNA). In embodiments, the antisense strand and sense strand of a compound are covalently linked by a nucleotide linker to form a short hairpin RNA (shRNA). In embodiments, the compound is a single-stranded nucleic acid comprising an antisense strand complementary to the mRNA (ssRNAi).

In embodiments, a nucleic acid is a double-stranded nucleic acid comprising an antisense strand and a sense strand hybridized to form a double-stranded region, each of the antisense strand and sense strands is 15 to 25 nucleotides in length, the nucleotide sequence of the antisense strand is at least 90% complementary to the nucleotide sequence of an mRNA, and the nucleotide sequence of the sense strand has no more than two mismatches to the nucleotide sequence of the antisense strand.

In embodiments, a nucleic acid is a single-stranded nucleic acid comprising an antisense strand, wherein the antisense strand is 15 to 25 nucleotides in length and the nucleotide sequence of the antisense strand is at least 90% complementary to the nucleotide sequence of an mRNA.

Provided below are features of compounds, such as length, nucleotide sequence, and nucleotide modifications. It is understood that an embodiment of an antisense strand may apply to the antisense strand of a single-stranded nucleic acid or a double-stranded nucleic acid. Further, it is understood that an embodiment of a sense strand may apply to a sense strand of any double-stranded nucleic acid provided herein, including siRNAs and shRNAs.

In embodiments, the nucleic acid is a double-stranded nucleic acid comprising an antisense strand hybridized to a sense strand, and wherein each of the antisense strand and sense strand is independently 15 to 25 nucleotides in length. In embodiments, each of the antisense strand and sense strand is independently 17 to 23 nucleotides in length. In embodiments, each of the antisense strand and sense strand is independently 19 to 21 nucleotides in length. In embodiments, each of the antisense strand and sense strand is independently 21 to 23 nucleotides in length.

In embodiments, the nucleic acid is a single-stranded nucleic acid and the single-stranded nucleic acid is 8 to 30 nucleotides in length. In embodiments, the single-stranded nucleic acid is 12 to 25 nucleotides in length. In embodiments, the single-stranded nucleic acid is 15 to 25 nucleotides in length. In embodiments, the single-stranded nucleic acid is 17 to 23 nucleotides in length. In embodiments, the single-stranded nucleic acid is 19 to 21 nucleotides in length. In embodiments, the single-stranded nucleic acid is 21 to 23 nucleotides in length.

In embodiments, an antisense strand is 15 to 25 nucleotides in length. In embodiments, an antisense strand is 17 to 23 nucleotides in length. In embodiments, an antisense strand is 19 to 21 nucleotides in length. In embodiments, an antisense strand is 21 to 23 nucleotides in length. In embodiments, an antisense strand is 15 nucleotides in length. In embodiments, an antisense strand is 16 nucleotides in length. In embodiments, an antisense strand is 17 nucleotides in length. In embodiments, an antisense strand is 18 nucleotides in length. In embodiments, an antisense strand is 19 nucleotides in length. In embodiments, an antisense strand is 20 nucleotides in length. In embodiments, an antisense strand is 21 nucleotides in length. In embodiments, an antisense strand is 22 nucleotides in length. In embodiments, an antisense strand is 23 nucleotides in length. In embodiments, an antisense strand is 24 nucleotides in length. In embodiments, an antisense strand is 25 nucleotides in length.

In embodiments, a sense strand is 15 to 25 nucleotides in length. In embodiments, a sense strand is 17 to 23 nucleotides in length. In embodiments, a sense strand is 19 to 21 nucleotides in length. In embodiments, a sense strand is 21 to 23 nucleotides in length. In embodiments, a sense strand is 15 nucleotides in length. In embodiments, a sense strand is 16 nucleotides in length. In embodiments, a sense strand is 17 nucleotides in length. In embodiments, a sense strand is 18 nucleotides in length. In embodiments, a sense strand is 19 nucleotides in length. In embodiments, a sense strand is 20 nucleotides in length. In embodiments, a sense strand is 21 nucleotides in length. In embodiments, a sense strand is 22 nucleotides in length. In embodiments, a sense strand is 23 nucleotides in length. In embodiments, a sense strand is 24 nucleotides in length. In embodiments, a sense strand is 25 nucleotides in length.

In embodiments, the length of the sense strand is identical to the length of the antisense strand. In embodiments, the length of the sense strand is greater than the length of the antisense strand. In embodiments, the length of the sense strand is less than the length of the antisense strand.

The double-stranded region of a double-stranded nucleic acid may be from 15 to 25 nucleobase pairs in length, depending on the lengths of the sense strand and the antisense strand. In embodiments, the double-stranded region is 17 to 23 nucleobase pairs in length. In embodiments, the double-stranded region is 19 to 21 nucleobase pairs in length. In embodiments, the double-stranded region is 21 to 23 nucleotides in length. In embodiments, the double-stranded region is 15 nucleobase pairs in length. In embodiments, the double-stranded region is 16 nucleobase pairs in length. In embodiments, the double-stranded region is 17 nucleobase pairs in length. In embodiments, the double-stranded region is 18 nucleobase pairs in length. In embodiments, the double-stranded region is 19 nucleobase pairs in length. In embodiments, the double-stranded region is 20 nucleobase pairs in length. In embodiments, the double-stranded region is 21 nucleobase pairs in length. In embodiments, the double-stranded region is 22 nucleobase pairs in length. In embodiments, the double-stranded region is 23 nucleobase pairs in length. In embodiments, the double-stranded region is 24 nucleobase pairs in length. In embodiments, the double-stranded region is 25 nucleobase pairs in length.

In embodiments, the nucleotide sequence of a sense strand has no more than one mismatch to the nucleotide sequence of an antisense strand of a double-stranded nucleic acid. In embodiments, the nucleotide sequence of a sense strand has no mismatches to the nucleotide sequence of an antisense strand of a double-stranded nucleic acid. Single-stranded nucleotide overhangs and nucleotide linkers are not considered for the purposes of determining the number of mismatches within the double-stranded region of a double-stranded nucleic acid provided herein. For example, a double-stranded nucleic acid comprising an antisense strand that is 23 nucleotides in length, and a sense strand that is 21 nucleotides in length have no mismatches over the double-stranded region, provided the nucleotide sequence of the sense strand is fully complementary over its length the nucleotide sequence of the antisense strand. Alternatively, a double-stranded nucleic acid comprising a sense strand that is 20 nucleotides in length, an antisense strand that is 22 nucleotides in length, and a nucleotide linker that is eight nucleotides in length, may have no mismatches over the double-stranded region provided the nucleotide sequence of the sense strand is fully complementary over its length to the nucleotide sequence of the antisense strand.

In embodiments, a double-stranded nucleic acid comprises an antisense strand of 19 nucleotides in length and a sense strand of 19 nucleotides in length. In embodiments, the antisense strand is 22 nucleotides in length and the sense strand is 20 nucleotides in length. In embodiments, the antisense strand is 23 nucleotides in length and the sense strand is 21 nucleotides in length. In embodiments, the antisense strand is 23 nucleotides in length including two deoxythymidines at the 3′ terminus, and the sense strand is 21 nucleotides in length including two deoxythymidines at the 3′ terminus.

In embodiments, the nucleotide sequence of the antisense strand is at least 90% complementary to at least 15, at least 16, at least 17, at least 18, at least 19, at least 20, at least 21, at least 22, at least 23, at least 24, or 25 nucleotides of a target mRNA. In embodiments, the nucleotide sequence of the antisense strand is at least 90% complementary to at least 15 contiguous nucleotides of a target mRNA. In embodiments, the nucleotide sequence of the antisense strand is at least 90% complementary to at least 16 contiguous nucleotides of a target mRNA. In embodiments, the nucleotide sequence of the antisense strand is at least 90% complementary to at least 17 contiguous nucleotides of a target mRNA. In embodiments, the nucleotide sequence of the antisense strand is at least 90% complementary to at least 18 contiguous nucleotides of a target mRNA. In embodiments, the nucleotide sequence of the antisense strand is at least 90% complementary to at least 19 contiguous nucleotides of a target mRNA. In embodiments, the nucleotide sequence of the antisense strand is at least 90% complementary to at least 20 contiguous nucleotides of a target mRNA. In embodiments, the nucleotide sequence of the antisense strand is at least 90% complementary to at least 21 contiguous nucleotides of a target mRNA. In embodiments, the antisense strand is at least 90% complementary to at least 22 contiguous nucleotides of a target mRNA. In embodiments, the nucleotide sequence of the antisense strand is at least 90% complementary to at least 23 contiguous nucleotides of a target mRNA. In embodiments, the nucleotide sequence of the antisense strand is at least 90% complementary to at least 24 contiguous nucleotides of a target mRNA. In embodiments, the nucleotide sequence of the antisense strand is at least 90% complementary to 25 contiguous nucleotides of a target mRNA. In embodiments, the nucleotide sequence of the antisense strand is at least 95% complementary to a target mRNA. In embodiments, the nucleotide sequence of the antisense strand is 100% complementary to a target mRNA.

In embodiments of compound comprising double-stranded nucleic acid where the antisense strand and sense strand are separate strands that are not covalently linked, the terminal nucleotides may form a nucleobase pair, in which case the end of the double-stranded nucleic acid is a blunt end. Alternatively, one or more unpaired nucleotides of an antisense strand and/or sense strand may extend beyond the terminus of the complementary strand, resulting in a nucleotide overhang of one or more terminal single-stranded nucleotides. In embodiments, at least one of the 5′ and 3′ terminus of a double-stranded nucleic acid is a blunt end. In a embodiments, both the 5′ terminus and 3′ terminus of the double-stranded nucleic acid are blunt ends. In embodiments, at least one end of the double-stranded nucleic acid comprises a nucleotide overhang. In embodiments, each end of the double-stranded nucleic acid comprises a nucleotide overhang. In embodiments, one end of the double-stranded nucleic acid is a blunt end and the other end of the double-stranded nucleic acid comprises a nucleotide overhang. In embodiments, the antisense strand comprises a nucleotide overhang at its 3′ terminus. In embodiments, the sense strand comprises a nucleotide overhang at its 3′ terminus. In embodiments, each of the antisense strand and sense strand comprises a nucleotide overhang at its 3′ terminus. In embodiments, at least one of the antisense strand and sense strand comprises a nucleotide overhang at its 5′ terminus. In embodiments, each of the antisense strand and sense strand comprises a nucleotide overhang at each 5′ terminus.

In embodiments, a nucleotide overhang is from one to five single-stranded nucleotides. In embodiments, a nucleotide overhang is one single-stranded nucleotide. In embodiments, a nucleotide overhang is two single-stranded nucleotides. In embodiments, a nucleotide overhang is three single-stranded nucleotides. In embodiments, a nucleotide overhang is three single-stranded nucleotides. In embodiments, a nucleotide overhang is four single-stranded nucleotides. In embodiments, a nucleotide overhang is five single-stranded nucleotides. In embodiments, at least one of the single-stranded nucleotides of a nucleotide overhang is a modified nucleotide. In embodiments, each of the single-stranded nucleotides of a nucleotide overhang is a modified nucleotide. In embodiments, the modified nucleotide is a 2′-O-methyl nucleotide. In embodiments, the nucleotide overhang is two single-stranded nucleotides and each nucleotide is a 2′-O-methoxyethyl nucleotide.

In embodiments, at least one nucleotide of the nucleotide overhang at the 3′ terminus of an antisense strand is complementary to a corresponding nucleotide of a target mRNA. In embodiments, each nucleotide of the nucleotide overhang at the 3′ terminus of an antisense strand is complementary to a corresponding nucleotide of a target mRNA. In some embodiment, at least one nucleotide of the nucleotide overhang at the 3′ terminus of an antisense strand is not complementary to a corresponding nucleotide of a target mRNA. In embodiments, each nucleotide of the nucleotide overhang at the 3′ terminus of an antisense strand is not complementary to a corresponding nucleotide a target mRNA.

In embodiments, at least one single-stranded nucleotide of a nucleotide overhang is a deoxythymidine nucleotide. In embodiments, a nucleotide overhang is two single-stranded nucleotides and each nucleotide is a deoxythymidine nucleotide. In embodiments, the nucleotide sequence of the antisense strand comprises a nucleotide overhang of two deoxythymidine nucleotides. In embodiments, the sense strand comprises a nucleotide overhang of two deoxythymidine nucleotides. In embodiments, the antisense strand and the sense strand comprise a nucleotide overhang of two deoxythymidine nucleotides.

Non-limiting examples of double-stranded nucleic acids comprising blunt ends or nucleotide overhangs are provided in Table 2 below.

In the first example, where the antisense strand is 21 nucleotides in length and the sense strand is 21 nucleotides in length, and the nucleotide sequence of the antisense strand is fully complementary to the nucleotide sequence of the sense strand over the double-stranded region, the length of the double-stranded region is 19 nucleobase pairs and each terminus of the double-stranded nucleic acid has a dTdT overhang.

In the second example, where the antisense strand is 21 nucleotides in length and the sense strand is 19 nucleotides in length, and the nucleotide sequence of the antisense strand is fully complementary to the nucleotide sequence of the sense strand over the double-stranded region, the length of the double-stranded region is 19 nucleobase pairs and the 3′ terminus of the antisense strand comprises a dTdT overhang.

In the third example, where the antisense strand is 19 nucleotides in length and the sense strand is 19 nucleotides in length, and the nucleotide sequence of the antisense strand is fully complementary to the nucleotide sequence of the sense strand over the double-stranded region, the length of the double-stranded region is 19 nucleobase pairs and each terminus is a blunt end.

In the fourth example, where the antisense strand is 23 nucleotides in length and the sense strand is 21 nucleotides in length, the length of the double-stranded region is 21 nucleobase pairs and 3′ terminus of the antisense strand comprises a two-nucleotide overhang.

TABLE 2
Examples of double-stranded nucleic acids

| | | | | | SEQ |
|---|---|---|---|---|---|
| | | Nb | Terminus | | ID |
| Strand | Length | Pairs | Type | Nucleotide sequence | NO: |
| Sense | 21 | 19 | Overhang/ | 5′-GAUGAUGUUUGAAACUAUUTT-3′ | 1 |
| | | | | \|\|\|\|\|\|\|\|\|\|\|\|\|\|\|\|\|\|\| | |
| Antisense | 21 | | Overhang | 3′-TTCUACUACAAACUUUGAUAA-5′ | 2 |
| Sense | 19 | 19 | Overhang/ | 5′-GAUGAUGUUUGAAACUAUU-3′ | 3 |
| | | | | \|\|\|\|\|\|\|\|\|\|\|\|\|\|\|\|\|\|\| | |
| Antisense | 21 | | Blunt | 3′-TTCUACUACAAACUUUGAUAA-5′ | 2 |
| Sense | 19 | 19 | Blunt/ | 5′-GAUGAUGUUUGAAACUAUU-3′ | 3 |
| | | | | \|\|\|\|\|\|\|\|\|\|\|\|\|\|\|\|\|\|\| | |
| Antisense | 19 | | Blunt | 3′-CUACUACAAACUUUGAUAA-5′ | 4 |
| Sense | 21 | 21 | Overhang/ | 5′-GAUGAUGUUUGAAACUAUU-3′ | 3 |
| | | | | \|\|\|\|\|\|\|\|\|\|\|\|\|\|\|\|\|\|\| | |
| Antisense | 23 | | Blunt | 3′-UUCUACUACAAACUUUGAUAA-5′ | 5 |

In embodiments of a double-stranded nucleic acid comprising a nucleotide linker, the termini that are not connected by the nucleotide linker may form a blunt end, or may form a nucleotide overhang of one or more single-stranded nucleotides. In embodiments, the non-linked end of the double-stranded nucleic acid is a blunt end. In embodiments, the non-linked end comprises a nucleotide overhang of one or more single-stranded nucleotides. In embodiments, the non-linked end of the guide strand comprises a nucleotide overhang. In embodiments, the non-linked end of the sense strand comprises a nucleotide overhang. In embodiments, the 3′ terminus of the guide strand comprises a nucleotide overhang. In embodiments, the 3′ terminus of the sense strand comprises a nucleotide overhang. In embodiments, the 5′ terminus of the sense strand comprises a nucleotide overhang. In embodiments, the 5′ terminus of the sense strand comprises a nucleotide overhang.

In embodiments of a double-stranded nucleic acid where the antisense and sense strand are covalently linked by a nucleotide linker, the nucleotide linker is four to 16 nucleotides in length. In embodiments, the nucleotide linker is four nucleotides in length. In embodiments, the nucleotide linker is four nucleotides in length. In embodiments, the nucleotide linker is five nucleotides in length. In embodiments, the nucleotide linker is six nucleotides in length. In embodiments, the nucleotide linker is seven nucleotides in length. In embodiments, the nucleotide linker is eight nucleotides in length. In embodiments, the nucleotide linker is nine nucleotides in length. In embodiments, the nucleotide linker is 10 nucleotides in length. In embodiments, the nucleotide linker is 11 nucleotides in length. In embodiments, the nucleotide linker is 12 nucleotides in length. In embodiments, the nucleotide linker is 13 nucleotides in length. In embodiments, the nucleotide linker is 14 nucleotides in length. In embodiments, the nucleotide linker is 15 nucleotides in length. In embodiments, the nucleotide linker is 16 nucleotides in length.

In embodiments, the nucleic acid of A comprises one or more modified nucleotides. A modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation.

In embodiments, a modified nucleotide comprises one or more of a modified sugar moiety, a modified internucleotide linkage, and a 5′-terminal modified phosphate group. In embodiments, a modified nucleotide comprises a modified sugar moiety. In embodiments, a modified nucleotide comprises a modified internucleotide linkage. In embodiments, a modified nucleotide comprises a modified nucleobase. In embodiments, a modified nucleotide comprises a modified 5′-terminal phosphate group. In embodiments, a modified nucleotide comprises a modification at the 5′ carbon of the pentafuranosyl sugar. In embodiments, a modified nucleotide comprises a modification at the 3′ carbon of the pentafuranosyl sugar. In embodiments, a modified nucleotide comprises a modification at the 2′ carbon of the pentafuranosyl sugar. In embodiments, a modified nucleotide is at the 5′ terminus of an antisense strand or sense strand. In embodiments, a modified nucleotide is at the 3′ terminus of an antisense strand or sense strand. In embodiments, a modified nucleotide is at an internal nucleotide of an antisense strand or sense strand. In embodiments, a modified nucleotide comprises a ligand attached to the 2′, 3, or 5′ carbon of the pentafuranosyl sugar. In embodiments, a nucleotide comprises a ligand attached to a nucleobase.

In embodiments, a nucleic acid comprises one or more modified sugar moieties. In embodiments, a modified sugar moiety comprises a 2′ modification, i.e. a modification at the 2′ carbon of the pentafuranosyl sugar, relative to the naturally occurring 2′-OH of RNA or the 2′-H of DNA. In embodiments, a modification at the 2′ carbon of the pentafuranosyl sugar is selected from F, OCF₃, OCH₃ (also referred to as “2′-OMe” or “2′-O-methyl), OCH₂CH₂OCH₃ (also referred to as “2′-O-methoxyethyl” or “2′-MOE”), 2′-O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In embodiments, a modified nucleotide comprises a modified sugar moiety, where the ribose has a covalent linkage between the 2′ and 4′ carbons. Such a modified sugar moiety may be referred to as a “bicyclic sugar modification.” In embodiments, the covalent linkage of a bicyclic sugar is a methyleneoxy linkage (4′-CH₂—O-2′), also known as “LNA.” In embodiments, the covalent linkage of a bicyclic sugar is an ethyleneoxy linkage (4′-(CH₂)₂—O-2′), also known as “ENA.” In embodiments, the covalent linkage of a bicyclic moiety is a methyl(methyleneoxy) linkage (4′-CH(CH₃)—O-2′), also known as “constrained ethyl” or “cEt.” In certain embodiments, the —CH(CH₃)— bridge is constrained in the S orientation (“S-cEt”). In certain embodiments, the —CH(CH₃)— bridge is constrained in the R orientation (“R-cEt”). In embodiments, the covalent linkage of a bicyclic sugar is a (4′-CH(CH₂—OMe)-O-2′ linkage, also known as “c-MOE.” In embodiments, the bicyclic sugar is a D sugar in the alpha configuration. In certain such embodiments, the bicyclic sugar is a D sugar in the beta configuration. In certain such embodiments, the bicyclic sugar is an L sugar in the alpha configuration. In certain such embodiments, the bicyclic sugar is an L sugar in the beta configuration.

In embodiments, a modified sugar moiety comprises a 2′-modification selected from a 2′-fluoro modification, a 2′-O-methyl modification, a 2′-O-methoxyethyl modification, and a bicyclic sugar modification.

In embodiments, a modified sugar moiety comprises a bicyclic sugar modification selected from a 4′-CH(CH₃)—O-2′ linkage, a 4′-(CH₂)₂—O-2′ linkage, a 4′-CH(CH₂—OMe)-O-2′ linkage, 4′-CH₂—N(CH₃)—O-2′ linkage, and 4′-CH₂—N(H)—O-2′ linkage.

In embodiments, a modified sugar moiety comprises an acyclic nucleoside derivative lacking the bond between the 2′ carbon and 3′ carbon of the sugar ring, also known as an “unlocked sugar modification.”

In embodiments, a modified sugar moiety is a 1,5-anhydrohexitol nucleic acid, also known as a “hexitol nucleic acid” or “HNA.”

In embodiments, the oxygen of the pentafuranosyl sugar is replace with a sulfur, to form a thio-sugar. In embodiments, a thio-sugar is modified at the 2′ carbon.

In embodiments, a modified sugar moiety is a morpholino moiety.

In embodiments, a modified nucleotide comprising a modified sugar moiety is selected from a 2′-fluoro nucleotide, a 2′-O-methyl nucleotide, a 2′-O-methoxyethyl nucleotide, and a bicyclic sugar nucleotide. In embodiments, a modified nucleotide is a 2′-fluoro nucleotide, where the 2′ carbon of the pentafuranosyl sugar has a fluoro substitution. In embodiments, a modified nucleotide is a 2′-O-methyl nucleotide, where the 2′ carbon of the pentafuranosyl sugar has a 2′-O methyl substitution. In embodiments, a modified nucleotide is a 2′-O-methoxyethyl nucleotide, where the 2′ carbon of the pentafuranosyl sugar has a 2′-O-methoxyethyl substitution. Other modified nucleotides may be similarly named.

In embodiments, a nucleic acid comprises one or more modified internucleotide linkages. In embodiments, a modified internucleotide linkage is a phosphorothioate internucleotide linkage. In embodiments, a modified internucleotide linkage is a methylphosphonate internucleotide linkage.

In embodiments, a nucleic acid comprises a hydroxyl group, a phosphate group, or modified phosphate group. In embodiments, a modified phosphate group is a 5′-(E)-vinylphosphonate.

In embodiments, at least one nucleotide of the antisense strand is a modified nucleotide. In embodiments, at least one nucleotide of the sense strand is a modified nucleotide. In embodiments, each nucleotide of the antisense strand forming the double-stranded region is a modified nucleotide. In embodiments, each nucleotide of the sense strand forming the double-stranded region comprises is a modified nucleotide.

In embodiments, the first two internucleotide linkages at the 5′ terminus of the sense strand and the last two internucleotide linkages at the 3′ terminus of the sense strand are phosphorothioate internucleotide linkages. In embodiments, the first two internucleotide linkages at the 5′ terminus of the antisense strand and the last two internucleotide linkages at the 3′ terminus of the antisense strand are phosphorothioate internucleotide linkages. In embodiments, the first two internucleotide linkages at the 5′ terminus of the sense strand and the last two internucleotide linkages at the 3′ terminus of the sense strand are phosphorothioate internucleotide linkages, and the first two internucleotide linkages at the 5′ terminus of the antisense strand and the last two internucleotide linkages at the 3′ terminus of the antisense strand are phosphorothioate internucleotide linkages.

In embodiments, a modified nucleobase is selected from 5-hydroxymethyl cytosine, 7-deazaguanine and 7-deazaadenine. In embodiments, a modified nucleobase is selected from 7-deaza-adenine, 7-deazaguanosine, 2-aminopyridine and 2-pyridone. In embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

In embodiments, the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern I:
5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}N_{F}N_{M}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern II:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N^{S}N-3′,

wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern III:

5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}^{S}N_{F}^{S}N_{M}-3′,

wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkages is a phosphodiester internucleotide linkage.

In embodiments, the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleotide linkages at the 5′ terminus and the last two internucleotide linkages at the 3′ terminus are phosphorothioate internucleotide linkages, and each other internucleotide is a phosphodiester internucleotide linkage. Such a modification pattern may be represented by the following Pattern IV:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphorodiester internucleotide linkage.

In embodiments, the antisense strand is 23 nucleotides in length and wherein counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern V:

5′-N_{M}^{S}N_{F}^{S}N_{M}N_{F}N_{M}N_{F}N_{M}NEN_{M}N_{F}N_{M}N_{F}N_{M}N_{F}N_{M}N_{F}N_{M}N_{F}N_{M}N_{F}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, wherein the sense strand is 21 nucleotides in length and wherein counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern VI:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the antisense strand is 23 nucleotides in length and wherein counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern VII:

5′-N_{M}^{S}N_{F}^{S}N_{M}N_{F}N_{M}NEN_{M}N_{F}N_{M}N_{F}N_{M}N_{M}N_{M}N_{F}N_{M}N_{F}N_{M}N_{F}N_{M}N_{F}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and wherein counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern VIII:

5′-N_{F}^{S}N_{M}^{S}N_{F}N_{M}N_{F}N_{M}N_{F}N_{M}N_{F}N_{F}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the antisense strand is 23 nucleotides in length and wherein counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern IX:

5′-N_{M}^{S}N_{F}^{S}N_{M}N_{F}N_{M}N_{F}N_{M}N_{F}N_{M}N_{M}N_{M}N_{F}N_{M}N_{F}N_{M}N_{F}N_{M}N_{F}N_{M}N_{F}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and wherein counting from the 5′ terminus of the sense strand, nucleotides 1, 3, 5, 7, 9, 11, 12, 13, 15, 17, 19, and 21 are 2′-fluoronucleotides, nucleotides 2, 4, 6, 8, 10, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern X:

5′-N_{F}^{S}N_{M}^{S}N_{F}N_{M}N_{F}N_{M}N_{F}N_{M}N_{F}N_{M}N_{F}N_{F}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, wherein the sense strand is 23 nucleotides in length and wherein counting from the 5′ terminus of the sense strand, nucleotides 1, 3, 5, 7, 9, 11, 13, 15, 17, 19, and 21 are 2′-fluoronucleotides, nucleotides 2, 4, 6, 8, 10, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XI:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 23 nucleotides in length and wherein counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XII:

5′-N_{F}^{S}N_{M}^{S}N_{F}N_{M}N_{F}N_{M}N_{F}N_{M}N_{F}N_{F}N_{F}N_{M}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 23 nucleotides in length and wherein counting from the 5′ terminus of the sense strand, nucleotides 1, 3, 5, 7, 9, 11, 12, 13, 15, 17, 19, and 21 are 2′-fluoronucleotides, nucleotides 2, 4, 6, 8, 10, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XIII:

5′-N_{F}^{S}N_{M}^{S}N_{F}N_{M}N_{F}N_{M}N_{F}N_{M}N_{F}N_{M}N_{F}N_{F}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, a compound comprises an antisense strand and a sense strand hybridized to form a double-stranded region, wherein the antisense strand and sense strand are not covalently linked (i.e. the antisense strand and sense strand form an siRNA), wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotide in length and the nucleotides of the sense strand are modified such that, counting from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern I and the sense strand has the modification pattern represented by Pattern II.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotide in length and the nucleotides of the sense strand are modified such that, counting from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern III and the sense strand has the modification pattern represented by Pattern II.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxy nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 19 nucleotide in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern I and the sense strand has the modification pattern represented by Pattern IV.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleotide linkages at the 5′ terminus and the last two internucleotide linkages at the 3′ terminus are phosphorothioate internucleotide linkages, and each other internucleotide is a phosphodiester internucleotide linkage; and wherein the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern III and the sense strand has the modification pattern represented by Pattern IV.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern V and the sense strand has the modification represented by Pattern VI.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern VII and the sense strand has the modification pattern represented by Pattern VIII.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and nucleotides of the sense strand are modified such that counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern IX and the sense strand has the modification pattern of Pattern X.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 23 nucleotides in length and wherein counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern V and the sense strand has the modification represented by Pattern XI.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 23 nucleotides in length and wherein counting from the 5′ terminus of the sense strand, nucleotides 1, 3, 5, 7, 9, 10, 11, 13, 15, 17, 19, and 21 are 2′-fluoro nucleotides, nucleotides 2, 4, 6, 8, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern VII and the sense strand has the modification pattern represented by Pattern XII.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 23 nucleotides in length and wherein counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern IX and the sense strand has the modification pattern of Pattern XIII.

In embodiments, a compound comprises an siRNA described herein, wherein each antisense strand has the modification pattern of Pattern I, and each sense strand of has the modification pattern of Pattern IV. In embodiments, a compound comprises an siRNA described herein, wherein each antisense strand has the modification pattern of Pattern V and each sense strand has the modification pattern of Pattern VI. In embodiments, a compound comprises an siRNA described herein, wherein each antisense strand has the modification pattern of Pattern VII and each sense strand has the modification pattern of Pattern VIII. In embodiments, a compound comprises an siRNA described herein, wherein each antisense strand has the modification pattern of Pattern IX and each sense strand has the modification pattern of Pattern X.

### Pharmaceutical Salts and Compositions

The compounds provided herein may be present as a pharmaceutical salt. In embodiments, the pharmaceutical salt is a sodium salt.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, lithium, ammonium, calcium, magnesium, iron, zinc, copper, manganese, aluminum, and the like; particularly preferred are the ammonium, potassium, s16 odium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

In embodiments, a non-bridging heteroatom (e.g., an S″ or O″) of a linkage of a compound provided herein may be protonated or associated with a counterion such as Na⁺, K⁺, etc. An acceptable salt (e.g. a pharmaceutically acceptable salt) of a compound may comprise fewer cationic counterions (such as Na⁺, K⁺, etc.) than there are non-bridging heteroatoms per molecule (i.e., some non-bridging heteroatoms are protonated and some are associated with counterions). In embodiments, a phosphate linkage attaching an -L⁶-L⁵-L⁴- to a carbon of a nucleotide includes a non-bridging heteroatom. In embodiments, a phosphodiester linkage of a nucleic acid includes a non-bridging heteroatom. In embodiments, a phosphorothioate linkage of a nucleic acid includes a non-bridging heteroatom.

The compounds provided herein may be present as a pharmaceutical composition comprising the compound and a pharmaceutically acceptable diluent. In embodiments, the compound is present in a pharmaceutically acceptable diluent. In embodiments, the pharmaceutically acceptable diluent is a sterile aqueous solution. In embodiments, the sterile aqueous solution is a sterile saline solution.

A pharmaceutical composition may be prepared so that it is compatible with the intended mode of administration of the compound. Routes of administration of compounds include intravenous, intradermal, subcutaneous, transdermal, intramuscular, topical, and ocular administration.

Pharmaceutical compositions may be prepared for ocular administration to the eye in the form of an injection. Pharmaceutical compositions suitable for injection include sterile aqueous solutions, including sterile saline solutions. Pharmaceutical compositions suitable for injection may also be a lyophilized compound that is subsequently reconstitute with a pharmaceutically acceptable diluent in preparation for injection.

Alternatively, pharmaceutical compositions may be prepared for ocular administration to the eye in the form of an ophthalmic suspension (i.e. eye drops). Additional pharmaceutical preparations suitable for ocular administration include emulsions, ointments, aqueous gels, nanomicelles, nanoparticles, liposomes, dendrimers, implants, contact lenses, nanosuspensions, microneedles, and in situ thermosensitive gels.

### Methods of Use

In embodiments, provided herein are methods comprising contacting a cell with a compound of Formula I. In embodiments, the contacting occurs in vitro. In embodiments, the contacting occurs ex vivo. In embodiments, the contacting occurs in vivo.

In embodiments, provided herein are methods comprising administering to a subject a compound of Formula I. In embodiments, the subject has a disease or disorder of the eye, liver, kidney, heart, adipose tissue, lung, muscle or spleen. In embodiments, compounds provided herein are administered by intravenous administration. In embodiments, compounds provided herein are administered by subcutaneous administration. In embodiments, compounds provided herein are administered by intrathecal administration. In embodiments compounds provided herein are administered by intracerebroventricular administration. In embodiments, compounds provided herein are administered via ocular administration. In embodiments, ocular administration is intraocular administration. Intraocular administration is achieved by injection into a specific area of the eye. In embodiments, intraocular administration is subconjunctival administration. In embodiments, intraocular administration is intravitreal administration. In embodiments, intraocular administration is retrobulbar administration. In embodiments, intraocular administration is intracameral administration. In embodiments, ocular administration is topical administration. In embodiments, topical administration comprises administration of an ophthalmic suspension. In embodiments, topical administration comprises administration of an ointment.

In embodiments, provided herein are compounds of Formula I for use in therapy. In embodiments, provided herein are compounds of Formula I for use in the preparation of a medicament.

In embodiments, provided herein are methods of a nucleic acid into a cell within a subject, the method comprising administering to said subject a compound of Formula I.

In embodiments, provided herein are cells comprising a compound of Formula I.

In an aspect is provided a method of treating a disease in a subject in need thereof, the method including administering to the subject in need thereof a therapeutically effective amount of a compound described herein, or a pharmaceutically acceptable salt thereof.

### Nucleotide Sequences

Although the sequence listing accompanying this filing identifies each nucleotide sequence as either “RNA” or “DNA” as required, in practice, those sequences may be modified with a combination of chemical modifications specified herein. One of skill in the art will readily appreciate that in the sequence listing, such designation as “RNA” or “DNA” to describe modified nucleotides is somewhat arbitrary. For example, a nucleic acid provided herein comprising a nucleotide comprising a 2′-O-methyl sugar moiety and a thymine base may described as a DNA residue in the sequence listing, even though the nucleotide is modified and is not a naturally-occurring DNA nucleotide.

Accordingly, nucleotide sequences provided in the sequence listing are intended to encompass nucleotide sequences containing any combination of natural or modified RNA and/or DNA, including, but not limited to such nucleotide sequences having modified nucleobases. By way of further example and without limitation, a nucleic acid having the nucleotide sequence “ATCGATCG” in the sequence listing encompasses any nucleic acid having such nucleotide sequence, whether modified or unmodified, including, but not limited to, such nucleic acids comprising RNA bases, such as those having sequence “AUCGAUCG” and those having some DNA bases and some RNA bases such as “AUCGATCG” and oligonucleotides having other modified bases, such as “ATmeCGAUCG,” wherein meC indicates a 5-methylcytosine.

### Formulations

Various formulations are available to facilitate compound use both in vitro and as therapeutic agents. Accordingly, in embodiments, a compound provided herein is present in a formulation.

Compounds may be formulated with cationic lipids to facilitate transfection into cells. Suitable cationic lipid reagents for transfection include Lipofectamine reagents, such as Lipofectamine RNAiMAX.

For use in vivo as therapeutic agents, nucleic acids compounds may be encapsulated into lipid nanoparticles. Lipid nanoparticles generally comprise a cationic lipid, a non-cationic lipid, and a lipid that prevents aggregation of the nanoparticle. Suitable cationic lipids include DLin-MC3-DMA ((6Z,9Z,28Z,31Z)-Heptatriaconta-6,9,28,31-tetraen-19-yl 4-(dimethylamino) butanoate), DLin-KC2-DMA (2,2-dilinoleyl-4-dimethylaminoethyl-[1,3]-dioxolane) and the lipidoid C12-200. Suitable non-cationic lipids include, for example, DOPC (1,2-dioleoyl-sn-glycero-3-phosphatidylcholine) and DSPC (1,2-distearoyl-sn-glycero-3-phosphocholine). Examples of lipids that prevent aggregation include, for example, polyethylene glycol (PEG)-lipids, such as PEG-C-DMA (3-N-[(ω-methoxypoly(ethylene glycol)2000)carbamoyl]-1,2-dimyristyloxy-propylamine), PEG2000-C-DMG (α-(3-{[1,2-di(myristyloxy)proponoxy]carbonylamino}propyl)-ω-methoxy, polyoxyethylene), and mPEG-DSPE (1,2-distearoyl-sn-glycero-3-phosphoethanolamine-N-[methoxy(polyethylene glycol)-2000]).

### Embodiments

Embodiment P1. A compound having the structure:

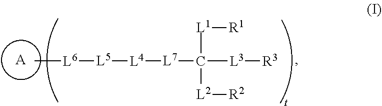

wherein
A is nucleic acid;
L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁶ is independently a bond, substituted or unsubstituted alkylene or substituted or unsubstituted heteroalkylene;
L¹ is independently a bond, substituted or unsubstituted 2 to 50 membered heteroalkylene or L^{1A}-L^{1B}-L^{1C}-L^{1D}-L^{1E}.

L² is independently a bond, substituted or unsubstituted 2 to 50 membered heteroalkylene or L^{2A}-L^{2B}-L^{2C}-L^{2D}-L^{2E}.

L³ is independently a bond, substituted or unsubstituted 2 to 50 membered heteroalkylene or L^{3A}-L^{3B}-L^{3C}-L^{3D}-L^{3E}.

L^{1A}, L^{1B}, L^{1C}, L^{1D}, L^{1E}, L^{2A}, L^{2B}, L^{2C}, L^{2D}, L^{2E}, L^{3A}, L^{3B}, L^{3C}, L^{3D} and L^{3E} are independently a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted or unsubstituted C₁-C₂₅ alkylene, substituted or unsubstituted 2 to 25 membered heteroalkylene, substituted or unsubstituted cycloalkylene, substituted or unsubstituted heterocycloalkylene, substituted or unsubstituted arylene or substituted or unsubstituted heteroarylene,
wherein L^{1A}-L^{1B}-L^{1C}-L^{1D}-L^{1E} is not a bond, substituted or unsubstituted alkylene or a substituted or unsubstituted heteroalkylene with a terminal carbon atom and at least one of L^{1A}, L^{1B}, L^{1C}, L^{1D} or L^{1E} is not a bond or substituted or unsubstituted 2 to 25 membered heteroalkylene,
wherein L^{2A}-L^{2B}-L^{2C}-L^{2D}-L^{2E} is not a bond, substituted or unsubstituted alkylene or a substituted or unsubstituted heteroalkylene with a terminal carbon atom and at least one of L^{2A}, L^{2B}, L^{2C}, L^{2D} or L^{2E} is not a bond or substituted or unsubstituted 2 to 25 membered heteroalkylene,
wherein L^{3A}-L^{3B}-L^{3C}-L^{3D}-L^{3E} is not a bond or substituted or unsubstituted alkylene or a substituted or unsubstituted heteroalkylene with a terminal carbon atom at least one of L^{3A}, L^{3B}, L^{3C}, L^{3D} or L^{3E} is not a bond or substituted or unsubstituted 2 to 25 membered heteroalkylene;
R¹, R² and R³ are independently unsubstituted C₈-C₂₀ alkyl; and
t is an integer from 1 to 5.
Embodiment P2. The compound of embodiment P1, wherein L¹, L² and L³ are independently substituted or unsubstituted 2 to 50 membered heteroalkylene.
Embodiment P3. The compound of embodiment P1, wherein L¹, L² and L³ are independently R¹⁰-substituted or unsubstituted 2 to 50 membered heteroalkylene, wherein R¹⁰ is oxo, hydroxyl, or unsubstituted C₁-C₄ alkyl.
Embodiment P4. The compound of embodiment P1, wherein t is 1.
Embodiment P5. The compound of embodiment P1, wherein t is 2.
Embodiment P6. The compound of embodiment P1, wherein t is 3.
Embodiment P7. The compound of any one of embodiments P1 to P6, wherein A is a double-stranded nucleic acid, or a single-stranded nucleic acid.
Embodiment P8. The compound of embodiment P7, wherein the double-stranded nucleic acid is a small interfering RNA, a short hairpin RNA or a microRNA mimic.
Embodiment P9. The compound of embodiment P7, wherein the single-stranded nucleic acid is a single-strand small interfering RNA, an RNaseH oligonucleotide, an anti-microRNA oligonucleotide, a steric blocking oligonucleotide, exon-skipping oligonucleotide, a CRISPR guide RNA, or an aptamer.
Embodiment P10. The compound of any one of embodiments P1 to P9, wherein the nucleic acid of A is comprises one or more modified nucleotides.
Embodiment P11. The compound of any one of embodiments P1 to P10, wherein the nucleic acid comprises one or more modified sugar moieties.
Embodiment P12. The compound of embodiment P10, wherein the modified sugar moiety comprises a 2′ modification or an unlocked sugar modification.
Embodiment P13. The compound of embodiment P12, wherein the 2′-modification is selected from 2′-fluoro modification, 2′-O-methyl modification, a 2′-O-methoxyethyl, and a bicyclic sugar modification.
Embodiment P14. The compound of embodiment P13, wherein the bicyclic sugar modification is selected from a 4′-CH(CH₃)—O-2′ linkage, a 4′-(CH₂)₂—O-2′ linkage, a 4′-CH(CH₂—OMe)-O-2′ linkage, 4′-CH₂—N(CH₃)—O-2′ linkage, and 4′-CH₂—N(H)—O-2′ linkage.
Embodiment P15. The compound of embodiment P11, wherein the modified sugar moiety is a morpholino moiety.
Embodiment P16. The compound of any one of embodiments P1 to P15, wherein the nucleic acid comprises one or more modified internucleotide linkages.
Embodiment P17. The compound of embodiment P16, wherein the modified internucleotide linkage selected from a phosphorothioate linkage and a phosphorodiamidite linkage.
Embodiment P18. The compound of embodiment P10, wherein the nucleic acid comprises a hydroxyl group, a phosphate group, or modified phosphate group.
Embodiment P19. The nucleic acid compound of embodiment P18, wherein the modified phosphate group is a 5′-(E)-vinylphosphonate.
Embodiment P20. The compound any one of embodiments P1 to P19, wherein the nucleic acid is a double-stranded nucleic acid comprising an antisense strand hybridized to a sense strand, and wherein each of the antisense strand and sense strand is independently 15 to 30 nucleotides in length.
Embodiment P21. The compound of embodiment P20, wherein each of the antisense strand and sense strand is independently 17 to 25 nucleotides in length.
Embodiment P22. The compound of embodiment P20, wherein each of the antisense strand and sense strand is independently 19 to 23 nucleotides in length.
Embodiment P23. The compound of any one of embodiments P1 to P19, wherein the nucleic acid is a single-stranded nucleic acid and the single-stranded nucleic acid is 8 to 30 nucleotides in length.
Embodiment P24. The compound of embodiment P23, wherein the single-stranded nucleic acid is 12 to 25 nucleotides in length.
Embodiment P25. The compound of embodiment P23, wherein the single-stranded nucleic acid is 15 to 25 nucleotides in length.
Embodiment P26. The compound of embodiment P23, wherein the single-stranded nucleic acid is 17 to 23 nucleotides in length.
Embodiment P27. The compound of any one of embodiments P7 to P26, wherein one L⁶ is attached to a 3′ carbon of the double-stranded nucleic acid or single-stranded nucleic acid.
Embodiment P28. The compound of embodiment P27, wherein the 3′ carbon is the 3′ carbon of a 3′ terminal nucleotide.
Embodiment P29. The compound of any one of embodiments P7 to P26, wherein one L⁶ is attached to a 5′ carbon of the double-stranded nucleic acid or single-stranded nucleic acid.
Embodiment P30. The compound of embodiment P29, wherein the 5′ carbon is the 5′ carbon of a 5′ terminal nucleotide.
Embodiment P31. The compound of any one of embodiments P7 to P26, wherein one L⁶ is attached to a 2′ carbon of the double-stranded nucleic acid or single-stranded nucleic acid.
Embodiment P32. The compound of any one of embodiments P7 to P26, wherein one L⁶ is attached to an oxygen of 2′ hydroxy of the double-stranded nucleic acid or single-stranded nucleic acid.
Embodiment P33. The compound of any one of embodiments P7 to P26, wherein the double-stranded nucleic acid or single-stranded nucleic acid comprises a morpholino moiety, and one L⁶ is attached to a 3′ nitrogen of morpholino moiety.
Embodiment P34. The compound of any one of embodiments P7 to P26, wherein the double-stranded nucleic acid or single-stranded nucleic acid comprises a morpholino moiety, and one L⁶ is attached to a 6′ carbon of the morpholino moiety.
Embodiment P35. The compound of any one of embodiments P7 to P26, wherein one L⁶ is attached to a nucleobase of the double-stranded nucleic acid or single-stranded nucleic acid.
Embodiment P36. The compound of any one of embodiments P1 to P35, wherein L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene or substituted or unsubstituted heteroalkylene.
Embodiment P37. The compound of any one of embodiments P1 to P35, wherein L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted C₁-C₈ alkylene or substituted or unsubstituted 2 to 8 membered heteroalkylene.
Embodiment P38. The compound of any one of embodiments P1 to P35, wherein L⁴ and L⁶ are independently —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, R²⁰-substituted or unsubstituted C₁-C₈ alkylene, or R²⁰-substituted or unsubstituted 2 to 8 membered heteroalkylene, wherein R²⁰ is oxo, hydroxyl, or substituted or unsubstituted C₁-C₄ alkyl.
Embodiment P39. The compound of embodiment P38, wherein R²⁰ is oxo, hydroxyl, or unsubstituted C₁-C₄ alkyl.
Embodiment P40. The compound of any one of embodiments P1 to P39, wherein L⁶ is independently

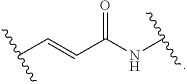 [SMILES: */C=C/C(=O)N*]

Embodiment P41. The compound of any one of embodiments P1 to P39, wherein L⁶ is independently —OPO₂—O—.
Embodiment P42. The compound of any one of embodiments P1 to P39, wherein L⁶ is independently —O—.
Embodiment P43. The compound of any one of embodiments P1 to P42, wherein L⁴ is independently substituted or unsubstituted alkylene, or substituted or unsubstituted hetoeroalkylene.
Embodiment P44. The compound of any one of embodiments P1 to P42, wherein L⁴ is independently -L¹⁴-NH—C(O)— or -L¹⁴-C(O)—NH—, wherein L¹⁴ is independently substituted or unsubstituted C₁-C₂₀ alkylene, or substituted or unsubstituted 2-20 membered heteroalkenylene.
Embodiment P45. The compound of any one of embodiments P1 to P42, wherein L⁴ is independently -L¹⁴-NH—C(O)— or -L¹⁴-C(O)—NH—, wherein L¹⁴ is substituted or unsubstituted C₁-C₈ alkylene.
Embodiment P46. The compound of one of embodiments P1 to P42, wherein L⁴ is independently

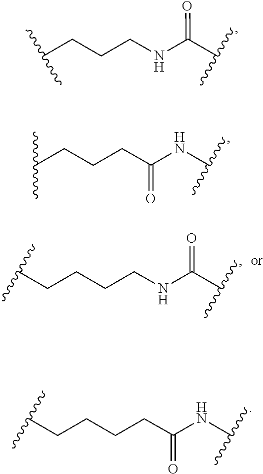 [SMILES: *CCCNC(*)=O ; *CCCC(=O)N* ; *CCCCNC(*)=O ; *CCCCC(=O)N*]

Embodiment P47. The compound of one of embodiments P1 to P46, wherein L⁵ is substituted or unsubstituted C₁-C₈alkylene, or substituted or unsubstituted 2 to 8 membered heteroalkylene.
Embodiment P48. The compound of one of embodiments P1 to P46, wherein L⁵ is substituted or unsubstituted C₃-C₆ cycloalkylene, or substituted or unsubstituted 4 to 6 membered heterocycloalkylene.
Embodiment P49. The compound of one of embodiments P1 to P46, wherein L⁵ is substituted or unsubstituted phenylene, or substituted or unsubstituted 4 to 6 heteroarylene.
Embodiment P50. The compound of one of embodiments P1 to P46, wherein L⁵ is independently a bond.
Embodiment P51. The compound of one of embodiments P1 to P50, wherein L⁶ is independently substituted or unsubstituted C₁-C₈ alkylene, or substituted or unsubstituted 2 to 8 membered heteroalkylene.
Embodiment P52. The compound of one of embodiments P1 to P50, wherein L⁷ is independently a bond.
Embodiment P53. The compound of one of embodiments P1 to P50, wherein L⁷ is independently -L¹⁷-NH—C(O)— or -L¹⁷-C(O)—NH—, wherein L¹⁷ is independently substituted or unsubstituted C₁-C₂₀ alkylene, substituted or unsubstituted 2-20 membered heteroalkylene, or substituted or unsubstituted 2-20 membered heteroalkenylene.
Embodiment P54. The compound of one of embodiments P1 to P50, wherein L⁷ is independently -L¹⁷-NH—C(O)— or -L¹⁷-C(O)—NH—, wherein L¹⁷ is substituted or unsubstituted C₁-C₈ alkylene.
Embodiment P55. The compound of any one of embodiments P1 to P50, wherein L⁷ is independently —NHC(O)—.
Embodiment P56. The compound of one of embodiments P1 to P50, wherein L⁷ is independently

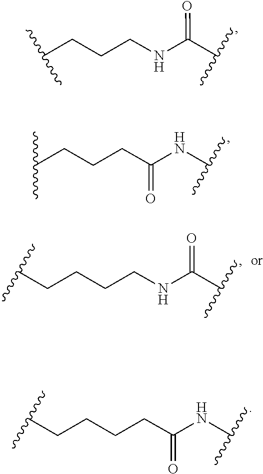 [SMILES: *CCCNC(*)=O ; *CCCC(=O)N* ; *CCCCNC(*)=O ; *CCCCC(=O)N*]

Embodiment P57. The compound of any one of embodiments P1 to P56, wherein -L⁶-L⁵-L⁴- is independently a bond, or substituted or unsubstituted 2 to 50 membered heteroalkylene.
Embodiment P58. The compound of any one of embodiments P1 to P56, wherein -L⁶-L⁵-L⁴- is R¹¹-substituted or unsubstituted 2 to 50 membered heteroalkylene, wherein R¹¹ is oxo, hydroxyl, or unsubstituted C₁-C₄ alkyl.
Embodiment P59. The compound of any one of embodiments P1 to P56, wherein -L⁶-L⁵-L⁴- is independently -L¹⁰-NH—C(O), or -L¹⁰-C(O)—NH—, wherein L¹⁰ is independently substituted or unsubstituted C₁-C₂₀ alkylene, substituted or unsubstituted heteroalkylene, or substituted or unsubstituted 2-20 membered heteroalkenylene.
Embodiment P60. The compound of any one of embodiments P1 to P56, wherein -L⁶-L⁵-L⁴- is independently -L¹⁰-NH—C(O)— or -L¹⁰-C(O)—NH—, wherein L¹⁰ is substituted or unsubstituted C₁-C₈ alkylene.
Embodiment P61. The compound of any one of embodiments P1 to P56, wherein -L⁶-L⁵-L⁴- is independently

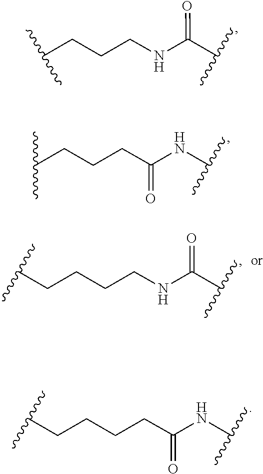 [SMILES: *CCCNC(*)=O ; *CCCC(=O)N* ; *CCCCNC(*)=O ; *CCCCC(=O)N*]

Embodiment P62. The compound of any one of embodiments P1 to P56, wherein -L⁶-L⁵-L⁴- is independently —O-L¹⁰-NH—C(O)— or —O-L¹⁰-C(O)—NH—, wherein L¹⁰ is independently substituted or unsubstituted C₁-C₂₀ alkylene, or substituted or unsubstituted 2-20 membered heteroalkenylene.
Embodiment P63. The compound of any one of embodiments P1 to P56, wherein -L⁶-L⁵-L⁴- is independently —O-L¹⁰-NH—C(O)—, or —O-L¹⁰-C(O)—NH—, wherein L¹⁰ is substituted or unsubstituted C₁-C₈ alkylene.
Embodiment P64. The compound of any one of embodiments P1 to P56, wherein -L⁶-L⁵-L⁴- is independently

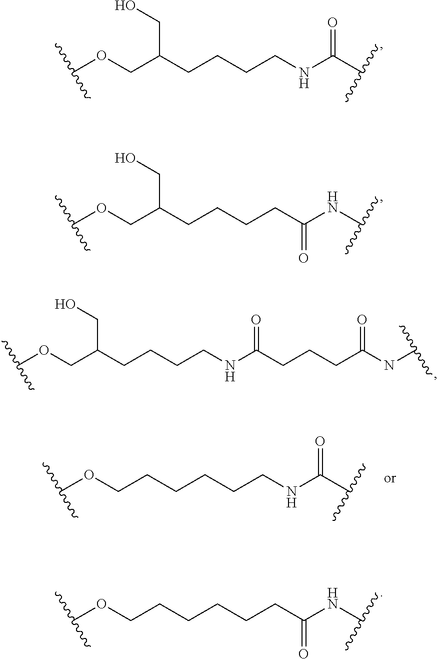 [SMILES: *OCC(CO)CCCCNC(*)=O ; *NC(=O)CCCCC(CO)CO* ; *NC(=O)CCCC(=O)NCCCCC(CO)CO* ; *OCCCCCCNC(*)=O ; *NC(=O)CCCCCCO*]

Embodiment P65. The compound of any one of embodiments P1 to P56, wherein -L⁶-L⁵-L⁴- is independently —OPO₂—O-L¹⁰-NH—C(O)— or —OPO₂—O-L¹⁰-C(O)—NH—, wherein L¹⁰ is independently substituted or unsubstituted C₁-C₂₀ alkylene, or substituted or unsubstituted 2-20 membered heteroalkenylene.
Embodiment P66. The compound of any one of embodiments P1 to P56, wherein -L⁶-L⁵-L⁴- is independently —OPO₂—O-L¹⁰-NH—C(O)— or —OPO₂—O-L¹⁰-C(O)—NH—, wherein L¹⁰ is substituted or unsubstituted C₁-C₈ alkylene.
Embodiment P67. The compound of any one of embodiments P1 to P56, wherein -L⁶-L⁵-L⁴- is independently

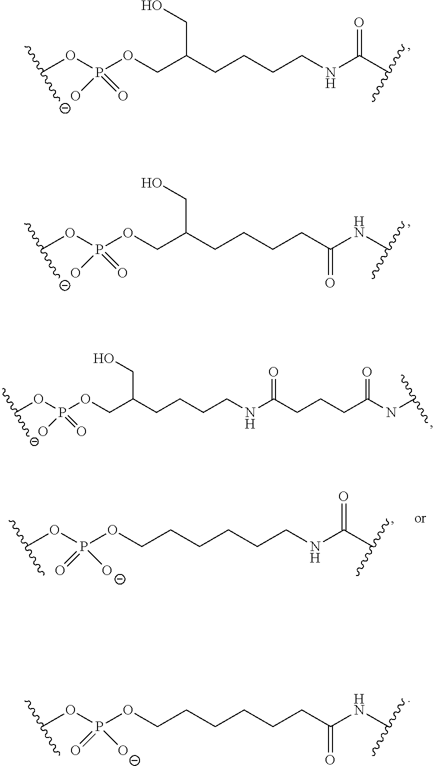 [SMILES: *OP(=O)([O-])OCC(CO)CCCCNC(*)=O ; *NC(=O)CCCCC(CO)COP(=O)([O-])O* ; *NC(=O)CCCC(=O)NCCCCC(CO)COP(=O)([O-])O* ; *OP(=O)([O-])OCCCCCCNC(*)=O ; *NC(=O)CCCCCCOP(=O)([O-])O*]

Embodiment P68. The compound of any one of embodiments P1 to P56, wherein -L⁶-L⁵-L⁴- is independently

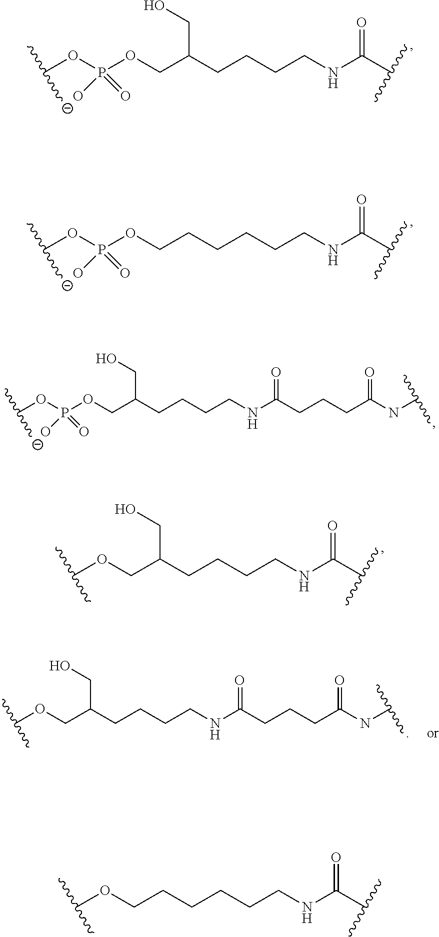 [SMILES: *OP(=O)([O-])OCC(CO)CCCCNC(*)=O ; *OP(=O)([O-])OCCCCCCNC(*)=O ; *NC(=O)CCCC(=O)NCCCCC(CO)COP(=O)([O-])O* ; *OCC(CO)CCCCNC(*)=O ; *NC(=O)CCCC(=O)NCCCCC(CO)CO* ; *OCCCCCCNC(*)=O]

and is attached to a 3′ carbon of the double-stranded nucleic acid or single-stranded nucleic acid.
Embodiment P69. The compound of embodiment P68, wherein the 3′ carbon is the 3′ carbon of a 3′ terminal nucleotide.
Embodiment P70. The compound of any one of embodiments P1 to P56, wherein -L⁶-L⁵-L⁴- is independently

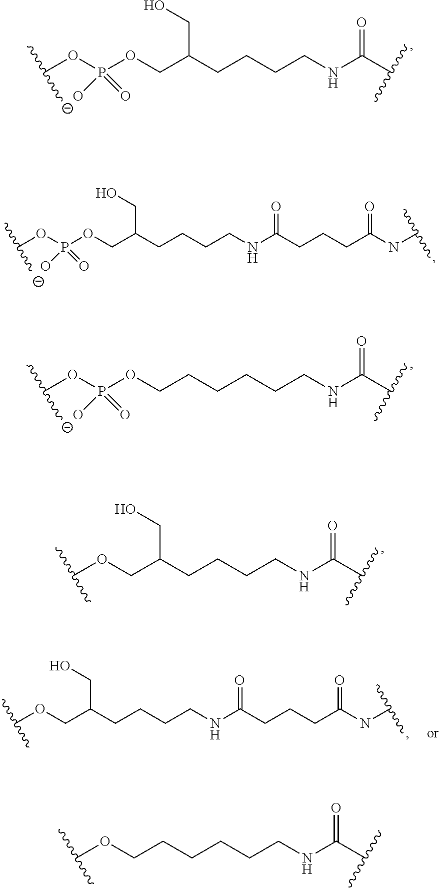 [SMILES: *OP(=O)([O-])OCC(CO)CCCCNC(*)=O ; *NC(=O)CCCC(=O)NCCCCC(CO)COP(=O)([O-])O* ; *OP(=O)([O-])OCCCCCCNC(*)=O ; *OCC(CO)CCCCNC(*)=O ; *NC(=O)CCCC(=O)NCCCCC(CO)CO* ; *OCCCCCCNC(*)=O]

and is attached to a 5′ carbon of the double-stranded nucleic acid or single-stranded nucleic acid.
Embodiment P71. The compound of embodiment P70, wherein the 5′ carbon is the 5′ carbon of a 5′ terminal nucleotide.
Embodiment P72. The compound of any one of embodiments P1 to P56, wherein -L⁶-L⁵-L⁴- is independently

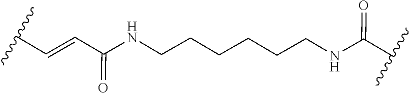 [SMILES: */C=C/C(=O)NCCCCCCNC(*)=O]

and is attached to a nucleotide base of the double-stranded nucleic acid or single-stranded nucleic acid.
Embodiment P73. The compound of any one of embodiments P1 to P72, wherein L¹ is independently —NHC(O)—, —C(O)NH—, substituted or unsubstituted alkylene, or substituted or unsubstituted heteroalkylene.
Embodiment P74. The compound of any one of embodiments P1 to P72, wherein L¹ is independently —NHC(O)—.
Embodiment P75. The compound of any one of embodiments P1 to P72, wherein
L^{1A} is independently a bond, —NHC(O)—, —C(O)NH—, substituted or unsubstituted alkylene, or substituted or unsubstituted heteroalkylene;
L^{1B} is independently a bond, —O—, —NHC(O)—, —C(O)NH—, substituted or unsubstituted alkylene, substituted or unsubstituted heteroalkylene, or substituted or unsubstituted arylene;
L^{1C} is independently a bond, —O—, —NHC(O)—, —C(O)NH—, substituted or unsubstituted alkylene, substituted or substituted or unsubstituted heteroalkylene, or substituted or unsubstituted arylene;
L^{1D} is independently a bond, —O—, —NHC(O)—, —C(O)NH—, substituted or unsubstituted alkylene, substituted or unsubstituted heteroalkylene, or substituted or unsubstituted arylene; and
L^{1E} is independently a bond, —NHC(O)—, —C(O)NH—, substituted or unsubstituted alkylene, or substituted or unsubstituted heteroalkylene.
Embodiment P76. The compound of any one of embodiments P1 to P72, wherein
L^{1A} is independently a bond, —NHC(O)—, —C(O)NH—, substituted or unsubstituted C₁-C₈ alkylene, or substituted or unsubstituted 2 to 8 membered heteroalkylene;
L^{1B} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene;
L^{1C} is independently a bond, —O—, —NHC(O)—, —C(O)NH—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene;
L^{1D} is independently a bond, —O—, —NHC(O)—, —C(O)NH—, substituted or unsubstituted C₁-C₈alkylene, substituted or unsubstituted 2 to 8 membered heteroalkylene, or substituted or unsubstituted phenylene; and
L^{1E} is independently a bond, —NHC(O)—, —C(O)NH—, substituted or unsubstituted C₁-C₈ alkylene, or substituted or unsubstituted 2 to 8 membered heteroalkylene.
Embodiment P77. The compound of any one of embodiments P1 to P72, wherein
L^{1A} is independently unsubstituted C₁-C₈ alkylene, or unsubstituted 2 to 8 membered heteroalkylene;
L^{1B} is independently a bond, —O—, —NHC(O)—, C(O)NH—, unsubstituted C₁-C₈ alkylene, unsubstituted C₂-C₈ alkynylene, unsubstituted 2 to 8 membered heteroalkylene, or unsubstituted phenylene;
L^{1C} is independently a bond, —O—, —NHC(O)—, —C(O)NH—, unsubstituted C₁-C₈ alkylene, unsubstituted 2 to 8 membered heteroalkylene, or unsubstituted phenylene;
L^{1D} is independently a bond, —O—, —NHC(O)—, —C(O)NH—, unsubstituted C₁-C₈ alkylene, or unsubstituted 2 to 8 membered heteroalkylene; and
L^{1E} is independently —NHC(O)—.
Embodiment P78. The compound of one of embodiment P1 to P72, wherein L¹ is independently -L¹¹-NH—C(O)— or -L¹¹-C(O)—NH—, wherein L¹¹ is independently substituted or unsubstituted C₁-C₂₀ alkylene, substituted or unsubstituted 2-20 membered heteroalkylene, or substituted or unsubstituted 2-20 membered heteroalkenylene.
Embodiment P79. The compound of one of embodiments P1 to P72, wherein L¹ is independently -L¹¹-NH—C(O)— or -L¹¹-C(O)—NH—, wherein L¹¹ is substituted or unsubstituted C₁-C₈ alkylene.
Embodiment P80. The compound of one of embodiments P1 to P72, wherein L¹ is independently

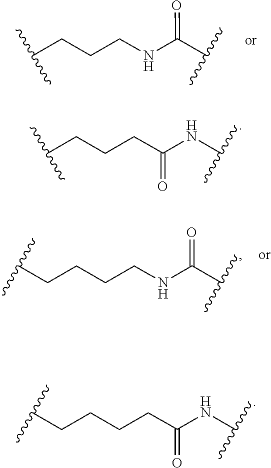 [SMILES: *CCCNC(*)=O ; *CCCC(=O)N* ; *CCCCNC(*)=O ; *CCCCC(=O)N*]

Embodiment P81. The compound of any one of embodiments P1 to P72, wherein L¹ is a bond,

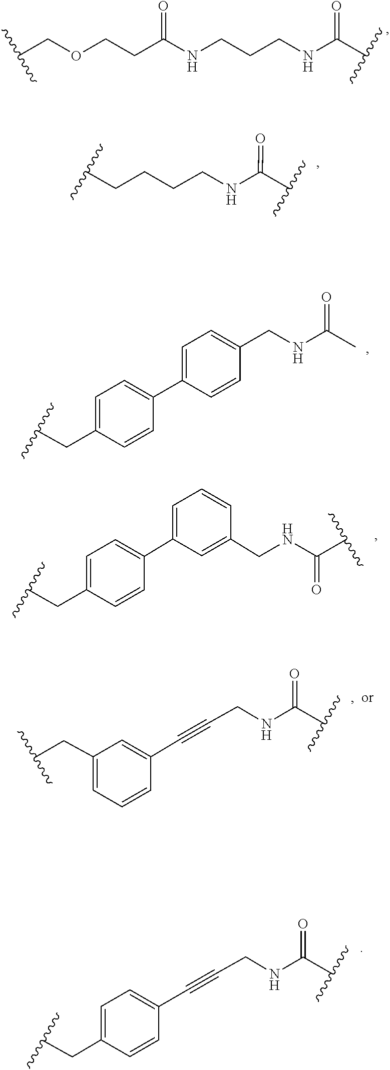 [SMILES: *COCCC(=O)NCCCNC(*)=O ; *CCCCNC(*)=O ; *Cc1ccc(-c2ccc(CNC(C)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment P82. The compound of any one of embodiments P1 to P72, wherein L¹ is

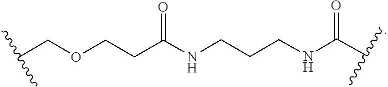 [SMILES: *COCCC(=O)NCCCNC(*)=O]

Embodiment P82. The compound of any one of embodiments P1 to P81, wherein L² is independently —NHC(O)—, —C(O)NH—, substituted or unsubstituted alkylene, or substituted or unsubstituted heteroalkylene.
Embodiment P84. The compound of any one of embodiments P1 to P81, wherein L² is independently —NHC(O)—.
Embodiment P85. The compound of any one of embodiments P1 to P81, wherein
L^{2A} is independently a bond, —NHC(O)—, —C(O)NH—, substituted or unsubstituted alkylene, or substituted or unsubstituted heteroalkylene;
L^{2B} is independently a bond, —O—, —NHC(O)—, C(O)NH—, substituted or unsubstituted alkylene, substituted or unsubstituted heteroalkylene, or substituted or unsubstituted arylene;
L^{2C} is independently a bond, —O—, —NHC(O)—, —C(O)NH—, substituted or unsubstituted alkylene, substituted or substituted or unsubstituted heteroalkylene, or substituted or unsubstituted arylene;
L^{2D} is independently a bond, —O—, —NHC(O)—, —C(O)NH—, substituted or unsubstituted alkylene, substituted or unsubstituted heteroalkylene, or substituted or unsubstituted arylene; and
L^{2E} is independently a bond, —NHC(O)—, —C(O)NH—, substituted or unsubstituted alkylene, or substituted or unsubstituted heteroalkylene.
Embodiment P86. The compound of any one of embodiments P1 to P81, wherein
L^{2A} is independently a bond, —NHC(O)—, —C(O)NH—, substituted or unsubstituted C₁-C₈ alkylene, or substituted or unsubstituted 2 to 8 membered heteroalkylene;
L^{2B} is independently a bond, —O—, —NHC(O)—, —C(O)NH—, substituted or unsubstituted C₁-C₈alkylene, substituted or unsubstituted 2 to 8 membered heteroalkylene, or substituted or unsubstituted phenylene;
L^{2C} is independently a bond, —O—, —NHC(O)—, —C(O)NH—, substituted or unsubstituted C₁-C₈alkylene, substituted or unsubstituted C₂-C₈ alkynylene, substituted or substituted or unsubstituted 2 to 8 membered heteroalkylene, or substituted or unsubstituted phenylene;
L^{2D} is independently a bond, —O—, —NHC(O)—, —C(O)NH—, substituted or unsubstituted C₁-C₈alkylene, substituted or unsubstituted 2 to 8 membered heteroalkylene, or substituted or unsubstituted phenylene; and
L^{2E} is independently a bond, —NHC(O)—, —C(O)NH—, substituted or unsubstituted C₁-C₈ alkylene, or substituted or unsubstituted 2 to 8 membered heteroalkylene.
Embodiment P87. The compound of any one of embodiments P1 to P81, wherein
L^{2A} is independently unsubstituted C₁-C₈ alkylene, or unsubstituted 2 to 8 membered heteroalkylene;
L^{2B} is independently a bond, —O—, —NHC(O)—, —C(O)NH—, unsubstituted C₁-C₈ alkylene, unsubstituted C₂-C₈ alkynylene, unsubstituted 2 to 8 membered heteroalkylene, or unsubstituted phenylene;
L^{2C} is independently a bond, —O—, —NHC(O)—, —C(O)NH—, unsubstituted C₁-C₈ alkylene, unsubstituted 2 to 8 membered heteroalkylene, or unsubstituted phenylene; L^{2D} is independently a bond, —O—, —NHC(O)—, —C(O)NH—, unsubstituted C₁-C₈ alkylene, or unsubstituted 2 to 8 membered heteroalkylene; and
L^{2E} is independently —NHC(O)—.
Embodiment P88. The compound of one of embodiments P1 to P81, wherein L² is independently -L¹²-NH—C(O)— or -L¹²-C(O)—NH—, wherein L¹² is substituted or unsubstituted C₁-C₂₀ alkylene, or substituted or unsubstituted 2-20 membered heteroalkenylene.
Embodiment P89. The compound of one of embodiments P1 to P81, wherein L² is independently -L¹²-NH—C(O)— or -L¹²-C(O)—NH—, wherein L¹² is substituted or unsubstituted C₅-C₈ alkylene.
Embodiment P90. The compound of one of embodiments P1 to P81, wherein L² is independently

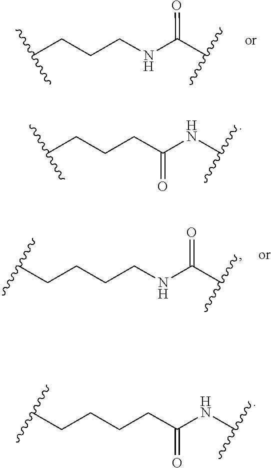 [SMILES: *CCCNC(*)=O ; *CCCC(=O)N* ; *CCCCNC(*)=O ; *CCCCC(=O)N*]

Embodiment P91. The compound of any one of embodiments P1 to P81, wherein L² is a bond,

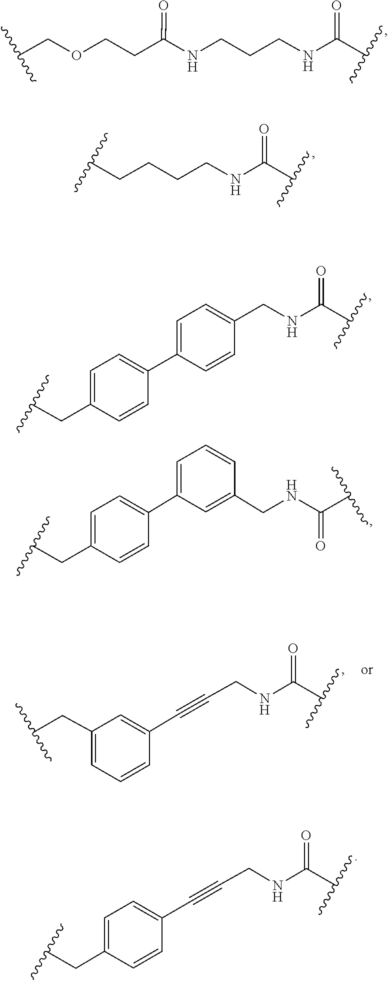 [SMILES: *COCCC(=O)NCCCNC(*)=O ; *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment P92. The compound of any one of embodiments P1 to P81, wherein L² is

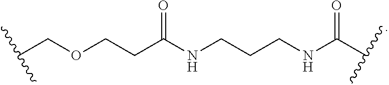 [SMILES: *COCCC(=O)NCCCNC(*)=O]

Embodiment P93. The compound of any one of embodiments P1 to P91, wherein L³ is independently —NHC(O)—, —C(O)NH—, substituted or unsubstituted alkylene, or substituted or unsubstituted heteroalkylene.
Embodiment P94. The compound of any one of embodiments P1 to P91, wherein L³ is independently —NHC(O)—.
Embodiment P95. The compound of any one of embodiments P1 to P91, wherein
L^{3A} is independently a bond, —NHC(O)—, —C(O)NH—, substituted or unsubstituted alkylene, or substituted or unsubstituted heteroalkylene;
L^{3B} is independently a bond, —O—, —NHC(O)—, —C(O)NH—, substituted or unsubstituted alkylene, substituted or unsubstituted heteroalkylene, or substituted or unsubstituted arylene;
L^{3C} is independently a bond, —O—, —NHC(O)—, —C(O)NH—, substituted or unsubstituted alkylene, substituted or substituted or unsubstituted heteroalkylene, or substituted or unsubstituted arylene;
L^{3D} is independently a bond, —O—, —NHC(O)—, —C(O)NH—, substituted or unsubstituted alkylene, substituted or unsubstituted heteroalkylene, or substituted or unsubstituted arylene; and
L^{3E} is independently a bond, —NHC(O)—, —C(O)NH—, substituted or unsubstituted alkylene, or substituted or unsubstituted heteroalkylene.
Embodiment P96. The compound of any one of embodiments P1 to P91, wherein
L^{3A} is independently a bond, —NHC(O)—, —C(O)NH—, substituted or unsubstituted C₁-C₈ alkylene, or substituted or unsubstituted 2 to 8 membered heteroalkylene;
L^{3B} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene;
L^{3C} is independently a bond, —O—, —NHC(O)—, —C(O)NH—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene;
L^{3D} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene; and
L^{3E} is independently a bond, —NHC(O)—, C(O)NH—, substituted or unsubstituted C₁-C₈ alkylene, or substituted or unsubstituted 2 to 8 membered heteroalkylene.
Embodiment P97. The compound of any one of embodiments P1 to P91, wherein
L^{3A} is independently unsubstituted C₁-C₈alkylene, or unsubstituted 2 to 8 membered heteroalkylene;
L^{3B} is independently a bond, —O—, —NHC(O)—, C(O)NH—, unsubstituted C₁-C₈ alkylene, unsubstituted C₂-C₈ alkynylene, unsubstituted 2 to 8 membered heteroalkylene, or unsubstituted phenylene;
L^{3C} is independently a bond, —O—, —NHC(O)—, —C(O)NH—, unsubstituted C₁-C₈ alkylene, unsubstituted 2 to 8 membered heteroalkylene, or unsubstituted phenylene;
L^{3D} is independently a bond, —O—, —NHC(O)—, —C(O)NH—, unsubstituted C₁-C₈ alkylene, or unsubstituted 2 to 8 membered heteroalkylene; and
L^{3E} is independently —NHC(O)—.
Embodiment P98. The compound of one of embodiments P1 to P91, wherein L³ is independently -L¹³-NH—C(O)— or -L¹³-C(O)—NH—, wherein L¹³ is substituted or unsubstituted C₁-C₂₀ alkylene, or substituted or unsubstituted 2-20 membered heteroalkenylene.
Embodiment P99. The compound of one of embodiments P1 to P91, wherein L³ is independently -L¹³-NH—C(O)— or -L¹³-C(O)—NH—, wherein L¹³ is substituted or unsubstituted C₅-C₈ alkylene.
Embodiment P100. The compound of one of embodiments P1 to P91, wherein L³ is independently

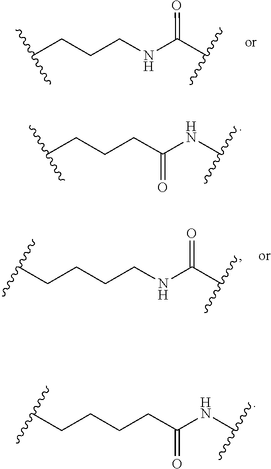 [SMILES: *CCCNC(*)=O ; *CCCC(=O)N* ; *CCCCNC(*)=O ; *CCCCC(=O)N*]

Embodiment P101. The compound of any one of embodiments P1 to P91, wherein L³ is a bond,

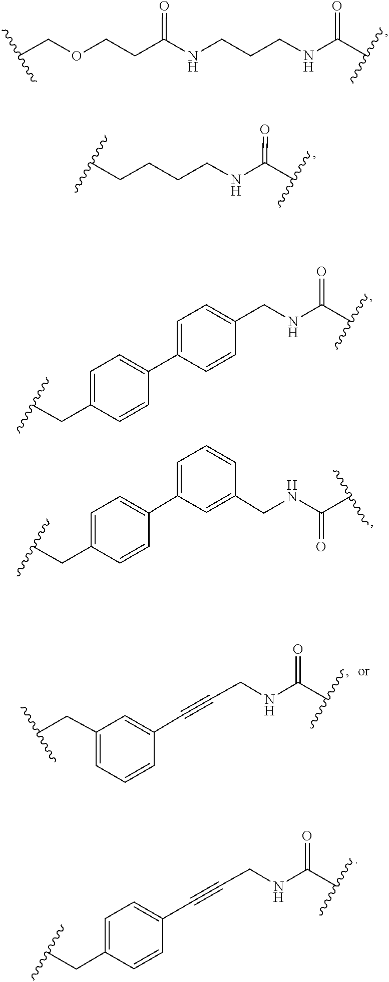 [SMILES: *COCCC(=O)NCCCNC(*)=O ; *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment P102. The compound of any one of embodiments P1 to P91, wherein L³ is

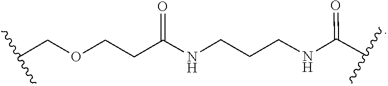 [SMILES: *COCCC(=O)NCCCNC(*)=O]

Embodiment P103. The compound of any one of embodiments P1 to P101, wherein R¹ is unsubstituted C₁-C₂₅ alkyl.
Embodiment P104. The compound of any one of embodiments P1 to P101, wherein R¹ is unsubstituted C₁₁-C₂₅ alkyl.
Embodiment P105. The compound of any one of embodiments P1 to P101, wherein R¹ is unsubstituted C₁₁-C₁₇ alkyl.
Embodiment P106. The compound of any one of embodiments P1 to P101, wherein R¹ is unsubstituted C₁₄-C₁₅ alkyl.
Embodiment P107. The compound of any one of embodiments P1 to P101, wherein R¹ is unsubstituted unbranched C₁-C₂₅ alkyl.
Embodiment P108. The compound of any one of embodiments P1 to P101, wherein R¹ is unsubstituted unbranched C₁₁-C₂₅ alkyl.
Embodiment P109. The compound of any one of embodiments P1 to P101, wherein R¹ is unsubstituted unbranched C₁₁-C₁₇ alkyl.
Embodiment P110. The compound of any one of embodiments P1 to P101, wherein R¹ is unsubstituted unbranched C₁₄-C₁₅ alkyl.
Embodiment P111. The compound of any one of embodiments P1 to P101, wherein R¹ is unsubstituted unbranched saturated C₁-C₂₅ alkyl.
Embodiment P112. The compound of any one of embodiments P1 to P101, wherein R¹ is unsubstituted unbranched saturated C₁₁-C₂₅ alkyl.
Embodiment P113. The compound of any one of embodiments P1 to P101, wherein R¹ is unsubstituted unbranched saturated C₁₁-C₁₇ alkyl.
Embodiment P114. The compound of any one of embodiments P1 to P101, wherein R¹ is unsubstituted unbranched saturated C₁₄-C₁₅ alkyl.
Embodiment P115. The compound of any one of embodiments P1 to P114, wherein R² is unsubstituted C₁-C₂₅ alkyl.
Embodiment P116. The compound of any one of embodiments P1 to P114, wherein R² is unsubstituted C₁₁-C₂₅ alkyl.
Embodiment P117. The compound of any one of embodiments P1 to P114, wherein R² is unsubstituted C₁₁-C₁₇ alkyl.
Embodiment P118. The compound of any one of embodiments P1 to P114, wherein R² is unsubstituted C₁₄-C₁₅ alkyl.
Embodiment P119. The compound of any one of embodiments P1 to P114, wherein R² is unsubstituted unbranched C₁-C₂₅ alkyl.
Embodiment P120. The compound of any one of embodiments P1 to P114, wherein R² is unsubstituted unbranched C₁₁-C₂₅ alkyl.
Embodiment P121. The compound of any one of embodiments P1 to P114, wherein R² is unsubstituted unbranched C₁₁-C₁₇ alkyl.
Embodiment P122. The compound of any one of embodiments P1 to P114, wherein R² is unsubstituted unbranched C₁₄-C₁₅ alkyl.
Embodiment P123. The compound of any one of embodiments P1 to P114, wherein R² is unsubstituted unbranched saturated C₁-C₂₅ alkyl.
Embodiment P124. The compound of any one of embodiments P1 to P114, wherein R² is unsubstituted unbranched saturated C₁₁-C₂₅ alkyl.
Embodiment P125. The compound of any one of embodiments P1 to P114, wherein R² is unsubstituted unbranched saturated C₁₁-C₁₇ alkyl.
Embodiment P126. The compound of any one of embodiments P1 to P114, wherein R² is unsubstituted unbranched saturated C₁₄-C₁₅ alkyl.
Embodiment P127. The compound of any one of embodiments P1 to P126, wherein R³ is unsubstituted C₁-C₂₅ alkyl.
Embodiment P128. The compound of any one of embodiments P1 to P126, wherein R³ is unsubstituted C₁₁-C₂₅ alkyl.
Embodiment P129. The compound of any one of embodiments P1 to P126, wherein R³ is unsubstituted C₁₁-C₁₇ alkyl.
Embodiment P130. The compound of any one of embodiments P1 to P126, wherein R³ is unsubstituted C₁₄-C₁₅ alkyl.
Embodiment P131. The compound of any one of embodiments P1 to P126, wherein R³ is unsubstituted unbranched C₁-C₂₅ alkyl.
Embodiment P132. The compound of any one of embodiments P1 to P126, wherein R³ is unsubstituted unbranched C₁₁-C₂₅ alkyl.
Embodiment P133. The compound of any one of embodiments P1 to P126, wherein R³ is unsubstituted unbranched C₁₁-C₁₇ alkyl.
Embodiment P134. The compound of any one of embodiments P1 to P126, wherein R³ is unsubstituted unbranched C₁₄-C₁₅ alkyl.
Embodiment P135. The compound of any one of embodiments P1 to P126, wherein R³ is unsubstituted unbranched saturated C₁-C₂₅ alkyl.
Embodiment P136. The compound of any one of embodiments P1 to P126, wherein R³ is unsubstituted unbranched saturated C₁₁-C₂₅ alkyl.
Embodiment P137. The compound of any one of embodiments P1 to P126, wherein R³ is unsubstituted unbranched saturated C₁₁-C₁₇ alkyl.
Embodiment P138. The compound of any one of embodiments P1 to P126, wherein R³ is unsubstituted unbranched saturated C₁₄-C₁₅ alkyl.
Embodiment P139. The compound of embodiment P1, wherein the compound comprises a structure of

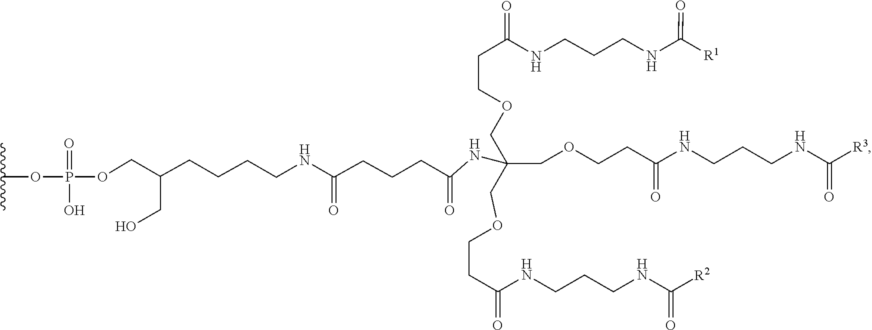

wherein the wavy line represents attachment point to the nucleic acid.
Embodiment P140. A method comprising contacting a cell with a compound of any one of embodiments P1 to P139.
Embodiment P141. The method of embodiment P140, wherein contacting occurs in vitro.
Embodiment P142. The method of embodiment P140, wherein the contacting occurs ex vivo.
Embodiment P143. The method of embodiment P140, wherein the contacting occurs in vivo.
Embodiment P144. A method comprising administering to a subject a compound of any one of embodiments P1 to P139.
Embodiment P145. The method of embodiment P144, wherein the subject has a disease or disorder of the eye, liver, kidney, heart, adipose tissue, lung, muscle or spleen.
Embodiment P146. A compound of any one of embodiments P1 to P139, for use in therapy.
Embodiment P147. A compound of any one of embodiments P1 to P139, for use in the preparation of a medicament.
Embodiment P148. A method of introducing a nucleic acid into a cell within a subject, the method comprising administering to said subject the compound of any one of embodiments P1 to P139.
Embodiment P149. A cell comprising the compound of any one of embodiments P1 to P139.
Embodiment P150. A pharmaceutical composition comprising a pharmaceutically acceptable excipient and the compound of embodiment P1.

### Additional Embodiments

Embodiment 1. A compound having the structure:

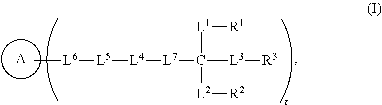

wherein
A is nucleic acid;
L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁶ is independently a bond, substituted or unsubstituted alkylene or substituted or unsubstituted heteroalkylene;
L¹ is independently a bond, substituted or unsubstituted 2 to 50 membered heteroalkylene or L^{1A}-L^{1B}-L^{1C}-L^{1D}-L^{1E};
L² is independently a bond, substituted or unsubstituted 2 to 50 membered heteroalkylene or L^{2A}-L^{2B}-L^{2C}-L^{2D}-L^{2E};
L³ is independently a bond, substituted or unsubstituted 2 to 50 membered heteroalkylene or L^{3A}-L^{3B}-L^{3C}-L^{3D}-L^{3E},
L^{1A}, L^{1B}, L^{1C}, L^{1D}, L^{1E}, L^{2A}, L^{2B}, L^{2C}, L^{2D}, L^{2E}, L^{3A}, L^{3B}, L^{3C}, L^{3D} and L^{3E} are independently a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted or unsubstituted C₁-C₂₅ alkylene, substituted or unsubstituted 2 to 25 membered heteroalkylene, substituted or unsubstituted cycloalkylene, substituted or unsubstituted heterocycloalkylene, substituted or unsubstituted arylene or substituted or unsubstituted heteroarylene,
wherein L^{1A}-L^{1B}-L^{1C}-L^{1D}-L^{1E} is not a bond, substituted or unsubstituted alkylene or a substituted or unsubstituted heteroalkylene with a terminal carbon atom and at least one of L^{1A}, L^{1B}, L^{1C}, L^{1D} or L^{1E} is not a bond or substituted or unsubstituted 2 to 25 membered heteroalkylene,
wherein L^{2A}-L^{2B}-L^{2C}-L^{2D}-L^{2E} is not a bond, substituted or unsubstituted alkylene or a substituted or unsubstituted heteroalkylene with a terminal carbon atom and at least one of L^{2A}, L^{2B}, L^{2C}, L^{2D} or L^{2E} is not a bond or substituted or unsubstituted 2 to 25 membered heteroalkylene,
wherein L^{3A}-L^{3B}-L^{3C}-L^{3D}-L^{3E} is not a bond or substituted or unsubstituted alkylene or a substituted or unsubstituted heteroalkylene with a terminal carbon atom at least one of L^{3A} L^{3B}, L^{3C}, L^{3D} or L^{3E} is not a bond or substituted or unsubstituted 2 to 25 membered heteroalkylene;
R¹, R² and R³ are independently unsubstituted C₁-C₂₅ alkyl; and
t is an integer from 1 to 5.
Embodiment 2. The compound of embodiment 1, wherein R¹, R² and R³ are independently unsubstituted C₇-C₂₀ alkyl.

Provided herein are Embodiments 3 to 141 that are embodiments of the compound of Embodiment 1 and the same scope as embodiments P1 to P139. Also provided are the following method embodiments:
Embodiment 142. A method comprising contacting a cell with a compound of any one of embodiments 1 to 141.
Embodiment 143. The method of embodiment 142, wherein contacting occurs in vitro.
Embodiment 144. The method of embodiment 142, wherein the contacting occurs ex vivo.
Embodiment 145. The method of embodiment 142, wherein the contacting occurs in vivo.
Embodiment 146. A method comprising administering to a subject a compound of any one of embodiments 1 to 141.
Embodiment 147. The method of embodiment 146, wherein the subject has a disease or disorder of the eye, liver, kidney, heart, adipose tissue, lung, muscle or spleen.
Embodiment 148. A compound of any one of embodiments 1 to 141, for use in therapy.
Embodiment 149. A compound of any one of embodiments 1 to 141, for use in the preparation of a medicament.
Embodiment 150. A method of introducing a nucleic acid into a cell within a subject, the method comprising administering to said subject the compound of any one of embodiments 1 to 141.
Embodiment 151. A cell comprising the compound of any one of embodiments 1 to 141.
Embodiment 152. A pharmaceutical composition comprising a pharmaceutically acceptable excipient and the compound of any one of embodiments 1 to 141.

### EXAMPLES

The following examples are presented to more fully illustrate some embodiments of the invention. They should not be construed, however, as limiting the scope of the invention. Variations of these examples within the scope of the claims are within the purview of one skilled in the art and are considered to fall within the scope of the embodiments as described and claimed herein. The reader will recognize that the skilled artisan, armed with the present disclosure and skill in the art, is able to prepare and use the invention without exhaustive examples.

### Example 1: Synthesis of Uptake Motifs

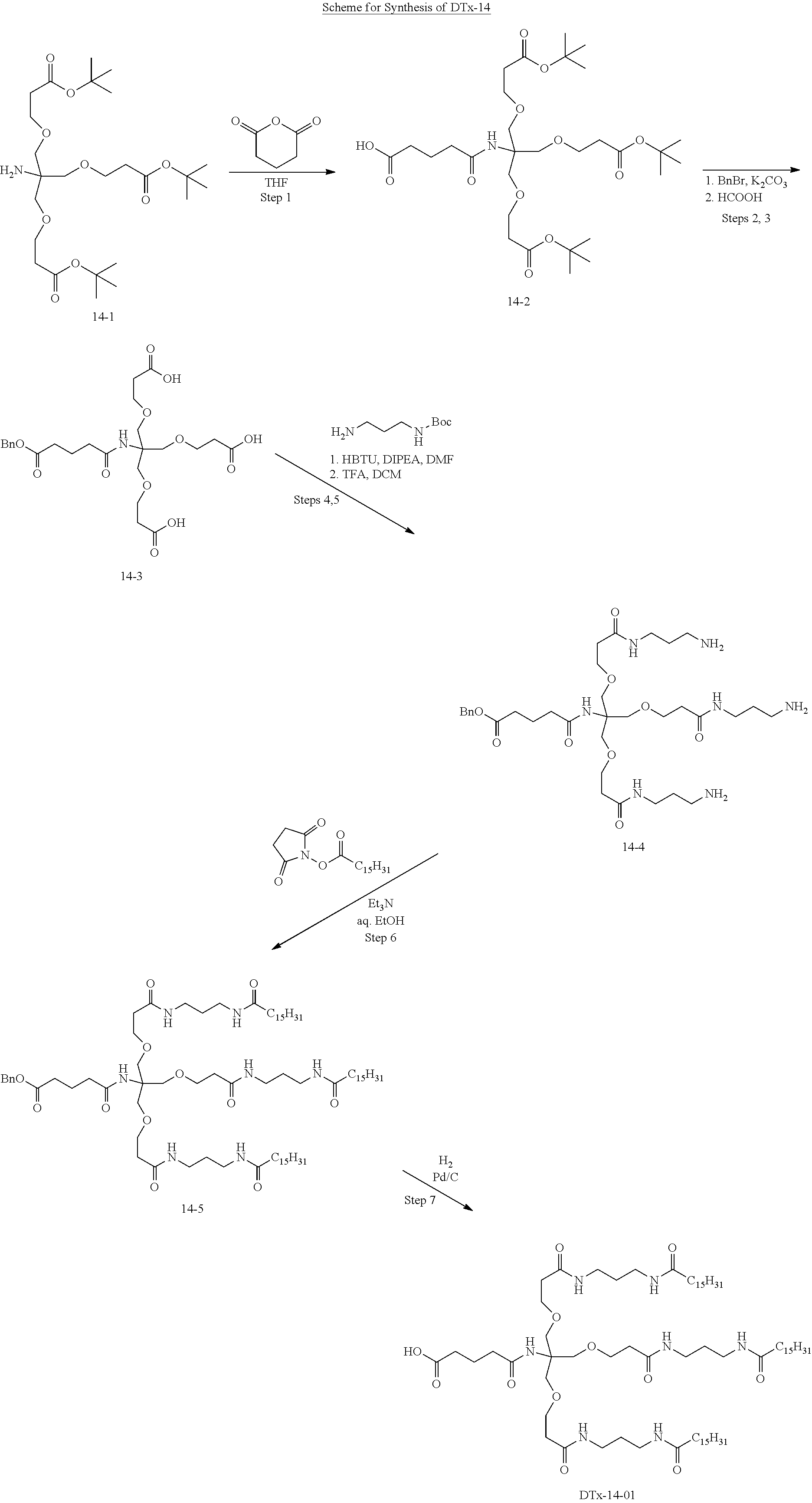

### Synthesis Procedures of DTx-14

Step 1: To a stirred solution of 14-1 (1 mmol) in THF (10 mL) is added glutaric anhydride (1.2 mmol). The resulting mixture is stirred at room temperature for 16 hours. The mixture is concentrated in vacuo to yield crude 14-2.
Steps 2 and 3: To a stirred solution of 14-2 (1 mmol) in acetonitrile (10 mL) is added K₂CO₃ (1.2 mmol) and benzyl bromide (1.2 mmol). The resulting mixture is stirred at room temperature for 16 hours. The mixture is concentrated under reduced pressure, triturated with diethyl ether, and dried in vacuo. The resulting oil is redissolved in 1,4-dioxane (10 mL) and treated with formic acid (0.5 mL). The resulting mixture is stirred at room temperature for 16 hours. The mixture is subsequently concentrated in vacuo to yield 14-3.
Steps 4 and 5: To a stirred solution of 14-3 (1 mmol) in DMF (5 mL) is added HBTU (4 mmol), DIPEA (8 mmol) and mono-N-Boc-protected 1,3-diamino propane (4 mmol). The resulting mixture is stirred at room temperature for 16 hours. The mixture is diluted with DCM, washed with saturated aq. NaHCO₃, dried over Na₂SO₄, filtered, and evaporated to an oil, which is subsequently purified by silica gel chromatography. The resulting material is dissolved with stirring in dichloromethane (5 mL) and treated with trifluoroacetic acid (0.5 mL). After 1 hour the material is concentrated in vacuo to yield 14-4.
Step 6: To a stirred solution of 14-4 (1 mmol) in aqueous ethanol (5 mL) is added Et₃N (6 mmol) and the NHS-ester of palmitic acid (4 mmol). The resulting mixture is stirred at room temperature for 8 hours. The mixture is subsequently diluted with DCM, washed with saturated aq. NaHCO₃, dried over anhydrous Na₂SO₄, filtered, and concentrated in vacuo. Purification by silica gel chromatography yields 14-5.
Step 7: To a stirred solution of 14-5 (1 mmol) in methanol (5 mL) is added Pd/C. The reaction vessel is maintained with an atmosphere of H₂ gas for 10 hours. The reaction mixture is filtered to remove the Pd/C catalyst. The clarified solution is concentrated in vacuo, to yield DTx-14-01 as a solid.

### Example 2: Conjugation of Uptake Motifs to Oligonucleotides

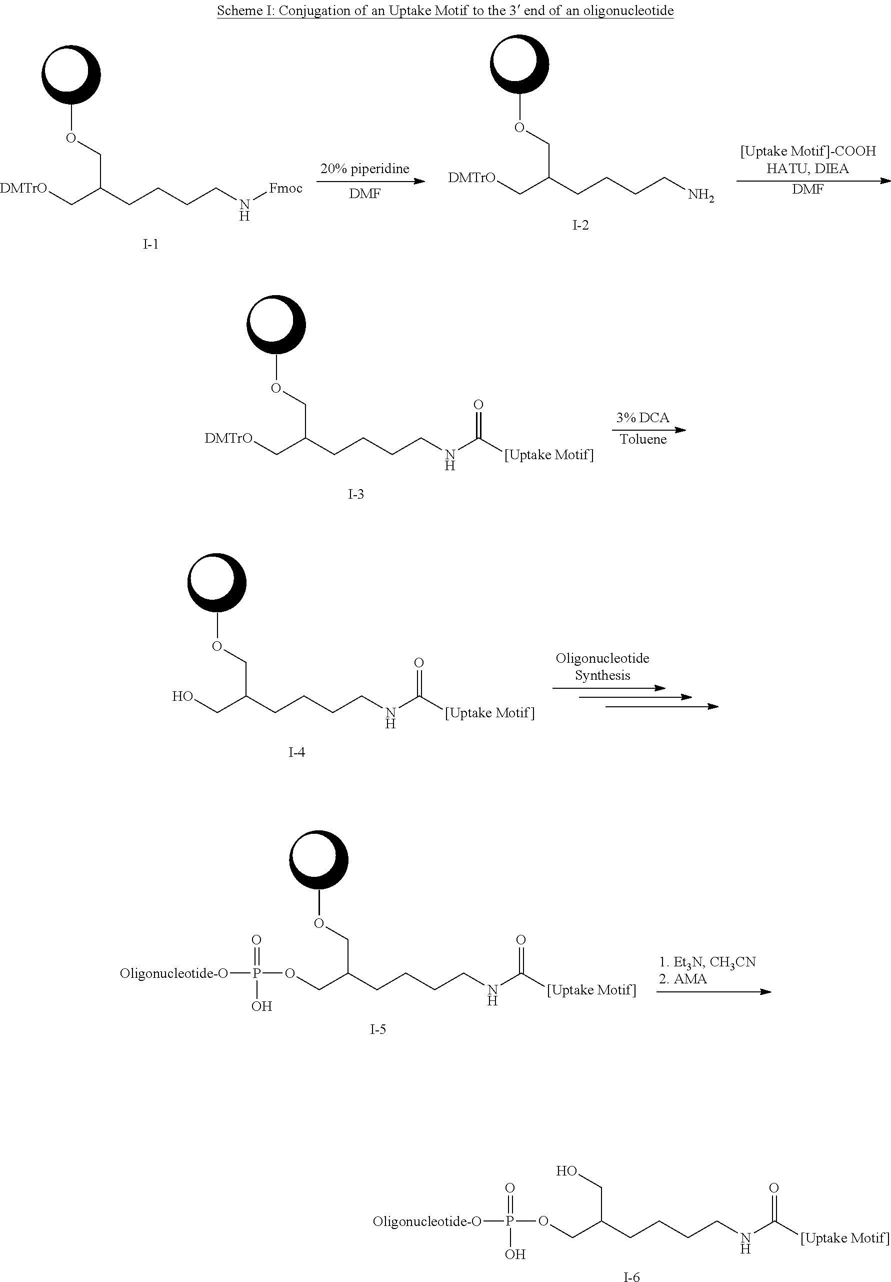

Scheme I above illustrates the preparation of a single-stranded oligonucleotide (sense or antisense strand of a double-stranded oligonucleotide) conjugated with a lipid uptake motif at the 3′ terminus. In summary, 3′-C7-amino CPG beads I-1 (Glen Research, Catalog No. 20-2958) bearing both DMTr and Fmoc protecting groups are treated with 20% piperidine in DMF to afford the free-amino structure I-2. The free-acid form of an uptake motif ([Uptake Motif]-COOH) is then coupled to I-2 using HATU and DIEA in DMF to produce uptake-motif loaded CPG beads I-3, which is subsequently treated with dichloroacetic acid (DCA) in toluene to remove the DMTr protecting group and afford I-4. Oligonucleotide synthesis from the liberated hydroxyl group is accomplished using standard phosphoramidite chemistry and yields the CPG-bound conjugated oligonucleotide structure I-5. Subsequent treatment with ammonium hydroxide and methylamine (AMA) yields the crude conjugated oligonucleotide I-6. Purification is accomplished through either ion-exchange or reverse-phase chromatography. Purity and identify are then measured and confirmed by LCMS analysis.

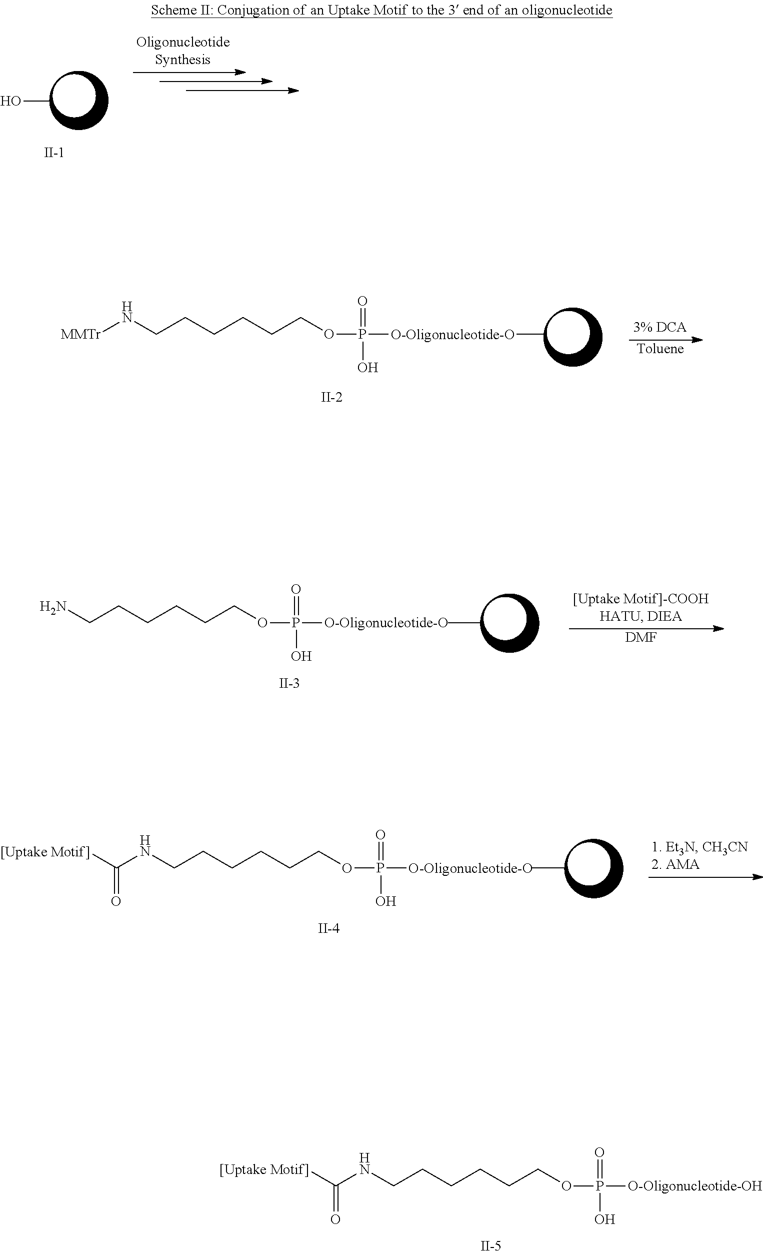

Scheme II above illustrates the preparation of a single-stranded oligonucleotide (sense or antisense strand of a double-stranded oligonucleotide) conjugated with a lipid uptake motif at the 5′ terminus. In summary, solid-phase oligonucleotide synthesis is performed on synthesis support II-1 (CPG, NittoPhase HL, or similar) via standard phosphoramidite chemistry. The final coupling is with a phosphoramidite (Glen Research, Catalog No. 10-1906) that incorporates an MMTr-protected six-carbon alkyl amine as shown in structure II-2. The MMTr protecting group is removed by treatment with DCA in toluene to yield structure II-3. The free amine is coupled to the Uptake Motif using HATU and DIEA in DMF to generate the resin-bound conjugated oligonucleotide II-4. Deprotection using triethylamine in acetonitrile followed by ammonium hydroxide/methylamine (AMA) yields the crude conjugated oligonucleotide II-5. Purification is accomplished through either ion-exchange or reverse-phase chromatography. Purity and identify are then measured and confirmed by LCMS analysis.

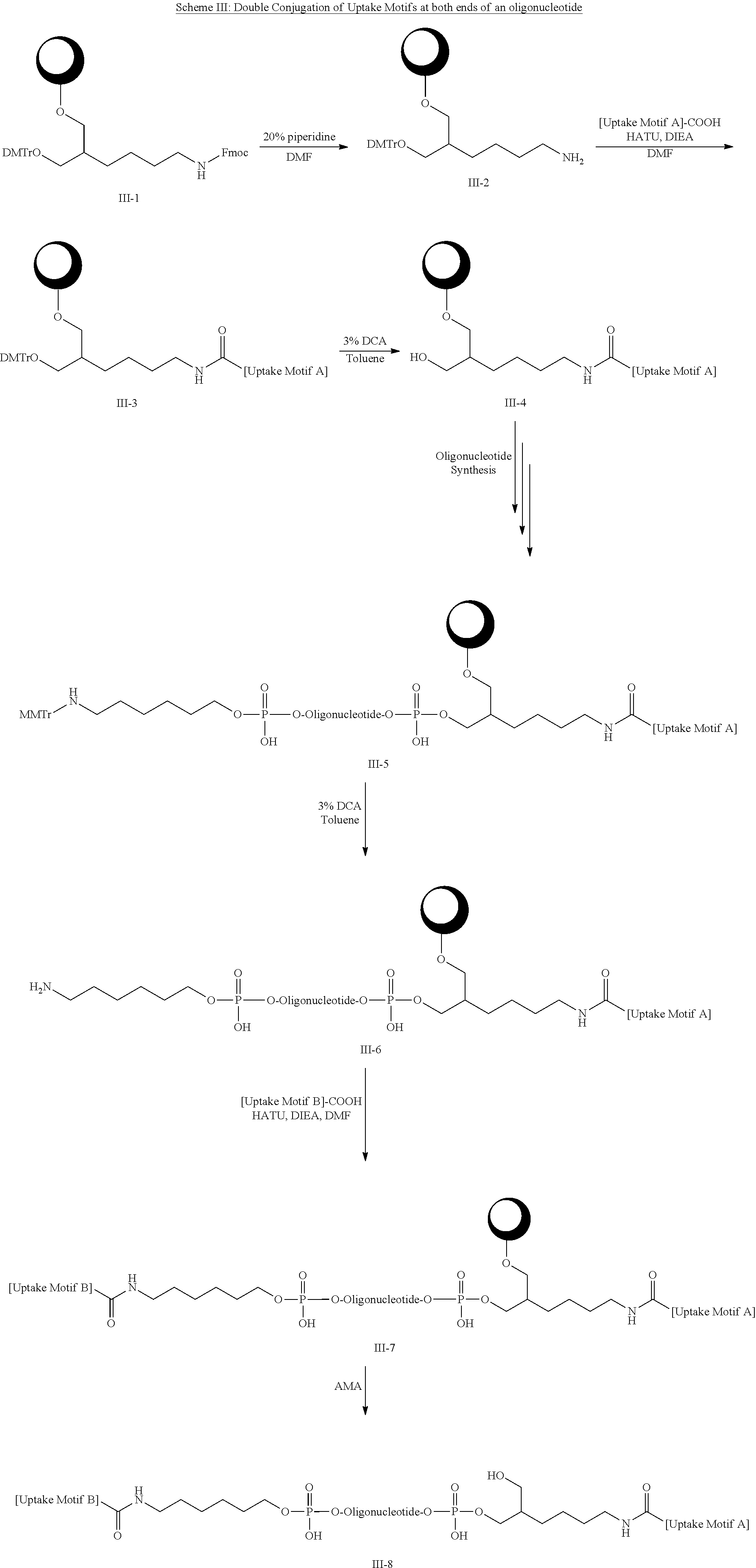

Scheme III above illustrates the preparation of a single-stranded oligonucleotide (sense or antisense strand of a double-stranded oligonucleotide) conjugated with lipid uptake motifs at both the 5′ terminus and the 3′ terminus. In summary, 3′-C7-amino CPG beads III-1 (Glen Research, Catalog No. 20-2958) bearing both DMTr and Fmoc protecting groups are treated with 20% piperidine in DMF to afford the free-amino structure III-2. The free-acid form of an uptake motif ([Uptake Motif]-COOH) is then coupled to III-2 using HATU and DIEA in DMF to produce uptake-motif loaded CPG beads III-3, which is subsequently treated with dichloroacetic acid (DCA) in toluene to remove the DMTr protecting group and afford III-4. Oligonucleotide synthesis from the liberated hydroxyl group is accomplished using standard phosphoramidite chemistry. The final coupling is with a phosphoramidite (Glen Research, Catalog No. 10-1906) that incorporates an MMTr-protected six-carbon alkyl amine as shown in structure III-5. The MMTr protecting group is removed by treatment with DCA in toluene to yield structure III-6. The liberated amine is coupled to the second Uptake Motif using HATU and DIEA in DMF to generate the resin-bound doubly-conjugated oligonucleotide III-7. Subsequent treatment with ammonium hydroxide and methylamine (AMA) yields the crude doubly-conjugated oligonucleotide III-8. Purification is accomplished through either ion-exchange or reverse-phase chromatography. Purity and identify are then measured and confirmed by LCMS analysis.

### Example 3: General Procedures and Methods

### Duplex Formation

For each of the sense strands synthesized by a Scheme provided herein, the complementary antisense strand was prepared via standard phosphoramidite chemistry, purified by IE-HPLC, and characterized by MALDI-TOF MS using the [M+H] peak. The duplex was formed by mixing equal molar equivalents of the sense strand and antisense strand, heating to 90° C. for 5 minutes, and then slowly cooling to room temperature. Duplex formation was confirmed by non-denaturing PAGE.

### Cell Culture

HUVEC cells were purchased from Cell Applications (San Diego, CA) and cultured in their proprietary HUVEC cell media containing 2% serum, 100 U/mL penicillin and 100 mg/mL streptomycin.

### Free Uptake Experiments

HUVEC cells were plated at 10,000 cells/well on 96 well collagen-coated plates. The day after plating, the HUVEC media was removed and the cells were washed twice with PBS containing calcium and magnesium. Following the last wash, cells were incubated with compounds at various concentrations in serum free HUVEC media for 24 hours. After 24 hours, compound containing media was removed and replaced with normal HUVEC media for 24 additional hours. Cells were then washed twice with PBS containing calcium and magnesium and then prepared for RNA isolation according to the manufacturer's protocol (see above). In an alternative paradigm when the effect of albumin was not of interest, cells were incubated with compounds at various concentrations in normal HUVEC media containing 2% serum for 48 hours. After 48 hours, cells were washed twice with PBS containing calcium and magnesium and then prepared for RNA isolation according to the manufacturer's protocol (see below).

### RNA Isolation, Reverse Transcription and Quantitative PCR

RNA was isolated utilizing the RNeasy 96 kit (Qiagen) according to the manufacturer's protocol. It was reverse transcribed to cDNA utilizing random primers and the high-capacity cDNA reverse transcription kit (ThermoFisher Scientific) in a SimpliAmp thermal cycler (ThermoFisher Scientific) according to manufacturer's instructions. Quantitative PCR was performed utilizing gene-specific primers (Thermofisher Scientific; IDTDNA), TaqMan probes (Thermofisher Scientific; IDTDNA) and TaqMan fast universal PCR master mix (Thermofisher scientific) on a StepOnePlus real-time PCR system (Thermofisher scientific) according to manufacturer's instructions. For analysis of quantitative PCR, mRNA expression was normalized to the expression of either 18s rRNA, β-actin or HPRT1 mRNA (housekeeping genes) utilizing the relative CT method according to the best practices proposed in Nature Protocols (Schmittgen, T. D. & Livak, K. J. Analyzing real-time PCR data by the comparative C (T) method. Nat Protoc 3, 1101-1108 (2008)).

### Systemic Delivery Studies

Following acclimatization for 7 days, mice were weighed the night before the study and sorted into groups based on body weight. The day of study initiation, the mice were injected with PBS or the compound of interest via intravenous injection. The dosing paradigm and duration of study are provided in the biological data section. After a period of time, the mice were euthanized by CO₂ asphyxiation followed by secondary confirmation of euthanasia via cervical dislocation seven days following either a single injection or seven days following the last dose when repeated injections were utilized. The tissues of interest were then removed and 30-300 mg placed in RNALater immediately following dissection. 24 hours later, the tissue was removed from the RNALater, blotted dry and placed into trizol in tubes containing lysing matrix D beads from MPBiomedical. The tissue was homogenized using the MPBio FastPrep-24 system. Chloroform extraction was then performed by adding 0.2 mL per 1 mL of Trizol. Samples were mixed thoroughly, spun at max speed in a microcentrifuge at 4° C. for 15 minutes and the aqueous layer. The RNA was then precipitated by adding 1.5 volumes of absolute ethanol to the aqueous phase. The precipitated RNA was then purified utilizing the RNeasy 96 kit from Qiagen according to the manufacturer's instructions, substituting RLT buffer for RW1 buffer.

### Example 4: Testing of Conjugated Compounds in Cell Culture

Uptake motifs described herein comprising three long chain fatty acids were tested for their ability to facilitate uptake of the conjugated siRNA into cells, relative to a structurally distinct uptake motif, without the aid of any transfection reagent.

Several different uptake motifs were conjugated to the sense strand of an siRNA targeting PTEN. The siRNA nucleobase sequence and chemical modifications are shown in Table A, where a nucleotide followed by the subscript “F” is a 2′-fluoro nucleotide, a nucleotide followed by the subscript “M” is a 2′-O-methyl nucleotide, and a nucleotide without a subscript is a beta-D-deoxyribonucleotide, a superscript “S” is a phosphorothioate internucleotide linkage, and all other internucleotide linkages are phosphodiester internucleotide linkages. A hydroxyl group is present at the terminal 5′ carbon of the sense strand. A phosphate group is present at the terminal 5′ carbon and a hydroxyl group is present at the terminal 3′ carbon of the antisense strand.

TABLE A
PTEN siRNA Nucleotide Sequence and Chemistry

| | | SEQ ID |
|---|---|---|
| | Nucleotide Sequence (5′ to 3′) | NO: |
| Sense | G_{F}^{S}A_{M}^{S}U_{F}G_{M}A_{F}U_{M}G_{F}U_{M}U_{F}U_{M}G_{F}A_{M}A_{F}A_{M}C_{F}U_{M}A_{F}^{S}U_{M}^{S}U_{F} | 3 |
| strand | | |
| Antisense | A^{MS}A^{FS}U^{M}A^{F}G^{M}U^{F}U^{M}U^{F}C^{M}A^{F}A^{M}A^{F}C^{M}A^{F}U^{M}C^{F}A^{M}U^{F}C^{MS}T^{S}T | 2 |
| strand | | |

Table B illustrates the uptake motif structures, each containing three LCFA moieties that were conjugated to an uptake motif through a linker as shown in the structure below, where the wavy line represents attachment of the phosphate group to the 3′ carbon of the 3′-terminal nucleotide of the sense strand of the PTEN siRNA. R¹, R² and R³ indicate the long chain fatty acids for each uptake motif.

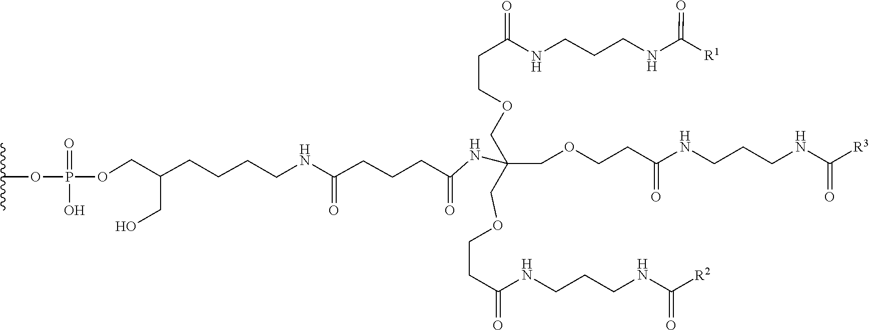

TABLE B
Uptake Motif-Conjugated siRNA

| | Uptake | | | |
|---|---|---|---|---|
| Compound ID | Motif | R¹ | R² | R³ |
| DT-000815 | DTx-14-02 | C7 | C7 | C7 |
| DT-000816 | DTx-14-03 | C9 | C9 | C9 |
| DT-000817 | DTx-14-04 | C11 | C11 | C11 |
| DT-000818 | DTx-14-05 | C13 | C13 | C13 |
| DT-000819 | DTx-14-01 | C15 | C15 | C15 |

Also tested was the PTEN siRNA conjugated to the uptake motif DTx-01-08, which has a scaffold structure different than that of the DTx-14 scaffold described herein, and also comprises only two LCFA motifs. DTx-01-08 has the following structure and was connected to the 3′ terminal carbon of the sense strand via a C₇ linker:

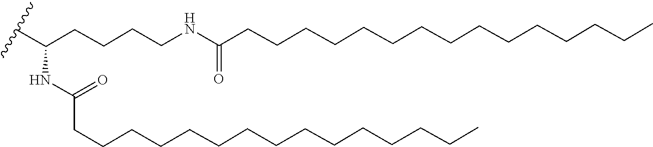 [SMILES: *[C@H](CCCCNC(=O)CCCCCCCCCCCCCCC)NC(=O)CCCCCCCCCCCCCCC]

Free uptake experiments were performed in HUVEC cells by incubating the cells with PBS or compound for 48 hours. RNA was then isolated and the mean PTEN mRNA expression across 4 replicates per treatment quantified by QT-PCR. In PBS-treated cells, the PTEN expression was 100.9% (S.E.M.=2.83). The mean PTEN mRNA expression in compound-treated cells is shown in Table C.

As shown in Table C, the activity of a triple-fatty acid uptake motif depends on the length of the fatty acid. DT-000817 and DT-000818 exhibited the greatest activity. Notably, DT-000819 was substantially less active than DT-000337.

TABLE C
Improved in vitro Activity with Certain Triple Fatty Acid Uptake Motifs

| | Fatty Acid | | | | | | | | |
|---|---|---|---|---|---|---|---|---|---|
| | Lengths | | | 3 nM | | 30 nM | | 300 nM | |
| | R¹ | R² | R³ | Mean | SEM | Mean | SEM | Mean | SEM |
| DT-000337 | | | | 70.3 | 0.29 | 17.9 | 1.21 | 0.18 | 0.04 |
| DT-000815 | C7 | C7 | C7 | 92.6 | 6.88 | 84.0 | 1.57 | 70.9 | 2.55 |
| DT-000816 | C9 | C9 | C9 | 104.1 | 3.15 | 101.5 | 4.87 | 37.4 | 3.15 |
| DT-000817 | C11 | C11 | C11 | 72.6 | 1.98 | 47.2 | 0.65 | 0.94 | 0.06 |
| DT-000818 | C13 | C13 | C13 | 83.2 | 2.51 | 43.6 | 2.10 | 1.37 | 0.07 |
| DT-000819 | C15 | C15 | C15 | 96.1 | 6.87 | 75.2 | 4.64 | 24.6 | 1.39 |

The three most active compounds in the HUVEC free uptake experiment were tested in vivo. C₅₇B16/J mice were injected intravenously with a single dose of either PBS, 10 mg/kg or 30 mg/kg of siRNAs containing uptake motifs. Seven days following injection, mice were euthanized. Heart, quadricep muscle, diaphragm, tibialis anterior (TA) muscle, liver and kidney tissues were collected for RNA extraction. Mean repression of PTEN mRNA expression was calculated from 4-5 animals per treatment.

The results for heart, quadricep, diaphragm, TA muscle, liver and kidney are shown in Tables D through I. The activity of DT-000817 was similar to that of DT-000337 in all tissues but kidney, where DT-000337 was slightly more active. Compounds DT-000818 and DT-000819 were similarly active in all tissues, and slightly less active than DT-000817. These results demonstrate that for the structure provided herein, in vivo activity varies depending upon the length of the fatty acids comprised in the uptake motif.

TABLE D
Activity in Heart with Certain Triple Fatty Acid Uptake Motifs

| | Fatty Acid | | | | | | | | |
|---|---|---|---|---|---|---|---|---|---|
| | Lengths | | | Vehicle | | 10 mg/kg | | 30 mg/kg | |
| | R¹ | R² | R³ | Mean | SEM | Mean | SEM | Mean | SEM |
| PBS | | | | 100.9 | 7.14 | — | — | — | — |
| DT-000337 | | | | | | 68.9 | 5.39 | 41.5 | 2.11 |
| DT-000817 | C11 | C11 | C11 | — | — | 66.2 | 3.74 | 46.6 | 3.26 |
| DT-000818 | C13 | C13 | C13 | — | — | 85.9 | 5.16 | 56.1 | 0.90 |
| DT-000819 | C15 | C15 | C15 | — | — | 82.2 | 5.21 | 59.1 | 6.40 |

TABLE E
Activity in Quadricep Muscle with Certain Triple Fatty Acid Uptake Motifs

| | Fatty Acid | | | | | | | | |
|---|---|---|---|---|---|---|---|---|---|
| | Lengths | | | Vehicle | | 10 mg/kg | | 30 mg/kg | |
| | R¹ | R² | R³ | Mean | SEM | Mean | SEM | Mean | SEM |
| PBS | | | | 100.1 | 2.48 | — | — | — | — |
| DT-000337 | | | | | | 69.1 | 2.84 | 44.4 | 2.54 |
| DT-000817 | C11 | C11 | C11 | — | — | 68.9 | 2.22 | 40.4 | 2.10 |
| DT-000818 | C13 | C13 | C13 | — | — | 90.6 | 5.28 | 65.1 | 4.35 |
| DT-000819 | C15 | C15 | C15 | — | — | 86.3 | 7.68 | 74.0 | 6.36 |

TABLE F
Activity in Diaphragm with Certain Triple Fatty Acid Uptake Motifs

| | Fatty Acid | | | | | | | | |
|---|---|---|---|---|---|---|---|---|---|
| | Lengths | | | Vehicle | | 10 mg/kg | | 30 mg/kg | |
| | R¹ | R² | R³ | Mean | SEM | Mean | SEM | Mean | SEM |
| PBS | | | | 102.0 | 10.0 | — | — | — | — |
| DT-000337 | | | | | | 54.9 | 9.31 | 45.8 | 3.08 |
| DT-000817 | C11 | C11 | C11 | — | — | 59.7 | 5.72 | 54.3 | 2.84 |
| DT-000818 | C13 | C13 | C13 | — | — | 78.8 | 6.20 | 50.3 | 2.13 |
| DT-000819 | C15 | C15 | C15 | — | — | 76.9 | 10.40 | 77.7 | 5.37 |

TABLE G
Activity in TA Muscle with Certain Triple Fatty Acid Uptake Motifs

| | Fatty Acid | | | | | | | | |
|---|---|---|---|---|---|---|---|---|---|
| | Lengths | | | Vehicle | | 10 mg/kg | | 30 mg/kg | |
| | R¹ | R² | R³ | Mean | SEM | Mean | SEM | Mean | SEM |
| PBS | | | | 100.5 | 5.11 | — | — | — | — |
| DT-000337 | | | | | | 82.6 | 5.29 | 55.9 | 5.15 |
| DT-000817 | C11 | C11 | C11 | — | — | 92.3 | 2.70 | 55.2 | 3.15 |
| DT-000818 | C13 | C13 | C13 | — | — | 100.2 | 3.91 | 80.6 | 3.46 |
| DT-000819 | C15 | C15 | C15 | — | — | 113.4 | 3.77 | 100.3 | 3.78 |

TABLE H
Activity in Liver with Certain Triple Fatty Acid Uptake Motifs

| | Fatty Acid | | | | | | | | |
|---|---|---|---|---|---|---|---|---|---|
| | Lengths | | | Vehicle | | 10 mg/kg | | 30 mg/kg | |
| | R¹ | R² | R³ | Mean | SEM | Mean | SEM | Mean | SEM |
| PBS | | | | 100.3 | 3.69 | — | — | — | — |
| DT-000337 | | | | | | 12.1 | 0.70 | 7.5 | 0.39 |
| DT-000817 | C11 | C11 | C11 | — | — | 18.2 | 2.21 | 9.2 | 0.39 |
| DT-000818 | C13 | C13 | C13 | — | — | 16.3 | 1.10 | 8.6 | 0.40 |
| DT-000819 | C15 | C15 | C15 | — | — | 15.8 | 0.98 | 10.2 | 0.71 |

TABLE I
Activity in Kidney with Certain Triple Fatty Acid Uptake Motifs

| | Fatty Acid | | | | | | | | |
|---|---|---|---|---|---|---|---|---|---|
| | Lengths | | | Vehicle | | 10 mg/kg | | 30 mg/kg | |
| | R¹ | R² | R³ | Mean | SEM | Mean | SEM | Mean | SEM |
| PBS | | | | 100.3 | 3.72 | — | — | — | — |
| DT-000337 | | | | | | 68.9 | 5.39 | 41.5 | 2.11 |
| DT-000817 | C11 | C11 | C11 | — | — | 99.1 | 2.09 | 98.8 | 5.17 |
| DT-000818 | C13 | C13 | C13 | — | — | 111.6 | 7.64 | 101.6 | 6.6 |
| DT-000819 | C15 | C15 | C15 | — | — | 125.9 | 5.97 | 95.5 | 4.49 |

1. A compound having the structure: 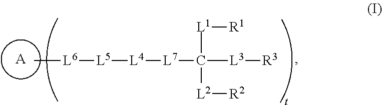
or a pharmaceutically acceptable salt thereof,
wherein
A is nucleic acid comprising a 5-(E)-vinylphosphonate group;
L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁷ is independently a bond, substituted or unsubstituted alkylene or substituted or unsubstituted heteroalkylene;
L¹ is independently a bond, substituted or unsubstituted 2 to 50 membered heteroalkylene or L^{1A}-L^{1B}-L^{1C}-L^{1D}-L^{1E};
L² is independently a bond, substituted or unsubstituted 2 to 50 membered heteroalkylene or L^{2A}-L^{2B}-L^{2C}-L^{2D}-L^{2E},
L³ is independently a bond, substituted or unsubstituted 2 to 50 membered heteroalkylene or L^{3A}-L^{3B}-L^{3C}-L^{3D}-L^{3E};
L^{1A}, L^{1B}, L^{1C}, L^{1D}, L^{1E}, L^{2A}, L^{2B}, L^{2C}, L^{2D}, L^{2E}, L^{3A}, L^{3B}, L^{3C}, L^{3D} and L^{3E} are independently a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted or unsubstituted C₁-C₂₅ alkylene, substituted or unsubstituted 2 to 25 membered heteroalkylene, substituted or unsubstituted cycloalkylene, substituted or unsubstituted heterocycloalkylene, substituted or unsubstituted arylene or substituted or unsubstituted heteroarylene,
wherein L^{1A}-L^{1B}-L^{1C}-L^{1D}-L^{1E} is not a bond, substituted or unsubstituted alkylene or a substituted or unsubstituted heteroalkylene with a terminal carbon atom and at least one of L^{1A}, L^{1B}, L^{1C}, L^{1D} or L^{1E} is not a bond or substituted or unsubstituted 2 to 25 membered heteroalkylene,
wherein L^{2A}-L^{2B}-L^{2C}-L^{2D}-L^{2E} is not a bond, substituted or unsubstituted alkylene or a substituted or unsubstituted heteroalkylene with a terminal carbon atom and at least one of L^{2A}, L^{2B}, L^{2C}, L^{2D} or L^{2E} is not a bond or substituted or unsubstituted 2 to 25 membered heteroalkylene,
wherein L^{3A}-L^{3B}-L^{3C}-L^{3D}-L^{3E} is not a bond or substituted or unsubstituted alkylene or a substituted or unsubstituted heteroalkylene with a terminal carbon atom at least one of L^{3A}, L^{3B}, L^{3C}, L^{3D} or L^{3E} is not a bond or substituted or unsubstituted 2 to 25 membered heteroalkylene;
R¹, R² and R³ are independently unsubstituted C₁-C₂₅ alkyl; and
t is an integer from 1 to 5.

2. The compound of claim 1, or a pharmaceutically acceptable salt thereof, wherein R¹, R² and R³ are independently unsubstituted C₇-C₂₀ alkyl.

3. A compound having the structure: 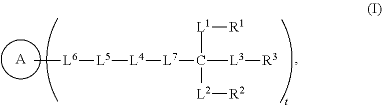
or a pharmaceutically acceptable salt thereof,
wherein
A is nucleic acid comprising a 5-(E)-vinylphosphonate group;
L⁴ and L⁶ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)—O—, —OP(S)₂—O—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁷ is independently a bond, substituted or unsubstituted alkylene or substituted or unsubstituted heteroalkylene;
L¹ is independently a bond, substituted or unsubstituted 2 to 50 membered heteroalkylene or L^{1A}-L^{1B}-L^{1C}-L^{1D}-L^{1E};
L² is independently a bond, substituted or unsubstituted 2 to 50 membered heteroalkylene or L^{2A}-L^{2B}-L^{2C}-L^{2D}-L^{2E};
L³ is independently a bond, substituted or unsubstituted 2 to 50 membered heteroalkylene or L^{3A}-L^{3B}-L^{3C}-L^{3D}-L^{3E};
L^{1A}, L^{1B}, L^{1C}, L^{1D}, L^{1E}, L^{2A}, L^{2B}, L^{2C}, L^{2D}, L^{2E}, L^{3A}, L^{3B}, L^{3C}, L^{3D} and L^{3E} are independently a bond, —NH—, —O—, —S—, —C(O)—, —NHC(O)—, —S(O)₂C(O)—, —OPO₂—O—, —OP(S)(O)—O—, —OP(S)₂—O—, —S(O)NH—, —NHC(O)NH—, —C(O)O—, —OC(O)—, —C(O)NH—, substituted or unsubstituted C₁-C₂₅ alkylene, substituted or unsubstituted 2 to 25 membered heteroalkylene, substituted or unsubstituted cycloalkylene, substituted or unsubstituted heterocycloalkylene, substituted or unsubstituted arylene or substituted or unsubstituted heteroarylene,
wherein L^{1A}-L^{1B}-L^{1C}-L^{1D}-L^{1E} is not a bond, substituted or unsubstituted alkylene or a substituted or unsubstituted heteroalkylene with a terminal carbon atom and at least one of L^{1A}, L^{1B}, L^{1C}, L^{1D} or L^{1E} is not a bond or substituted or unsubstituted 2 to 25 membered heteroalkylene,
wherein L^{2A}-L^{2B}-L^{2C}-L^{2D}-L^{2E} is not a bond, substituted or unsubstituted alkylene or a substituted or unsubstituted heteroalkylene with a terminal carbon atom and at least one of L^{2A}, L^{2B}, L^{2C}, L^{2D} or L^{2E} is not a bond or substituted or unsubstituted 2 to 25 membered heteroalkylene,
wherein L^{3A}-L^{3B}-L^{3C}-L^{3D}-L^{3E} is not a bond or substituted or unsubstituted alkylene or a substituted or unsubstituted heteroalkylene with a terminal carbon atom at least one of L^{3A}, L^{3B}, L^{3C}, L^{3D} or L^{3E} is not a bond or substituted or unsubstituted 2 to 25 membered heteroalkylene;
R¹, R² and R³ are independently unsubstituted C₈-C₂₀ alkyl; and
t is an integer from 1 to 5.

4. The compound of claim 3, or a pharmaceutically acceptable salt thereof, wherein L¹, L² and L³ are independently substituted or unsubstituted 2 to 50 membered heteroalkylene.

5. (canceled)

6. The compound of claim 3, or a pharmaceutically acceptable salt thereof, wherein tis 1, 2, or 3.

7-8. (canceled)

9. The compound of claim 3, or a pharmaceutically acceptable salt thereof, wherein A is a double-stranded nucleic acid, or a single-stranded nucleic acid.

10. The compound of claim 9, or a pharmaceutically acceptable salt thereof, wherein the double-stranded nucleic acid is a small interfering RNA, a short hairpin RNA or a microRNA mimic.

11. (canceled)

12. The compound of claim 3, or a pharmaceutically acceptable salt thereof, wherein the nucleic acid of A comprises one or more modified nucleotides or one or more modified internucleotide linkages.

13. The compound of claim 3, or a pharmaceutically acceptable salt thereof, wherein the nucleic acid comprises one or more modified sugar moieties.

14. The compound of claim 12, or a pharmaceutically acceptable salt thereof, wherein the modified sugar moiety comprises a 2′ modification or an unlocked sugar modification.

15. The compound of claim 14, or a pharmaceutically acceptable salt thereof, wherein the 2′-modification is selected from 2′-fluoro modification, 2′-O-methyl modification, a 2′-O-methoxyethyl, and a bicyclic sugar modification.

16. The compound of claim 15, or a pharmaceutically acceptable salt thereof, wherein the bicyclic sugar modification is selected from a 4′-CH(CH₃)—O-2′ linkage, a 4′-(CH₂)₂—O-2′ linkage, a 4′-CH(CH₂—OMe)-O-2′ linkage, 4′-CH₂—N(CH₃)—O-2′ linkage, and 4′-CH₂—N(H)—O-2′ linkage.

17. The compound of claim 13, or a pharmaceutically acceptable salt thereof, wherein the modified sugar moiety is a morpholino moiety.

18. (canceled)

19. The compound of claim 12, or a pharmaceutically acceptable salt thereof, wherein the modified internucleotide linkage selected from a phosphorothioate linkage and a phosphorodiamidite linkage.

20-21. (canceled)

22. The compound of claim 9, or a pharmaceutically acceptable salt thereof, wherein the nucleic acid is a double-stranded nucleic acid comprising an antisense strand hybridized to a sense strand, and wherein each of the antisense strand and sense strand is independently 15 to 30 nucleotides in length.

23-28. (canceled)

29. The compound of claim 9, or a pharmaceutically acceptable salt thereof, wherein one L⁶ is attached to a 3′ carbon of a 3′ terminal nucleotide of the double-stranded nucleic acid or single-stranded nucleic acid, or to a 5′ carbon of a 5′ terminal nucleotide of the double-stranded nucleic acid or single-stranded nucleic acid.

30-32. (canceled)

33. The compound of claim 9, or a pharmaceutically acceptable salt thereof, wherein one L⁶ is attached to a 2′ carbon of the double-stranded nucleic acid or single-stranded nucleic acid.

34. The compound of claim 9, or a pharmaceutically acceptable salt thereof, wherein one L⁶ is attached to an oxygen of 2′ hydroxy of the double-stranded nucleic acid or single-stranded nucleic acid.

35. The compound of claim 9, or a pharmaceutically acceptable salt thereof, wherein the double-stranded nucleic acid or single-stranded nucleic acid comprises a morpholino moiety, and one L⁶ is attached to a 3′ nitrogen of morpholino moiety.

36. The compound of claim 9, or a pharmaceutically acceptable salt thereof, wherein the double-stranded nucleic acid or single-stranded nucleic acid comprises a morpholino moiety, and one L⁶ is attached to a 6′ carbon of the morpholino moiety.

37. The compound of claim 9, or a pharmaceutically acceptable salt thereof, wherein one L⁶ is attached to a nucleobase of the double-stranded nucleic acid or single-stranded nucleic acid.

38. (canceled)

39. The compound of claim 3, or a pharmaceutically acceptable salt thereof, wherein L⁴ and L⁶ are independently a bond, —NH—, —O—, —S—, —C(O)—, —NHC(O)—, —NHC(O)NH—, —C(O)O—, —OC(O)—, —C(O)NH, —OPO₂—O—, —OP(S)(O)—O—, —OP(S)₂—O—, —S(O)₂NH—, substituted or unsubstituted C₁-C₈ alkylene or substituted or unsubstituted 2 to 8 membered heteroalkylene.

40-41. (canceled)

42. The compound of claim 3, or a pharmaceutically acceptable salt thereof, wherein L⁶ is independently 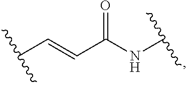 [SMILES: */C=C/C(=O)N*]
—OPO₂—O—, or —O—.

43-45. (canceled)

46. The compound of claim 3, or a pharmaceutically acceptable salt thereof, wherein L⁴ is independently -L¹⁴-NH—C(O)— or -L¹⁴-C(O)—NH—, wherein L¹⁴ is independently substituted or unsubstituted C₁-C₂₀ alkylene, or substituted or unsubstituted 2-20 membered heteroalkenylene.

47. (canceled)

48. The compound of claim 3, or a pharmaceutically acceptable salt thereof, wherein L⁴ is independently

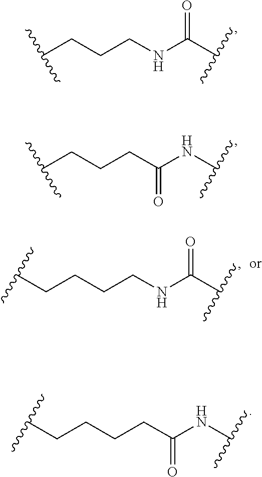 [SMILES: *CCCNC(*)=O ; *CCCC(=O)N* ; *CCCCNC(*)=O ; *CCCCC(=O)N*]

49. The compound of claim 3, or a pharmaceutically acceptable salt thereof, wherein L⁵ is a bond, substituted or unsubstituted C₁-C₈alkylene, substituted or unsubstituted 2 to 8 membered heteroalkylene, substituted or unsubstituted C₃-C₆ cycloalkylene, substituted or unsubstituted 4 to 6 membered heterocycloalkylene, substituted or unsubstituted phenylene, or substituted or unsubstituted 4 to 6 heteroarylene.

50-52. (canceled)

53. The compound of claim 3, or a pharmaceutically acceptable salt thereof, wherein L⁷ is independently a bond, substituted or unsubstituted C₁-C₈ alkylene, or substituted or unsubstituted 2 to 8 membered heteroalkylene.

54. (canceled)

55. The compound of claim 3, or a pharmaceutically acceptable salt thereof, wherein L⁷ is independently -L¹⁷-NH—C(O)— or -L¹⁷-C(O)—NH—, wherein L¹⁷ is independently substituted or unsubstituted C₁-C₂₀ alkylene, substituted or unsubstituted 2-20 membered heteroalkylene, or substituted or unsubstituted 2-20 membered heteroalkenylene.

56-57. (canceled)

58. The compound of claim 3, or a pharmaceutically acceptable salt thereof, wherein L⁷ is independently —NHC(O)—,

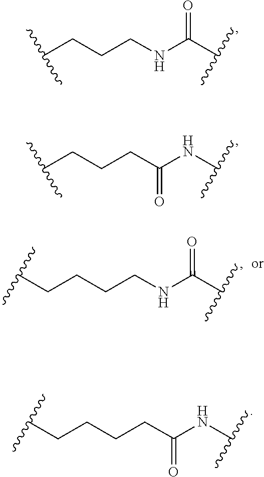 [SMILES: *CCCNC(*)=O ; *CCCC(=O)N* ; *CCCCNC(*)=O ; *CCCCC(=O)N*]

59. The compound of claim 3, or a pharmaceutically acceptable salt thereof, wherein-L⁶-L⁵-L⁴- is independently a bond, or substituted or unsubstituted 2 to 50 membered heteroalkylene.

60-62. (canceled)

63. The compound of claim 3, or a pharmaceutically acceptable salt thereof, wherein-L⁶-L⁵-L⁴- is independently 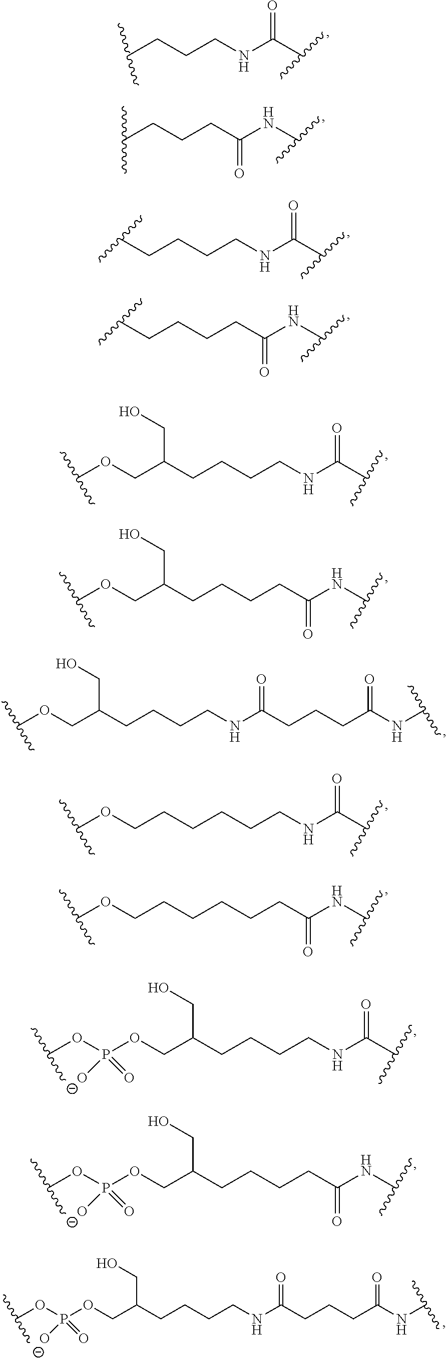 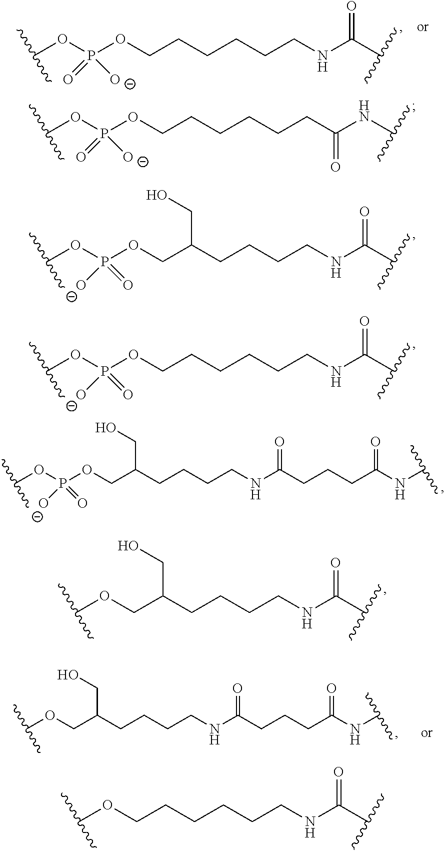
and is attached to a 3′ carbon of the double-stranded nucleic acid or single-stranded nucleic acid; 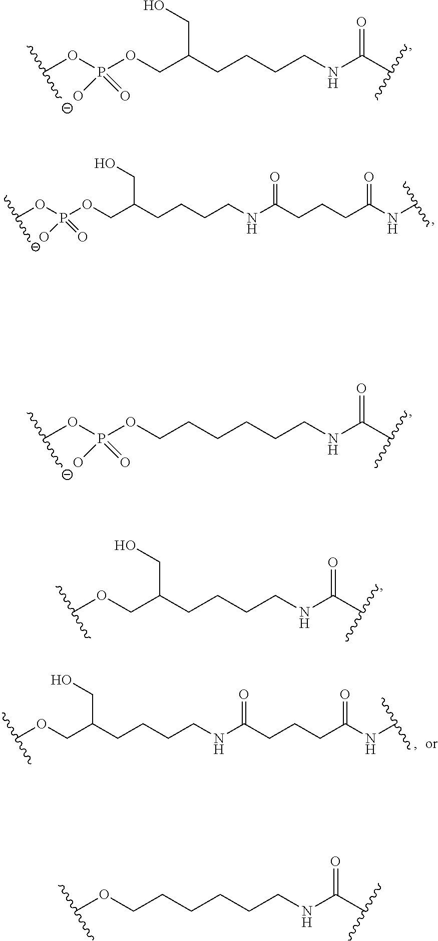 [SMILES: *OP(=O)([O-])OCC(CO)CCCCNC(*)=O ; *NC(=O)CCCC(=O)NCCCCC(CO)COP(=O)([O-])O* ; *OP(=O)([O-])OCCCCCCNC(*)=O ; *OCC(CO)CCCCNC(*)=O ; *NC(=O)CCCC(=O)NCCCCC(CO)CO* ; *OCCCCCCNC(*)=O]
and is attached to a 5′ carbon of the double-stranded nucleic acid or single-stranded nucleic acid;
or 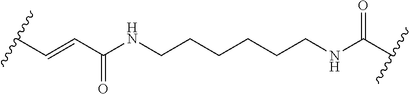 [SMILES: */C=C/C(=O)NCCCCCCNC(*)=O]
and is attached to a nucleotide base of the nucleic acid.

64-66. (canceled)

67. The compound of claim 3, or a pharmaceutically acceptable salt thereof, wherein-L⁶-L⁵-L⁴- is independently —OPO₂—O-L¹⁰-NH—C(O)— or —OPO₂—O-L¹⁰-C(O)—NH—, wherein L¹⁰ is independently substituted or unsubstituted C₁-C₂₀ alkylene, or substituted or unsubstituted 2-20 membered heteroalkenylene.

68-77. (canceled)

78. The compound of claim 3, or a pharmaceutically acceptable salt thereof, wherein
L^{1A} is independently a bond, —NHC(O)—, —C(O)NH—, substituted or unsubstituted C₁-C₈ alkylene, or substituted or unsubstituted 2 to 8 membered heteroalkylene;
L^{1B} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene;
L^{1C} is independently a bond, —O—, —NHC(O)—, —C(O)NH—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene;
L^{1D} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene; and
L^{1E} is independently a bond, —NHC(O)—, —C(O)NH—, substituted or unsubstituted C₁-C₈ alkylene, or substituted or unsubstituted 2 to 8 membered heteroalkylene.

79. The compound of claim 3, or a pharmaceutically acceptable salt thereof, wherein
L^{1A} is independently unsubstituted C₁-C₈ alkylene, or unsubstituted 2 to 8 membered heteroalkylene;
L^{1B} is independently a bond, —O—, —NHC(O)—, —C(O)NH—, unsubstituted C₁-C₈ alkylene, unsubstituted C₂-C₈ alkynylene, unsubstituted 2 to 8 membered heteroalkylene, or unsubstituted phenylene;
L^{1C} is independently a bond, —O—, —NHC(O)—, —C(O)NH—, unsubstituted C₁-C₈ alkylene, unsubstituted 2 to 8 membered heteroalkylene, or unsubstituted phenylene;
L^{1D} is independently a bond, —O—, —NHC(O)—, —C(O)NH—, unsubstituted C₁-C₈ alkylene, or unsubstituted 2 to 8 membered heteroalkylene; and
L^{1E} is independently —NHC(O)—.

80-81. (canceled)

82. The compound of claim 3, or a pharmaceutically acceptable salt thereof, wherein L¹ is independently a bond,

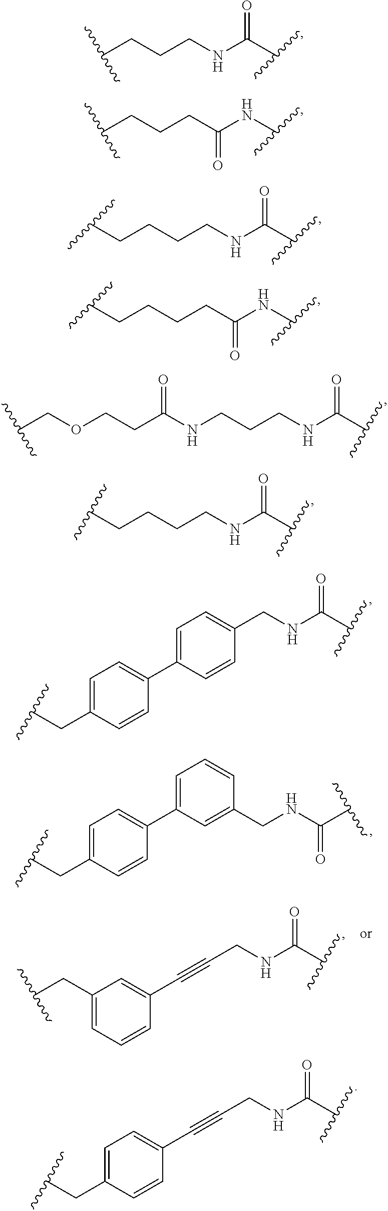

83-87. (canceled)

88. The compound of claim 3, or a pharmaceutically acceptable salt thereof, wherein
L^{2A} is independently a bond, —NHC(O)—, —C(O)NH—, substituted or unsubstituted C₁-C₈ alkylene, or substituted or unsubstituted 2 to 8 membered heteroalkylene;
L^{2B} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene;
L^{2C} is independently a bond, —O—, —NHC(O)—, —C(O)NH—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene;
L^{2D} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene; and
L^{2E} is independently a bond, —NHC(O)—, —C(O)NH—, substituted or unsubstituted C₁-C₈ alkylene, or substituted or unsubstituted 2 to 8 membered heteroalkylene.

89. The compound of claim 3, or a pharmaceutically acceptable salt thereof, wherein
L^{2A} is independently unsubstituted C₁-C₈ alkylene, or unsubstituted 2 to 8 membered heteroalkylene;
L^{2B} is independently a bond, —O—, —NHC(O)—, —C(O)NH—, unsubstituted C₁-C₈ alkylene, unsubstituted C₂-C₈ alkynylene, unsubstituted 2 to 8 membered heteroalkylene, or unsubstituted phenylene;
L^{2C} is independently a bond, —O—, —NHC(O)—, —C(O)NH—, unsubstituted C₁-C₈ alkylene, unsubstituted 2 to 8 membered heteroalkylene, or unsubstituted phenylene;
L^{2D} is independently a bond, —O—, —NHC(O)—, —C(O)NH—, unsubstituted C₁-C₈ alkylene, or unsubstituted 2 to 8 membered heteroalkylene; and
L^{2E} is independently —NHC(O)—.

90-91. (canceled)

92. The compound of claim 3, or a pharmaceutically acceptable salt thereof, wherein L² is independently a bond,

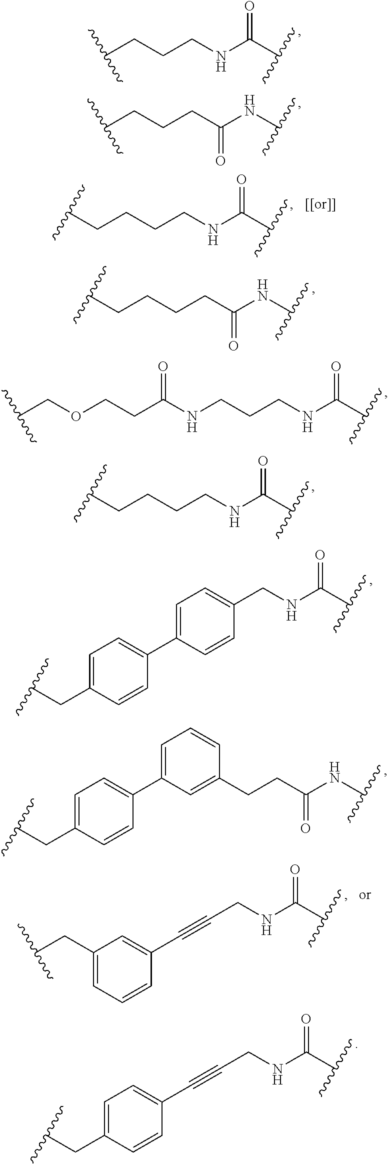

93-97. (canceled)

98. The compound of claim 3, or a pharmaceutically acceptable salt thereof, wherein
L^{3A} is independently a bond, —NHC(O)—, —C(O)NH—, substituted or unsubstituted C₁-C₈ alkylene, or substituted or unsubstituted 2 to 8 membered heteroalkylene;
L^{3B} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene;
L^{3C} is independently a bond, —O—, —NHC(O)—, —C(O)NH—, substituted or unsubstituted C₁-C₈ alkylene, substituted or unsubstituted C₂-C₈ alkynylene, substituted or substituted or unsubstituted 2 to 8 membered heteroalkylene, or substituted or unsubstituted phenylene;
L^{3D} is independently a bond, —O—, —NHC(O)—, —C(O)NH—, substituted or unsubstituted C₁-C₈ alkylene, substituted or unsubstituted 2 to 8 membered heteroalkylene, or substituted or unsubstituted phenylene; and
L^{3E} is independently a bond, —NHC(O)—, —C(O)NH—, substituted or unsubstituted C₁-C₈ alkylene, or substituted or unsubstituted 2 to 8 membered heteroalkylene.

99. The compound of claim 3, or a pharmaceutically acceptable salt thereof, wherein
L^{3A} is independently unsubstituted C₁-C₈alkylene, or unsubstituted 2 to 8 membered heteroalkylene;
L^{3B} is independently a bond, —O—, —NHC(O)—, —C(O)NH—, unsubstituted C₁-C₈ alkylene, unsubstituted C₂-C₈ alkynylene, unsubstituted 2 to 8 membered heteroalkylene, or unsubstituted phenylene;
L^{3C} is independently a bond, —O—, —NHC(O)—, —C(O)NH—, unsubstituted C₁-C₈ alkylene, unsubstituted 2 to 8 membered heteroalkylene, or unsubstituted phenylene;
L^{3D} is independently a bond, —O—, —NHC(O)—, —C(O)NH—, unsubstituted C₁-C₈ alkylene, or unsubstituted 2 to 8 membered heteroalkylene; and
L^{3E} is independently —NHC(O)—.

100-101. (canceled)

102. The compound of claim 3, or a pharmaceutically acceptable salt thereof, wherein L³ is independently a bond,

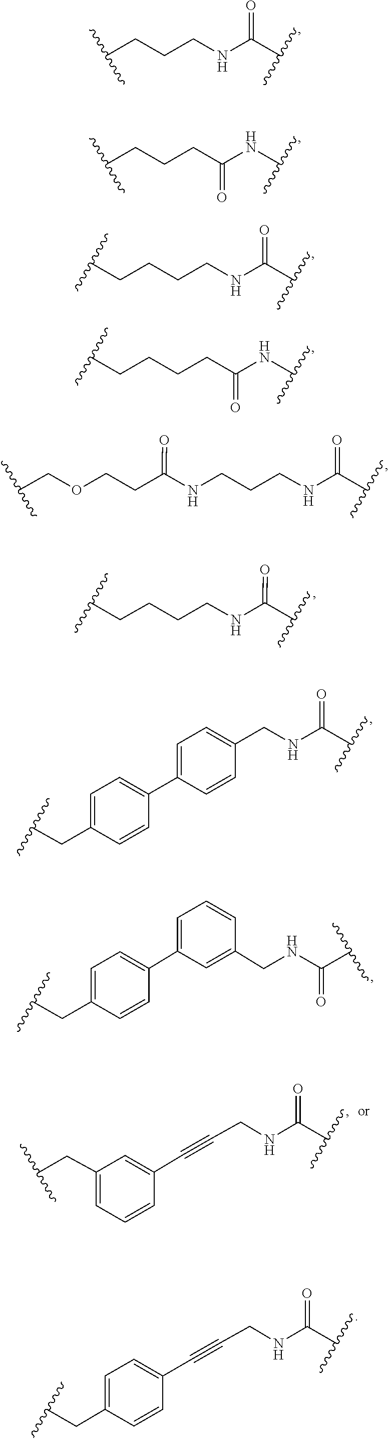

103-105. (canceled)

106. The compound of claim 1, or a pharmaceutically acceptable salt thereof, wherein each of R¹, R², and R³ is independently unsubstituted C₁₁-C₂₅ alkyl.

107-109. (canceled)

110. The compound of claim 1, or a pharmaceutically acceptable salt thereof, wherein each of R¹, R², and R³ is independently unsubstituted unbranched C₁₁-C₂₅ alkyl.

111-113. (canceled)

114. The compound of claim 1, or a pharmaceutically acceptable salt thereof, wherein each of R¹, R², and R³ is independently unsubstituted unbranched saturated C₁₁-C₂₅ alkyl.

115-140. (canceled)

141. The compound of claim 3, or a pharmaceutically acceptable salt thereof, wherein the compound comprises a structure of 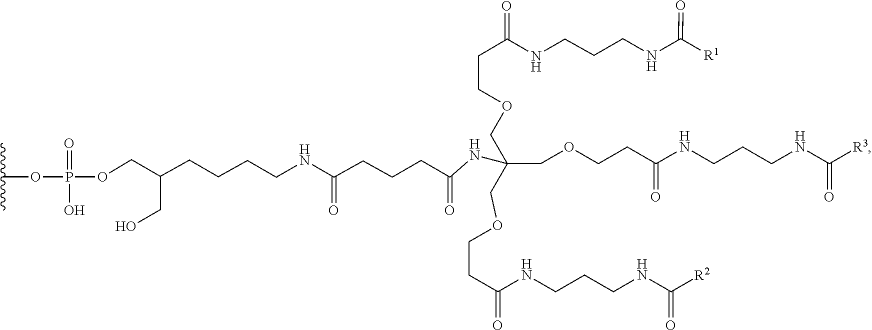
wherein the wavy line represents attachment point to the nucleic acid.

142. A method comprising contacting a cell with a compound of claim 1 or a pharmaceutically acceptable salt thereof any one of claims 1 to 141.

143-145. (canceled)

146. A method comprising administering to a subject a compound of claim 1 or a pharmaceutically acceptable salt thereof, the subject having a disease of the eye, liver, kidney, heart, adipose tissue, lung, muscle or spleen.

147-149. (canceled)

150. A method of introducing a nucleic acid into a cell within a subject, the method comprising administering to said subject the compound of claim 1 or a pharmaceutically acceptable salt thereof.

151. A cell comprising the compound of claim 1 or a pharmaceutically acceptable salt thereof.

152. A pharmaceutical composition comprising a pharmaceutically acceptable excipient and the compound of claim 1 or a pharmaceutically acceptable salt thereof.
